(19) United States
(12) Patent Application Publication
(10) Pub. No.: US 2014/0288024 A1
(43) Pub. Date: Sep. 25, 2014

(54) CYCLODEXTRIN-BASED POLYMERS FOR THERAPEUTICS DELIVERY

(71) Applicant: John Ryan, Philadelphia, PA (US)

(72) Inventor: John Ryan, Philadelphia, PA (US)

(73) Assignee: CERULEAN PHARMA INC., Cambridge, MA (US)

(21) Appl. No.: 14/063,165

(22) Filed: Oct. 25, 2013

Related U.S. Application Data
(63) Continuation of application No. 13/208,848, filed on Aug. 12, 2011, now abandoned.
Application No. 13/208,848 is a continuation-in-part of application No. 12/878,688, filed on Sep. 9, 2010, now abandoned.
(60) Provisional application No. 61/254,011, filed on Oct. 22, 2009.
(60) Provisional application No. 61/241,021, filed on Sep. 9, 2009.

(51) Int. Cl. A61K 47/48; A61K 31/428; A61K 31/427

(52) U.S. Cl. CPC A61K 47/4823; A61K 31/427; A61K 31/428
USPC 514/58; 536/46

(57) ABSTRACT

Methods and compositions relating to CDP-epothilone conjugates are described herein.

### CLAIM OF PRIORITY

This application is a continuation and claims priority to U.S. Ser. No. 13/208,848, filed Aug. 12, 2011, which is a continuation-in-part and claims priority to U.S. Ser. No. 12/878,688, filed Sep. 9, 2010, which claims priority to U.S. Ser. No. 61/241,021, filed on Sep. 9, 2009, and U.S. Ser. No. 61/254,011, filed on Oct. 22, 2009, the contents of each of which are incorporated herein by reference.

### BACKGROUND OF THE INVENTION

Drug delivery of some small molecule therapeutic agents, such as epothilones, has been problematic due to their poor pharmacological profiles. These therapeutic agents often have low aqueous solubility, their bioactive forms exist in equilibrium with an inactive form, or high systemic concentrations of the therapeutic agents lead to toxic side-effects. Some approaches to circumvent the problem of delivery of these agents have been to conjugate the agent directly to a water-soluble polymer such as hydroxypropyl methacrylate (HPMA), polyethyleneglycol, and poly-L-glutamic acid. In some cases, such conjugates have been successful in solubilizing or stabilizing the bioactive form of the therapeutic agent, or achieving a sustained release formulation which circumvents complications associated with high systemic concentrations of the agent. Another approach to the drug delivery problem has been to form host/guest inclusion complexes between the therapeutic agent and cyclodextrins or derivatives thereof. Cyclodextrins (alpha, beta, and gamma) and their oxidized forms have unique physico-chemical properties such as good water solubility, low toxicity and low immune response. To date, most of the drug delivery studies with cyclodextrins have focused on their ability to form supra-molecular complexes, wherein cyclodextrins form host/guest inclusion complexes with therapeutic molecules and thus alter the physical, chemical, and/or biological properties of these guest molecules.

### SUMMARY OF THE INVENTION

In one aspect, the disclosure features a CDP-epothilone conjugate described herein, and methods of making the CDP-epothilone conjugates described herein. Other aspects of the invention include compositions comprising a CDP-epothilone conjugate described herein and nanoparticles comprising a CDP-epothilone conjugates described herein.

In one embodiment, CDP is not biodegradable.

In one embodiment, CDP is biocompatible.

In one embodiment, the CDP-epothilone conjugate includes an inclusion complex between an epothilone attached or conjugated to the polymer, e.g., via a covalent linkage, and another moiety in the polymer, e.g., a cyclic moiety, such as a cyclodextrin.

In one embodiment, the CDP-epothilone conjugate forms a nanoparticle or is a component of a nanoparticle. In one embodiment, the CDP-epothilone conjugate including an inclusion complex forms a nanoparticle or is a component of a nanoparticle. In general, the nanoparticle ranges in size from 10 to 300 nm in diameter, e.g., 20 to 280, 30 to 250, 30 to 200, 20 to 150, 30 to 100, 20 to 80, 30 to 70, 30 to 60 or 30 to 50 nm diameter. In one embodiment, the nanoparticle is 30 to 60 nm in diameter. In one embodiment, the nanoparticles described herein are a component of a composition such as a pharmaceutical composition. Exemplary nanoparticle containing compositions include compositions having a population or a plurality of nanoparticles with an average diameter from 10 to 300 nm, e.g., 20 to 280, 30 to 250, to 200, 20 to 150, 30 to 100, 20 to 80, 30 to 70, 30 to 60 or 30 to 50 nm. In one embodiment, the average nanoparticle diameter is from 30 to 60 nm. In one embodiment, the surface charge of the nanoparticle is neutral, or slightly negative. In some embodiments, the zeta potential of the particle surface (e.g., nanoparticle surface) is from about −80 mV to about 50 mV, about −20 mV to about 20 mV, about −20 mV to about −10 mV, or about −10 mV to about 0.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one embodiment, the cyclodextrin is beta-cyclodextrin.

In certain embodiments, the polymer is linear or branched. Typically, the polymer is linear.

In one embodiment, the therapeutic agent, e.g., an epothilone, conjugated to the polymer is more soluble when conjugated to the polymer, than when not conjugated to the polymer. For example, the epothilone is more soluble in aqueous media when conjugated to a CDP than when not conjugated to a CDP.

Also described herein are compositions comprising a plurality of CDP-epothilone conjugates (e.g., a reaction mixture comprising a plurality of CDP-epothilone conjugates or a pharmaceutical composition comprising a plurality of CDP-epothilone conjugates). In one embodiment, the composition comprises a population, mixture or plurality of CDP-epothilone conjugates. In one embodiment, the population, mixture or plurality of CDP-epothilone conjugates comprises a plurality of different epothilones conjugated to a CDP (e.g., two different epothilones are in the composition such that two different epothilones are attached to a single CDP; or a first epothilone is attached to a first CDP and a second epothilone is attached to a second CDP and both CDP-epothilone conjugates are present in the composition). In one embodiment, the population, mixture or plurality of CDP-epothilone conjugates comprises a CDP having a single epothilone attached thereto in a plurality of positions (e.g., a CDP has a single epothilone attached thereto such that the single epothilone for some occurrences is attached through a first position (e.g., a 3-OH) and for other occurrences is attached through a second position (e.g., a 7-OH) to thereby provide a CDP having single epothilone attached through a plurality of positions on the epothilone). In one embodiment, the population, mixture or plurality of CDP-epothilones comprises a first CDP attached to an epothilone through a first position (e.g., a 3-OH) and a second CDP attached to the same epothilone through a second position (e.g., a 7-OH) and both CDP-epothilone conjugates are present in the composition. In one embodiment, the composition comprising the CDP-epothilone conjugates comprises a single epothilone conjugated to the CDP in a plurality of positions on the CDP (e.g., through the same or different positions of the epothilone).

In one aspect, the disclosure features a method of treating a proliferative disorder, e.g., a cancer, in a subject, e.g., a human, the method comprises: administering a composition that comprises a CDP-epothilone conjugate, e.g., a CDP-epothilone conjugate described herein, to a subject in an amount effective to treat the disorder, to thereby treat the proliferative disorder. In an embodiment, the CDP-epothilone conjugate comprises epothilone molecules, coupled, e.g., directly or via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is administered in combination with one or more additional chemotherapeutic agents, e.g., a chemotherapeutic agent or combination of chemotherapeutic agents described herein. For example, the composition can be administered in combination with an anti-metabolite such as capecitabine.

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney, liver, lung (including small and non-small cell lung cancer, lung adenocarcinoma and squamous cell cancer), genitourinary tract, e.g., ovary (including fallopian tube and peritoneal cancers), cervix, prostate and testes, lymphatic system, rectum, larynx, pancreas (including exocrine pancreatic carcinoma), esophagus, stomach, gall bladder, thyroid, skin (including squamous cell carcinoma), brain (including glioblastoma multiforme), and head and neck. Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., advanced non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma, gliomas, melanoma (e.g., advanced or metastatic melanoma), germ cell tumors, ovarian cancer (e.g., advanced ovarian cancer, e.g., advanced fallopian tube or peritoneal cancer) and gastrointestinal cancer.

In one embodiment, the composition is administered by intravenous administration, e.g., an intravenous administration that is completed in a period equal to or less than 2 hours, 1.5 hours, 1 hour, 45 minutes or 30 minutes. In one embodiment, the composition is administered as a bolus infusion or intravenous push, e.g., over a period of 15 minutes, 10 minutes, 5 minutes or less.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1, and e.g., the composition is administered to the subject in an amount of the composition that includes 40 mg/m² or greater (e.g., 45 mg/m², 48 mg/m², 50 mg/m², 60 mg/m², 70 mg/m², 80 mg/m², 85 mg/m², or 90 mg/m²), of an epothilone, e.g., ixabepilone, to thereby treat the disorder. In one embodiment, the composition is administered by intravenous administration over a period of about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the composition such that the composition includes 40 mg/m² or greater (e.g., 45 mg/m², 48 mg/m², 50 mg/m², 60 mg/m², 70 mg/m², 80 mg/m², 85 mg/m², or 90 mg/m²) of an epothilone, e.g., ixabepilone. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1, and the composition is administered to the subject in an amount of the composition that includes 40 mg/m² or greater (e.g., 45 mg/m², 48 mg/m², 50 mg/m², 60 mg/m², 70 mg/m², 80 mg/m², 85 mg/m², or 90 mg/m²), of ixabepilone, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1, and at least two, three, four, five, six, seven or eight doses are administered to the subject and each dose is an amount of the composition that includes 40 mg/m² or greater (e.g., 45 mg/m², 48 mg/m², 50 mg/m², 60 mg/m², 70 mg/m², 80 mg/m², 85 mg/m², or 90 mg/m²) of ixabepilone, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period of about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the composition includes a CDP-epothilone B conjugate, e.g., a CDP-epothilone B conjugate described herein and, e.g., a CDP-epothilone B conjugate comprising epothilone B molecules, coupled, e.g., via linkers, to a CDP moiety referred to in FIG. 1, e.g., the composition is administered in an amount of the composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², or 25 mg/m²) of epothilone B, to thereby treat the disorder. In one embodiment, the composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², or 25 mg/m²) of the epothilone, e.g. epothilone B. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1, and the composition is administered to the subject in an amount of the composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², or 25 mg/m²) of epothilone B, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-epothilone B conjugate, e.g., a CDP-epothilone B conjugate described herein, a e.g., a CDP-epothilone B conjugate comprising epothilone B molecules, coupled, e.g., via linkers, to a CDP moiety referred to in FIG. 1, and at least two, three, four, five, six, seven or eight doses are administered to the subject and each dose is an amount of the composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², or 25 mg/m²) of epothilone B, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1, and, e.g., the composition is administered in an amount of the composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², or 280 mg/m²) of the epothilone, e.g., epothilone D, to thereby treat the disorder. In one embodiment, the composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, an additional dose (or additional doses) is administered in an amount of the composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², or 280 mg/m²) of epothilone D. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1, and the composition is administered to the subject in an amount of the composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², or 280 mg/m²) of epothilone D, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1, and at least two, three, four, five, six, seven or eight doses are administered to the subject and each dose is an amount of the composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², or 280 mg/m²) of epothilone D, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1, and, e.g., the composition is administered in an amount of the composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², or 105 mg/m²) of the epothilone, e.g., BMS310705, to thereby treat the disorder. In one embodiment, the composition is administered by intravenous administration over a period equal to or less than about 30 minutes, 45 minutes, 60 minutes, 90 minutes, 120 minutes, 150 minutes or 180 minutes.

In one embodiment, the subject is administered at least one additional dose of the composition, e.g., the subject is administered at least two, three, four, five, six, seven or eight additional doses of the composition. In one embodiment, the dosing schedule is not changed between doses. For example, when the dosing schedule is once every three weeks, an additional dose (or doses) is administered in three weeks. In one embodiment, the composition is administered once every one, two, three, four, five or six weeks. In one embodiment, when at least one additional dose is administered, the additional dose (or additional doses) is administered in an amount of the composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 m g/m², or 105 mg/m²) of the epothilone, e.g., BMS310705. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1, and the composition is administered to the subject in an amount of the composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², or 105 mg/m²) of BMS310705, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1, and at least two, three, four, five, six, seven or eight doses are administered to the subject and each dose is an amount of the composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², or 105 mg/m²) of BMS310705, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1, and, e.g., the composition is administered in an amount of the composition that includes 0.5 to 35 mg/m² (e.g., 0.8 mg/m², 1 mg/m², 5 mg/m², 10 mg/m², 15 mg/m², 20 mg/m², or 25 mg/m²) of the epothilone, e.g., dehydelone, to thereby treat the disorder. In one embodiment, the composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the composition that includes 0.5 to 35 mg/m² (e.g., 0.8 mg/m², 1 mg/m², 5 mg/m², 10 mg/m², 15 mg/m², 20 mg/m², or 25 mg/m²) of dehydelone. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1, and the composition is administered to the subject in an amount of the composition that includes 0.5 to 35 mg/m² (e.g., 0.8 mg/m², 1 mg/m², 5 mg/m², 10 mg/m², 15 mg/m², 20 mg/m², or 25 mg/m²) of dehydelone, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1, and at least two, three, four, five, six, seven or eight doses are administered to the subject and each dose is an amount of the composition that includes 0.5 to 35 mg/m² (e.g., 0.8 mg/m², 1 mg/m², 5 mg/m², 10 mg/m², 15 mg/m², 20 mg/m², or 25 mg/m²) of dehydelone to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the composition includes a CDP-ZK-EPO conjugate, e.g., a CDP-ZK-EPO conjugate described herein, e.g., a CDP-ZK-EPO conjugate comprising CDP-ZK-EPO molecules, coupled, e.g., via linkers, to a CDP moiety referred to in FIG. 1, and, e.g., the composition is administered in an amount of the composition that includes 1 to 40 mg/m² (e.g., 2 mg/m², 5 mg/m², 10 mg/m², 16 mg/m², 20 mg/m², 25 mg/m², 30 mg/m², or 35 mg/m²) of the epothilone, e.g., ZK-EPO, to thereby treat the disorder. In one embodiment, the composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the composition, e.g., the subject is administered at least two, three, four, five, six, seven or eight additional doses of the composition. In one embodiment, the composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the composition that includes 1 to 40 mg/m² (e.g., 2 mg/m², 5 mg/m², 10 mg/m², 16 mg/m², 20 mg/m², 25 mg/m², 30 mg/m², or 35 mg/m²) of ZK-EPO. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the composition includes a CDP-ZK-EPO conjugate, e.g., a CDP-ZK-EPO conjugate described herein, e.g., a CDP-ZK-EPO conjugate comprising CDP-ZK-EPO molecules, coupled, e.g., via linkers, to a CDP moiety referred to in FIG. 1, and the composition is administered to the subject in an amount of the composition that includes 1 to 40 mg/m² (e.g., 2 mg/m², 5 mg/m², 10 mg/m², 16 mg/m², 20 mg/m², 25 mg/m², 30 mg/m², or 35 mg/m²) ZK-EPO, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the composition once every one, two, three, four, five or six weeks.

In one embodiment, the composition includes a CDP-ZK-EPO conjugate, e.g., a CDP-ZK-EPO conjugate described herein, e.g., a CDP-ZK-EPO conjugate comprising CDP-ZK-EPO molecules, coupled, e.g., via linkers, to a CDP moiety referred to in FIG. 1, and at least two, three, four, five, six, seven or eight doses are administered to the subject and each dose is an amount of the composition that includes 1 to 40 mg/m² (e.g., 2 mg/m², 5 mg/m², 10 mg/m², 16 mg/m², 20 mg/m², 25 mg/m², 30 mg/m², or 35 mg/m²) of ZK-EPO, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the CDP-epothilone conjugate, e.g., a CDP-epothilone conjugate comprising epothilone molecules, coupled, e.g., via linkers, to a CDP moiety referred to in FIG. 1, is administered once every three weeks in combination with one or more additional chemotherapeutic agent(s) that is also administered once every three weeks. In one embodiment, the CDP-epothilone conjugate is administered once every three weeks in combination with one or more of the following chemotherapeutic agents: an antimetabolite (e.g., floxuridine, pemetrexed 5FU); an anthracycline (e.g., daunorubicin, epirubicin, idarubicin, mitoxantrone, or valrubicin); a vinca alkaloid (e.g., vinblastine, vincristine, vindesine or vinorelbine); a topoisomerase inhibitor (e.g., topotecan, irinotecan, etoposide, teniposide, or lamellarin D, camptothecin (e.g., IT-101)); and a platinum-based agent (e.g., cisplatin, carboplatin, or oxaliplatin).

In one embodiment, the CDP-epothilone conjugate, e.g., a CDP-epothilone conjugate comprising epothilone molecules, coupled, e.g., via linkers, to a CDP moiety referred to in FIG. 1, is administered once every two weeks in combination with one or more additional chemotherapeutic agent that is administered orally. In one embodiment, the CDP-epothilone conjugate is administered once every two weeks in combination with one or more of the following chemotherapeutic agents: capecitabine, estramustine, erlotinib, rapamycin, SDZ-RAD, CP-547632; AZD2171, sunitinib, sorafenib or everolimus.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of treating a chemotherapeutic sensitive, a chemotherapeutic refractory, a chemotherapeutic resistant, and/or a relapsed cancer. The method comprises: administering a composition comprising a CDP-epothilone conjugate, e.g., a CDP-epothilone conjugate described herein, to a subject, e.g., a human, in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the cancer is refractory to, resistant to and/or relapsed during or after, treatment with, one or more of: a taxane (e.g., paclitaxel, or docetaxel), an anthracycline (e.g., daunorubicin, epirubicin, idarubicin, mitoxantrone, or valrubicin), an antimetabolite (e.g., an antifolate, a purine analogue, a pyrimidine analogue (e.g., capecitabine)), a vinca alkaloid (e.g., vinblastine, vincristine, vindesine or vinorelbine), a topoisomerase inhibitor (e.g., topotecan, irinotecan, etoposide, teniposide, lamellarin D, or camptothecin (e.g., IT-101)) and a platinum-based agent (e.g., cisplatin, carboplatin, or oxaliplatin). In one embodiment, the cancer is resistant to more than one chemotherapeutic agent, e.g., the cancer is a multidrug resistant cancer. In one embodiment, the cancer is resistant to one or more of a taxane, a platinum based agent and a vinca alkaloid, e.g., a taxane, a platinum based agent and a vinca alkaloid described herein.

In one embodiment, the composition is administered in combination with a second chemotherapeutic agent, e.g., a chemotherapeutic agent described herein. For example, the composition can be administered in combination with an antimetabolite such as capecitabine.

In one embodiment, the cancer is a cancer described herein. For example, the cancer can be carcinoma, including that of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney, liver, lung (including small and non-small cell lung cancer, lung adenocarcinoma and squamous cell cancer), genitourinary tract, e.g., ovary (including fallopian tube and peritoneal cancers) cervical, prostate and testes, lymphatic system, rectum, larynx, pancreas (including exocrine pancreatic carcinoma), esophagus, stomach, gall bladder, cervix, thyroid, and skin (including squamous cell carcinoma), brain (including glioblastoma multiforme), and head and neck. Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma and squamous cell cancer, e.g., advanced non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's or non-Hodgkin's lymphoma), renal cell carcinoma, carcinoma of the urothelium, soft tissue sarcoma, gliomas, melanoma (e.g., advanced or metastatic melanoma), germ cell tumors, ovarian cancer (e.g., advanced ovarian cancer, e.g., advanced fallopian tube or peritoneal cancer), glioblastoma and gastrointestinal cancer.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1.

In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising CDP-ZK-EPO molecules, coupled, e.g., via linkers, to a CDP moiety referred to in FIG. 1. In one embodiment, the CDP-ZK-EPO conjugate is administered at a dose and/or dosing schedule described herein.

In one aspect, the disclosure features a method of treating metastatic or locally advanced breast cancer in a subject, e.g., a human. The method comprises: administering a composition comprising a CDP-epothilone conjugate, e.g., a CDP-epothilone conjugate described herein, to a subject in an amount effective to treat the cancer, to thereby treat the cancer. In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the breast cancer is estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer) or inflammatory breast cancer.

In one embodiment, the composition is not administered in combination with a taxane.

In one embodiment, the composition is administered in combination with a HER-2 pathway inhibitor, e.g., a HER-2 inhibitor or a HER-2 receptor inhibitor. For example, the composition is administered with trastuzumab.

In some embodiments, the composition is administered in combination with a second chemotherapeutic agent. For example, the composition is administered in combination with a vascular endothelial growth factor (VEGF) pathway inhibitor, e.g., a VEGF inhibitor (e.g., bevacizumab) or VEGF receptor inhibitor (e.g., CP-547632 or AZD2171). In one embodiment, the composition is administered in combination with bevacizumab.

In some embodiments, the composition is administered in combination with an anthracycline (e.g., daunorubicin, doxorubicin, epirubicin, valrubicin or idarubicin).

In some embodiments, the composition is administered in combination with an anti-metabolite, e.g., an antifolate (e.g., floxuridine, or pemetrexed) or pyrimidine analogue (e.g., 5FU)).

In some embodiments, the composition is administered in combination with an anthracycline (e.g., daunorubicin, doxorubicin, epirubicin, valrubicin and idarubicin) and an anti-metabolite (e.g., floxuridine, pemetrexed, or 5FU).

In some embodiments, the composition is administered in combination with a platinum-based agent (e.g., cisplatin, carboplatin, or oxaliplatin).

In some embodiments, the composition is administered in combination with an mTOR inhibitor. Non-limiting examples of mTOR inhibitors include rapamycin, everolimus, AP23573, CCI-779 and SDZ-RAD.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises ixabepilone, coupled via a linker shown in FIG. 2 to a CDP moiety, e.g., a CDP moiety referred to in FIG. 1. In an embodiment, the CDP-ixabepilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone B conjugate is a CDP-epothilone conjugate shown in FIG. 2.

In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone D conjugate is a CDP-epothilone conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising CDP-ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating metastatic or locally advanced breast cancer, e.g. a breast cancer described herein, in a subject, e.g., a human. The method comprises:

providing a subject that has metastatic or locally advanced breast cancer and has been treated with a chemotherapeutic agent which did not effectively treat the cancer (e.g., the subject has a chemotherapeutic refractory, a chemotherapeutic resistant and/or a relapsed cancer) or which had an unacceptable side effect (e.g., the subject has a chemotherapeutic sensitive cancer), and

administering a composition comprising a CDP-epothilone conjugate, e.g., a CDP-epothilone conjugate described herein, to the subject in an amount effective to treat the cancer, to thereby treat the cancer. In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the cancer is refractory to, resistant to, and/or relapsed with treatment with one or more of: a taxane, an anthracycline, pyrimidine analog, a vinca alkaloid (e.g., vinblastine, vincristine, vindesine or vinorelbine) and a platinum-based agent (e.g., cisplatin, carboplatin, or oxaliplatin).

In one embodiment, the cancer is a multidrug resistant cancer.

In one embodiment, the composition is administered in combination with a pyrimidine analogue, e.g., a pyrimidine analogue described herein (e.g., capecitabine).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising -dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating hormone refractory prostate cancer in a subject, e.g., a human. The method comprises: administering a composition comprising a CDP-epothilone conjugate, e.g., a CDP-epothilone conjugate described herein, to a subject in an amount effective to treat the cancer, to thereby treat the cancer. In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is not administered in combination with a taxane.

In one embodiment, the composition is administered in combination with prednisone.

In one embodiment, the composition is administered in combination with estramustine.

In one embodiment, the composition is administered in combination with an anthracenedione (e.g., mitoxantrone) and prednisone.

In one embodiment, the composition is administered in combination with a vascular endothelial growth factor (VEGF) pathway inhibitor, e.g., a VEGF inhibitor (e.g., bevacizumab) or VEGF receptor inhibitor (e.g., CP-547632 or AZD2171).

In one embodiment, the composition is administered in combination with an mTOR inhibitor. Non-limiting examples of mTOR inhibitors include rapamycin, everolimus, AP23573, CCI-779, and SDZ-RAD.

In one embodiment, the composition is administered in combination with a platinum-based agent (e.g., cisplatin, carboplatin, or oxaliplatin).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating hormone refractory prostate cancer in a subject, e.g., a human. The method comprises:

providing a subject who has hormone refractory prostate cancer and has been treated with a chemotherapeutic agent that did not effectively treat the cancer (e.g., the subject has a chemotherapeutic refractory, chemotherapeutic resistant and/or relapsed cancer) or who had unacceptable side effect (e.g., the subject has a chemotherapeutic sensitive cancer), and

administering a composition comprising a CDP-epothilone conjugate, e.g., a CDP-epothilone conjugate described herein, to the subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject has been treated with a taxane which did not effectively treat the cancer (e.g., the subject has a taxane refractory, a taxane resistant and/or a relapsed cancer).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone B conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating renal cell carcinoma in a subject, e.g., a human. The method comprises:

administering a composition comprising a CDP-epothilone conjugate, e.g., a CDP-epothilone conjugate described herein, to a subject in an amount effective to treat the carcinoma, to thereby treat the carcinoma.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is administered in combination with an mTOR inhibitor. Exemplary mTOR inhibitors include rapamycin, everolimus, AP23573, CCI-779 and SDZ-RAD.

In one embodiment, the composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632, AZD2171, sorafinib and sunitinib.

In one embodiment, the composition is administered in combination with interleukin-2.

In one embodiment, the composition is administered in combination with interferon.

In one embodiment, the composition is administered in combination with a pyrimidine analogue, e.g., capecitabine.

In one embodiment, the composition is administered in combination with an anti-metabolite, e.g., an antifolate, e.g., floxuridine or pyrimidine analogue, e.g., 5FU, and/or a nucleoside analog, e.g., gemcitabine.

In one embodiment, the composition is administered in combination with an anthracycline (e.g., daunorubicin, doxorubicin, epirubicin, valrubicin or idarubicin).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. 1. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecules, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating renal cell carcinoma in a subject, e.g., a human, the method comprises:

providing a subject who has renal cell carcinoma and has been treated with a chemotherapeutic agent that did not effectively treat the carcinoma (e.g., the subject has a chemotherapeutic refractory, a chemotherapeutic resistant and/or a relapsed carcinoma) or who had an unacceptable side effect (e.g., the subject has a chemotherapeutic sensitive carcinoma), and

administering a composition comprising a CDP-epothilone conjugate, e.g., a CDP-epothilone conjugate described herein, to the subject in an amount effective to treat the carcinoma, to thereby treat the carcinoma.

In an embodiment, the CDP-epothilone conjugate comprises epothilone molecules, coupled, e.g., via linkers, to a CDP moiety referred to in FIG. 1.

In one embodiment, the subject has been treated with a taxane which did not effectively treat the carcinoma (e.g., the subject has a taxane refractory, a taxane resistant and/or a relapsed carcinoma).

In one embodiment, the subject has been treated with an mTOR inhibitor which did not effectively treat the carcinoma (e.g., the subject has been treated with rapamycin, everolimus, AP23573, CCI-779 or SDZ-RAD which did not effectively treat the carcinoma).

In one embodiment, the subject has been treated with a vascular endothelial growth factor (VEGF) pathway inhibitor (e.g., a VEGF inhibitor or a VEGF receptor inhibitor) which did not effectively treat the carcinoma (e.g., the subject has been treated with bevacizumab, CP-547632, AZD2171, sunitinib or sorafinib which did not effectively treat the carcinoma).

In one embodiment, the subject has been treated with interleukin-2 which did not effectively treat the carcinoma.

In one embodiment, the subject has been treated with a nucleoside analog which did not effectively treat the carcinoma (e.g., the subject has been treated with gemcitabine which did not effectively treat the carcinoma).

In one embodiment, the subject has been treated with an anti-metabolite which did not effectively treat the carcinoma (e.g., the subject has been treated with an antifolate, e.g., floxuridine, pemetrexed, or a pyrimidine analog, e.g., capecitabine or SFU, which did not effectively treat the carcinoma).

In one embodiment, the subject has been treated with an anthracycline which did not effectively treat the carcinoma (e.g., the subject has been treated with daunorubicin, doxorubicin, epirubicin, valrubicin or idarubicin which did not effectively treat the carcinoma).

In one embodiment, the composition is administered in combination with an mTOR inhibitor, e.g., rapamycin, everolimus, AP23573, CCI-779 or SDZ-RAD. In one embodiment, the subject has been treated with a VEGF pathway inhibitor (e.g., a VEGF inhibitor or a VEGF receptor inhibitor) which did not effectively treat the cancer (e.g., the subject has been treated with bevacizumab, CP-547632, AZD2171, sunitinib or sorafinib which did not effectively treat the carcinoma), and the composition is administered to the subject in combination with an mTOR inhibitor, e.g., everolimus.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced non small cell lung cancer or small cell lung cancer in a subject, e.g., a human. The method comprises: administering a composition comprising a CDP-epothilone conjugate, e.g., a CDP-epothilone conjugate described herein, to a subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is not administered in combination with a taxane.

In one embodiment, the composition is administered in combination with a vascular endothelial (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the composition is administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In one embodiment, the composition is administered in combination with a platinum-based agent (e.g., cisplatin, carboplatin, oxaliplatin). In one embodiment, the composition is administered in combination with a platinum-based agent (e.g., cisplatin, carboplatin, oxaliplatin) and a nucleoside analog (e.g., gemcitabine). In one embodiment, the composition is administered in combination with a platinum-based agent (e.g., cisplatin, carboplatin, oxaliplatin) and an anti-metabolite, e.g., an antifolate (e.g., floxuridine, or pemetrexed) or pyrimidine analogue (e.g., 5FU). In one embodiment, the composition is administered in combination with a platinum-based agent (e.g., cisplatin, carboplatin or oxaliplatin) and a vinca alkaloid (e.g., vinblastine, vincristine, vindesine or vinorelbine).

In one embodiment, the composition is administered in combination with an mTOR inhibitor, e.g., rapamycin, everolimus, AP23573, CCI-779 or SDZ-RAD.

In one embodiment, the composition, either alone or with any of the combinations described herein, is administered in combination with radiation.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced non small cell lung cancer or small cell lung cancer in a subject, e.g., a human. The method comprises:

providing a subject who has advanced non small cell lung cancer or small cell lung cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a composition comprising a CDP-epothilone conjugate, e.g., a CDP-epothilone conjugate described herein, to the subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject has been treated with a taxane which did not effectively treat the cancer (e.g., the subject has a taxane refractory, a taxane resistant and/or a relapsed cancer).

In one embodiment, the subject has been treated with a vascular endothelial growth factor (VEGF) pathway inhibitor (e.g., a VEGF inhibitor or VEGF receptor inhibitor) which did not effectively treat the cancer (e.g., the subject has been treated with bevacizumab CP-547632 or AZD2171 which did not effectively treat the cancer).

In one embodiment, the subject has been treated with an endothelial growth factor (EGF) pathway inhibitor (e.g., an EGF inhibitor or an EGF receptor inhibitor) which did not effectively treat the cancer (e.g., the subject has been treated with cetuximab, erlotinib, or gefitinib which did not effectively treat the cancer).

In one embodiment, the subject has been treated with a platinum-based agent, which did not effectively treat the cancer (e.g., the subject has been treated with cisplatin, carboplatin or oxaliplatin which did not effectively treat the cancer).

In one embodiment, the composition is administered in combination with an anti-metabolite, e.g., an antifolate, e.g., floxuridine, pemetrexed or pyrimidine analogue (e.g., 5FU).

In one embodiment, the composition is administered in combination with an EGF pathway inhibitor, e.g., an EGF inhibitor or EGF receptor inhibitor. The EGF receptor inhibitor can be, e.g., cetuximab, erlotinib or gefitinib.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced ovarian cancer (e.g., peritoneal or fallopian tube cancer) in a subject, e.g., a human. The method comprises: administering a composition comprising a CDP-epothilone conjugate, e.g., a CDP-epothilone conjugate described herein, to a subject in an amount effective to treat the cancer, to thereby treat the cancer.

In one embodiment, the composition is not administered in combination with a taxane.

In one embodiment, the composition is administered in combination with a taxane (e.g., paclitaxel or docetaxel).

In one embodiment, the composition is administered in combination with a platinum-based agent (e.g., cisplatin, carboplatin or oxaliplatin).

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is administered in combination with one or more of: an anti-metabolite, e.g., an antifolate (e.g., pemetrexed, floxuridine, or raltitrexed) or pyrimidine analog (e.g., capecitabine, cytrarabine, gemcitabine, or 5FU); an alkylating agent (e.g., cyclophosphamide, dacarbazine, melphalan, ifosfamide, temozolomide; a topoisomerase inhibitor (e.g., etoposide, topotecan, irinotecan, tenoposide, or lamellarin D); a platinum based agent (e.g., carboplatin, cisplatin, or oxaliplatin); a vinca alkaloid (e.g., vinblastine, vincristine, vindesine, or vinorelbine). In one embodiment, the composition is administered in combination with one or more of: capecitabine, cyclophosphamide, etoposide, gemcitabine, ifosfamide, irinotecan, melphalan, oxaliplatin, vinorelbine, vincristine or pemetrexed.

In one embodiment, the composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the composition is administered in combination with an mTOR inhibitor, e.g., rapamycin, everolimus, AP23573, CCI-779 or SDZ-RAD.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced ovarian cancer (e.g., peritoneal or fallopian tube cancer) in a subject, e.g., a human. The method comprises:

providing a subject who has advanced ovarian cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer), and

administering a composition comprising a CDP-epothilone conjugate, e.g., a CDP-epothilone conjugate described herein, to the subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject has been treated with a platinum-based agent that did not effectively treat the cancer (e.g., the subject has been treated with cisplatin, carboplatin or oxaliplatin which did not effectively treat the cancer). In one embodiment, the subject has been treated with cisplatin or carboplatin which did not effectively treat the cancer.

In one embodiment, the subject has been treated with a taxane that did not effectively treat the cancer (e.g., the subject has been treated with paclitaxel or docetaxel which did not effectively treat the cancer). In one embodiment, the subject has been treated with paclitaxel or docetaxel which did not effectively treat the cancer.

In one embodiment, the composition is administered in combination with a nucleoside analog, e.g., gemcitabine.

In one embodiment, the composition is administered in combination with a pyrimidine analog, e.g., capecitabine.

In one embodiment, the composition is administered in combination with a pyrimidine analog, e.g., capecitabine, and a nucleoside analog, e.g., gemcitabine.

In one embodiment, the composition is administered in combination with an anthracycline, e.g., daunorubicin, doxorubicin, epirubicin, valrubicin or idarubicin. In one embodiment, the anthracycline is doxorubicin, e.g., liposomal doxorubicin.

In one embodiment, the composition is administered in combination with a topoisomerase I inhibitor, e.g., irinotecan, topotecan, tenoposide, lamellarin D, or camptothecin (e.g., IT-101). In one embodiment the topoisomerase I inhibitor is topotecan. In another embodiment, the topoisomerase I inhibitor is irinotecan or etoposide.

In one embodiment, the composition is administered in combination with one or more of: an anti-metabolite, e.g., an antifolate (e.g., pemetrexed, floxuridine, or raltitrexed) or pyrimidine analog (e.g., capecitabine, cytrarabine, gemcitabine, or 5FU); an alkylating agent (e.g., cyclophosphamide, dacarbazine, melphalan, ifosfamide, or temozolomide); a platinum based agent (carboplatin, cisplatin, or oxaliplatin); and a vinca alkaloid (e.g., vinblastine, vincristine, vindesine or vinorelbine). In one embodiment, the composition is administered in combination with one or more of: capecitabine, cyclophosphamide, etoposide, gemcitabine, ifosfamide, irinotecan, melphalan, oxaliplatin, vinorelbine, vincristine or pemetrexed.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced or metastatic melanoma in a subject, e.g., a human. The method comprises: administering a composition comprising a CDP-epothilone conjugate, e.g., a CDP-epothilone conjugate described herein, to a subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is not administered in combination with a taxane.

In one embodiment, the composition is administered in combination with a platinum-based agent (e.g., cisplatin, carboplatin, or oxaliplatin). In one embodiment, the composition is administered in combination with a platinum-based agent (e.g., cisplatin, carboplatin, or oxaliplatin) and a tetrazine, e.g., dacarbazine, mitozolomide or temozolomide. In one embodiment, the composition is administered in combination with cisplatin or carboplatin and dacarbazine or temozolomide.

In one embodiment, the composition is administered in combination with a tetrazine, e.g., dacarbazine, mitozolomide or temozolomide. In one embodiment, the tetrazine is dacarbazine or temozolomide.

In one embodiment, the composition is administered with interleukin-2.

In one embodiment, the composition is administered in combination with interferon.

In one embodiment, the composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171.

In one embodiment, the composition is administered in combination with an mTOR inhibitor. The mTOR inhibitor can be, e.g., rapamycin, everolimus, AP23573, CCI-779, or SDZ-RAD.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced or metastatic melanoma in a subject, e.g., a human, the method comprising:

providing a subject who has advanced or metastatic melanoma and has been treated with a chemotherapeutic agent that did not effectively treat the cancer (e.g., the subject has a chemotherapeutic refractory, a chemotherapeutic resistant and/or a relapsed cancer) or who had unacceptable side effects (e.g., the subject has a chemotherapeutic sensitive cancer), and

administering a composition comprising a CDP-epothilone conjugate, e.g., a CDP-epothilone conjugate described herein, to the subject in an amount effective to treat the melanoma, to thereby treat the melanoma.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject has been treated with a platinum-based agent, which did not effectively treat the cancer (e.g., the subject has been treated with cisplatin, carboplatin or oxaliplatin which did not effectively treat the cancer).

In one embodiment, the subject has been treated with a taxane which did not effectively treat the cancer (e.g., the subject has a taxane refractory, taxane resistant and/or relapsed cancer). In one embodiment, the taxane is paclitaxel.

In one embodiment, the subject has been treated with a tetrazine which did not effectively treat the cancer (e.g., the subject has a dacarbazine, mitozolomide or temozolomide refractory, a dacarbazine, mitozolomide or temozolomide resistant and/or relapsed cancer).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced or metastatic colorectal cancer in a subject, e.g., a human, the method comprising: administering a composition comprising a CDP-epothilone conjugate, e.g., a CDP-epothilone conjugate described herein, to a subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is not administered in combination with a taxane.

In one embodiment, the composition is administered in combination with an antimetabolite, e.g., an antifolate (e.g., pemetrexed, or raltitrexed). In one embodiment, the composition is administered in combination with an antimetabolite, e.g., SFU, and leucovorin. In one embodiment, the composition is further administered in combination with a platinum-based agent (e.g., cisplatin, carboplatin, or oxaliplatin). For example, in one embodiment, the composition is administered in combination with an antimetabolite, e.g., SFU, leucovorin, and a platinum-based agent, e.g., oxaliplatin.

In another embodiment, the antimetabolite is a pyrimidine analog, e.g., capecitabine.

In one embodiment, the composition is administered in combination with a platinum-based agent (e.g., cisplatin, carboplatin, or oxaliplatin).

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced or metastatic colorectal cancer in a subject, e.g., a human. The method comprises: administering a composition comprising a CDP-epothilone conjugate, e.g., a CDP-epothilone conjugate described herein, to a subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the composition is not administered in combination with a taxane.

In one embodiment, the composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the composition is administered with a VEGF pathway inhibitor, e.g., bevacizumab, an antimetabolite, e.g., a pyrimidine analogue (e.g., 5FU), and leucovorin. In another embodiment, the composition is administered with a VEGF pathway inhibitor, e.g., bevacizumab, an antimetabolite, e.g., a pyrimidine analogue (e.g., 5FU), leucovorin, a platinum-based agent (e.g., cisplatin, carboplatin, or oxaliplatin) and/or a topoisomerase inhibitor (e.g., irinotecan, topotecan, etoposide, teniposide, lamellarin D, or camptothecin (e.g., IT-101)). For example, in one embodiment, the composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the composition is administered in combination with a VEGF pathway inhibitor, e.g., bevacizumab, and an antimetabolite wherein the antimetabolite is a pyrimidine analog, e.g., capecitabine. In one embodiment, the composition is further administered in combination with a platinum-based agent (e.g., cisplatin, carboplatin, or oxaliplatin) or a topoisomerase inhibitor (e.g., irinotecan, topotecan, etoposide, teniposide, lamellarin D, or camptothecin (e.g., IT-101)). For example, in one embodiment, the composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, or panitumumab. In one embodiment, the composition is administered in combination with an EGF pathway inhibitor, e.g., cetuximab or panitumumab, and a VEGF pathway inhibitor, e.g., bevacizumab.

In one embodiment, the composition is administered in combination with a topoisomerase inhibitor (e.g., irinotecan, topotecan, etoposide, teniposide, lamellarin D, or camptothecin (e.g., IT-101)). In one embodiment, the composition is administered in combination with a topoisomerase inhibitor (e.g., irinotecan) and a VEGF pathway inhibitor, e.g., bevacizumab.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating advanced or metastatic colorectal cancer in a subject, e.g., a human, the method comprising:

providing a subject who has advanced or metastatic colorectal cancer and has been treated with a chemotherapeutic agent that did not effectively treat the cancer (e.g., the subject has a chemotherapeutic refractory, chemotherapeutic resistant and/or relapsed cancer) or who had unacceptable side effects (e.g., the subject has a chemotherapeutic sensitive cancer), and

administering a composition comprising a CDP-epothilone conjugate, e.g., a CDP-epothilone conjugate described herein, to the subject in an amount effective to treat the cancer, to thereby treat the cancer.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject has been treated with an anti-metabolite, e.g., a pyrimidine analogue which did not effectively treat the cancer (e.g., the subject has a capecitabine and/or 5FU refractory, a capecitabine and/or 5FU resistant and/or relapsed cancer).

In one embodiment, the subject has been treated with a pyrimidine analog which did not effectively treat the cancer (e.g., the subject has a capecitabine refractory, a capecitabine resistant and/or relapsed cancer).

In one embodiment, the composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the composition is administered with a VEGF pathway inhibitor, e.g., bevacizumab, an antimetabolite (e.g., 5FU) and leucovorin. In another embodiment, the composition is administered with a VEGF pathway inhibitor, e.g., bevacizumab, an antimetabolite (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, camptothecin (e.g., IT-101)). For example, in one embodiment, the composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the composition is administered in combination with a VEGF pathway inhibitor, e.g., bevacizumab, and an antimetabolite wherein the antimetabolite is a pyrimidine analog, e.g., capecitabine. In one embodiment, the composition is further administered in combination with a platinum-based agent (e.g., cisplatin, carboplatin, or oxaliplatin) or a topoisomerase inhibitor (e.g., irinotecan, topotecan, etoposide, teniposide, lamellarin D, or camptothecin (e.g., IT-101)). For example, in one embodiment, the composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, or panitumumab. In one embodiment, the composition is administered in combination with an EGF pathway inhibitor, e.g., cetuximab or panitumumab, and a VEGF pathway inhibitor, e.g., bevacizumab.

In one embodiment, the composition is administered in combination with a topoisomerase inhibitor (e.g., irinotecan, topotecan, etoposide, teniposide, lamellarin D, or camptothecin (e.g., IT-101)). In one embodiment, the composition is administered in combination with a topoisomerase inhibitor (e.g., irinotecan) and a VEGF pathway inhibitor, e.g., bevacizumab.

In one embodiment, the composition includes a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the composition includes a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the composition includes a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the composition includes a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the composition includes a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises ea dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the composition includes a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for selecting a subject, e.g., a human, with a proliferative disorder, e.g., cancer, for treatment with a CDP-epothilone conjugate, e.g., a CDP-epothilone conjugate described herein. The method comprises:

determining whether a subject with a proliferative disorder, e.g., cancer, has diabetes; and

selecting the subject for treatment with a CDP-epothilone conjugate, e.g., a CDP-ixabepilone conjugate described herein, on the basis that the subject has diabetes.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone D conjugate at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate at a dose and/or dosing schedule described herein.

In one embodiment, the cancer is a cancer described herein. In one embodiment, the subject is selected for treatment with the CDP-epothilone conjugate in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for treating a subject, e.g., a human, with a proliferative disorder, e.g., cancer, comprising:

selecting a subject with a proliferative disorder who has diabetes; and

administering an epothilone, e.g., a CDP-epothilone conjugate, e.g., a CDP-epothilone conjugate described herein, to the subject in an amount effective to treat the disorder, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the CDP-epothilone conjugate is a CDP-dehydelone conjugate e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the CDP-epothilone conjugate is a CDP-ZK-EPO conjugate e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the cancer is a cancer described herein. In one embodiment, the CDP-epothilone conjugate is administered in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for selecting a subject, e.g., a human, with a proliferative disorder, e.g., cancer, for treatment with a CDP-epothilone conjugate e.g., a CDP-epothilone conjugate described herein, comprising:

determining whether a subject with a proliferative disorder, e.g., cancer, has experienced neuropathy from treatment with a chemotherapeutic agent, e.g., a taxane, a vinca alkaloid, a platinum-based agent or an epothilone; and

selecting the subject for treatment with a CDP-epothilone conjugate, e.g., a CDP-epothilone conjugate described herein, on the basis that the subject has experienced neuropathy from treatment with a chemotherapeutic agent, e.g., a taxane, a vinca alkaloid, a platinum-based agent or an epothilone.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate at a dose and/or dosing schedule described herein.

In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the subject is selected for treatment with the CDP-epothilone conjugate in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for treating a subject, e.g., a human, with a proliferative disorder, e.g., cancer, comprising:

selecting a subject with a proliferative disorder, e.g., cancer, who has experienced one or more symptom of neuropathy from treatment with a chemotherapeutic agent, e.g., a taxane, a vinca alkaloid, a platinum-based agent or an epothilone; and

administering a CDP-epothilone conjugate, e.g., a CDP-epothilone conjugate described herein, to the subject in an amount effective to treat the disorder, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the subject has experienced neuropathy after two, three fours, five cycles of treatment with an epothilone.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the cancer is a cancer described herein. In one embodiment, the CDP-epothilone conjugate is administered in combination with one or more additional chemotherapeutic agent(s), e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for selecting a subject, e.g., a human, with a proliferative disorder, e.g., cancer, for treatment with a CDP-epothilone conjugate, e.g., a CDP-epothilone conjugate described herein, comprising:

determining whether a subject with a proliferative disorder has moderate to severe neuropathy; and

selecting the subject for treatment with a CDP-epothilone conjugate on the basis that the subject has moderate to severe neuropathy.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the method further comprises administering a CDP-epothilone conjugate, e.g., a CDP-epothilone conjugate described herein, to the subject.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, a dose and/or dosing schedule described herein is selected for administration to the subject.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, a dose and/or dosing schedule described herein is selected for administration to the subject.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, a dose and/or dosing schedule described herein is selected for administration to the subject.

In one embodiment, the CDP-epothilone conjugate is a CDP-BMS310705 conjugate e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, a dose and/or dosing schedule described herein is selected for administration to the subject.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, a dose and/or dosing schedule described herein is selected for administration to the subject.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, a dose and/or dosing schedule described herein is selected for administration to the subject.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the CDP-epothilone conjugate is administered in combination with one or more additional chemotherapeutic agent(s), e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method for treating a subject, e.g., a human, with a proliferative disorder, e.g., cancer, comprising:

selecting a subject with a proliferative disorder, e.g., cancer, who has moderate to severe neuropathy; and

administering a CDP-epothilone conjugate, e.g., a CDP-epothilone conjugate described herein, to the subject in an amount effective to treat the disorder, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises epothilone molecules, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising an ixabepilone molecule, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising an epothilone B molecule, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising an epothilone D molecule, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the CDP-epothilone conjugate is a CDP-dehydelone conjugate e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the CDP-epothilone conjugate is a CDP-ZK-EPO conjugate e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the CDP-epothilone conjugate is administered in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method for selecting a subject, e.g., a human, with a proliferative disorder, e.g., cancer, for treatment with a CDP-epothilone conjugate, e.g., a CDP-epothilone conjugate described herein, comprising:

determining whether a subject with a proliferative disorder, e.g., cancer, has experienced an infusion site reaction (e.g., during or within 12 hours of infusion of an epothilone (e.g., ixabepilone)) to treatment with an epothilone (e.g., ixabepilone), and

selecting the subject for treatment with a CDP-epothilone conjugate on the basis that the subject is in need of reduced infusion site reaction (e.g., reduced as compared to the reaction associated with or caused by the treatment with an epothilone (e.g., ixabepilone)).

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the cancer is a cancer described herein. In one embodiment, the CDP-epothilone conjugate is selected for administration in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, with a proliferative disorder, e.g., cancer, comprising:

selecting a subject with a proliferative disorder, e.g., cancer, who has experienced an infusion site reaction to treatment with an epothilone; and

administering a CDP-epothilone conjugate, e.g., a CDP-epothilone conjugate described herein, to the subject in an amount effective to treat the disorder, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the cancer is a cancer described herein. In one embodiment, the CDP-epothilone conjugate is administered in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, with a proliferative disorder, e.g., cancer, comprising:

administering a CDP-epothilone, e.g., a CDP-epothilone conjugate described herein, to a subject with a proliferative disorder, e.g., cancer, in an amount effective to treat the disorder and in the absence of administration of an H1 antagonist or an H2 antagonist, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the cancer is a cancer described herein. In one embodiment, the CDP-epothilone conjugate is administered in combination with one or more additional chemotherapeutic agent(s), e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, with a proliferative disorder, e.g., cancer, comprising:

administering a CDP-epothilone conjugate, e.g., a CDP-epothilone conjugate described herein, to a subject with a proliferative disorder, e.g., cancer, in an amount effective to treat the disorder and in combination with an H1 antagonist or an H2 antagonist, wherein the H1 antagonist is administered at a dose of less than 40 mg, 30 mg, 20 mg, 15 mg, 10 mg, or 5 mg and/or the H2 antagonist is administered at a dose of less than 140 mg, 130 mg, 120 mg, 100 mg, 90 mg, 80 mg, 70 mg, 60 mg, or 50 mg to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the cancer is a cancer described herein. In one embodiment, the CDP-epothilone conjugate is administered in combination with one or more additional chemotherapeutic agent(s), e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining alanine aminotransferase (ALT), aspartate aminotransferase (AST) and/or bilirubin levels in a subject having a proliferative disorder; and

selecting the subject having ALT and/or AST levels greater than 2.5 times the upper limit of normal (ULN) and/or bilirubin levels greater than 1 times the ULN for treatment with an epothilone, e.g., a CDP-epothilone conjugate, e.g., a CDP-epothilone conjugate described herein, and capecitabine.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone D conjugate at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-ZK-EPO conjugate at a dose and/or dosing schedule described herein.

In one embodiment, the cancer is a cancer described herein. In one embodiment, the subject is selected for treatment with the CDP-epothilone conjugate in combination with one or more additional chemotherapeutic agent(s), e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder who has alanine aminotransferase (ALT) and/or aspartate aminotransferase (AST) levels greater than 2.5 times the upper limit of normal (ULN) and/or bilirubin levels greater than 1 time the ULN; and

administering an epothilone, e.g., a CDP-epothilone conjugate, e.g., a CDP-epothilone conjugate described herein, to the subject in an amount effective to treat the disorder, to thereby treat the proliferative disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone D conjugate is administered at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered at a dose and/or dosing schedule described herein.

In one embodiment, the CDP-epothilone conjugate is a CDP-BMS310705 conjugate e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered at a dose and/or dosing schedule described herein.

In one embodiment, the cancer is a cancer described herein. In one embodiment, the CDP-epothilone conjugate is administered in combination with one or more additional chemotherapeutic agent, e.g., a chemotherapeutic agent or combination of chemotherapeutic agents described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining alanine aminotransferase (ALT), aspartate aminotransferase (AST) and/or bilirubin levels in a subject having a proliferative disorder; and

selecting a subject having ALT and/or AST levels less than or equal to 10 times the upper limit of normal (ULN) and bilirubin levels are less than or equal to 1.5 times the ULN for treatment with an epothilone, e.g., a CDP-epothilone conjugate, e.g., a CDP-epothilone conjugate described herein, at a dose of 40 mg/m² or greater.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein. In one embodiment, the subject is selected for treatment with the CDP-ixabepilone conjugate in combination with one or more additional agent, e.g., one or more chemotherapeutic agent described herein. In one embodiment, the subject is selected for administration of at least two doses of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who has alanine aminotransferase (ALT) and/or aspartate aminotransferase (AST) levels less than or equal to 10 times the upper limit of normal (ULN) and bilirubin levels are less than or equal to 1.5 times the ULN; and

administering an epothilone, e.g., a CDP-epothilone conjugate, e.g., a CDP-epothilone conjugate described herein, to the subject at dose of 40 mg/m², to thereby treat the disorder.

In one embodiment, the CDP-epothilone conjugate is a CDP-ixabepilone conjugate, e.g., a CDP-ixabepilone conjugate described herein. In one embodiment, the CDP-ixabepilone conjugate is administered in combination with one or more additional agent, e.g., one or more chemotherapeutic agent described herein. In one embodiment, the subject is administered at least an additional dose of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining alanine aminotransferase (ALT), aspartate aminotransferase (AST) and/or bilirubin levels in a subject having a proliferative disorder; and

selecting a subject having alanine aminotransferase and/or aspartate aminotransferase levels less than or equal to 10 times the upper limit of normal (ULN) and bilirubin levels in the range of greater than 1.5 times the ULN to less than or equal to 3 times the ULN for treatment with an epothilone, e.g., a CDP-epothilone conjugate, e.g., a CDP-epothilone conjugate described herein, at a dose of 40 mg/m².

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for treatment with at least two doses of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who has alanine aminotransferase (ALT) and/or aspartate aminotransferase (AST) levels less than or equal to 10 times the upper limit of normal (ULN) and bilirubin levels in the range of greater than 1.5 times the ULN to less than or equal to 3 times the ULN; and

administering an epothilone, e.g., a CDP-epothilone conjugate, e.g., a CDP-epothilone conjugate described herein, to the subject at dose of 40 mg/m², to thereby treat the disorder.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, the subject is administered at least an additional dose of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject having a proliferative disorder is currently being administered (e.g., the subject has been administered a CYP3A4 inhibitor the same day as chemotherapy treatment or within 1, 2, 3, 4, 5, 6, or 7 days before chemotherapy treatment) or will be administered (e.g., will be administered on the same day as the chemotherapy treatment or within 1, 2, 3, 4, 5, 6, or 7 days after chemotherapy treatment) a CYP3A4 inhibitor (e.g., ketoconazole, itraconazole, clarithromycin, atazanavir, nefazodone, saquinavir, telithromycin, ritonavir, amprenavir, indinavir, nelfinavir, delavirdine or voriconazole); and

selecting a subject with a proliferative disorder, e.g., cancer, that is currently being administered or will be administered a CYP3A4 inhibitor for treatment with an epothilone, e.g., a CDP-epothilone conjugate, e.g., a CDP-epothilone conjugate described herein, at a dose of 40 mg/m².

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least two doses of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of treating a subject, e.g., a human, having a proliferative disorder, e.g., cancer, comprising:

selecting a subject with a proliferative disorder, e.g., cancer, who is currently being administered or will be, administered a CYP3A4 inhibitor; and

administering an epothilone, e.g., a CDP-epothilone conjugate, e.g., a CDP-epothilone conjugate described herein, to the subject at dose of 40 mg/m², to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, the subject is administered at least an additional dose of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of selecting a subject, e.g., a human, with a proliferative disorder, e.g., cancer, for treatment with an epothilone, e.g., a CDP-epothilone, e.g., a CDP-epothilone conjugate described herein, comprising:

determining if a subject having a proliferative disorder is currently being administered or will be administered an anti-depressant, and

selecting a subject who is currently being administered or will be administered an anti-depressant, e.g., St. John's Wort, for treatment with an epothilone, e.g., a CDP-epothilone conjugate, e.g., a CDP-epothilone conjugate described herein.

In one embodiment, the anti-depressant is one or more of a monoamine oxidase inhibitor (MAOI), a tricyclic antidepressant (TCA), a tetracyclic antidepressant (TeCA), a selective serotonin reuptake inhibitor (SSRI), and a serotonin-norepinephrine reuptake inhibitor (SNRI). In one embodiment, the anti-depressant is St. John's wort.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least two doses of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of treating a subject, e.g., a human, having a proliferative disorder, e.g., cancer, comprising:

selecting a subject with a proliferative disorder, e.g., cancer, that is currently being administered or will be administered an antidepressant; and

administering an epothilone, e.g., a CDP-epothilone conjugate, e.g., a CDP-epothilone conjugate described herein, to the subject in an amount effective to treat the disorder, to thereby treat the disorder.

In one embodiment, the anti-depressant is one or more of a monoamine oxidase inhibitor (MAOI), a tricyclic antidepressant (TCA), a tetracyclic antidepressant (TeCA), a selective serotonin reuptake inhibitor (SSRI), and a serotonin-norepinephrine reuptake inhibitor (SNRI). In one embodiment, the anti-depressant is St. John's wort.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, the subject is administered at least an additional dose of the CDP-ixabepilone conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of selecting a subject, e.g., a human, with a proliferative disorder, e.g., cancer, for treatment with a CDP-epothilone conjugate, e.g., a CDP-epothilone conjugate described herein, comprising:

determining if a subject having a proliferative disorder is 65 or older, and

selecting a subject who is 65 or older for treatment with a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least an additional dose of the CDP-ixabepilone conjugate, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate in combination with one or more additional agent, e.g., one or more chemotherapeutic agent described herein. In one embodiment, the subject is selected for administration of at least an additional dose of the CDP-epothilone D conjugate, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate in combination with one or more additional agent, e.g., one or more chemotherapeutic agent described herein. In one embodiment, the subject is selected for administration of at least an additional dose of the CDP-BMS310705 conjugate, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate in combination with one or more additional agent, e.g., one or more chemotherapeutic agent described herein. In one embodiment, the subject is selected for administration of at least an additional dose of the CDP-dehydelone conjugate, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate in combination with one or more additional agent, e.g., one or more chemotherapeutic agent described herein. In one embodiment, the subject is selected for administration of at least an additional dose of the CDP-ZK-EPO conjugate, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., a cancer, who is 65 or older; and

administering a CDP-epothilone conjugate, e.g., a CDP-epothilone conjugate described herein, and capecitabine to the subject in an amount effective to treat the disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject with a proliferative disorder, e.g., a cancer, is at risk for or has or previously had a cardiac adverse reaction, and

selecting a subject who is at risk for or has or previously had a cardiac adverse reaction for treatment with an epothilone, e.g., a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least an additional dose of the CDP-ixabepilone conjugate, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2.

In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein. In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate in combination with one or more additional agent, e.g., one or more chemotherapeutic agent described herein. In one embodiment, the subject is selected for administration of at least an additional dose of the CDP-epothilone D conjugate, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate in combination with one or more additional agent, e.g., one or more chemotherapeutic agent described herein. In one embodiment, the subject is selected for administration of at least an additional dose of the CDP-BMS310705 conjugate, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate in combination with one or more additional agent, e.g., one or more chemotherapeutic agent described herein. In one embodiment, the subject is selected for administration of at least an additional dose of the CDP-dehydelone conjugate, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate in combination with one or more additional agent, e.g., one or more chemotherapeutic agent described herein. In one embodiment, the subject is selected for administration of at least an additional dose of the CDP-ZK-EPO conjugate, e.g., at a dose and/or dosing schedule described herein.

In one embodiment, a cardiac adverse reaction includes, e.g., myocardial ischemia, ventricular dysfunction, impaired cardiac function, myocardial infarction, supraventricular arrhythmia, left ventricular dysfunction, angia pectoris, atrial flutter, congestive heart failure (e.g., New York Heart Association class III or class IV heart failure), cardiac insufficiency, congenital long QT syndrome and/or cardiomyopathy.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who is at risk for or has or previously had a cardiac adverse reaction; and

administering an epothilone, e.g., a CDP-epothilone conjugate, e.g., a CDP-epothilone conjugate described herein, to the subject in an amount effective to treat the disorder, to thereby treat In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, a cardiac adverse reaction includes, e.g., myocardial ischemia, ventricular dysfunction, impaired cardiac function, myocardial infarction, supraventricular arrhythmia, left ventricular dysfunction, angia pectoris, artrial flutter, congestive heart failure (e.g., New York Heart Association class III or class IV heart failure), cardiac insufficiency, congenital long QT syndrome and/or cardiomyopathy.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of identifying a subject, e.g., a human, having a proliferative disorder, e.g., cancer, for treatment with a CDP-epothilone conjugate, e.g., a CDP-epothilone conjugate described herein, the method comprising

identifying a subject having a proliferative disorder who has received an epothilone (e.g., ixabepilone, epothilone B, epothilone D, BMS310705, dehydelone or ZK-EPO) and has a platelet count less than a standard; and

identifying the subject as suitable for treatment with a CDP-epothilone conjugate, e.g., a CDP-epothilone conjugate described herein.

In one embodiment, the method further comprising administering a CDP-epothilone conjugate, e.g., a CDP-epothilone conjugate described herein in an amount effective to treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, the method comprising

selecting a subject having a proliferative disease who has received an epothilone (e.g., ixabepilone, epothilone B, epothilone D, BMS310705, dehydelone or ZK-EPO) and has a platelet count less than a standard; and

administering a CDP-epothilone conjugate, e.g., a CDP-epothilone conjugate described herein, to the subject in an amount effective to treat the proliferative disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-epothilone dehydelone shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the standard is a platelet count below or equal to 50×10³ platelets/mm³. In some embodiments, the standard is platelet count prior to receiving an epothilone treatment. In one embodiment, the standard is a decrease from the mean platelet count prior to initiation of the treatment with an epothilone, e.g., by at least 20%, 30%, 40% or 50%.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of identifying a subject, e.g., a human, having a proliferative disorder, e.g., cancer, for treatment with a CDP-epothilone conjugate, e.g., a CDP-epothilone conjugate described herein, the method comprising

identifying a subject having a proliferative disorder who has received an epothilone (e.g., ixabepilone, epothilone B, epothilone D, BMS310705, dehydelone or ZK-EPO) and has a neutrophil count less than a standard; and

identifying the subject as suitable for treatment with a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the method further comprising administering a CDP-epothilone conjugate, e.g., a CDP-epothilone conjugate described herein in an amount effective to treat the disorder.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of treating a subject, e.g., a human, with a proliferative disorder, e.g., cancer, the method comprising

selecting a subject having a proliferative disease who has received an epothilone (e.g., ixabepilone) and has a neutrophil count less than a standard; and

administering a CDP-epothilone conjugate, e.g., a CDP-epothilone conjugate described herein, to the subject in an amount effective to treat the proliferative disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the standard is a neutrophil count below or equal to 1500 cells/mm³. In some embodiments, the standard is based on a neutrophil count prior to receiving an epothilone treatment, e.g., mean neutrophil count decreased from the mean neutrophil count prior to treatment with the epothilone, e.g., by at least 20%, 30%, 40% or 50% after administration of the epothilone.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of identifying a subject, e.g., a human, having a proliferative disorder, e.g., cancer, for treatment with a CDP-epothilone conjugate, e.g., a CDP-epothilone conjugate described herein, the method comprising

identifying a subject having a proliferative disorder who has received an epothilone (e.g., ixabepilone) and had one or more symptom of febrile neutropenia; and

identifying the subject as suitable for treatment with a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the method further comprises administering a CDP-epothilone conjugate, e.g., a CDP-epothilone conjugate described herein, in an amount effective to treat the disorder.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2.

In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the symptom of febrile neutropenia is one or more of: fever, infection or a low neutrophil count in the blood.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, with a proliferative disorder, e.g., cancer, the method comprising

selecting a subject having a proliferative disease who has received an epothilone (e.g., ixabepilone, epothilone B, epothilone D, BMS 310705, dehydelone or ZK-EPO) and had one or more symptom of febrile neutropenia; and

administering a CDP-epothilone conjugate, e.g., a CDP-epothilone conjugate described herein, to the subject in an amount effective to treat the proliferative disorder, to thereby treat the disorder.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the symptom of febrile neutropenia is one or more of: fever, infection, or a low neutrophil count in the blood.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of identifying a subject, e.g., a human, having a proliferative disorder, e.g., cancer, for treatment with a CDP-epothilone conjugate, e.g., a CDP-epothilone conjugate described herein, the method comprising

identifying a subject having a proliferative disorder who has one or more symptom of febrile neutropenia; and

identifying the subject as suitable for treatment with a CDP-epothilone conjugate, e.g., a CDP-epothilone conjugate described herein, at a dose of 40 mg/m².

In one embodiment, the method further comprises administering a CDP-epothilone conjugate, e.g., a CDP-epothilone conjugate described herein, in an amount effective to treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for administration of the CDP-ixabepilone conjugate in combination with one or more additional agent, e.g., one or more chemotherapeutic agent described herein. In one embodiment, the subject is selected for administration of at least one additional dose of the CDP-ixabepilone conjugate, e.g., at a dose and/or dosing schedule described herein.

In one embodiment, the symptom of febrile neutropenia is one or more of: fever, infection, or a low neutrophil count in the blood.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, with a proliferative disorder, e.g., cancer, the method comprising

selecting a subject having a proliferative disease who has one or more symptom of febrile neutropenia; and

administering a CDP-epothilone conjugate, e.g., a CDP-epothilone conjugate described herein, to the subject at a dose 40 mg/m², to thereby treat the disorder.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the symptom of febrile neutropenia is one or more of: fever, infection, or a low neutrophil count in the blood.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject with a proliferative disorder, e.g., a cancer, is at risk for or has diarrhea or has experienced diarrhea from treatment with an epothilone, e.g., epothilone B, and

selecting a subject who is at risk for or has diarrhea or has experienced diarrhea from treatment with an epothilone for treatment with an epothilone, e.g., a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein.

In one embodiment, the epothilone, e.g., the CDP-epothilone conjugate, is administered in combination with an anti-diarrheal agent. The anti-diarrheal agent can be, e.g., an opioid (e.g., codeine, oxicodeine, Percocet, paregoric, tincture of opium, diphenoxylate, or diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide or a DPP-IV inhibitor.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who is at risk for or has diarrhea or has experienced diarrhea from treatment with an epothilone, e.g., epothilone B; and

administering an epothilone, e.g., a CDP-epothilone conjugate, e.g., a CDP-epothilone conjugate described herein, to the subject in an amount effective to treat the disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the epothilone, e.g., the CDP-epothilone conjugate, is administered in combination with an anti-diarrheal agent. The anti-diarrheal agent can be, e.g., an opioid (e.g., codeine, oxicodeine, Percocet, paregoric, tincture of opium, diphenoxylate, or diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide or a DPP-IV inhibitor.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject with a proliferative disorder, e.g., a cancer, has a catheter or port, e.g., an in-dwelling catheter or port, and

selecting a subject who has a catheter or port, e.g., an in-dwelling catheter or port, for treatment with an epothilone, e.g., a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least an additional dose of the CDP-ixabepilone conjugate, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate in combination with one or more additional agent, e.g., one or more chemotherapeutic agent described herein. In one embodiment, the subject is selected for administration of at least an additional dose of the CDP-epothilone D conjugate, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate in combination with one or more additional agent, e.g., one or more chemotherapeutic agent described herein. In one embodiment, the subject is selected for administration of at least an additional dose of the CDP-BMS310705 conjugate, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate in combination with one or more additional agent, e.g., one or more chemotherapeutic agent described herein. In one embodiment, the subject is selected for administration of at least an additional dose of the CDP-dehydelone conjugate, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate in combination with one or more additional agent, e.g., one or more chemotherapeutic agent described herein. In one embodiment, the subject is selected for administration of at least an additional dose of the CDP-ZK-EPO conjugate, e.g., at a dose and/or dosing schedule described herein.

In one embodiment, the subject is also being administered an anticoagulant such as heparin or warfarin.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who has a catheter or port, e.g., an in-dwelling catheter or port; and

administering an epothilone, e.g., a CDP-epothilone conjugate, e.g., a CDP-epothilone conjugate described herein, to the subject in an amount effective to treat the disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the subject is also administered an anticoagulant such as heparin or warfarin.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject with a proliferative disorder, e.g., a cancer, is at risk for needing an anticoagulant or is currently being administered an anticoagulant, and

selecting a subject who is at risk for needing an anticoagulant or is currently being administered an anticoagulant for treatment with an epothilone, e.g., a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least an additional dose of the CDP-ixabepilone conjugate, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate in combination with one or more additional agent, e.g., one or more chemotherapeutic agent described herein. In one embodiment, the subject is selected for administration of at least an additional dose of the CDP-epothilone D conjugate, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate in combination with one or more additional agent, e.g., one or more chemotherapeutic agent described herein. In one embodiment, the subject is selected for administration of at least an additional dose of the CDP-BMS310705 conjugate, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate in combination with one or more additional agent, e.g., one or more chemotherapeutic agent described herein. In one embodiment, the subject is selected for administration of at least an additional dose of the CDP-dehydelone conjugate, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate in combination with one or more additional agent, e.g., one or more chemotherapeutic agent described herein. In one embodiment, the subject is selected for administration of at least an additional dose of the CDP-ZK-EPO conjugate, e.g., at a dose and/or dosing schedule described herein.

In one embodiment, the anticoagulant is a heparin or warfarin.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who is at risk for needing an anticoagulant or is currently being administered an anticoagulant; and

administering an epothilone, e.g., a CDP-epothilone conjugate, e.g., a CDP-epothilone conjugate described herein, to the subject in an amount effective to treat the disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the anticoagulant is a heparin or warfarin.

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the disclosure features a method of selecting a subject, e.g., a human, with a proliferative disorder, e.g., cancer, for treatment with an epothilone, e.g., a CDP-epothilone conjugate, e.g., a CDP-epothilone conjugate described herein, comprising:

determining if a subject with a proliferative disorder, e.g., a cancer, is at risk for needing a hematopoietic growth factor or is currently being administered a hematopoietic growth factor, and

selecting a subject who is at risk for needing a hematopoietic growth factor or is currently being administered a hematopoietic growth factor for treatment with an epothilone, e.g., a CDP-epothilone conjugate, e.g., a CDP-epothilone conjugate described herein.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the subject is selected for treatment with the CDP-ixabepilone conjugate in combination with one or more additional agent, e.g., one or more chemotherapeutic agent described herein. In one embodiment, the subject is selected for administration of at least an additional dose of the CDP-ixabepilone conjugate, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the subject is selected for treatment with the CDP-epothilone B conjugate in combination with one or more additional agent, e.g., one or more chemotherapeutic agent described herein. In one embodiment, the subject is selected for administration of at least an additional dose of the CDP-epothilone B conjugate, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the subject is selected for treatment with the CDP-epothilone D conjugate in combination with one or more additional agent, e.g., one or more chemotherapeutic agent described herein. In one embodiment, the subject is selected for administration of at least an additional dose of the CDP-epothilone D conjugate, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the subject is selected for treatment with the CDP-BMS310705 conjugate in combination with one or more additional agent, e.g., one or more chemotherapeutic agent described herein. In one embodiment, the subject is selected for administration of at least an additional dose of the CDP-BMS310705 conjugate, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the subject is selected for treatment with the CDP-dehydelone conjugate in combination with one or more additional agent, e.g., one or more chemotherapeutic agent described herein. In one embodiment, the subject is selected for administration of at least an additional dose of the CDP-dehydelone conjugate, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the subject is selected for treatment with the CDP-ZK-EPO conjugate in combination with one or more additional agent, e.g., one or more chemotherapeutic agent described herein. In one embodiment, the subject is selected for administration of at least an additional dose of the CDP-ZK-EPO conjugate, e.g., at a dose and/or dosing schedule described herein.

In one embodiment, the hematopoietic growth factor is a colony stimulating factor such as granulocyte colony stimulating factor (G-CSF) or granulocyte macrophage-colony stimulating factor (GM-CSF).

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In one aspect, the disclosure features a method of treating a subject, e.g., a human, having a proliferative disorder, e.g., cancer, comprising:

selecting a subject with a proliferative disorder, e.g., cancer, who is at risk for needing a hematopoietic growth factor or is currently being administered a hematopoietic growth factor; and

administering an epothilone, e.g., a CDP-epothilone conjugate, e.g., a CDP-epothilone conjugate described herein, to the subject in an amount effective to treat the disorder, to thereby treat the disorder.

In an embodiment, the CDP-epothilone conjugate comprises epothilone molecules, coupled, e.g., via linkers, to a CDP moiety referred to in FIG. 1. In an embodiment, the CDP-epothilone conjugate comprises an epothilone molecule, coupled via a linker shown in FIG. 2 to a CDP moiety, e.g., a CDP moiety referred to in FIG. 1. In an embodiment, the CDP-epothilone conjugate is a CDP-epothilone conjugate shown in FIG. 2.

In one embodiment, the CDP-epothilone conjugate is a CDP-ixabepilone conjugate, e.g., a CDP-ixabepilone conjugate described herein, e.g., a CDP-ixabepilone conjugate comprising ixabepilone molecules, coupled, e.g., via linkers, to a CDP moiety referred to in FIG. 1. In an embodiment, the CDP-epothilone conjugate comprises an ixabepilone molecule, coupled via a linker shown in FIG. 2 to a CDP moiety, e.g., a CDP moiety referred to in FIG. 1. In an embodiment, the CDP-epothilone conjugate is a CDP-ixabepilone conjugate shown in FIG. 2. In one embodiment, the CDP-ixabepilone conjugate is administered in combination with one or more additional agent, e.g., one or more chemotherapeutic agent described herein. In one embodiment, at least an additional dose of the CDP-ixabepilone conjugate is administered, e.g., at a dose and/or dosing schedule described herein.

In one embodiment, the CDP-epothilone conjugate is a CDP-epothilone B conjugate, e.g., a CDP-epothilone B conjugate described herein, e.g., a CDP-epothilone B conjugate comprising epothilone B molecules, coupled, e.g., via linkers, to a CDP moiety referred to in FIG. 1. In an embodiment, the CDP-epothilone conjugate comprises an epothilone B molecule, coupled via a linker shown in FIG. 2 to a CDP moiety, e.g., a CDP moiety referred to in FIG. 1. In an embodiment, the CDP-epothilone conjugate is a CDP-epothilone B conjugate shown in FIG. 2. In one embodiment, the CDP-epothilone B conjugate is administered in combination with one or more additional agent, e.g., one or more chemotherapeutic agent described herein. In one embodiment, at least an additional dose of the CDP-epothilone B conjugate is administered, e.g., at a dose and/or dosing schedule described herein.

In one embodiment, the CDP-epothilone conjugate is a CDP-epothilone D conjugate, e.g., a CDP-epothilone D conjugate described herein, e.g., a CDP-epothilone D conjugate comprising epothilone D molecules, coupled, e.g., via linkers, to a CDP moiety referred to in FIG. 1. In an embodiment, the CDP-epothilone conjugate comprises an epothilone D molecule, coupled via a linker shown in FIG. 2 to a CDP moiety, e.g., a CDP moiety referred to in FIG. 1. In an embodiment, the CDP-epothilone conjugate is a CDP-epothilone D conjugate shown in FIG. 2. In one embodiment, the CDP-epothilone D conjugate is administered in combination with one or more additional agent, e.g., one or more chemotherapeutic agent described herein. In one embodiment, at least an additional dose of the CDP-epothilone D conjugate is administered, e.g., at a dose and/or dosing schedule described herein.

In one embodiment, the CDP-epothilone conjugate is a CDP-BMS310705 conjugate, e.g., a CDP-BMS310705 conjugate described herein, e.g., a CDP-BMS310705 conjugate comprising BMS310705 molecules, coupled, e.g., via linkers, to a CDP moiety referred to in FIG. 1. In an embodiment, the CDP-epothilone conjugate comprises a BMS310705 molecule, coupled via a linker shown in FIG. 2 to a CDP moiety, e.g., a CDP moiety referred to in FIG. 1. In an embodiment, the CDP-epothilone conjugate is a CDP-BMS310705 conjugate shown in FIG. 2. In one embodiment, the CDP-BMS310705 conjugate is administered in combination with one or more additional agent, e.g., one or more chemotherapeutic agent described herein. In one embodiment, at least an additional dose of the CDP-BMS310705 conjugate is administered, e.g., at a dose and/or dosing schedule described herein.

In one embodiment, the CDP-epothilone conjugate is a CDP-dehydelone conjugate, e.g., a CDP-dehydelone conjugate described herein, e.g., a CDP-dehydelone conjugate comprising dehydelone molecules, coupled, e.g., via linkers, to a CDP moiety referred to in FIG. 1. In an embodiment, the CDP-epothilone conjugate comprises a dehydelone molecule, coupled via a linker shown in FIG. 2 to a CDP moiety, e.g., a CDP moiety referred to in FIG. 1. In an embodiment, the CDP-epothilone conjugate is a CDP-dehydelone conjugate shown in FIG. 2. In one embodiment, the CDP-dehydelone conjugate is administered in combination with one or more additional agent, e.g., one or more chemotherapeutic agent described herein. In one embodiment, at least an additional dose of the CDP-dehydelone conjugate is administered, e.g., at a dose and/or dosing schedule described herein.

In one embodiment, the CDP-epothilone conjugate is a CDP-ZK-EPO conjugate, e.g., a CDP-ZK-EPO conjugate described herein, e.g., a CDP-ZK-EPO conjugate comprising ZK-EPO molecules, coupled, e.g., via linkers, to a CDP moiety referred to in FIG. 1. In an embodiment, the CDP-epothilone conjugate comprises a ZK-EPO molecule, coupled via a linker shown in FIG. 2 to a CDP moiety, e.g., a CDP moiety referred to in FIG. 1. In an embodiment, the CDP-epothilone conjugate is a CDP-ZK-EPO conjugate shown in FIG. 2. In one embodiment, the CDP-ZK-EPO conjugate is administered in combination with one or more additional agent, e.g., one or more chemotherapeutic agent described herein. In one embodiment, at least an additional dose of the CDP-ZK-EPO conjugate is administered, e.g., at a dose and/or dosing schedule described herein.

In one embodiment, the hematopoietic growth factor is a colony stimulating factor such as granulocyte colony stimulating factor (G-CSF) or granulocyte macrophage colony stimulating factor (GM-CSF).

In one embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms a particle or nanoparticle having a conjugate number described herein. By way of example, a CDP-therapeutic agent conjugate, forms, or is provided in, a particle or nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment, the CDP-therapeutic agent conjugate is administered as a nanoparticle or preparation of nanoparticles, e.g., a pharmaceutical preparation, wherein at least 60% of the particles in the preparation have a conjugate number of 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 5 to 10; 10 to 15; 15-20; 20-25; 1 to 40; 1 to 30; 1 to 20; 1 to 15; 10 to 40; 10 to 30; 10 to 20; 10 to 15; 20 to 40; 20 to 30; or 20 to 25; 1-100; 25 to 100; 50 to 100; 75-100; 25 to 75, 25 to 50, or 50 to 75; 25 to 40; 25 to 50; 30 to 50; 30 to 40; or 30 to 75.

The details of one or more embodiments of the invention are set forth in the description below. Other features, objects, and advantages of the invention will be apparent from the description and the drawings, and from the claims.

### BRIEF DESCRIPTION OF THE FIGURES

FIG. 1 depicts a cyclodextrin containing polymer (CDP).

FIG. 2 depicts a table which shows 400 exemplary CDP-epothilone conjugates.

FIG. 3 depicts exemplary epothilone structures.

FIG. 4 depicts a general strategy for synthesizing linear, branched or grafted cyclodextrin-containing polymers (CDPs) for loading an epothilone, and an optional targeting ligand.

FIG. 5 depicts a general scheme for graft polymers.

FIG. 6 depicts a general scheme of preparing linear CDPs.

FIG. 7 depicts CRLX101 particle size dependency on conjugate number.

### DETAILED DESCRIPTION OF THE INVENTION

The present invention relates to cyclodextrin-containing polymers conjugated to an epothilone, compositions of therapeutic cyclodextrin-containing polymers conjugated to an epothilone, particles (e.g., nanoparticles) including cyclodextrin-containing polymers conjugated to an epothilone, and methods of use thereof. In certain embodiments, these cyclodextrin-containing polymers improve epothilone stability and/or epothilone solubility, and/or reduce epothilone toxicity, and/or improve efficacy of the epothilone when used in vivo.

By selecting from a variety of linker groups or a direct bond used to attach an epothilone to a CDP, the rate of epothilone release from the CDP can be attenuated for controlled delivery.

The invention also relates to methods of treating subjects, e.g., humans, with a CDP-epothilone conjugate described herein. The invention further relates to methods for conducting a pharmaceutical business comprising manufacturing, licensing, or distributing kits containing or relating to the CDP-epothilone conjugates described herein.

More generally, the present invention provides cyclodextrin containing polymer-epothilone conjugates. Exemplary cyclodextrin containing polymer-epothilone conjugates include water-soluble, biocompatible polymer conjugates comprising a water-soluble, biocompatible cyclodextrin containing polymer covalently attached to an epothilone through attachments (e.g., linkages) that are cleaved under biological conditions to release the epothilone.

Polymeric conjugates (e.g., CDP-conjugates) featured in the present invention may be useful to improve solubility and/or stability of a bioactive/therapeutic agent, such as epothilone, reduce drug-drug interactions, reduce interactions with blood elements including plasma proteins, reduce or eliminate immunogenicity, protect the agent (e.g., epothilone) from metabolism, modulate drug-release kinetics (e.g., epothilone-release kinetics), improve circulation time, improve drug half-life (e.g., epothilone half-life in the serum, or in selected tissues, such as tumors), attenuate toxicity, improve efficacy, normalize drug (e.g., epothilone) metabolism across subjects of different species, ethnicities, and/or races, and/or provide for targeted delivery into specific cells or tissues. Poorly soluble and/or toxic compounds, such as an epothilone described herein, may benefit particularly from incorporation into polymeric compounds of the invention (e.g., a CDP described herein, forming a CPD-epothilone conjugate described herein).

An “effective amount” or “an amount effective” refers to an amount of the compound, which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition is outweighed by the therapeutically beneficial effects.

As used herein the term “low aqueous solubility” refers to water insoluble compounds having poor solubility in water, that is <5 mg/ml at physiological pH (6.5-7.4). Preferably, their water solubility is <1 mg/ml, more preferably <0.1 mg/ml. It is desirable that the drug is stable in water as a dispersion; otherwise a lyophilized or spray-dried solid form may be desirable.

As used herein, the term “treat” or “treating” a subject having a disease refers to subjecting the subject to a treatment, e.g., the administration of a compound, such that at least one symptom of the disease or disorder is cured, healed, alleviated, relieved, altered, remedied, ameliorated, or improved. Treating includes administering an amount effective to alleviate, relieve, alter, remedy, ameliorate, improve or affect the disorder, the symptoms of the disease or disorder or the predisposition toward the disease or disorder. The treatment may also delay onset, e.g., prevent onset, or prevent deterioration or worsening of a symptom of a disease or disorder.

Epothilone B is also referred to herein as patupilone and EP0906.

Ixabepilone is also referred to herein as Ixempra™.

Epothilone D is also referred to herein as KOS-862.

Dehydelone is also referred to herein as KOS-1584.

ZK-EPO is also referred to herein as sagopilone.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

CDP-Epothilone Conjugates

Described herein are cyclodextrin containing polymer (“CDP”)-epothilone conjugates, wherein one or more epothilones are covalently attached to the CDP (e.g., either directly or through a linker). The CDP-epothilone conjugates include linear or branched cyclodextrin-containing polymers, and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952.

Accordingly, in one embodiment the CDP-epothilone conjugate is represented by Formula I:

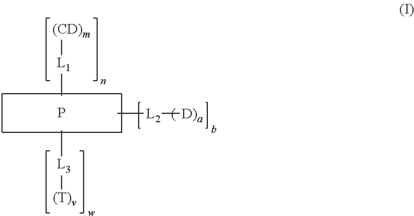

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents an epothilone or a prodrug thereof;

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the epothilone moieties in the CDP-epothilone conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In certain embodiments, P contains a plurality of cyclodextrin moieties within the polymer chain as opposed to the cyclodextrin moieties being grafted on to pendant groups off of the polymeric chain. Thus in certain embodiments, the polymer chain of formula I further comprises n′ units of U, wherein n′ represents an integer in the range of 1 to about 30,000, e.g., from 4-100, 4-50, 4-25, 4-15, 6-100, 6-50, 6-25, or 6-15 (preferably <25,000, <20,000, <15,000, <10,000, <5,000, <1,000, <500, <100, <50, <25, <20, <15, <10, or even <5); and U is represented by one of the general formulae below:

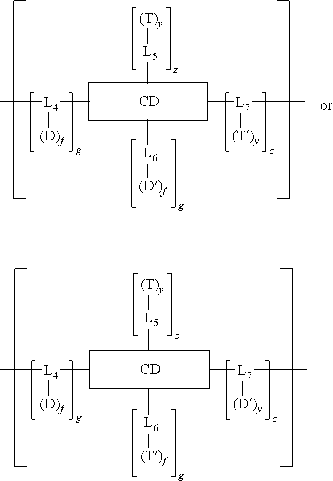

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different epothilone or prodrug forms thereof;

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. In some embodiments, at least 50% of the U units have at least one D or D′. In some embodiments, one or more of the epothilone moieties in the CDP-epothilone conjugate can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-epothilone conjugate is represented by Formula II:

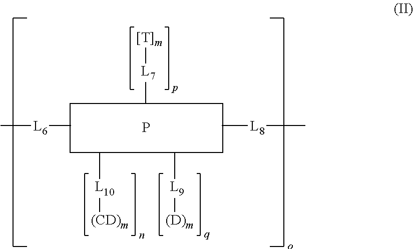

wherein

P represents a monomer unit of a polymer that comprises cyclodextrin moieties;

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents an epothilone or a prodrug form thereof;

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the epothilone moieties in the CDP-epothilone conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-epothilone conjugate is represented either of the formulae below:

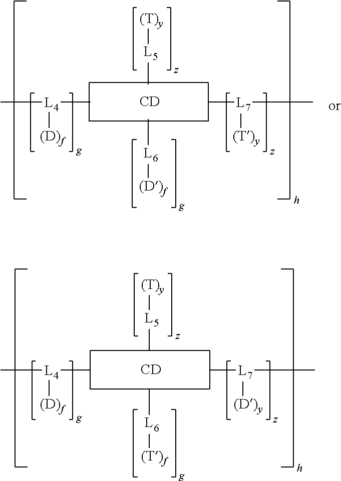

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different epothilone or prodrug thereof;

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

Preferably the polymer has a plurality of D or D′ moieties. In some embodiments, at least 50% of the polymer repeating units have at least one D or D′. In some embodiments, one or more of the epothilone moieties in the CDP-epothilone conjugate can be replaced with another therapeutic agent, e.g., another anticancer agent or anti-inflammatory agent.

In preferred embodiments, L4 and L7 represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6,15-crown-5,12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL

Thus, in certain embodiments, the invention relates to novel compositions of therapeutic cyclodextrin-containing polymeric compounds designed for drug delivery of an epothilone. In certain embodiments, these CDPs improve drug stability and/or solubility, and/or reduce toxicity, and/or improve efficacy of the epothilone when used in vivo. Furthermore, by selecting from a variety of linker groups or direct bonds, and/or targeting ligands, the rate of epothilone release from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

Described herein are CDP-epothilone conjugates, wherein one or more epothilones are covalently attached to the CDP. The CDP can include linear or branched cyclodextrin-containing polymers and/or polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952, which are incorporated herein by reference in their entirety.

In some embodiments, the CDP-epothilone conjugate comprises a water soluble linear polymer conjugate comprising: cyclodextrin moieties; comonomers which do not contain cyclodextrin moieties (comonomers); and a plurality of epothilones; wherein the CDP-epothilone conjugate comprises at least four, five six, seven, eight, etc., cyclodextrin moieties and at least four, five six, seven, eight, etc., comonomers. In some embodiments, the epothilone is an epothilone described herein, for example, the epothilone is epothilone B, ixabepilone, BMS310705, epothilone D, dehydelone, or sagopilone. The epothilone can be attached to the CDP via a functional group such as a hydroxyl group, or where appropriate, an amino group.

In some embodiments, one or more of the epothilone moieties in the CDP-epothilone conjugate can be replaced with another therapeutic agent, e.g., another anticancer agent or anti-inflammatory agent.

In some embodiments, the least four cyclodextrin moieties and at least four comonomers alternate in the CDP-epothilone conjugate. In some embodiments, said epothilones are cleaved from said CDP-epothilone conjugate under biological conditions to release epothilones. In some embodiments, the cyclodextrin moieties comprise linkers to which epothilones are linked. In some embodiments, the epothilones are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of an epothilone was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring. In some embodiments, the cyclodextrin moiety comprises an alpha, beta, or gamma cyclodextrin moiety. In some embodiments, the epothilone is at least 5%, 10%, 15%, 20%, 25%, 30%, or 35% by weight of CDP-epothilone conjugate.

In some embodiments, the comonomer comprises polyethylene glycol of molecular weight 3,400 Da, the cyclodextrin moiety comprises beta-cyclodextrin, the theoretical maximum loading of the epothilone on the CDP-epothilone conjugate is 13% by weight, and the epothilone is 6-10% by weight of CDP-epothilone conjugate. In some embodiments, the epothilone is poorly soluble in water. In some embodiments, the solubility of the epothilone is <5 mg/ml at physiological pH. In some embodiments, the epothilone is a hydrophobic compound with a log P>0.4, >0.6, >0.8, >1, >2, >3, >4, or >5.

In some embodiments, the epothilone is attached to the CDP via a second compound.

In some embodiments, administration of the CDP-epothilone conjugate to a subject results in release of the epothilone over a period of at least 6 hours. In some embodiments, administration of the CDP-epothilone conjugate to a subject results in release of the epothilone over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-epothilone conjugate to a subject the rate of epothilone release is dependent primarily upon the rate of hydrolysis as opposed to enzymatic cleavage.

In some embodiments, the CDP-epothilone conjugate has a molecular weight of 10,000-500,000. In some embodiments, the cyclodextrin moieties make up at least about 2%, 5%, 10%, 20%, 30%, 50% or 80% of the CDP-epothilone conjugate by weight.

In some embodiments, the CDP-epothilone conjugate is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-epothilone conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-epothilone conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O—, —NR₁—, —NR₁CO—, —C(O)NR₁—, —S(O)ₙ— (wherein n is 0, 1, or 2), —OC(O)—NR₁—, —NR₁—C(O)—NR₁—, —NR₁—C(NR₁)—NR₁—, and —B(OR₁)—; and R₁, independently for each occurrence, represents H or a lower alkyl.

In some embodiments, the CDP-epothilone conjugate is a polymer having attached thereto a plurality of D moieties of the following formula:

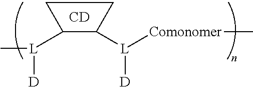

wherein each L is independently a linker, and each D is independently an epothilone, a prodrug derivative thereof, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one epothilone and in some embodiments, at least two epothilone moieties. In some embodiments, the molecular weight of the comonomer is from about 2000 to about 5000 Da (e.g., from about 3000 to about 4000 Da (e.g., about 3400 Da).

In some embodiments, the epothilone is an epothilone described herein, for example, the epothilone is epothilone B, ixabepilone, BMS310705, epothilone D, dehydelone, or sagopilone. The epothilone can be attached to the CDP via a functional group such as a hydroxyl group, or where appropriate, an amino group. In some embodiments, one or more of the epothilone moieties in the CDP-epothilone conjugate can be replaced with another therapeutic agent, e.g., another anticancer agent or anti-inflammatory agent.

In some embodiments, the CDP-epothilone conjugate is a polymer having attached thereto a plurality of D moieties of the following formula:

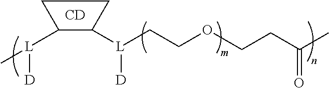

wherein each L is independently a linker, and each D is independently an epothilone, a prodrug derivative thereof, or absent, provided that the polymer comprises at least one epothilone and in some embodiments, at least two epothilone moieties; and wherein the group

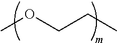

has a Mw of 3.4 kDa or less and n is at least 4, 5, 6, 7, 8, 9, 10, 11, 12, 13, 14, 15, 16, 17, 18, 19 or 20.

In some embodiments, the epothilone is an epothilone described herein, for example, the epothilone is epothilone B, ixabepilone, BMS310705, epothilone D, dehydelone, or sagopilone. The epothilone can be attached to the CDP via a functional group such as a hydroxyl group, or where appropriate, an amino group. In some embodiments, one or more of the epothilone moieties in the CDP-epothilone conjugate can be replaced with another therapeutic agent, e.g., another anticancer agent or anti-inflammatory agent.

In some embodiments, less than all of the L moieties are attached to D moieties, meaning in some embodiments, at least one D is absent. In some embodiments, the loading of the D moieties on the CDP-epothilone conjugate is from about 1 to about 50% by weight (e.g., from about 1 to about 25%, from about 5 to about 20% or from about 5 to about 15%). In some embodiments, each L independently comprises an amino acid or a derivative thereof. In some embodiments, each L independently comprises a plurality of amino acids or derivatives thereof. In some embodiments, each L is independently a dipeptide or derivative thereof.

In some embodiments, the CDP-epothilone conjugate is a polymer having attached thereto a plurality of L-D moieties of the following formula:

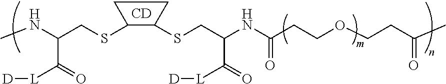

wherein each L is independently a linker or absent and each D is independently an epothilone, a prodrug derivative thereof, or absent and wherein the group

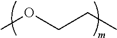

has a Mw of 3.4 kDa or less and n is at least 4, 5, 6, 7, 8, 9, 10, 11, 12, 13, 14, 15, 16, 17, 18, 19 or 20, provided that the polymer comprises at least one epothilone and in some embodiments, at least two epothilone moieties.

In some embodiments, less than all of the C(═O) moieties are attached to L-D moieties, meaning in some embodiments, at least one L and/or D is absent. In some embodiments, the loading of the L, D and/or L-D moieties on the CDP-epothilone conjugate is from about 1 to about 50% by weight (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the epothilone moieties in the CDP-epothilone conjugate can be replaced with another therapeutic agent, e.g., another anticancer agent or anti-inflammatory agent.

In some embodiments, the CDP-epothilone conjugate is a polymer having the following formula:

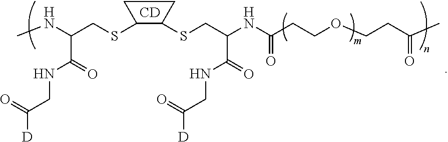

In some embodiments, less than all of the C(═O) moieties are attached to

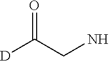

moieties, meaning in some embodiments,

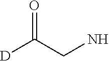

is absent, provided that the polymer comprises at least one epothilone and in some embodiments, at least two epothilone moieties. In some embodiments, the loading of the

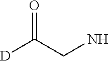

moieties on the CDP-epothilone conjugate is from about 1 to about 50% by weight (e.g., from about 1 to about 25%, from about 5 to about 20% or from about 5 to about 15%).

In some embodiments, one or more of the epothilone moieties in the CDP-epothilone conjugate can be replaced with another therapeutic agent, e.g., another anticancer agent or anti-inflammatory agent.

In some embodiments, the CDP-epothilone conjugate will contain an epothilone and at least one additional therapeutic agent. For instance, an epothilone and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

Cyclodextrins

In certain embodiments, the cyclodextrin moieties make up at least about 2%, 5% or 10% by weight, up to 20%, 30%, 50% or even 80% of the CDP by weight. In certain embodiments, the epothilones, or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 daltons, preferably from about 5000 to about 200,000 daltons and, even more preferably, from about 10,000 to about 100,000. Most preferably, Mₙ varies between about 12,000 and 65,000 daltons. In certain other embodiments, Mₙ varies between about 3000 and 150,000 daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

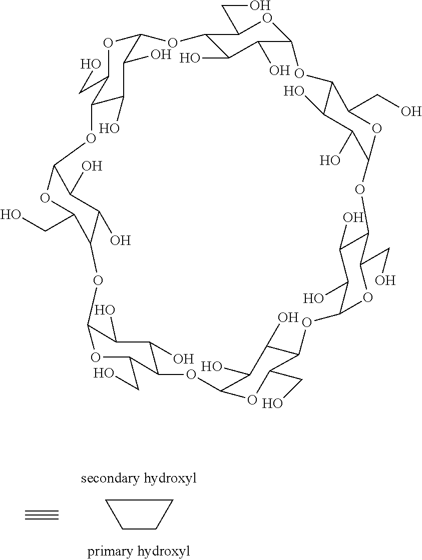

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-epothilone conjugate is oxidized. In certain embodiments, the cyclodextrin moieties of P alternate with linker moieties in the polymer chain.

### Comonomers

In addition to a cyclodextrin moiety, the CDP can also include a comonomer, for example, a comonomer described herein. In some embodiments, a comonomer of the CDP-epothilone conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-epothilone conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR₁, O or S), —OC(O)—, —C(═O)O—, —NR₁—, —NR₁CO—, —C(O)NR₁—, —S(O)ₙ— (wherein n is 0, 1, or 2), —OC(O)—NR₁—, —NR₁—C(O)—NR₁—, —NR₁—C(NR₁)—NR₁—, and —B(OR₁)—; and R₁, independently for each occurrence, represents H or a lower alkyl.

In some embodiments, a comonomer can be and/or can comprise a linker such as a linker described herein.

### Linkers/Tethers

The CDPs described herein can include one or more linkers. In some embodiments, a linker, such as a linker described herein, can link a cyclodextrin moiety to a comonomer. In some embodiments, a linker can link an epothilone to a CDP. In some embodiments, for example, when referring to a linker that links an epothilone to the CDP, the linker can be referred to as a tether.

In certain embodiments, a plurality of the linker moieties are attached to an epothilone or prodrug thereof and are cleaved under biological conditions.

Described herein are CDP-epothilone conjugates that comprise a CDP covalently attached to epothilones through attachments that are cleaved under biological conditions to release the epothilone. In certain embodiments, a CDP-epothilone conjugate comprises an epothilone covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the epothilone.

In some embodiments, such epothilones are covalently attached to a CDP through functional groups comprising one or more heteroatoms (e.g., a linker described herein), for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-epothilone conjugate comprises an epothilone covalently attached to the CDP through a tether, wherein the tether comprises a self-cyclizing moiety. In some embodiments, the tether further comprises a selectivity-determining moiety. Thus, one aspect of the invention relates to a polymer conjugate comprising a therapeutic agent covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, the invention contemplates any combination of the foregoing. Those skilled in the art will recognize that, for example, any CDP of the invention in combination with any linker (e.g., self-cyclizing moiety, any selectivity-determining moiety, and/or any epothilone) are within the scope of the invention.

In certain embodiments, the selectivity-determining moiety or linker is selected such that the bond is cleaved under acidic conditions.

In certain embodiments where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

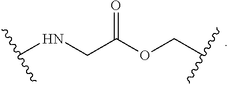 [SMILES: *COC(=O)CN*]

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA and GFLG, preferably GFLG.

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by Formula A:

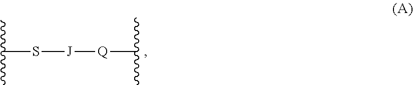

wherein
S a sulfur atom that is part of a disulfide bond;
J is optionally substituted hydrocarbyl; and
Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR³⁰, O or S), —OC(O)—, —C(═O)O—, —NR³⁰—, —NR₁CO—, —C(O)NR³⁰—, —S(O)ₙ— (wherein n is 0, 1, or 2), —OC(O)—NR³⁰—, NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

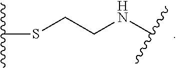 [SMILES: *NCCS*]

In certain embodiments, the selectivity-determining moiety is represented by Formula B:

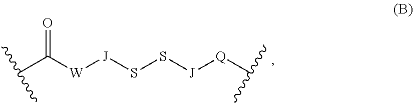

wherein
W is either a direct bond or selected from lower alkyl, NR¹⁴, S, O;
S is sulfur;
J, independently and for each occurrence, is hydrocarbyl or polyethylene glycol;
Q is O or NR¹³, wherein R¹³ is hydrogen or alkyl; and
R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{X}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{X}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR³⁰, O or S), —OC(O)—, —C(═O)O—, —NR³⁰—, —NR₁CO—, —C(O)NR³⁰—, —S(O)ₙ— (wherein n is 0, 1, or 2), —OC(O)—NR³⁰—, —NR³⁰—C(O)—NR³⁰—, —NR³⁰—C(NR³⁰)—NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

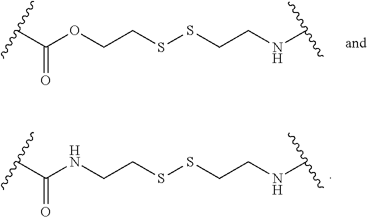 [SMILES: *NCCSSCCOC(*)=O ; *NCCSSCCNC(*)=O]

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

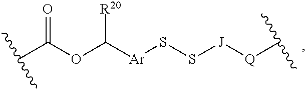

wherein
Ar is a substituted or unsubstituted benzo ring;
J is optionally substituted hydrocarbyl; and

### Q is O or NR¹³,

wherein R¹³ is hydrogen or alkyl.

In certain embodiments, Ar is unsubstituted. In certain embodiments, Ar is a 1,2-benzo ring. For example, suitable moieties within Formula B include

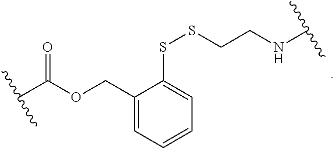 [SMILES: *NCCSSc1ccccc1COC(*)=O]

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone.

In certain embodiments, the self-cyclizing moiety has a structure

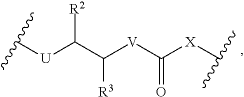

wherein
U is selected from NR¹ and S;
X is selected from O, NR⁵, and S, preferably O or S;
V is selected from O, S and NR⁴, preferably O or NR⁴;
R² and R³ are independently selected from hydrogen, alkyl, and alkoxy; or R² and R³ together
with the carbon atoms to which they are attached form a ring; and
R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl. In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together are —(CH₂)ₙ— wherein n is 3 or 4, thereby forming a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments, U is bonded to the self-cyclizing moiety.

In certain embodiments, the self-cyclizing moiety is selected from

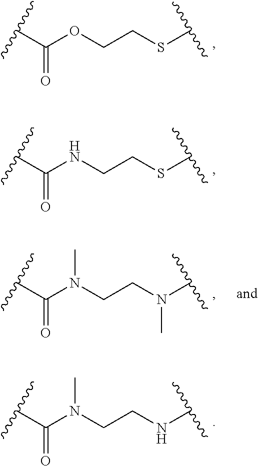

In certain embodiments, the selectivity-determining moiety may connect to the self-cyclizing moiety through carbonyl-heteroatom bonds, e.g., amide, carbamate, carbonate, ester, thioester, and urea bonds.

In certain embodiments, an epothilone is covalently attached to a polymer through a tether, wherein the tether comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the epothilone. As an illustration, ABC may be a selectivity-determining moiety, and DEFGH may be be a self-cyclizing moiety, and ABC may be selected such that enzyme Y cleaves between C and D. Once cleavage of the bond between C and D progresses to a certain point, D will cyclize onto H, thereby releasing epothilone X, or a prodrug thereof.

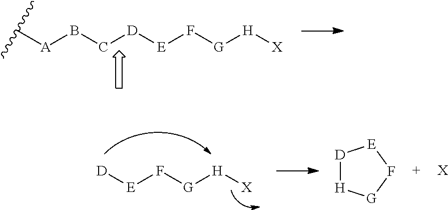

In certain embodiments epothilone X may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker may be and/or comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR₁, O or S), —OC(O)—, —C(═O)O—, —NR₁—, —NR₁CO—, —C(O)NR₁—, —S(O)ₙ— (wherein n is 0, 1, or 2), —OC(O)—NR₁—, —NR₁—C(NR₁)—NR₁—, and —B(OR₁)—; and R₁, independently for each occurrence, represents H or a lower alkyl.

In certain embodiments, a linker group, e.g., between an epothilone and the CDP, comprises a self-cyclizing moiety. In certain embodiments, a linker group, e.g., between an epothilone and the CDP, comprises a selectivity-determining moiety.

In certain embodiments as disclosed herein, a linker group, e.g., between an epothilone and the CDP, comprises a self-cyclizing moiety and a selectivity-determining moiety.

In certain embodiments as disclosed herein, the epothilone or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to epothilones or prodrugs thereof that are cleaved under biological conditions.

In certain embodiments, at least one linker that connects the epothilone or prodrug thereof to the polymer comprises a group represented by the formula

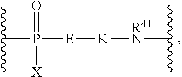

wherein
P is phosphorus;
O is oxygen;
E represents oxygen or NR⁴⁰;
K represents hydrocarbyl;
X is selected from OR⁴² or NR⁴³R⁴⁴; and
R⁴⁰, R⁴¹, R⁴², R⁴³, and R⁴⁴ independently represent hydrogen or optionally substituted alkyl.

In certain embodiments, E is NR⁴⁰ and R⁴⁰ is hydrogen.

In certain embodiments, K is lower alkylene (e.g., ethylene).

In certain embodiments, at least one linker comprises a group selected from

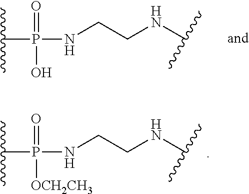

In certain embodiments, X is OR⁴².

In certain embodiments, the linker group comprises an amino acid or peptide, or derivative thereof (e.g., a glycine or cysteine).

In certain embodiments as disclosed herein, the linker is connected to the epothilone through a hydroxyl group. In certain embodiments as disclosed herein, the linker is connected to the epothilone through an amino group.

In certain embodiments, the linker group that connects to the epothilone may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X)— (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

In certain embodiments, the present invention contemplates a CDP, wherein a plurality of epothilones are covalently attached to the polymer through attachments that are cleaved under biological conditions to release the therapeutic agents as discussed above, wherein administration of the polymer to a subject results in release of the therapeutic agent over a period of at least 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month.

In some embodiments, the conjugation of the epothilone to the CDP improves the aqueous solubility of the epothilone and hence the bioavailability. Accordingly, in one embodiment of the invention, the epothilone has a log P>0.4, >0.6, >0.8, >1, >2, >3, >4, or even >5.

The CDP-epothilone of the present invention preferably has a molecular weight in the range of 10,000 to 500,000; 30,000 to 200,000; or even 70,000 to 150,000 amu.

In certain embodiments, the present invention contemplates attenuating the rate of release of the epothilone by introducing various tether and/or linking groups between the therapeutic agent and the polymer. Thus, in certain embodiments, the CDP-epothilone conjugates of the present invention are compositions for controlled delivery of the epothilone.

Epothilones

The term “epothilone,” as used herein, refers to any naturally occurring, synthetic, or semi-synthetic epothilone structure, for example, known in the art. The term epothilone also includes structures falling within the generic formulae X, XI, XII, XIII, XIV, XV, and XVI as provided herein.

Exemplary epothilones include those described generically and specifically herein. In some embodiments, the epothilone is epothilone B, ixabepilone, BMS-310705, epothilone D, dehydelone, or sagopilone. The structures of all of these epothilones are provided below:

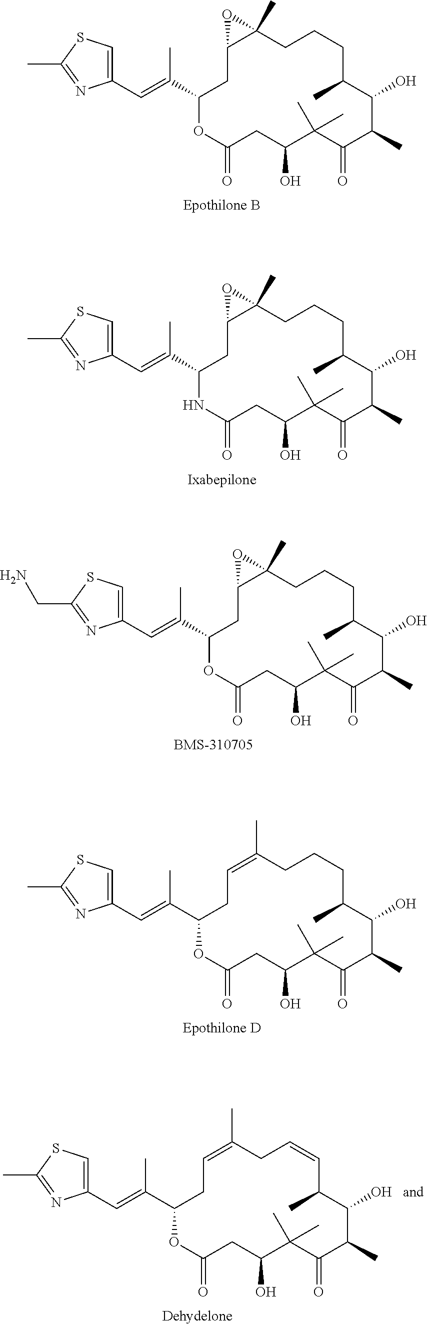

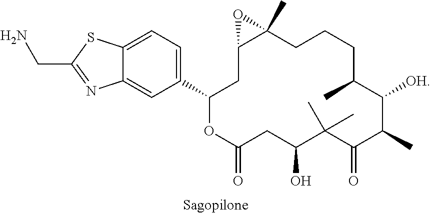 [SMILES: C[C@H]1CCC[C@@]2(C)O[C@H]2C[C@@H](c2ccc3sc(CN)nc3c2)OC(=O)C[C@H](O)C(C)(C)C(=O)[C@H](C)[C@H]1O]

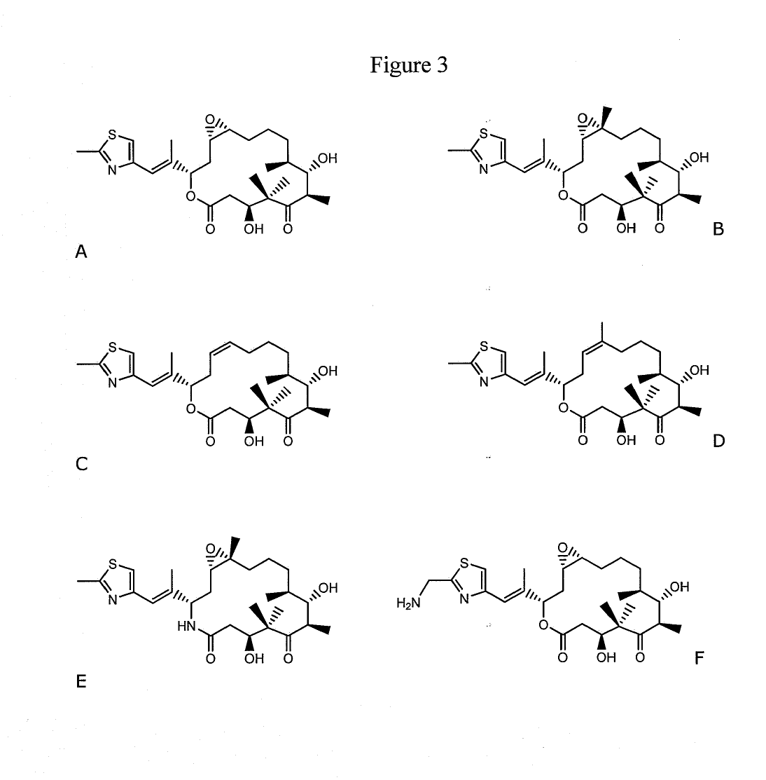

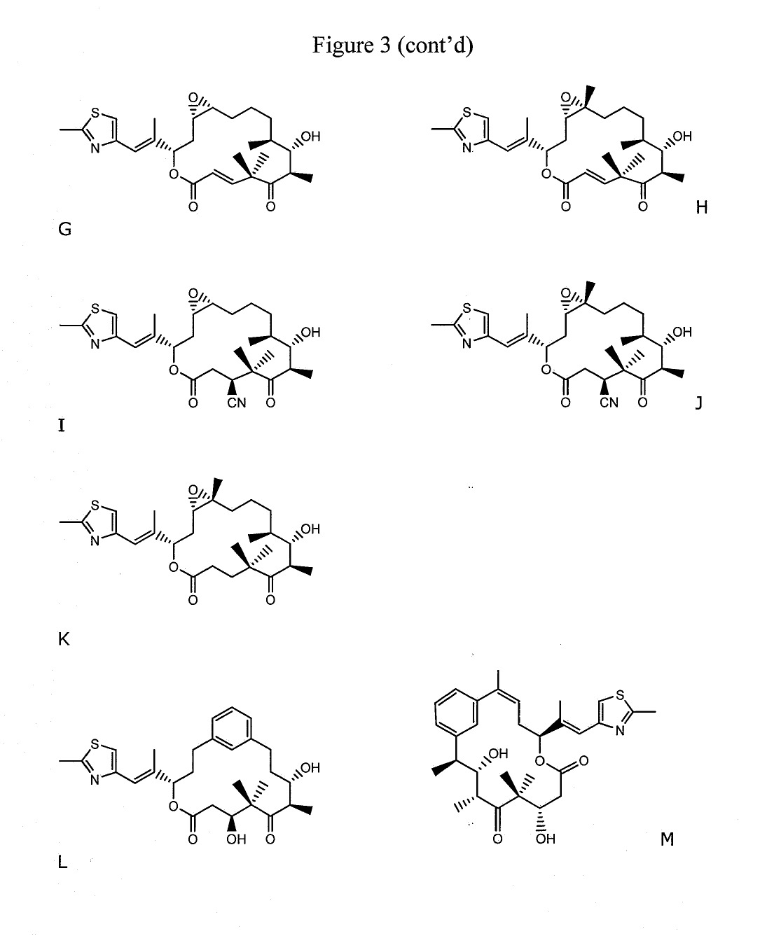

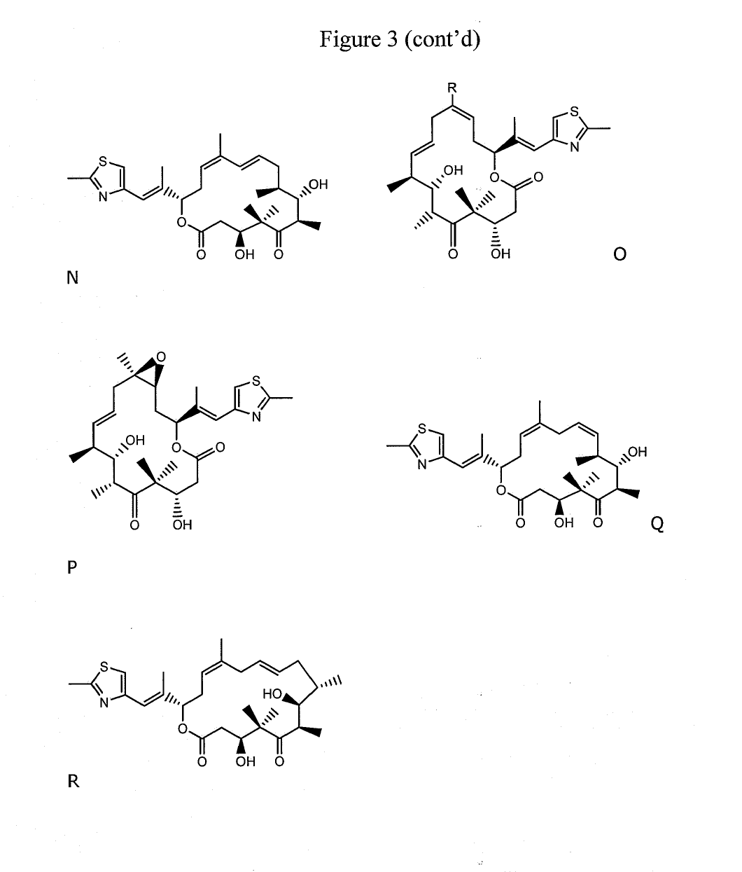

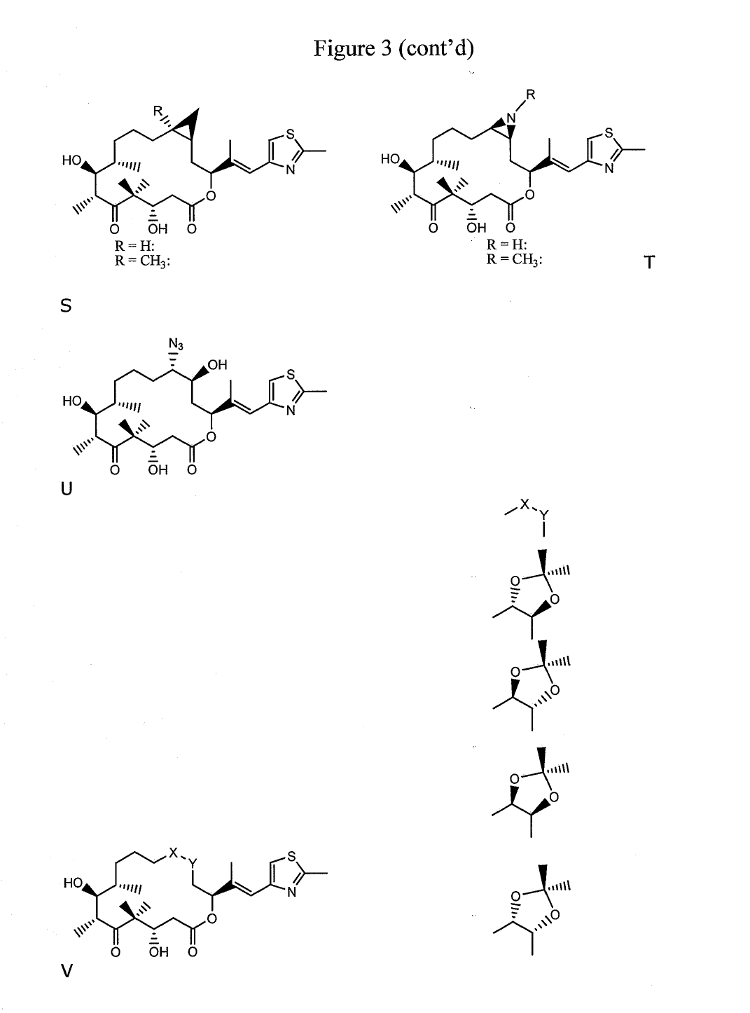

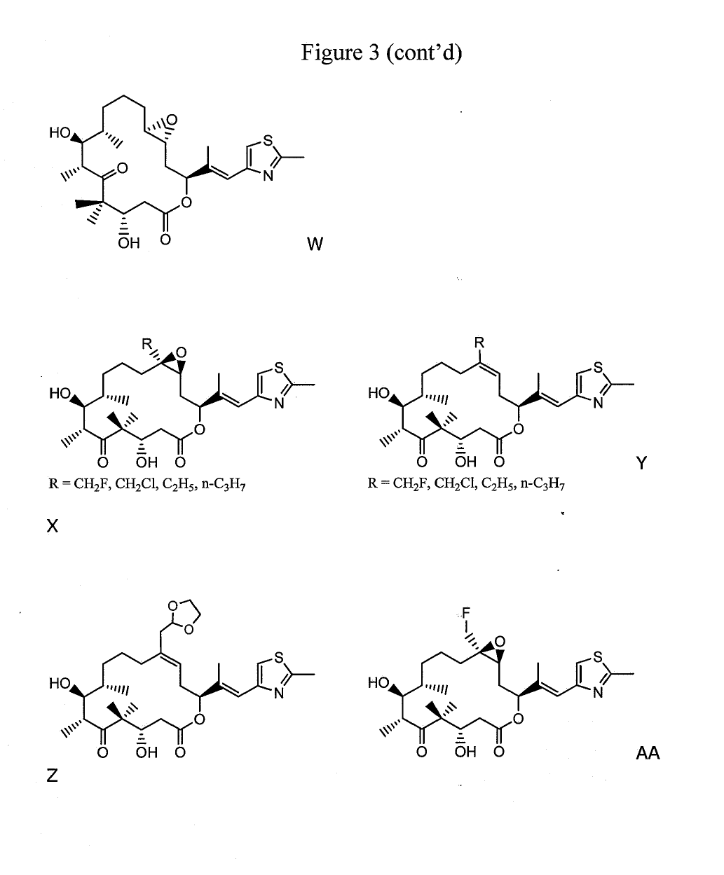

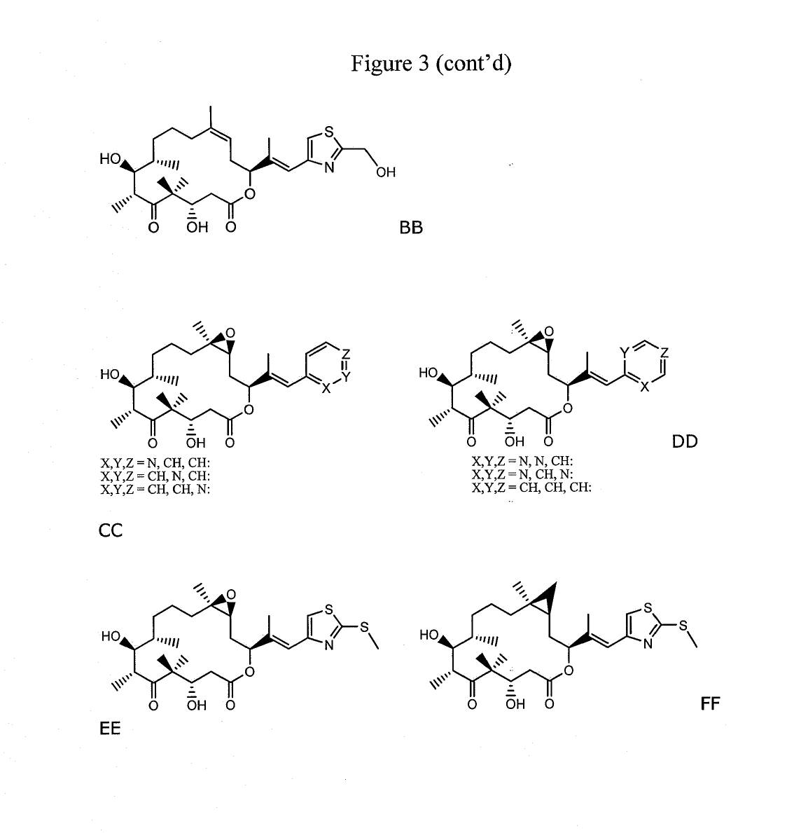

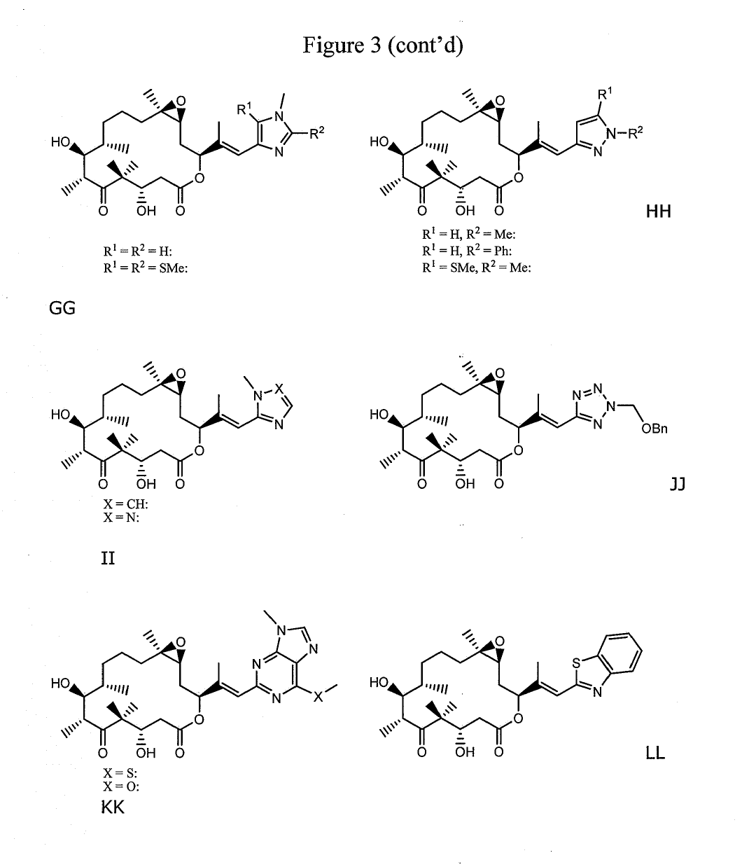

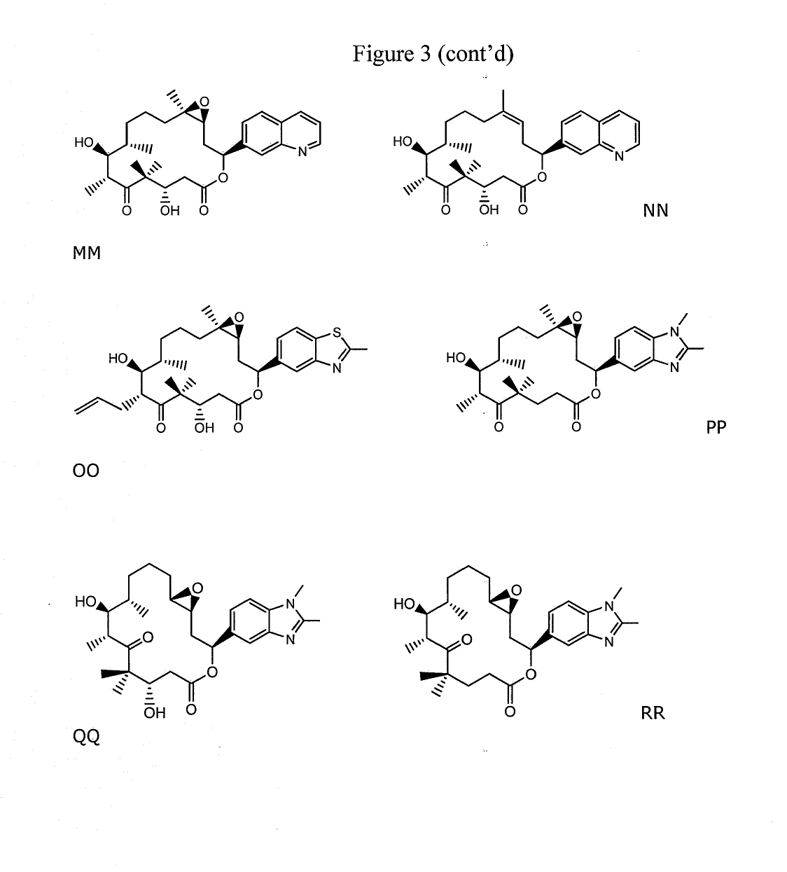

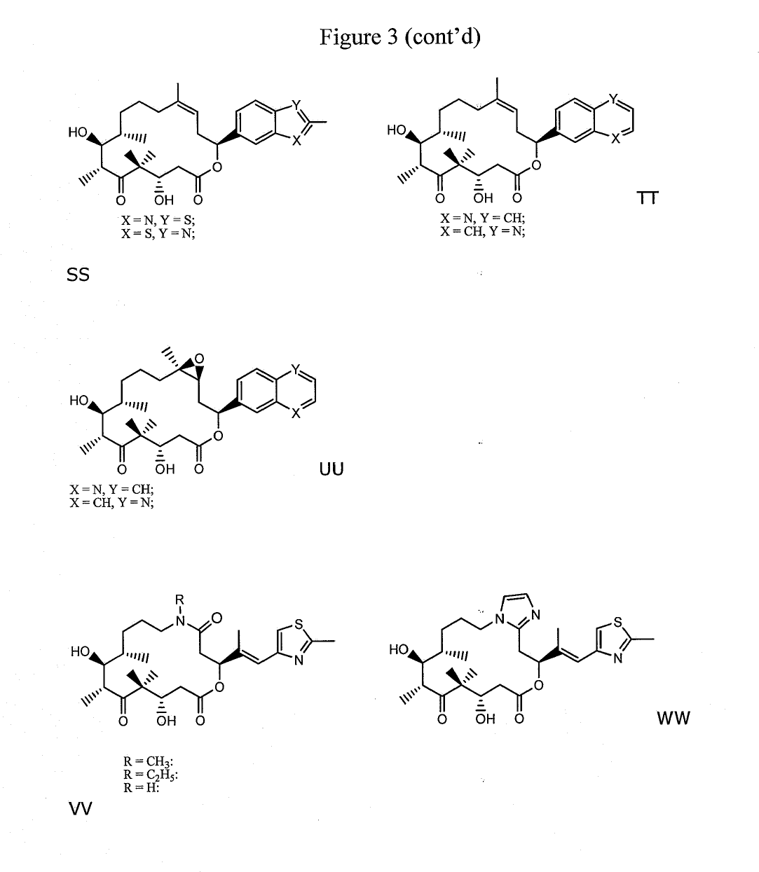

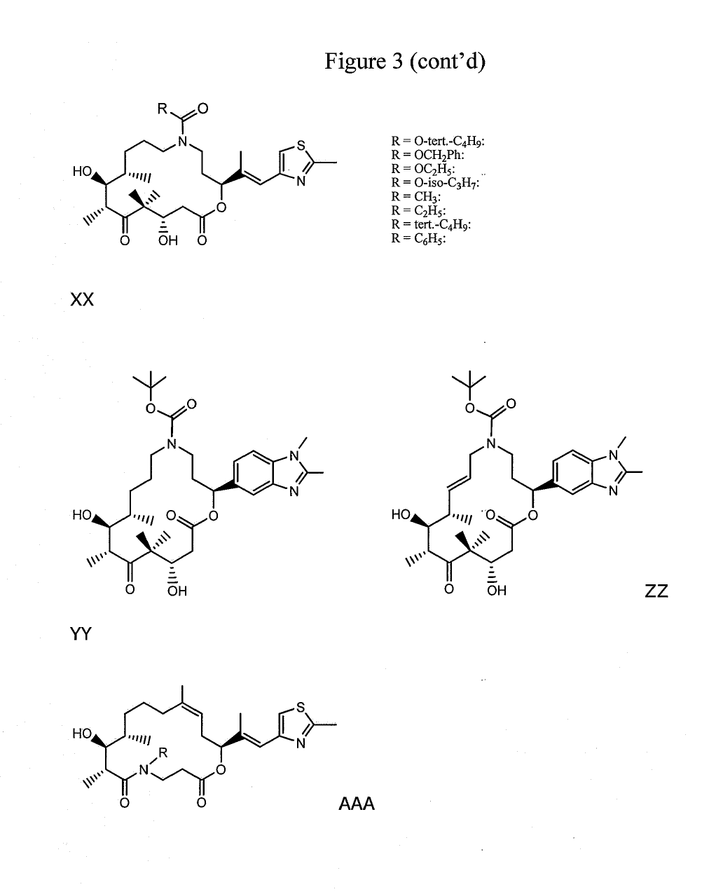

Other exemplary epothilones are also provided in FIG. 3 and disclosed in Altmann et al. “Epothilones as Lead Structures for New Anticancer Drugs-Pharmacology, Fermentation, and Structure-activity-relationships;” Progress in Drug Research (2008) Vol. 66, page 274-334, which is incorporated herein by reference.

Additionally, epothilones may be found, for example, in U.S. Pat. No. 7,317,100; U.S. Pat. No. 6,946,561; U.S. Pat. No. 6,350,878; U.S. Pat. No. 6,302,838; U.S. Pat. No. 7,030,147; U.S. Pat. No. 6,387,927; U.S. Pat. No. 6,346,404; US 2004/0038324; US 2009/0041715; US 2007/0129411; US 2005/0271669; US 2008/0139587; US 2004/0235796; US 2005/0282873; US 2006/0089327; WO 2008/071404; WO 2008/019820; WO 2007/121088; WO 1998/08849; EP 1198225; EP 1420780; EP 1385522; EP 1539768; EP 1485090; and EP 1463504, the contents of these references are incorporated herein in their entireties.

Further epothilones may be found, for example, in U.S. Pat. No. 6,410,301; U.S. Pat. No. 7,091,193; U.S. Pat. No. 7,402,421; U.S. Pat. No. 7,067,286; U.S. Pat. No. 6,489,314; U.S. Pat. No. 6,589,968; U.S. Pat. No. 6,893,859; U.S. Pat. No. 7,176,235; U.S. Pat. No. 7,220,560; U.S. Pat. No. 6,280,999; U.S. Pat. No. 7,070,964; US 2005/0148543; US 2005/0215604; US 2003/0134883; US 2008/0319211; US 2005/0277682; US 2005/0020558; US 2005/0203174; US 20020045609, US 2004/0167097; US 2004/0072882; US 2002/0137152; WO 2009/064800; and WO 2002/012534, the contents of these references are incorporated herein in their entireties.

Further epothilones may be found, for example, in U.S. Pat. No. 6,537,988; U.S. Pat. No. 7,312,237; U.S. Pat. No. 7,022,330; U.S. Pat. No. 6,670,384; U.S. Pat. No. 6,605,599; U.S. Pat. No. 7,125,899; U.S. Pat. No. 6,399,638; U.S. Pat. No. 7,053,069; U.S. Pat. No. 6,936,628; U.S. Pat. No. 7,211,593; U.S. Pat. No. 6,686,380; U.S. Pat. No. 6,727,276; U.S. Pat. No. 6,291,684; U.S. Pat. No. 6,780,620; U.S. Pat. No. 6,719,540; US 2009/0004277; US 2007/0276018; WO 2004/078978; and EP 1157023, the contents of these references are incorporated herein in their entireties.

Further epothilones may be found, for example, in US 2008/0146626; US 2009/0076098; WO 2009/003706 and WO 2009/074274, the contents of these references are incorporated herein in their entireties.

Further epothilones may be found, for example, in U.S. Pat. No. 7,169,930; U.S. Pat. No. 6,294,374; U.S. Pat. No. 6,380,394; and U.S. Pat. No. 6,441,186, the contents of these references are incorporated herein in their entireties.

Further epothilones may be found, for example, in U.S. Pat. No. 7,119,071; and German Application Serials Nos. DE 197 13 970.1, DE 100 51 136.8, DE 101 34 172.5, and DE 102 32 094.2, the contents of these references are incorporated herein in their entireties.

In some embodiments, the epothilone or CDP-epothilone conjugate is attached to a targeting moiety such as a folate moiety. In some embodiments, the targeting moiety (e.g., a folate) is attached to a functional group on the epothilone such as a hydroxyl group or an amino group where appropriate. In some embodiments, the folate is attached to the epothilone directly.

In some embodiments, the folate is attached to the epothilone via a linker Epofolate (BMS-753493) is an example an epothilone attached to a folate, see, for example, U.S. Pat. No. 7,033,594, which is incorporated herein by reference.

In one embodiment, the epothilone is a compound of formula (X)

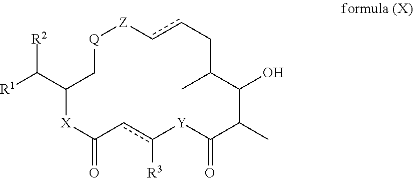

wherein

R¹ is aryl, heteroaryl, arylalkenyl or heteroarylalkenyl; each of which is optionally substituted with 1-3 R⁸;

R² is H or alkyl (e.g., a methyl); or

R¹ and R², when taken together with the carbon to which they are attached, form an aryl or a heteroaryl moiety optionally substituted with 1-3 R⁸;

R³ is H, OH, NH₂, or CN;

X is O or NR⁴;

R⁴ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

each R⁸, for each occurrence, is independently alkyl, aminoalkyl, hydroxyalkyl, alkylthiol, aryl, arylalkyloxyalkyl or alkoxy;

Q-Z, when taken together, form

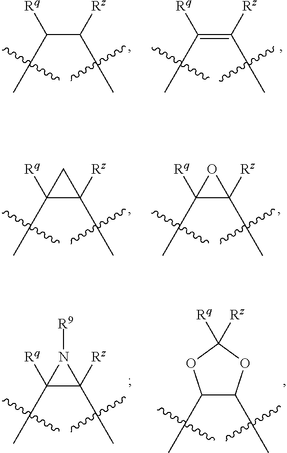

heteroarylenyl, C(O)NR⁴, NR⁴C(O), CR⁵R⁶NR⁴, or NR⁴CR⁵R⁶;

R^{q} is H, alkyl (e.g., methyl) or hydroxy;

R^{z} is H, alkyl (e.g., methyl), haloalkyl (e.g., CF₃), heterocyclylalkyl or N₃;

R⁹ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl; and

each [symbol] , for each occurrence, is independently a single or double bond.

In some embodiments, R¹ is

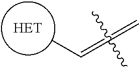 [SMILES: *=CC]

optionally substituted with 1-3 R⁸.

In some embodiments, HET is a five membered ring heteroaryl optionally substituted with 1-3 R⁸.

In some embodiments, HET is a thiazolyl optionally substituted with 1-3 R⁸. In some embodiments, HET is substituted with alkyl (e.g., methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl).

In some embodiments, HET is substituted with alkyl (e.g., methyl) or amino alkyl.

In some embodiments, HET is

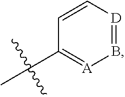

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

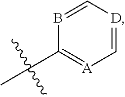

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

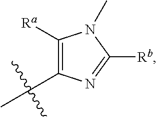

wherein each R^{a} and R^{b} is independently H or —SMe.

In some embodiments, HET is

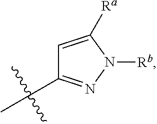

wherein each R^{a} is H, alkyl or —Salkyl; and R^{b} is H, alkyl (e.g., methyl) or aryl (e.g., phenyl).

In some embodiments, HET is

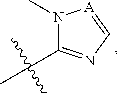

wherein A is CH or N.

In some embodiments, HET is

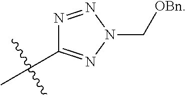 [SMILES: *c1nnn(COCc2ccccc2)n1]

In some embodiments, HET is

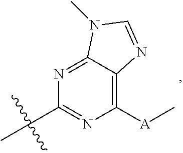

wherein A is S or O.

In some embodiments, HET is

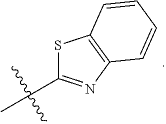 [SMILES: *c1nc2ccccc2s1]

In some embodiments R² is H.

In some embodiments, R² is alkyl (e.g., methyl).

In some embodiments, R¹ and R², when taken together with the carbon to which they are attached, form an aryl or a heteroaryl moiety optionally substituted with 1-3 R⁸.

In some embodiments, R¹ and R², when taken together with the carbon to which they are attached, form a heteroaryl moiety optionally substituted with 1-3 R⁸.

In some embodiments, the heteroaryl moiety is a bicyclic heteroaryl moiety.

In some embodiments, R¹ and R², when taken together with the carbon to which they are attached, are

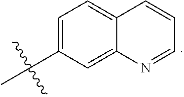 [SMILES: *c1ccc2cccnc2c1]

In some embodiments, R¹ and R², when taken together with the carbon to which they are attached, are

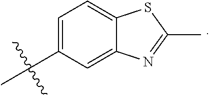 [SMILES: *c1ccc2sc(C)nc2c1]

In some embodiments, R¹ and R², when taken together with the carbon to which they are attached, are

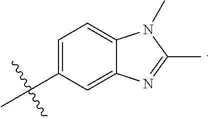 [SMILES: *c1ccc2c(c1)nc(C)n2C]

In some embodiments, R¹ and R², when taken together with the carbon to which they are attached, are

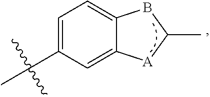

wherein A is N and B is S or wherein A is S and B is N.

In some embodiments, R¹ and R², when taken together with the carbon to which they are attached, are

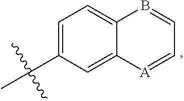

wherein A is N and B is CH or wherein A is CH and B is N.

In some embodiments,

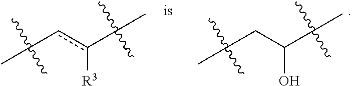

In some embodiments,

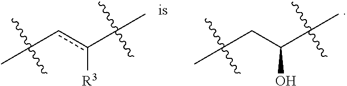

In some embodiments,

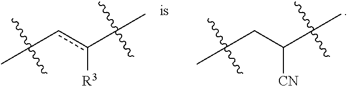

In some embodiments,

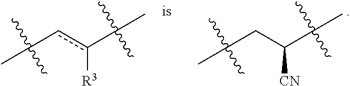

In some embodiments,

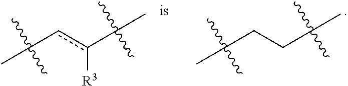

In some embodiments,

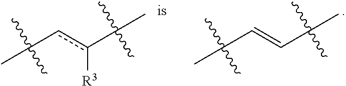

In some embodiments, X is O.

In some embodiments, X is NR⁴ (e.g., NH).

In some embodiments, Y is CR⁵R⁶. In some embodiments, Y is

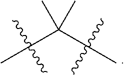 [SMILES: *C(*)(C)C]

In some embodiments, Y is CH₂.

In some embodiments, Y is NR⁷ (e.g., NH or NMe).

In some embodiments, Q-Z, when taken together, form

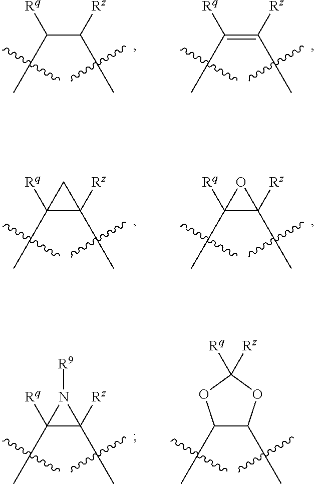

or heteroarylenyl.

In some embodiments, Q-Z, when taken together, form

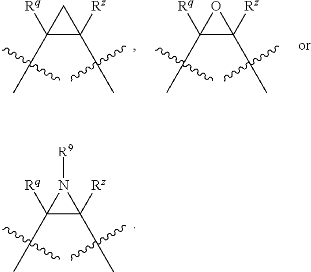

In some embodiments, Q-Z, when taken together, form

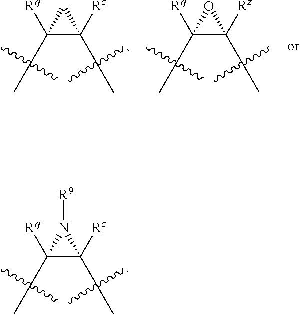

In some embodiments, Q-Z, when taken together, form

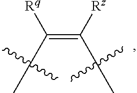

wherein R^{q} is H and R^{z} is H or alkyl (e.g., methyl).

In some embodiments, Q-Z, when taken together, form

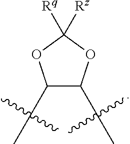

In some embodiments, both R^{q} and R^{z} are methyl. In some embodiments,

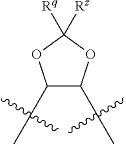

is selected from

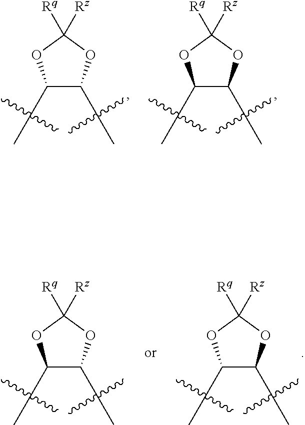

In some embodiments, both R^{q} and R^{z} are methyl.

In some embodiments, Q-Z, when taken together, form a heteroarylenyl. In some embodiments, Q-Z, when taken together, form

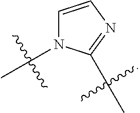 [SMILES: *c1nccn1*]

In some embodiments, Q-Z, when taken together, form C(O)NR⁴. In some embodiments, R⁴ is H or alkyl (e.g., methyl or ethyl).

In some embodiments, Q-Z, when taken together, form NR⁴C(O). In some embodiments, R⁴ is H or alkyl (e.g., methyl or ethyl).

In some embodiments, Q-Z, when taken together, form CH₂NR⁴. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, Q-Z, when taken together, form NR⁴CH₂. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, the compound of formula (X) is a compound of formula (Xa)

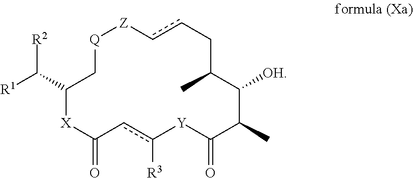

In some embodiments, the compound of formula (X) is a compound of formula (Xb)

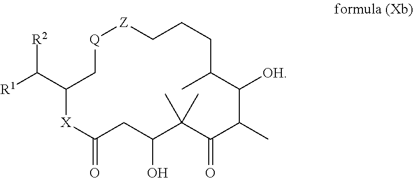

In some embodiments, the compound of formula (X) is a compound of formula (XC)

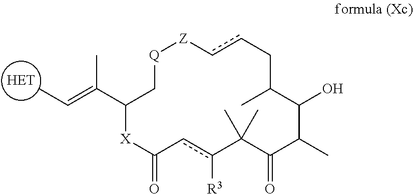

wherein HET is an optionally substituted heteroaryl.

In some embodiments, HET is an optionally substituted 5 membered ring.

In some embodiments, the compound of formula (X) is a compound of formula (Xd)

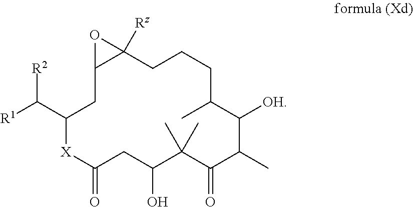

In some embodiments, the compound of formula (X) is a compound of formula (Xe)

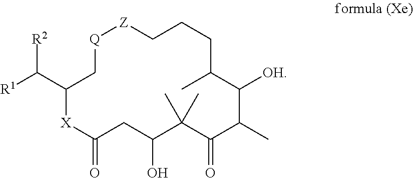

In some embodiments, the compound of formula (X) is a compound of formula (Xf)

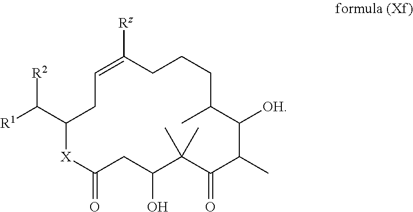

In some embodiments, the compound of formula (X) is a compound of formula (Xg)

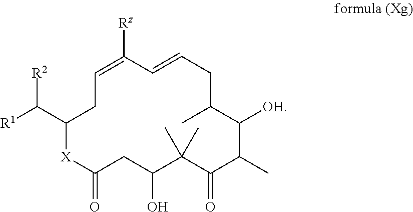

In one embodiment, the epothilone is a compound of formula (XI)

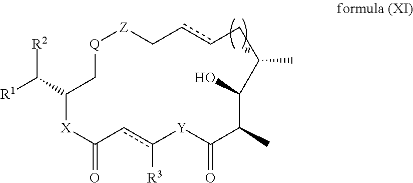

wherein

R¹ is aryl, heteroaryl, arylalkenyl, or heteroarylalkenyl; each of which is optionally substituted with 1-3 R⁸;

R² is H or alkyl (e.g., methyl); or

R¹ and R², when taken together with the carbon to which they are attached, form an aryl or a heteroaryl moiety optionally substituted with 1-3 R⁸;

R³ is H, OH, NH₂ or CN;

X is O or NR⁴;

R⁴ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

each R⁸, for each occurrence, is independently alkyl, aminoalkyl, hydroxyalkyl, alkylthiol, aryl, arylalkyloxyalkyl or alkoxy;

Q-Z, when taken together, form

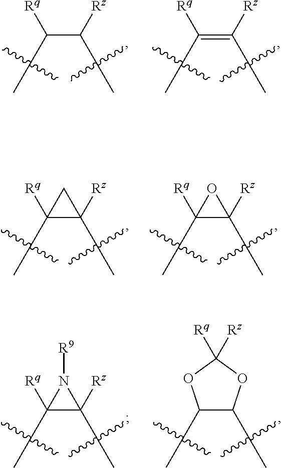

heteroarylenyl, C(O)NR⁴, NR⁴C(O), CR⁵R⁶NR⁴, or NR⁴CR⁵R⁶NR⁴;

R^{q} is H, alkyl (e.g., methyl) or hydroxy;

R^{z} is H, alkyl (e.g., methyl), haloalkyl (e.g., CF₃), heterocyclylalkyl or N₃;

R⁹ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

each [symbol] , for each occurrence, is independently a single or double bond; and

n is 0, 1 or 2.

In some embodiments, R¹ is

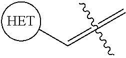

optionally substituted with 1-3 R⁸. In some embodiments, HET is a five membered ring heteroaryl optionally substituted with 1-3 R⁸. In some embodiments, HET is a thiazolyl optionally substituted with 1-3 R⁸. In some embodiments, HET is substituted with alkyl (e.g., a methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl). In some embodiments, HET is substituted with alkyl (e.g., methyl) or aminoalkyl.

In some embodiments, HET is

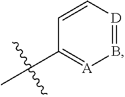

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

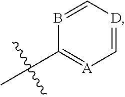

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

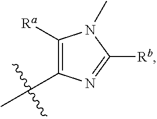

wherein each R^{a} and R^{b} is independently —H or —SMe.

In some embodiments, HET is

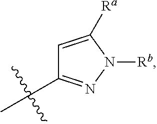

wherein each R^{a} is H, alkyl or —Salkyl; and R^{b} is H, alkyl (e.g., methyl) or aryl (e.g., phenyl).

In some embodiments, HET is

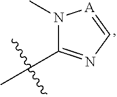

wherein A is CH or N.

In some embodiments, HET is

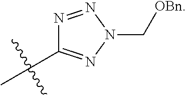 [SMILES: *c1nnn(COCc2ccccc2)n1]

In some embodiments, HET is

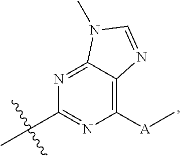

wherein A is S or O.

In some embodiments, HET is

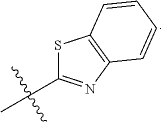 [SMILES: *c1nc2ccccc2s1]

In some embodiments R² is H.

In some embodiments, R² is alkyl (e.g., methyl).

In some embodiments, R¹ and R², when taken together with the carbon to which they are attached, form an aryl or a heteroaryl moiety optionally substituted with 1-3 R⁸. In some embodiments, the heteroaryl moiety is a bicyclic heteroaryl moiety.

In some embodiments, R¹ and R², when taken together with the carbon to which they are attached, are

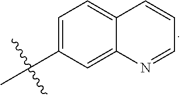 [SMILES: *c1ccc2cccnc2c1]

In some embodiments, R¹ and R², when taken together with the carbon to which they are attached, are

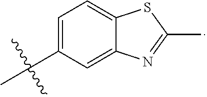 [SMILES: *c1ccc2sc(C)nc2c1]

In some embodiments, R¹ and R², when taken together with the carbon to which they are

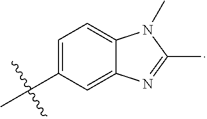 [SMILES: *c1ccc2c(c1)nc(C)n2C]

attached, are

In some embodiments, R¹ and R², when taken together with the carbon to which they are attached, are

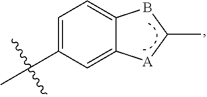

wherein A is N and B is S or wherein A is S and B is N.

In some embodiments, R¹ and R², when taken together with the carbon to which they are attached, are

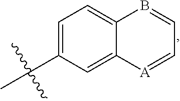

wherein A is N and B is CH or wherein A is CH and B is N.

In some embodiments,

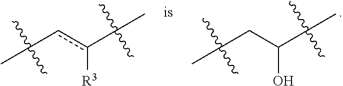

In some embodiments,

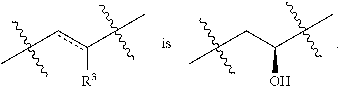

In some embodiments,

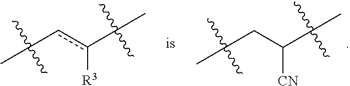

In some embodiments,

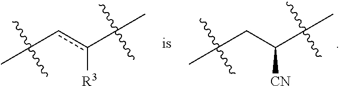

In some embodiments,

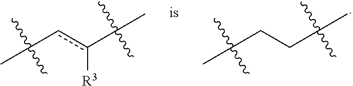

In some embodiments,

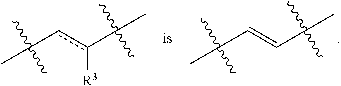

In some embodiments, X is O.

In some embodiments, X is NR⁴ (e.g., NH).

In some embodiments, Y is CR⁵R⁶.

In some embodiments, Y is

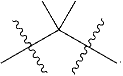 [SMILES: *C(*)(C)C]

In some embodiments, Y is CH₂.

In some embodiments, Y is NR⁷ (e.g., NH or NMe).

In some embodiments, Q-Z, when taken together, form

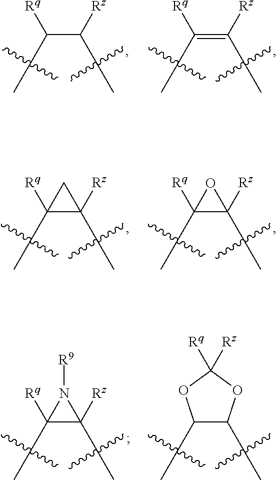

or heteroarylenyl.

In some embodiments, Q-Z, when taken together, form

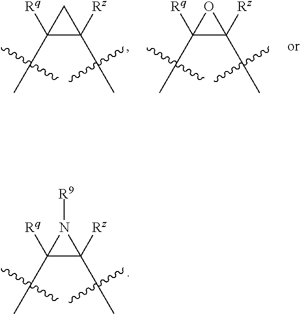

In some embodiments, Q-Z, when taken together, form

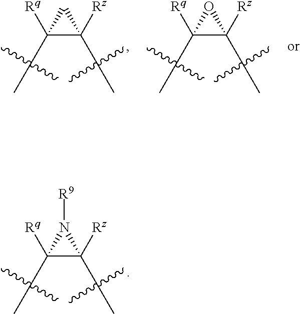

In some embodiments, Q-Z, when taken together, form

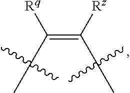

wherein R^{q} is H and R^{z} is H or alkyl (e.g., methyl).

In some embodiments, Q-Z, when taken together, form

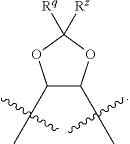

In some embodiments, both R^{q} and R^{z} are methyl.

In some embodiments,

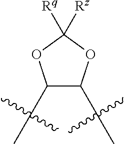

is selected from

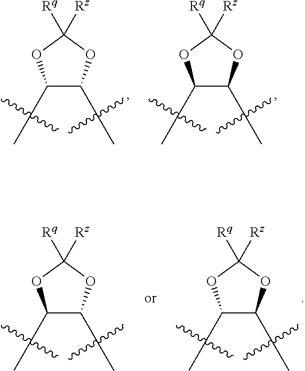

In some embodiments, both R^{q} and R^{z} are methyl.

In some embodiments, Q-Z, when taken together, form a heteroarylenyl. In some embodiments, Q-Z, when taken together, form

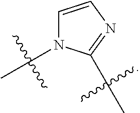 [SMILES: *c1nccn1*]

In some embodiments, Q-Z, when taken together, form C(O)NR⁴. In some embodiments, R⁴ is H or alkyl (e.g., methyl or ethyl).

In some embodiments, Q-Z, when taken together, form NR⁴C(O). In some embodiments, R⁴ is H or alkyl (e.g., methyl or ethyl).

In some embodiments, Q-Z, when taken together, form CH₂NR⁴. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, Q-Z, when taken together, form NR⁴—CH₂. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, n is 0.

In some embodiments, n is 1.

In some embodiments, the compound of formula (XI) is a compound of formula (XIa)

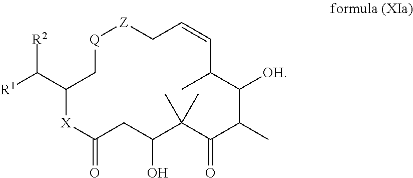

In some embodiments, the compound of formula (XI) is a compound of formula (XIb)

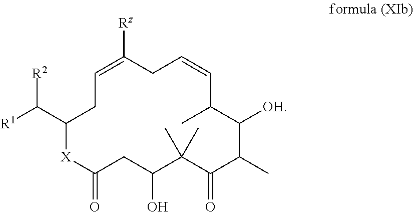

In some embodiments, the compound of formula (XI) is a compound of formula (XIc)

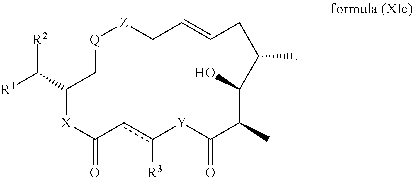

In some embodiments, the epothilone is a compound of formula (XII)

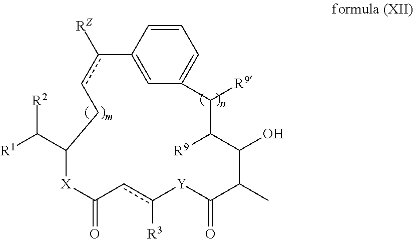

wherein,

R¹ is aryl, heteroaryl, arylalkenyl or heteroarylalkenyl; each of which is optionally substituted with 1-3 R⁸;

R² is H or alkyl (e.g., methyl); or

R¹ and R², when taken together with the carbon to which they are attached, form an aryl or a heteroaryl moiety optionally substituted with 1-3 R⁸;

R³ is H, OH, NH₂, or CN;

X is O or NR⁴;

R⁴ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

each R⁸, for each occurrence, is independently alkyl, aminoalkyl or hydroxyalkyl;

each R⁹ and R⁹′ is independently H or alkyl (e.g., methyl);

R^{z} is H, alkyl (e.g., methyl), haloalkyl (e.g., CF₃), heterocyclylalkyl or N₃;

each [symbol] , for each occurrence, is independently a single or double bond;

m is 0, 1 or 2; and

n is 0, 1 or 2.

In some embodiments, R¹ is

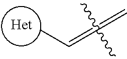

optionally substituted with 1-3 R⁸. In some embodiments, HET is a five membered ring heteroaryl optionally substituted with 1-3 R⁸. In some embodiments, HET is thiazolyl optionally substituted with 1-3 R⁸. In some embodiments, HET is substituted with alkyl (e.g., methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl). In some embodiments, HET is substituted with alkyl (e.g., methyl) or amino alkyl.

In some embodiments, HET is

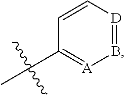

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

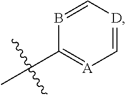

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

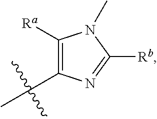

wherein each R^{a} and R^{b} is independently H or —SMe.

In some embodiments, HET is

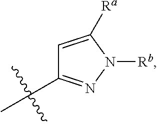

wherein each R^{a} is H, an alkyl or —Salkyl; and R^{b} is H, alkyl (e.g., methyl) or aryl (e.g., phenyl).

In some embodiments, HET is

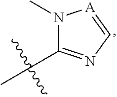

wherein A is CH or N.

In some embodiments, HET is

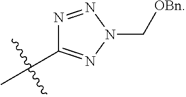 [SMILES: *c1nnn(COCc2ccccc2)n1]

In some embodiments, HET is

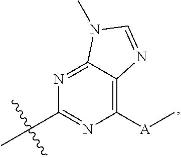

wherein A is S or 0.

In some embodiments, HET is

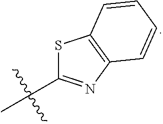 [SMILES: *c1nc2ccccc2s1]

In some embodiments R² is H.

In some embodiments, R² is alkyl (e.g., methyl).

In some embodiments, R¹ and R², when taken together with the carbon to which they are attached, form an aryl or a heteroaryl moiety optionally substituted with 1-3 R⁸.

In some embodiments, the heteroaryl moiety is a bicyclic heteroaryl moiety.

In some embodiments, R¹ and R², when taken together with the carbon to which they are attached, are

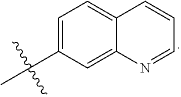 [SMILES: *c1ccc2cccnc2c1]

In some embodiments, R¹ and R², when taken together with the carbon to which they are attached, are

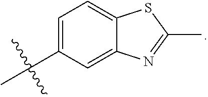 [SMILES: *c1ccc2sc(C)nc2c1]

In some embodiments, R¹ and R², when taken together with the carbon to which they are attached, are

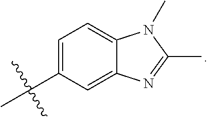 [SMILES: *c1ccc2c(c1)nc(C)n2C]

In some embodiments, R¹ and R², when taken together with the carbon to which they are attached, are

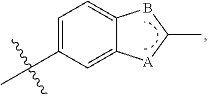

wherein A is N and B is S or wherein A is S and B is N.

In some embodiments, R¹ and R², when taken together with the carbon to which they are attached, are

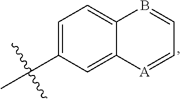

wherein A is N and B is CH or wherein A is CH and B is N.

In some embodiments,

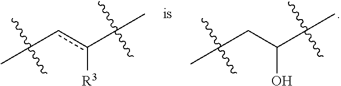

In some embodiments,

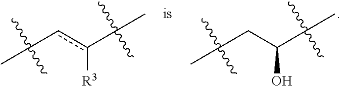

In some embodiments,

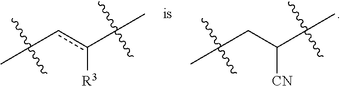

In some embodiments,

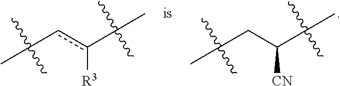

In some embodiments,

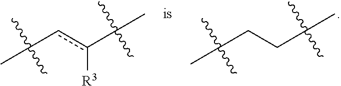

In some embodiments,

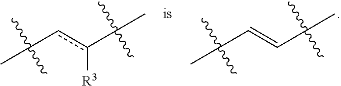

In some embodiments, X is O.

In some embodiments, X is NR⁴ (e.g., NH).

In some embodiments, Y is CR⁵R⁶. In some embodiments, Y is

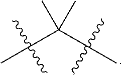 [SMILES: *C(*)(C)C]

In some embodiments, Y is CH₂.

In some embodiments, Y is NR⁷ (e.g., NH or NMe).

In some embodiments, R⁹ is H.

In some embodiments, R⁹ is Me.

In some embodiments,

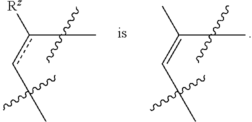

In some embodiments, m is 1.

In some embodiments,

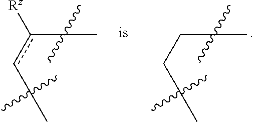

In some embodiments, m is 0.

In some embodiments, n is 0.

In some embodiments,

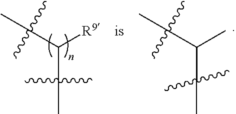

In some embodiments, compound of formula (XII) is a compound of formula (XIIa)

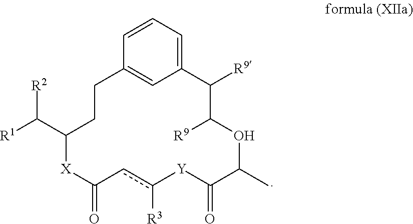

In some embodiments, compound of formula (XII) is a compound of formula (XIIb)

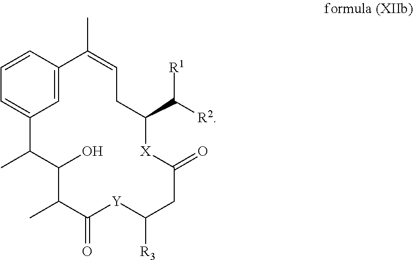

In some embodiments, the epothilone is a compound of formula (XIII):

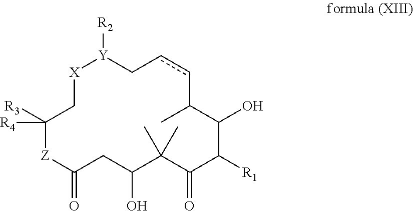

wherein

[symbol] represents a single or double bond;

R₁ is C₁₋₆alkyl, C₂₋₆alkynyl or C₂₋₆alkenyl radical;

R₂ is H or C₁₋₆alkyl radical;

X—Y is selected from the following groups:

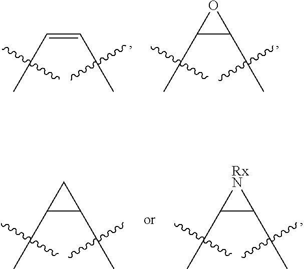

preferably

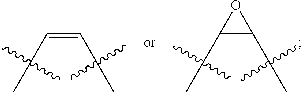

Z is O or NRₓ, wherein Rₓ is hydrogen, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;

R₃ is halogen atom or C₁₋₆alkyl, C₂₋₆alkenyl or C₁₋₆-heteroalkyl radical;

R₄ is bicycloaryl, bicycloheteroaryl or a group of formula —C(R₅)═CHR₆;

R₅ is H or methyl; and

R₆ is an optionally substituted aryl or a heteroaryl group.

In certain embodiments, R₄ is

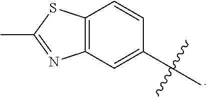 [SMILES: *c1ccc2sc(C)nc2c1]

In some embodiments, Z is O. In some embodiments, Z is NH.

In certain embodiments, the compound of formula (XIII) can be represented by the following structures:

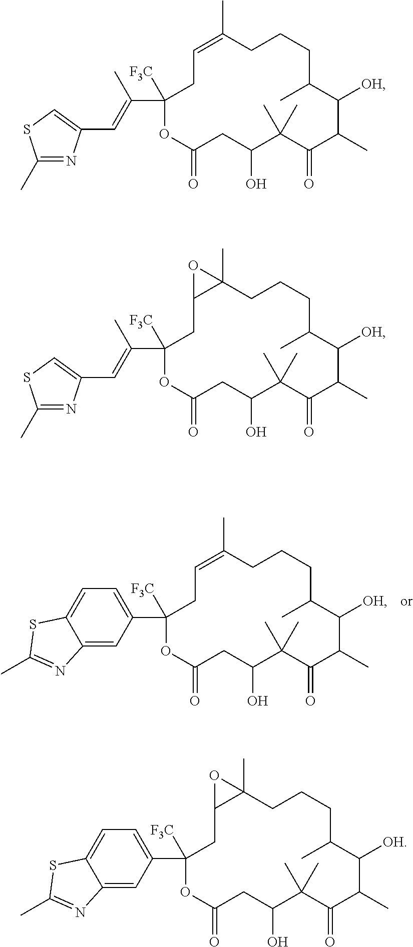 [SMILES: C/C1=C/CC(/C(C)=C/c2csc(C)n2)(C(F)(F)F)OC(=O)CC(O)C(C)(C)C(=O)C(C)C(O)C(C)CCC1 ; C/C(=C\c1csc(C)n1)C1(C(F)(F)F)CC2OC2(C)CCCC(C)C(O)C(C)C(=O)C(C)(C)C(O)CC(=O)O1 ; C/C1=C/CC(c2ccc3sc(C)nc3c2)(C(F)(F)F)OC(=O)CC(O)C(C)(C)C(=O)C(C)C(O)C(C)CCC1 ; Cc1nc2cc(C3(C(F)(F)F)CC4OC4(C)CCCC(C)C(O)C(C)C(=O)C(C)(C)C(O)CC(=O)O3)ccc2s1]

In some embodiments, the epothilone is a compound of formula (XIV):

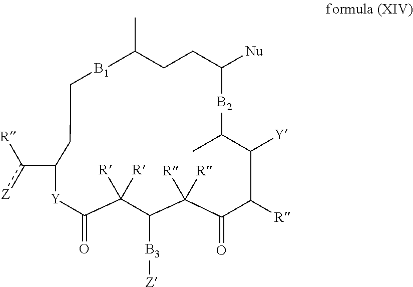

wherein

B₁, B₂, B₃ are selected from single bonds; double bonds in the E(trans) form, the Z(cis) form or as an E/Z mixture; epoxide rings in the E(trans) form, the Z(cis) form or an E/Z mixture; aziridine rings in the E(trans) form, the Z(cis) form or an E/Z mixture; cyclopropane rings in the E(trans) form, the Z(cis) form or an E/Z mixture; and/or combinations thereof; and being preferably selected from single and double bonds; and particularly preferably being selected from B₁ as Z double bonds or epoxide and B₂ and B₃ as single bond;

R is selected from H, alkyl, aryl, aralkyl (such as —CH₂-aryl, —C₂H₄-aryl and the like), alkenyl (such as vinyl), cycloalkyl (preferably a 3- to 7-membered cycloalkyl), CHₙF₃₋ₙ wherein n=0 to 3, oxacycloalkyl (preferably a 3- to 7-membered oxacycloalkyl) and/or combinations thereof. Preferably R is selected from H, methyl, ethyl, phenyl, benzyl and combinations thereof, and more preferably R is selected from H, methyl, ethyl and combinations thereof;

R′ is selected from the same group as R, and is preferably H;

R″ is selected from the same group as R, and is preferably methyl;

Y is selected from S, NH, N-PG, NR and O; preferably Y is selected from NH, N-PG, NR and O, and more preferably Y is O;

Y′ is selected from H, OH, OR, O-PG, NH₂, NR₂, N(PG)₂, SR and SH; preferably Y′ is O-PG and/or OH;

Nu is selected from R, O-PG, OR, N(PG)₂, NR₂, S-PG, SR, SeR, CN, N₃, aryl and heteroaryl; Nu is preferably selected from R, O-PG, OR, N(PG)₂ and NR₂, and more preferably Nu is H;

Z is selected from —OH, —O-PG, —OR, ═O, ═N-Nu, ═CH-heteroaryl, ═CH-aryl and ═PR₃, where all previously mentioned double bound groups may be present in the E(trans) form, the Z(cis) form or as an E/Z mixture; preferably Z is ═CH-heteroaryl; and more preferably Z is selected from ═O, (E)-(2-methylthiazol-4-yl)-CH═ and (E)-(2-methyloxazol-4-yl)-CH═;

Z′ is selected from O, OH, OR, O-PG, N(H)₁₋₂, N(R)₁₋₂, N(PG)₁₋₂, SR, S-PG and R; preferably Z′ is O, O-PG and/or OR;

B₃ is selected from single or double bonds in the E(trans) form, the Z(cis) form or as an E/Z mixture; preferably B₃ is selected from single and double bonds with heteroatoms such as O, S and N; and more preferably B₃ is a single bond to O-PG and/or OH;

PG, as referred to herein, is a protecting group, and is preferably selected from allyl, methyl, t-butyl (preferably with electron withdrawing group), benzyl, silyl, acyl and activated methylene derivative (e.g., methoxymethyl), alkoxyalkyl or 2-oxacycloalkyl. Exemplary protecting groups for alcohol and amines include trimethylsilyl, triethylsilyl, dimethyl-tert-butylsilyl, acetyl, propionyl, benzoyl, or a tetrahydropyranyl protecting group. Protecting groups can also be used to protect two neighboring groups (e.g., —CH(OH)—CH(OH)—), or bivalent groups (PG₂). Such protecting groups can form a ring such as a 5- to 7-membered ring. Exemplary protecting groups include succinyl, phthalyl, methylene, ethylene, propylene, 2,2-dimethylpropa-1,3-diyl, and acetonide. Any combination of protecting groups described herein can be used as determined by one of skill in the art.

In some embodiments, the epothilone is a compound of formula (XV):

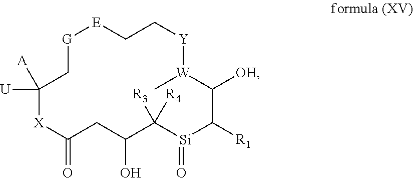

wherein

A is heteroalkyl, heterocycloalkyl, heteroalkylcycloalkyl, heteroaryl, heteroaralkenyl or heteroaralkyl group;

U is hydrogen, halogen, alkyl, heteroalkyl, heterocycloalkyl, heteroalkylcycloalkyl, heteroaryl or heteroaralkyl;

G-E is selected from the following groups,

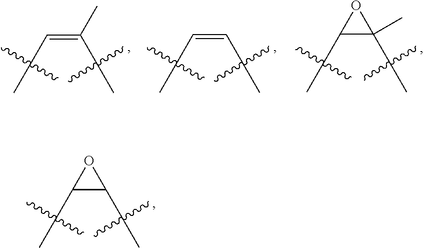

or is part of an optionally substituted phenyl ring;

R₁ is C₁-C₄-alkyl, C₂-C₄-alkenyl, C₂-C₄-alkynyl, or C₃-C₄-cycloalkyl group;

V—W is selected from the group consisting of CH₂CH or CH═C;

X is oxygen or a group of the formula NR₂, wherein R₂ is hydrogen, alkyl, alkenyl, alkynyl, heteroalkyl, aryl, heteroaryl, cycloalkyl, alkylcycloalkyl, heteroalkylcycloalkyl, heterocycloalkyl, aralkyl, or heteroaralkyl; and

each of R₃ and R₄, independently from each other, is hydrogen, C₁-C₄-alkyl or R₃ and R₄ together are part of a cycloalkyl group with 3 or 4 ring atoms.

In certain embodiments of formula (XV), A is a group of Formula (XVII) or (XVIII),

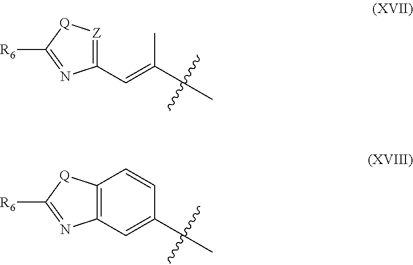

wherein

Q is sulfur, oxygen or NR₇ (preferably oxygen or sulfur), wherein R₇ is hydrogen, C₁-C₄ alkyl or C₁-C₄ heteroalkyl;

Z is nitrogen or CH (preferably CH); and

R₆ is OR₈, NHR₈, C₁-C₄ alkyl, C₁-C₄ alkenyl, C₁-C₄ alkynyl or C₁-C₆ heteroalkyl (preferably methyl, CH₂OR₈ or CH₂NHR₈), wherein R₈ is hydrogen, C₁-C₄ alkyl or C₁-C₄ heteroalkyl (preferably hydrogen).

In some embodiments, the epothilone is a compound of formula (XVI):

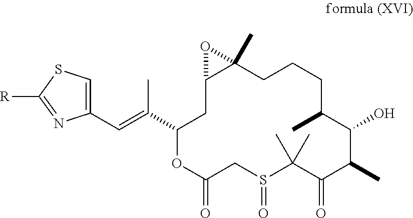

wherein R is selected from OR¹, NHR¹, alkyl, alkenyl, alkynyl and heteroalkyl (e.g., CH₂OR¹ or CH₂NHR¹) and R¹ is selected from hydrogen, C₁₋₄ alkyl and C₁₋₄ heteroalkyl (preferably hydrogen).

In certain embodiments, R is selected from methyl, CH₂OH and CH₂NH₂.

Preparation of naturally occurring and semi-synthetic epothilones and corresponding derivatives is known in the art. Epothilones A & B were first extracted from Sorangium cellulosum So ce90 which exists at the German Collection of Microorganisms as DMS 6773 and DSM 11999. It has been reported that DSM 6773 allegedly displays increased production of epothilones A and B over the wild type strain. Representative fermentation conditions for Sorangium are described, for example, in U.S. Pat. No. 6,194,181 and various international PCT publications including WO 98/10121, WO 97/19086, WO 98/22461 and WO 99/42602. Methods of preparing epothilones are also described in WO 93/10121.

In addition, epothilones can be obtained via de novo synthesis. The total synthesis of epothilones A and B have been reported by a number of research groups including Danishefsky, Schinzer and Nicolaou. These total syntheses are described, for example, in U.S. Pat. Nos. 6,156,905, 6,043,372, and 5,969,145 and in international PCT publications WO 98/08849, WO 98/25929, and WO 99/01124. Additional synthetic methods for making epothilone compounds are also described in PCT publications WO 97/19086, WO 98/38192, WO 99/02514, WO 99/07692, WO 99/27890, WO 99/28324, WO 99/43653, WO 99/54318, WO 99/54319, WO 99/54330, WO 99/58534, WO 59985, WO 99/67252, WO 99/67253, WO 00/00485, WO 00/23452, WO 00/37473, WO 00/47584, WO 00/50423, WO 00/57874, WO 00/58254, WO 00/66589, WO 00/71521, WO 01/07439 and WO 01/27308.

Exemplary CDP-Epothilone Conjugates

CDP-epothilone conjugates can be made using many different combinations of components described herein. For example, various combinations of cyclodextrins (e.g., beta-cyclodextrin), comonomers (e.g., PEG containing comonomers), linkers linking the cyclodextrins and comonomers, and/or linkers tethering the epothilone to the CDP are described herein.

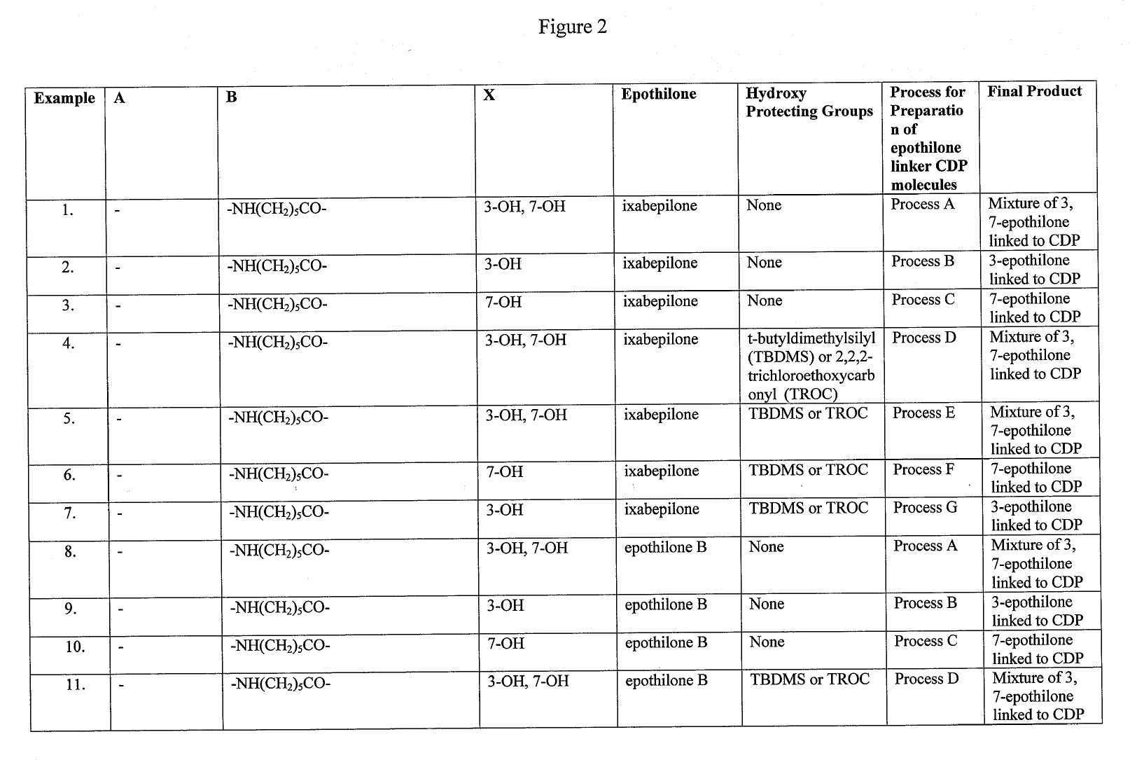

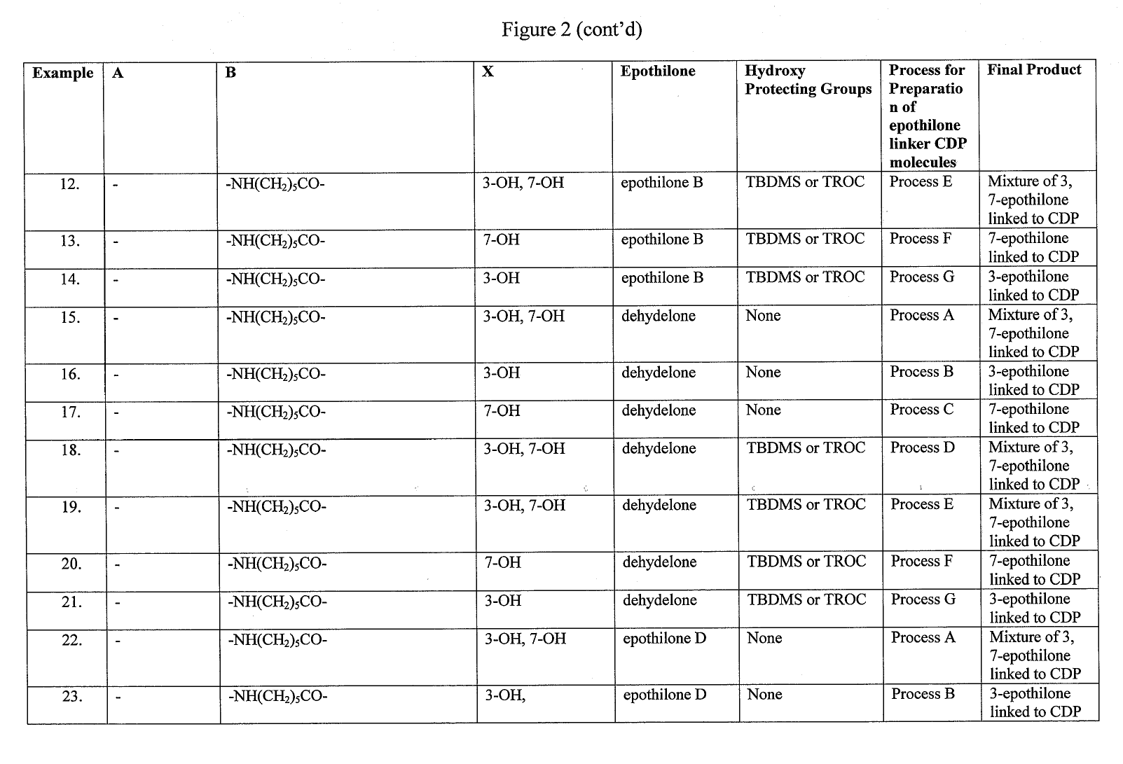

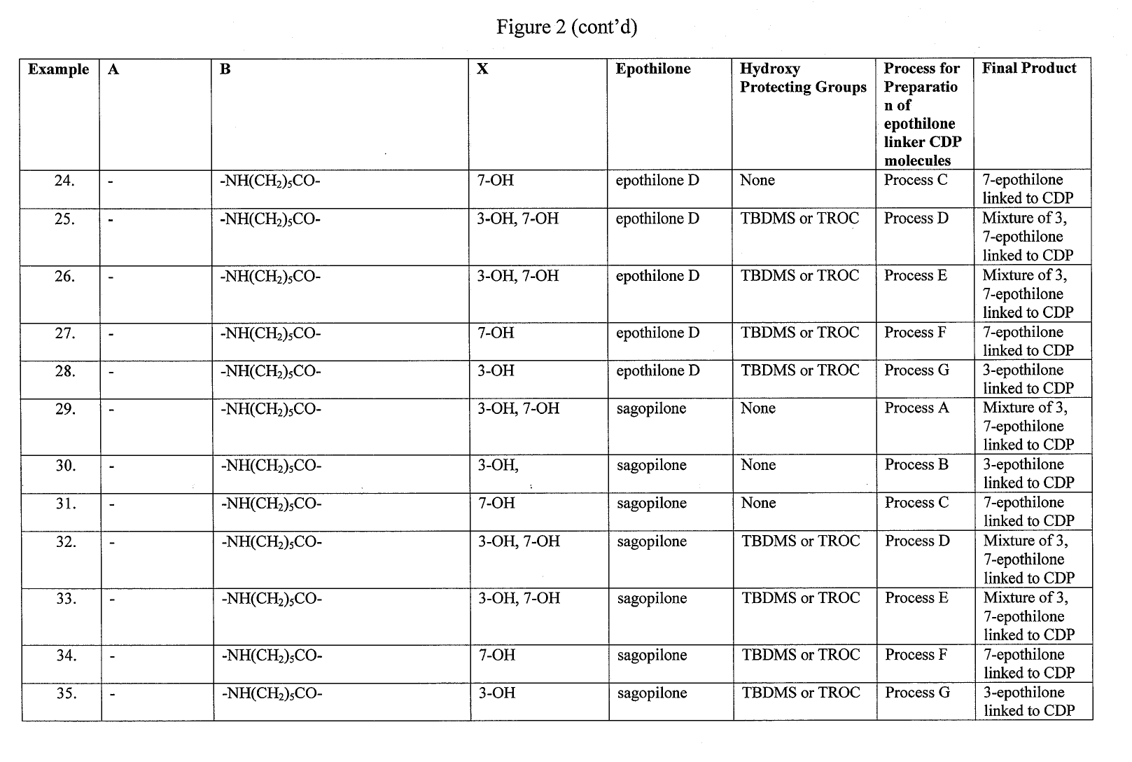

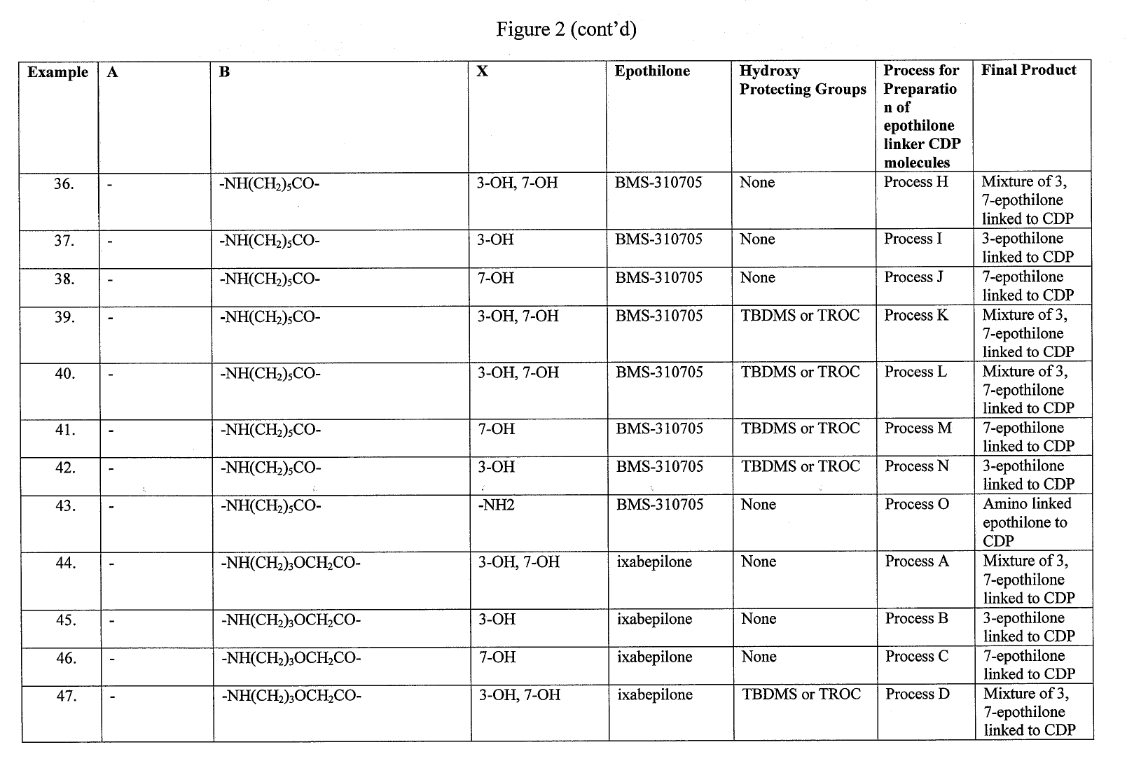

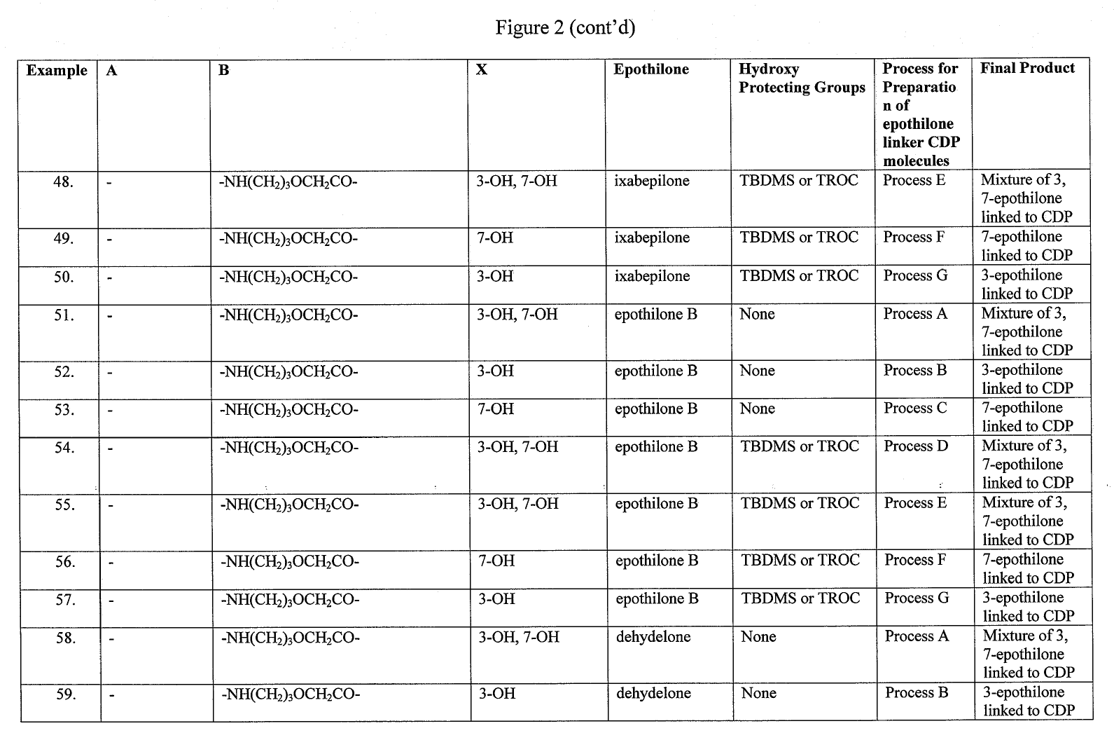

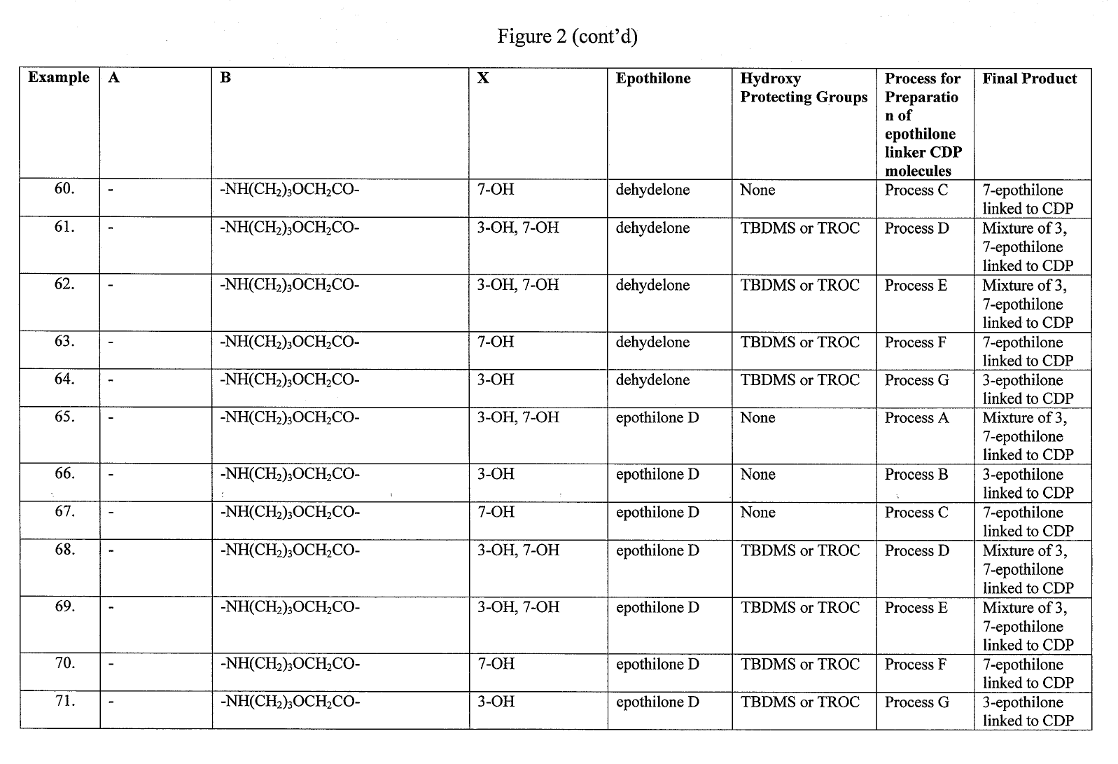

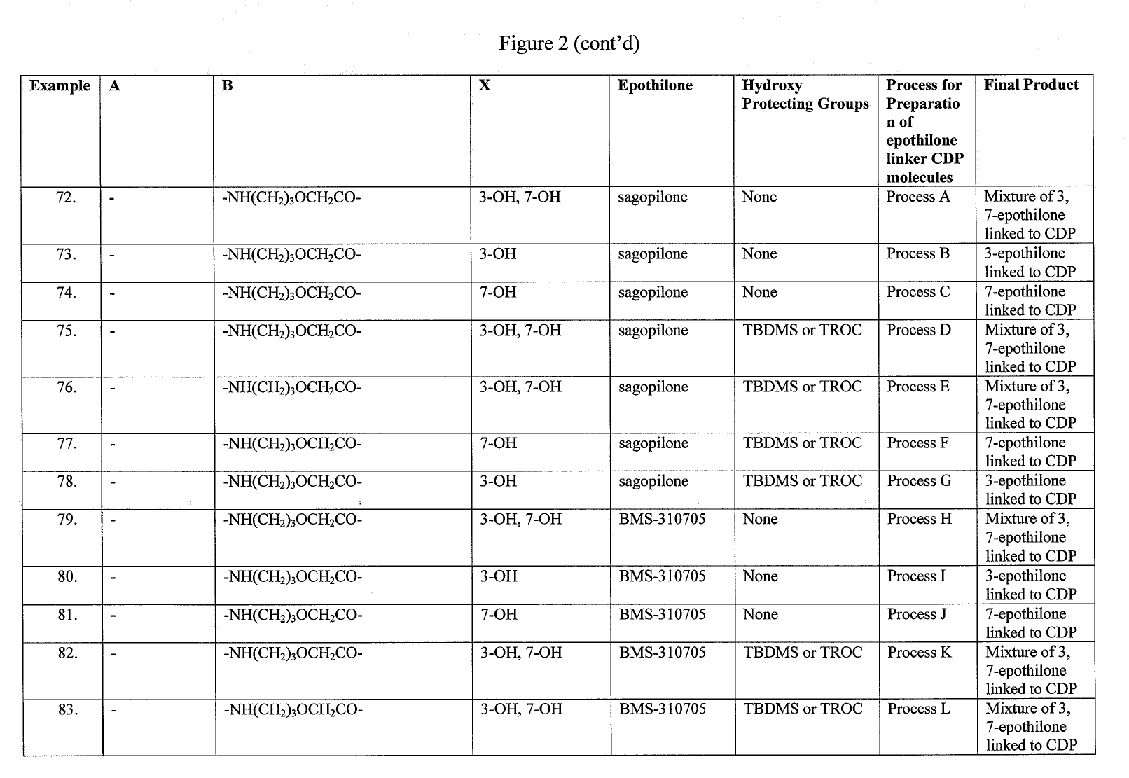

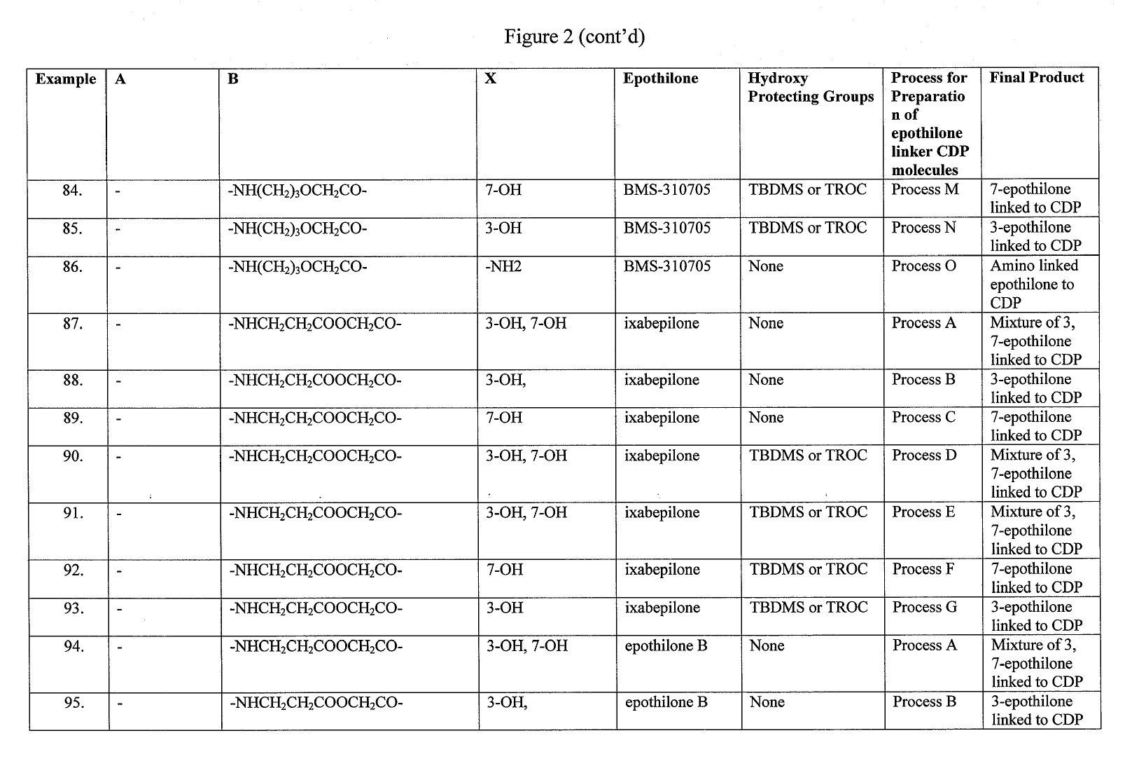

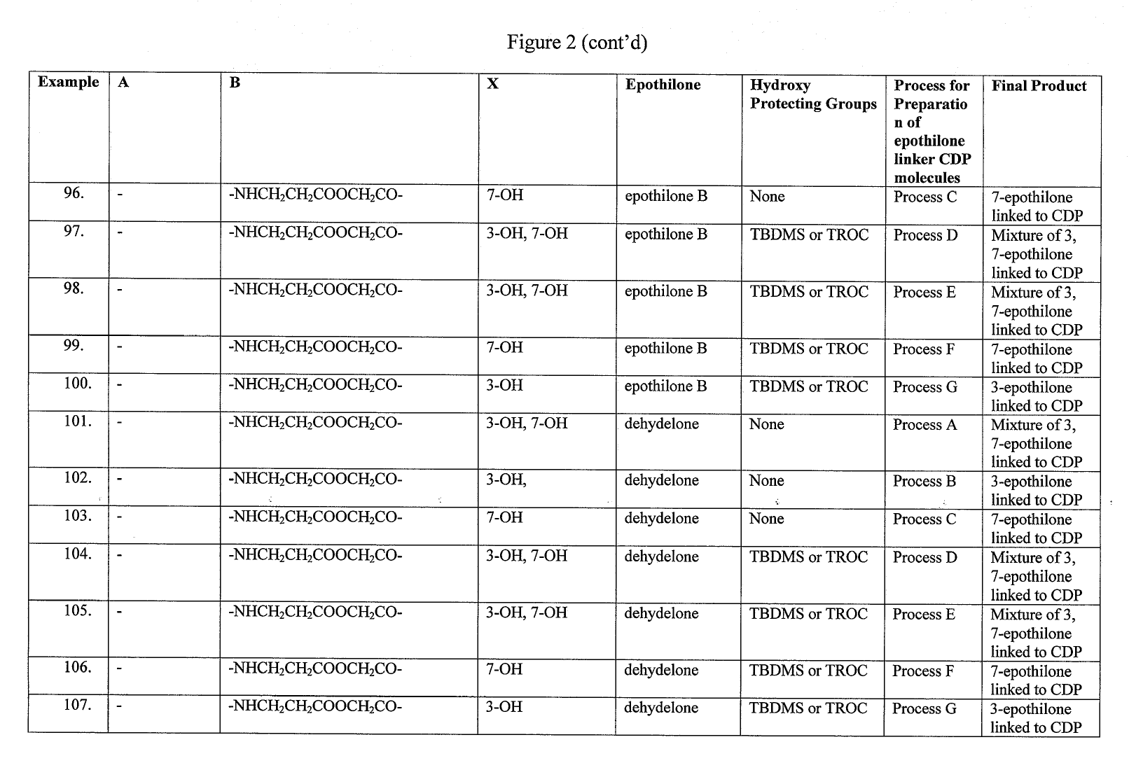

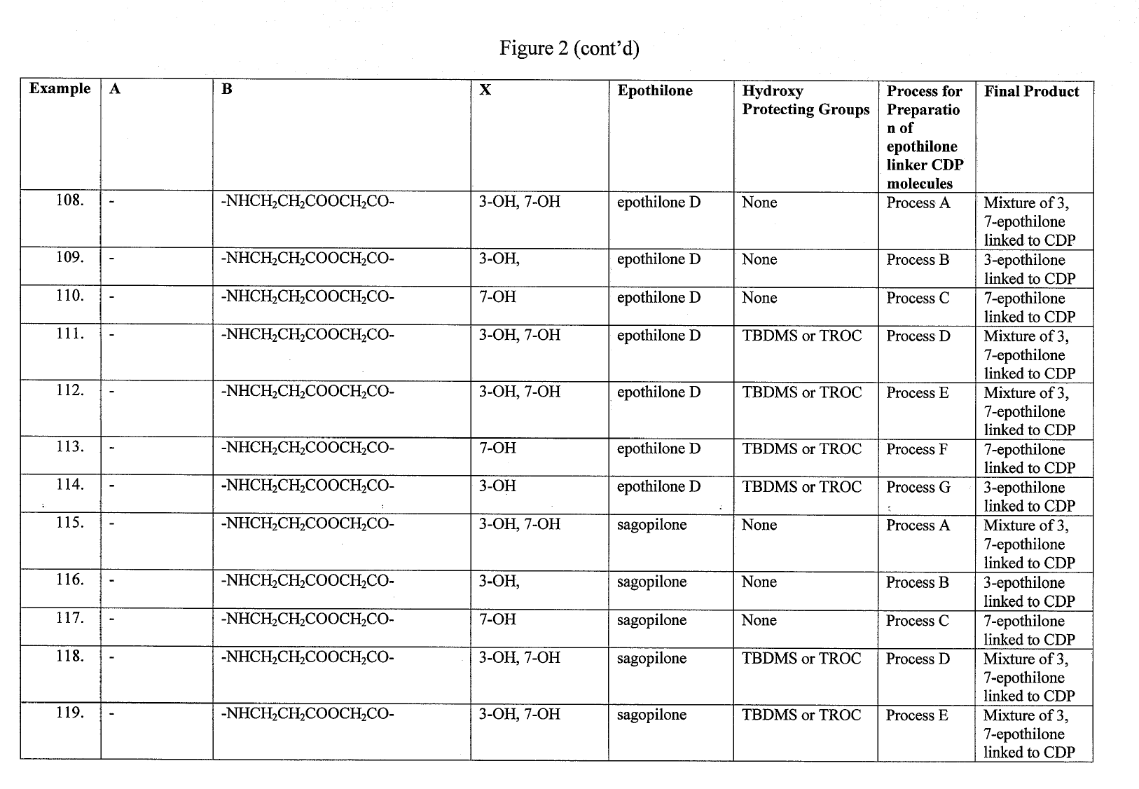

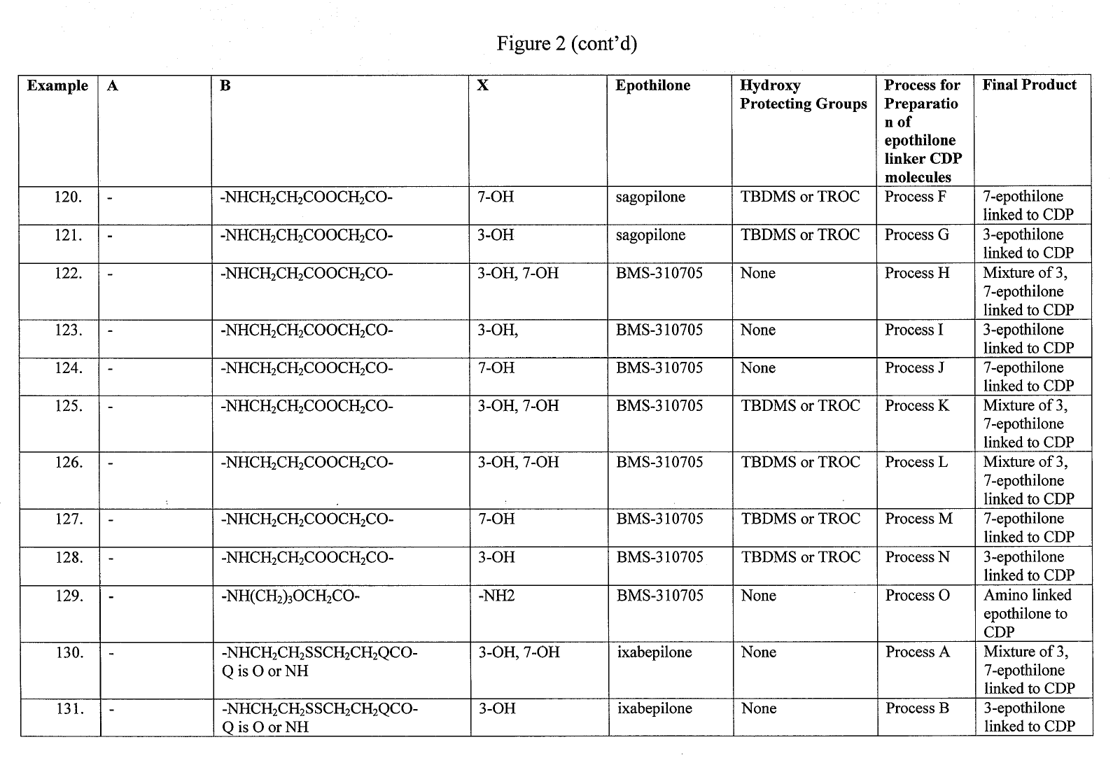

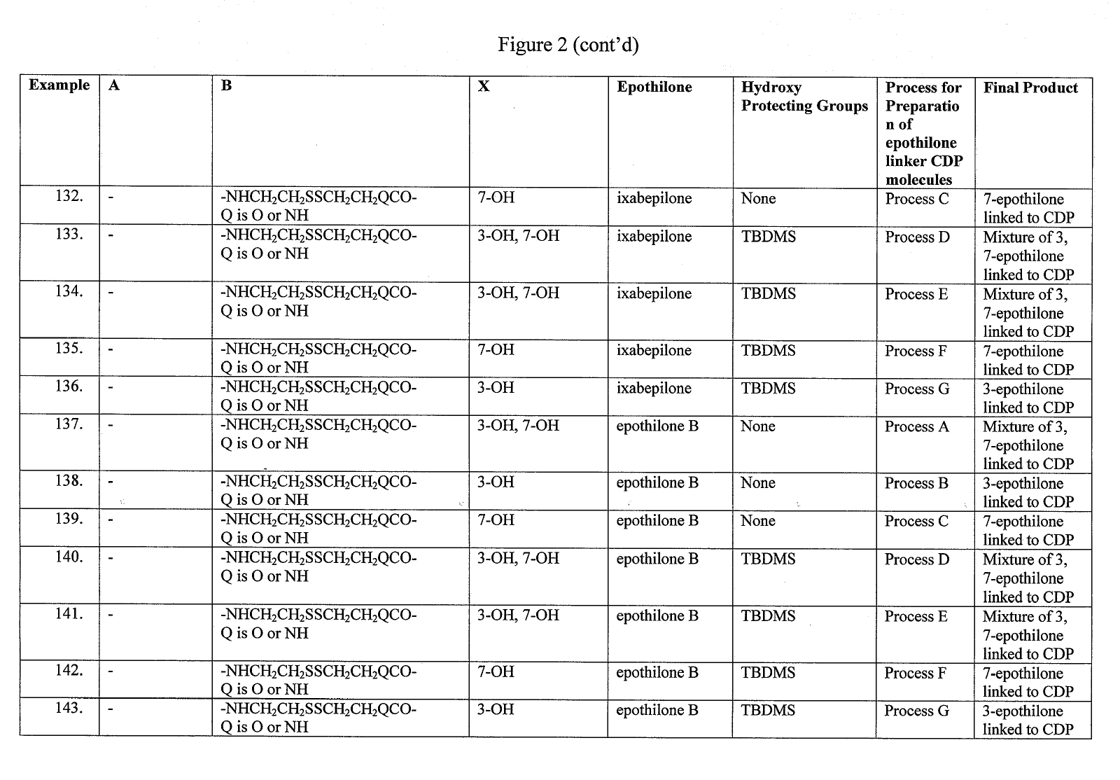

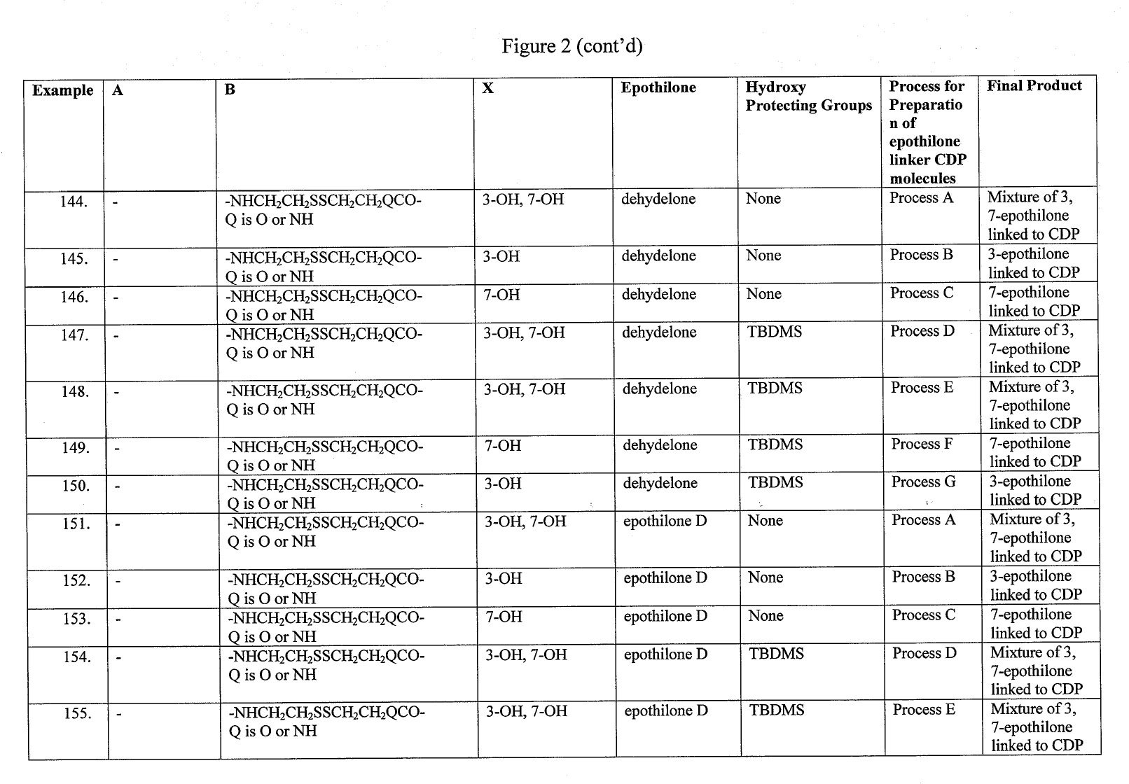

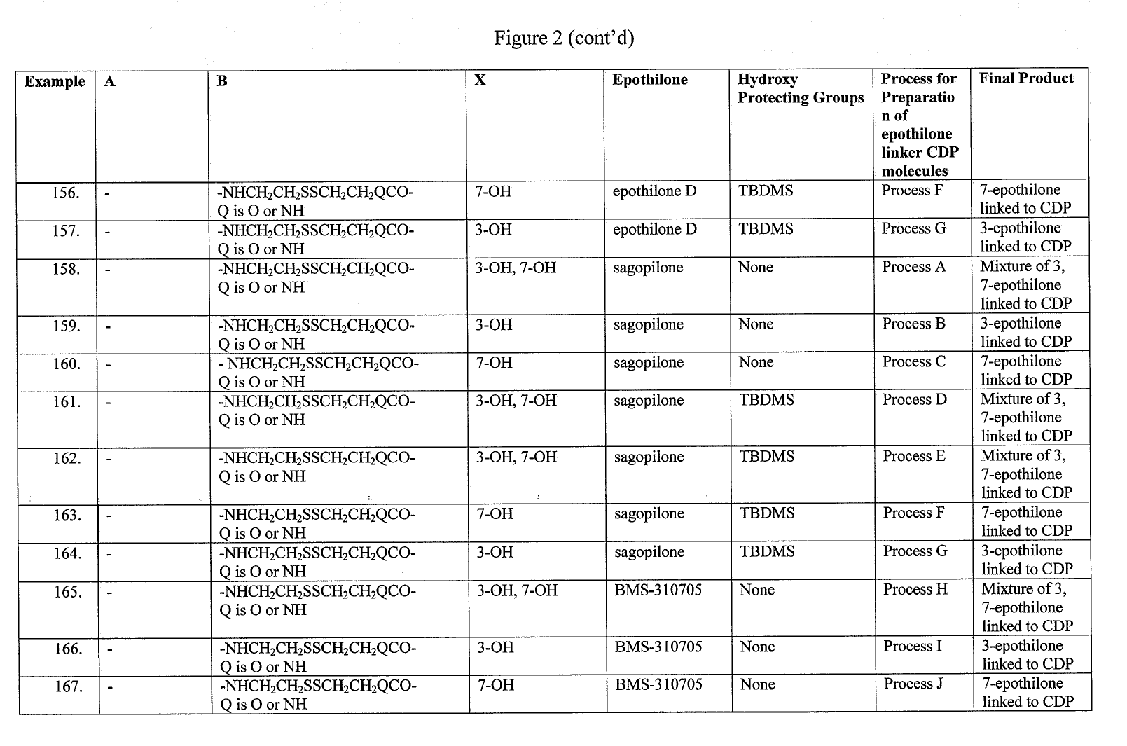

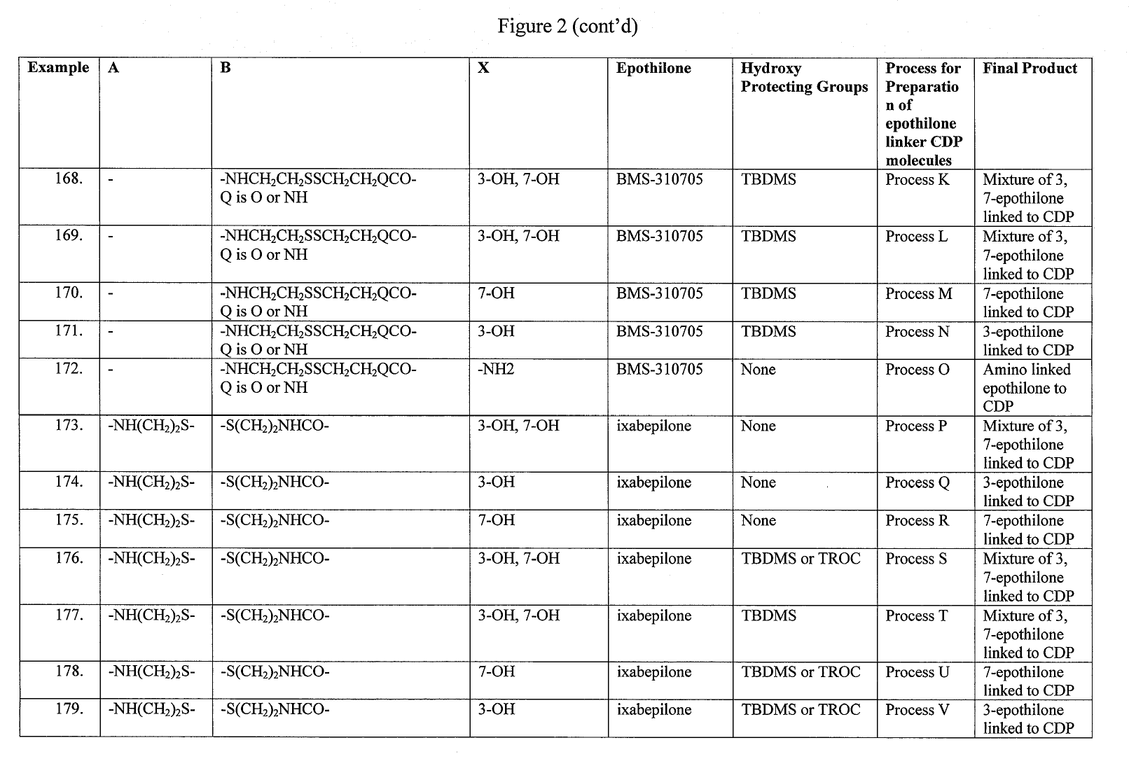

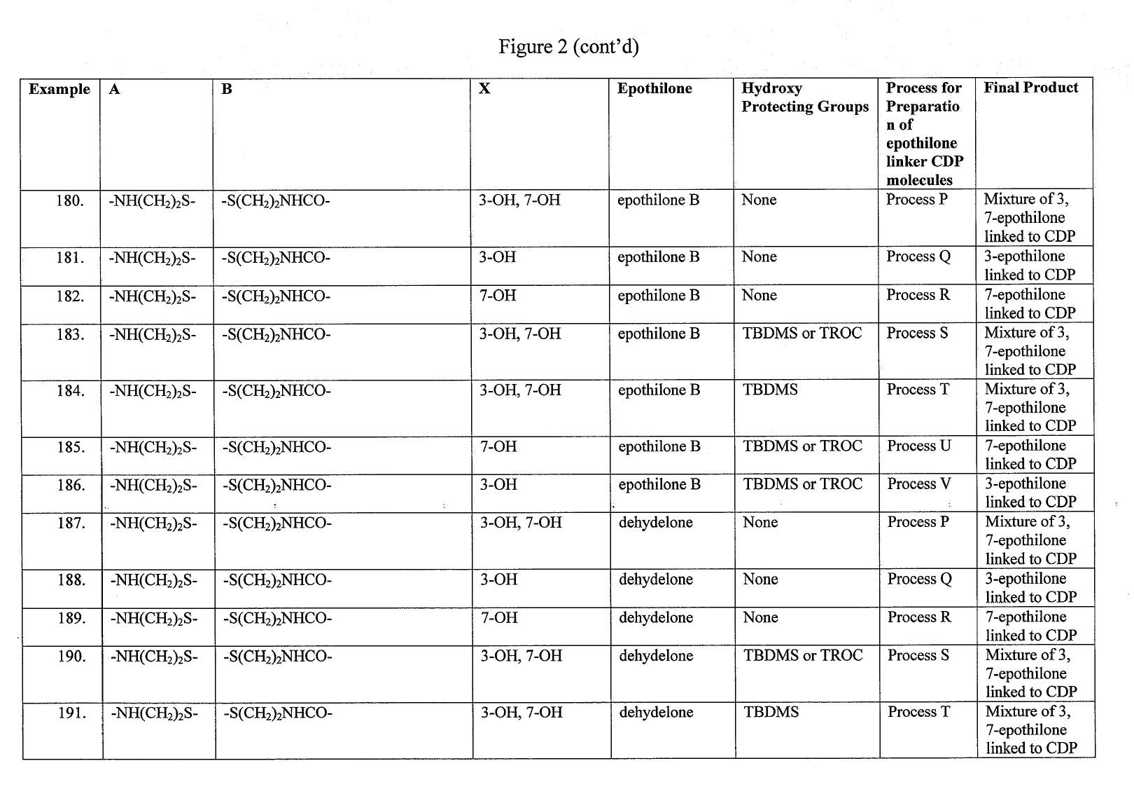

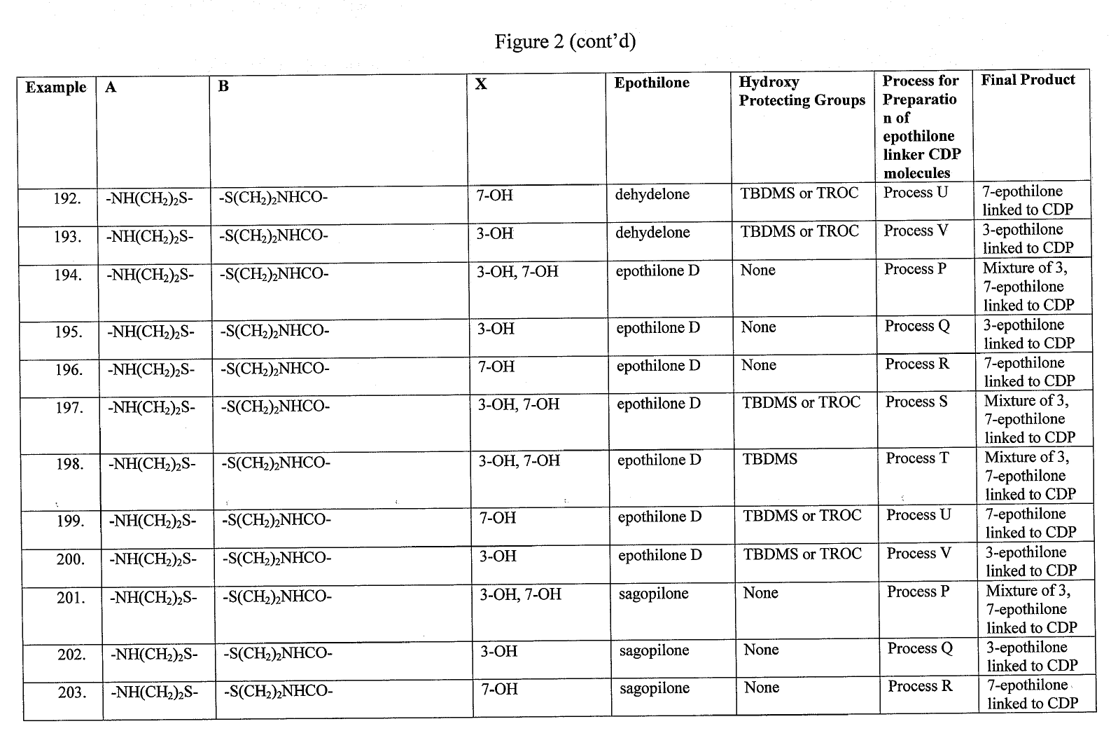

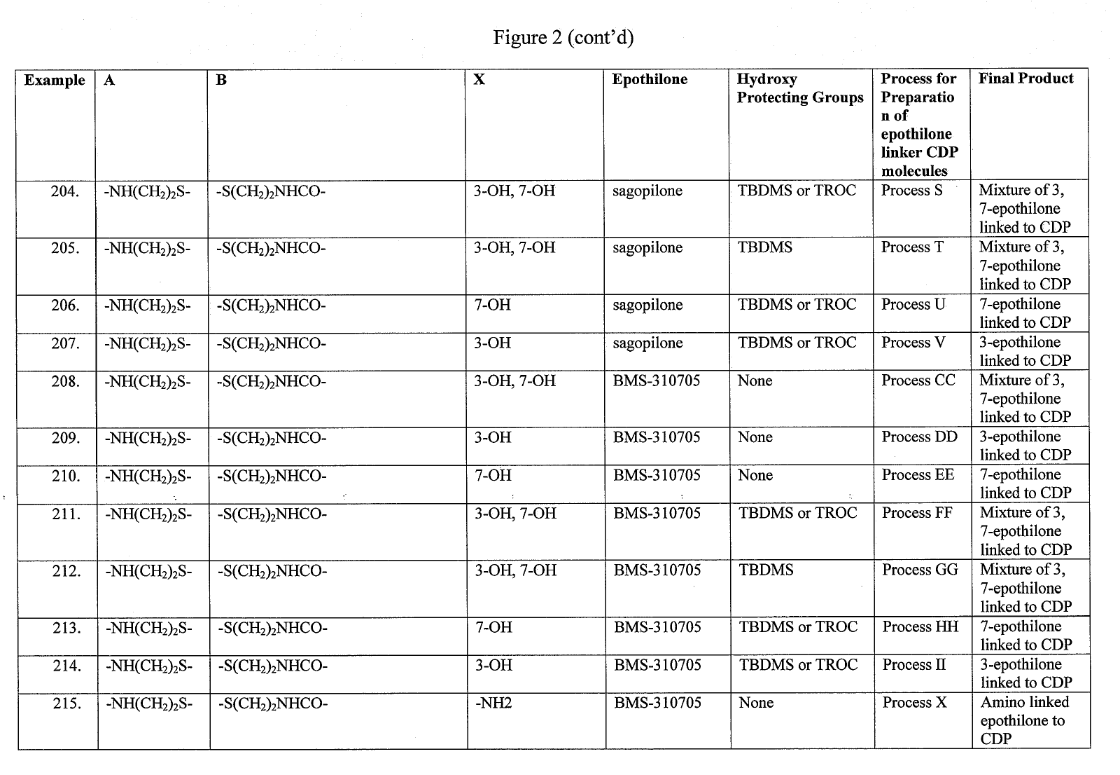

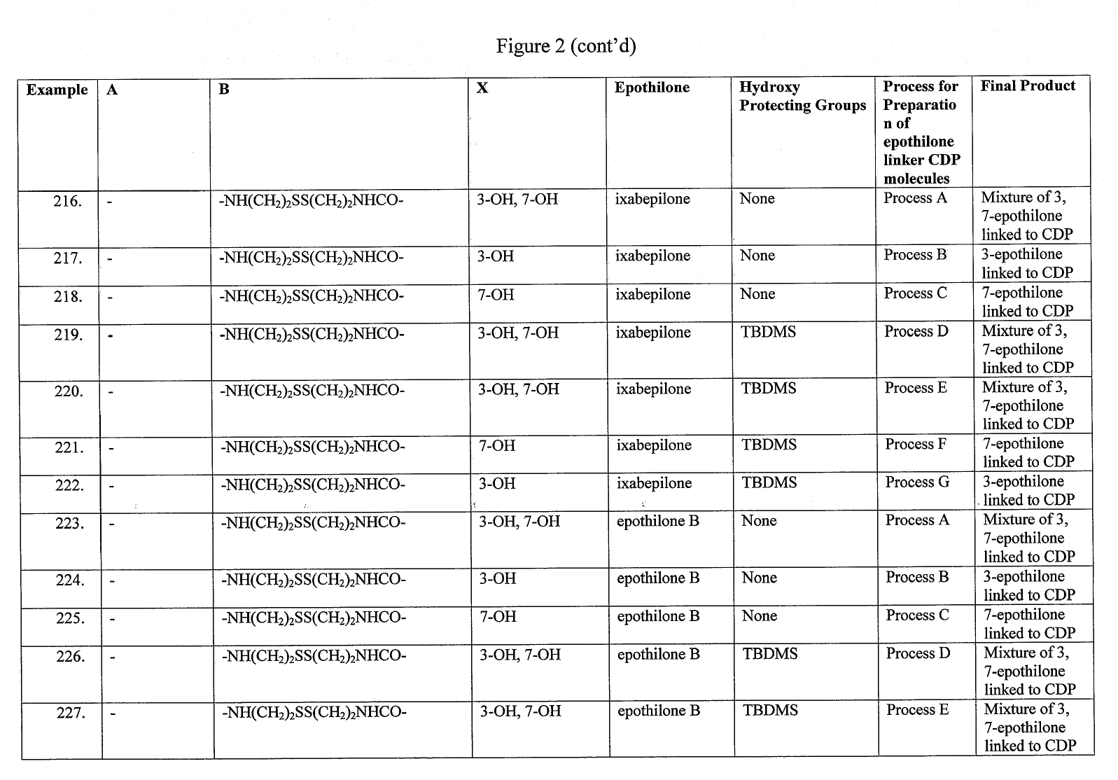

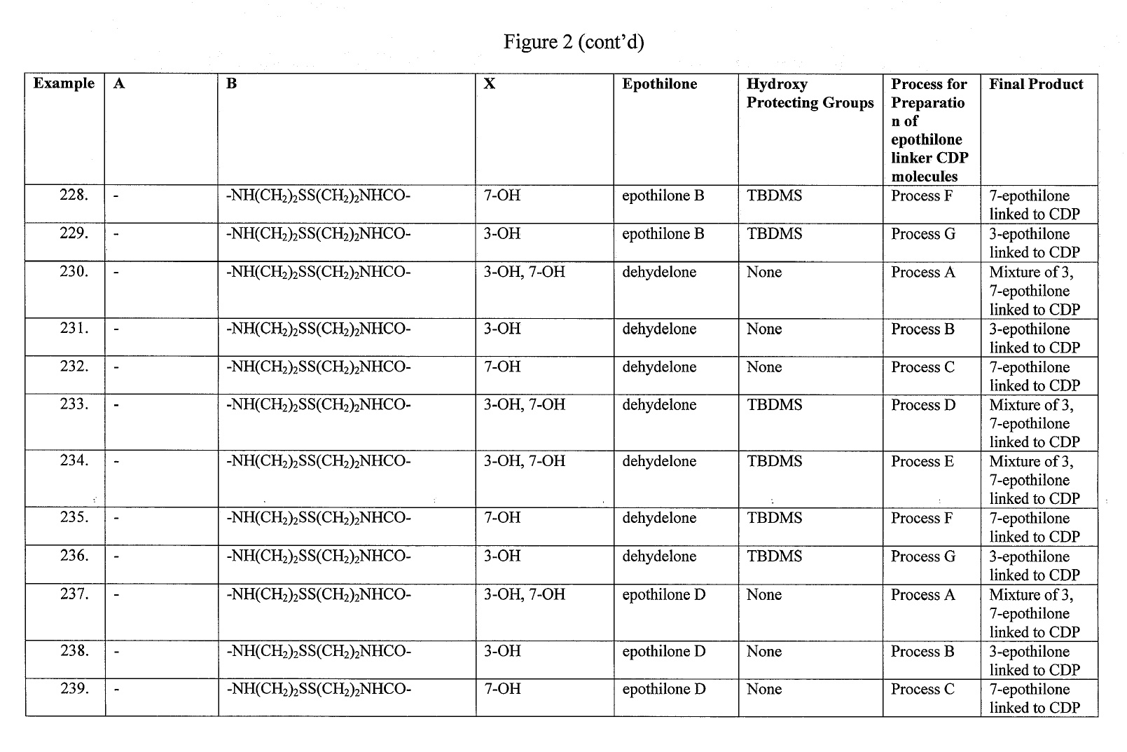

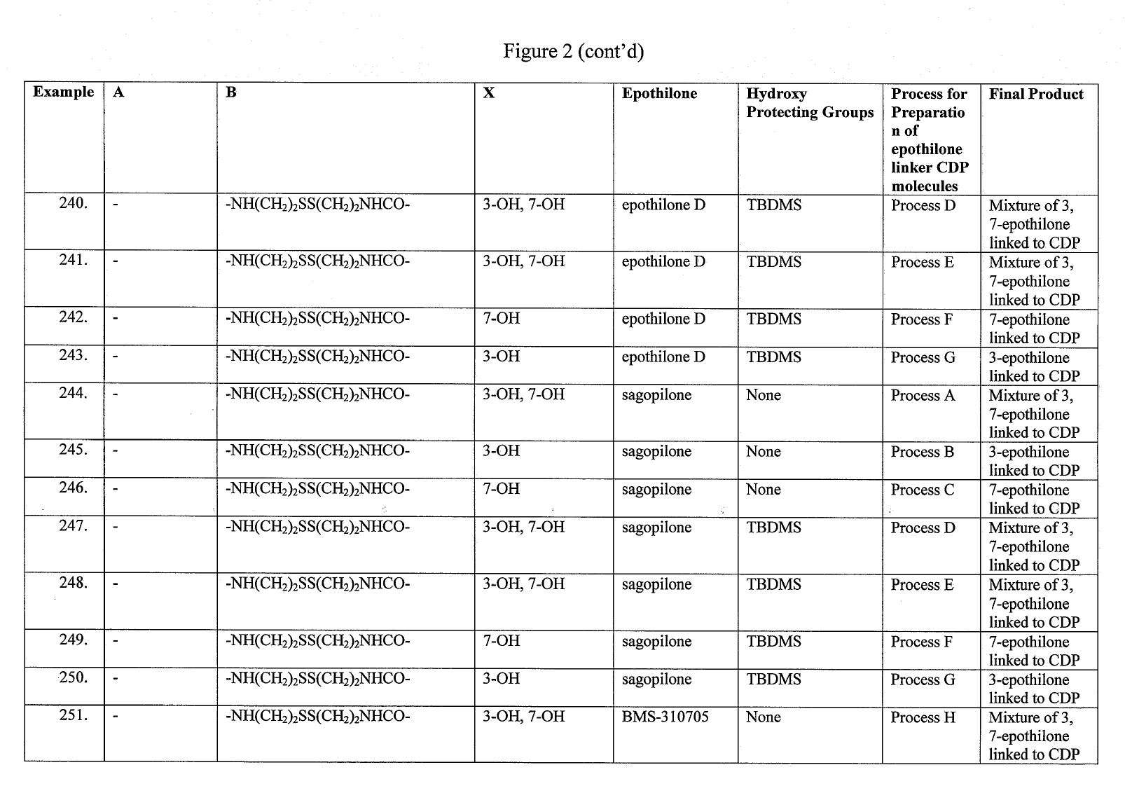

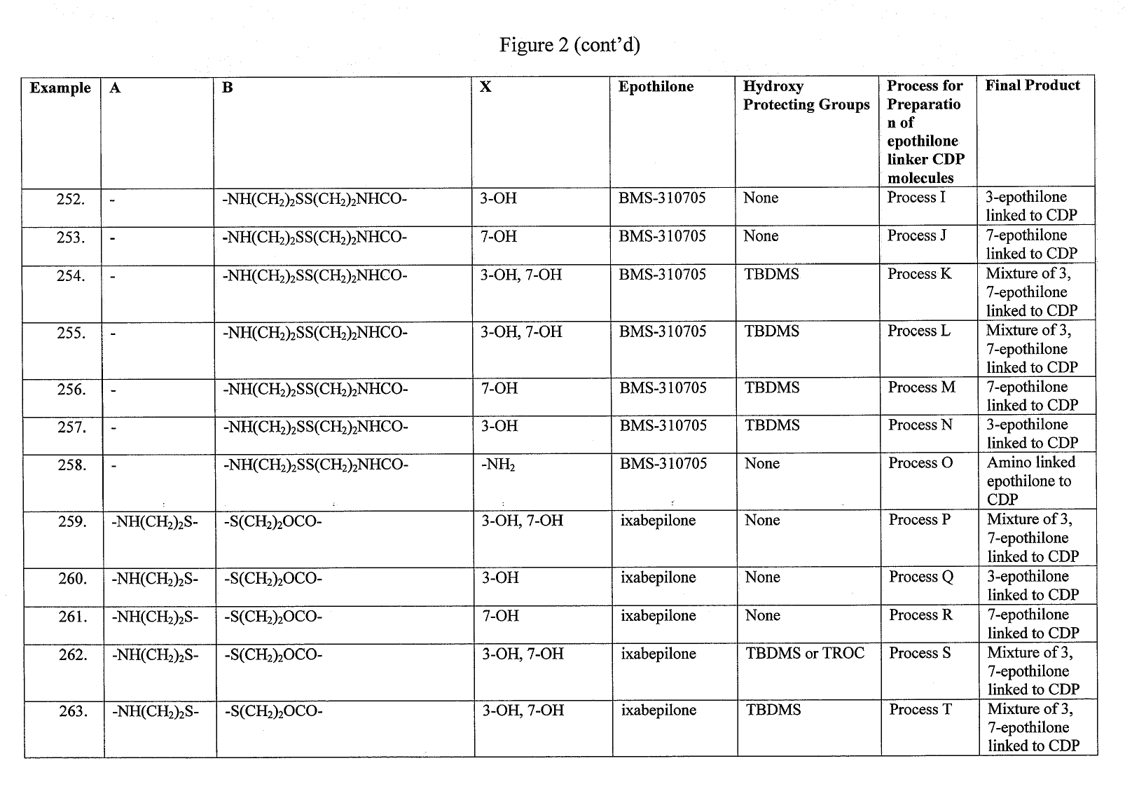

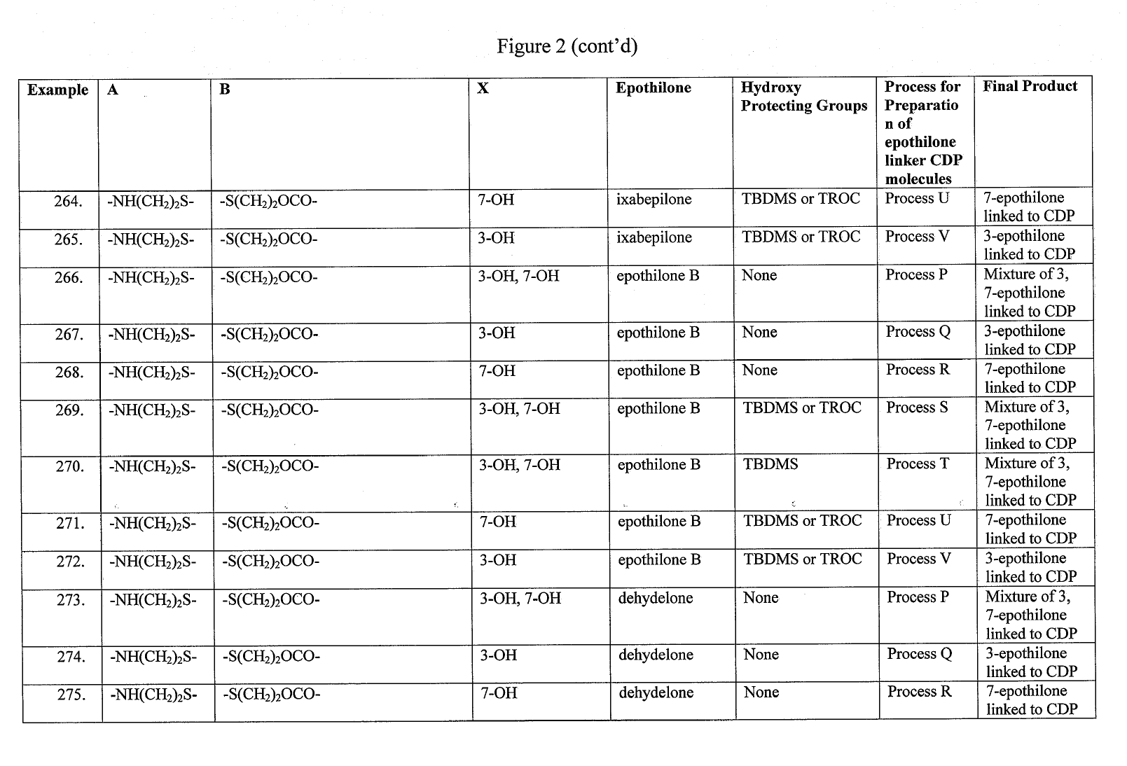

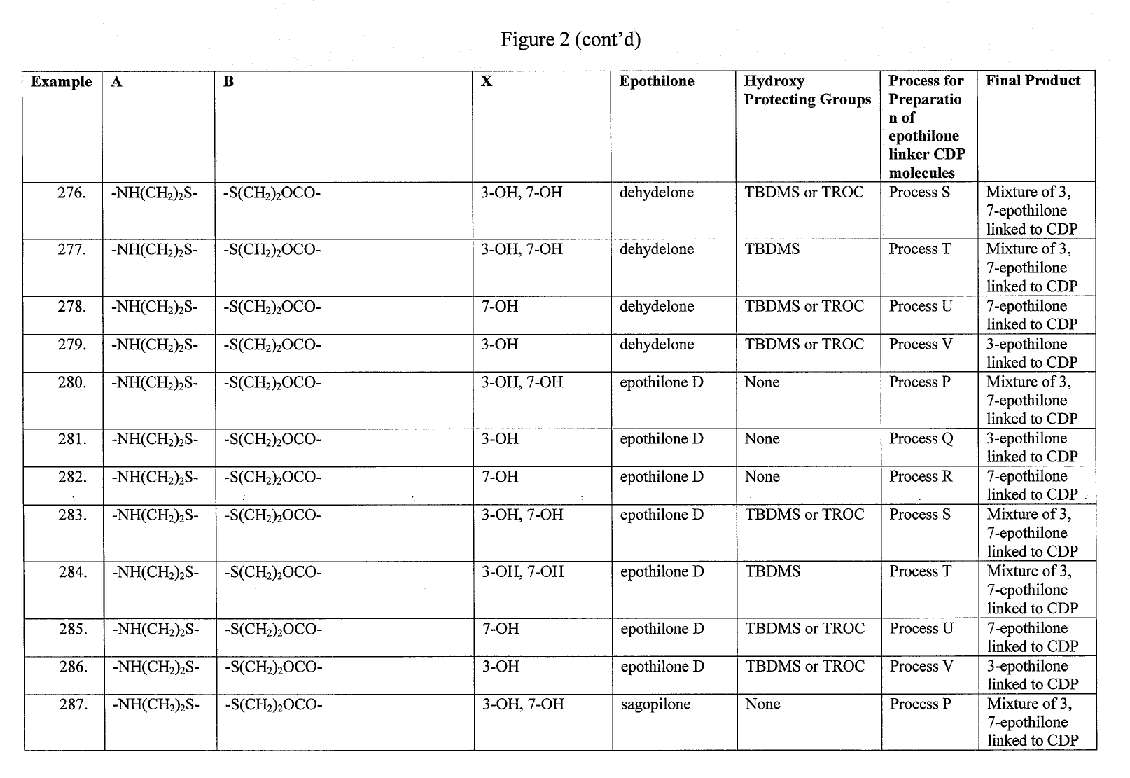

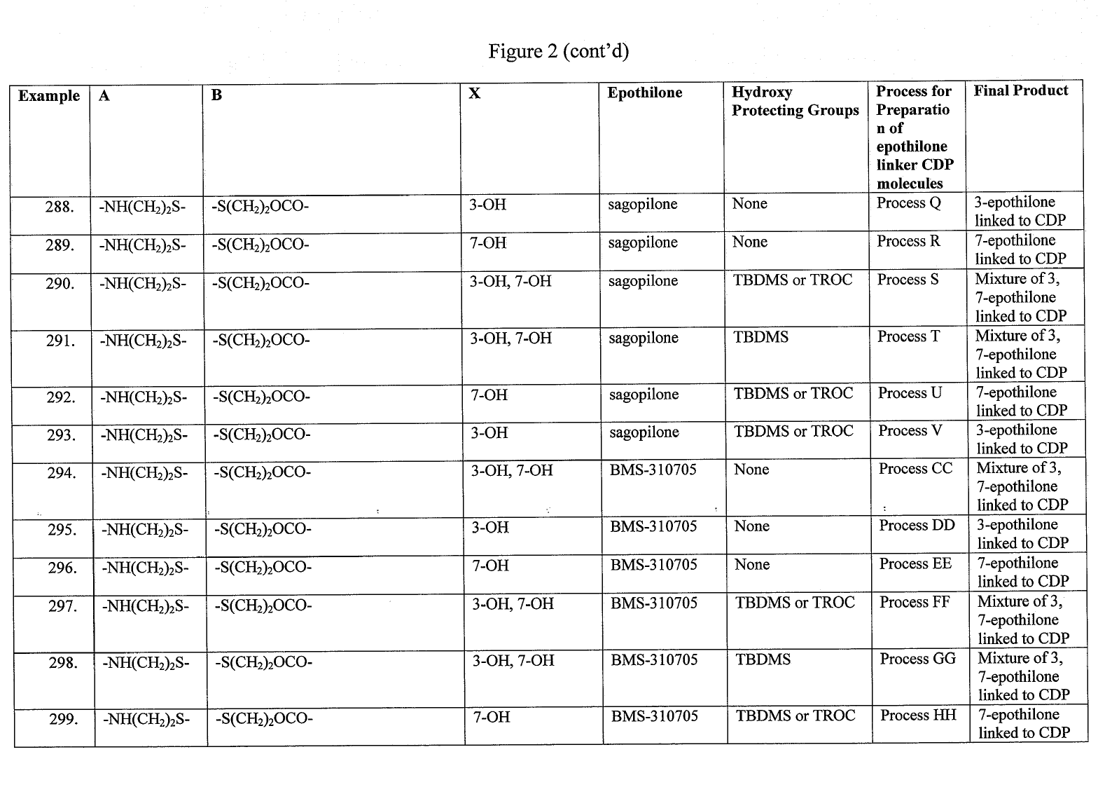

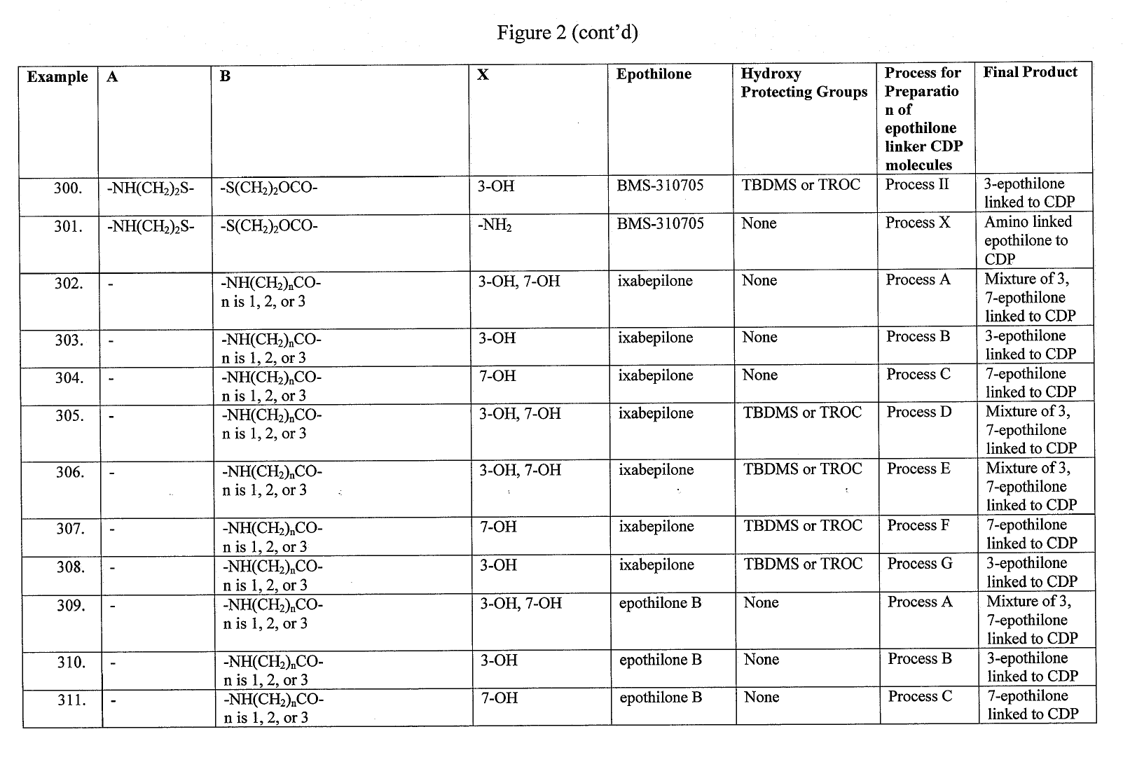

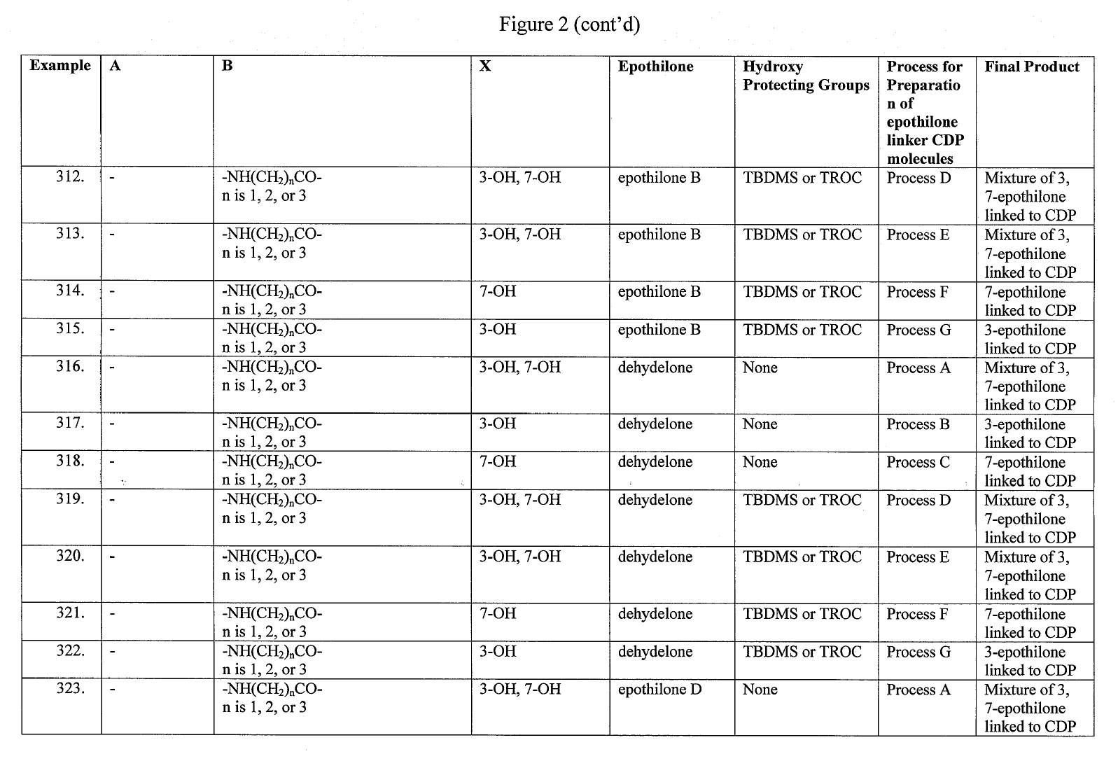

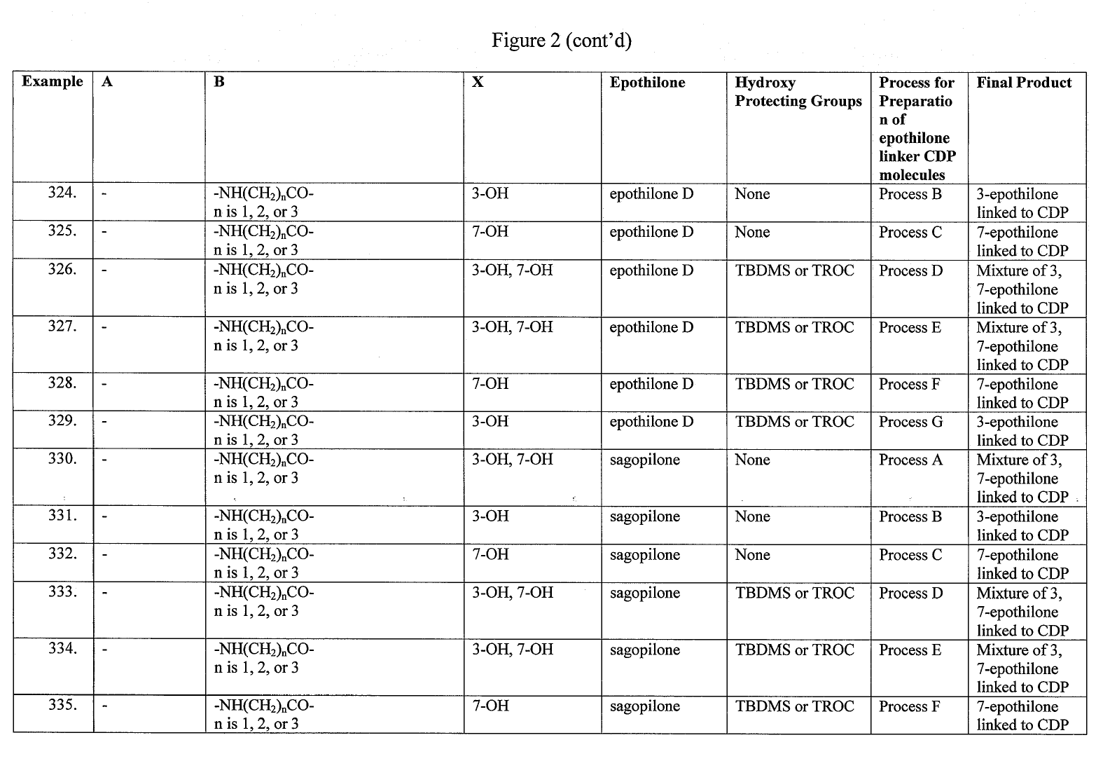

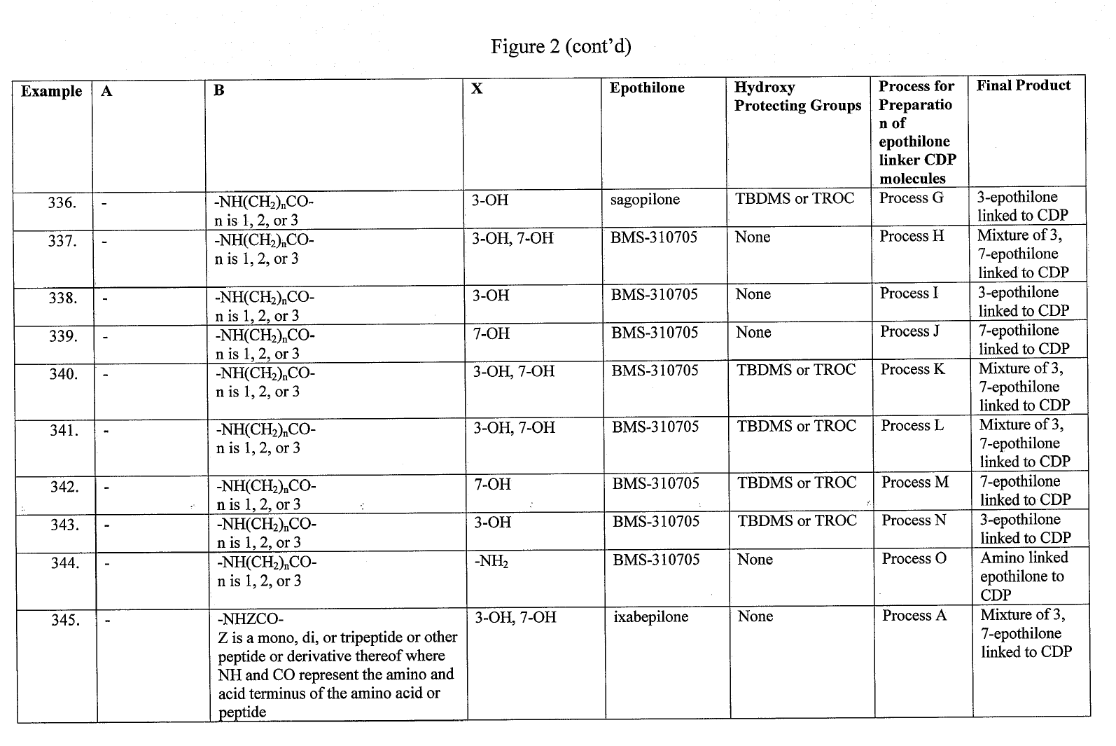

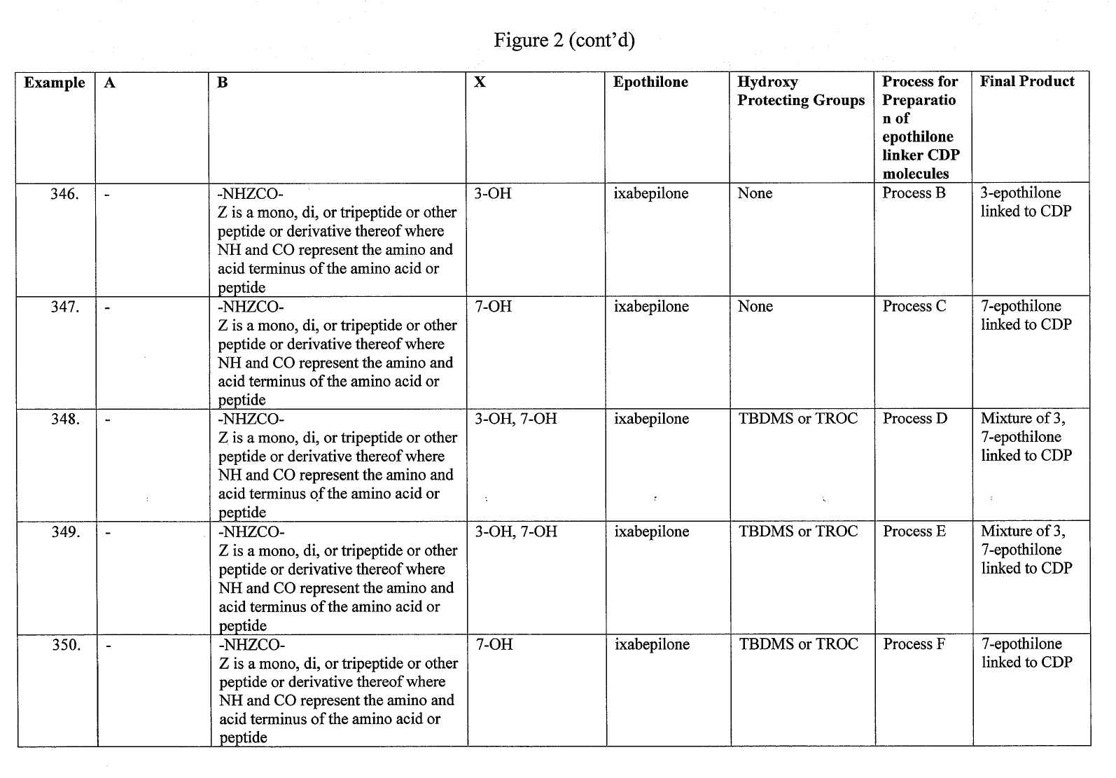

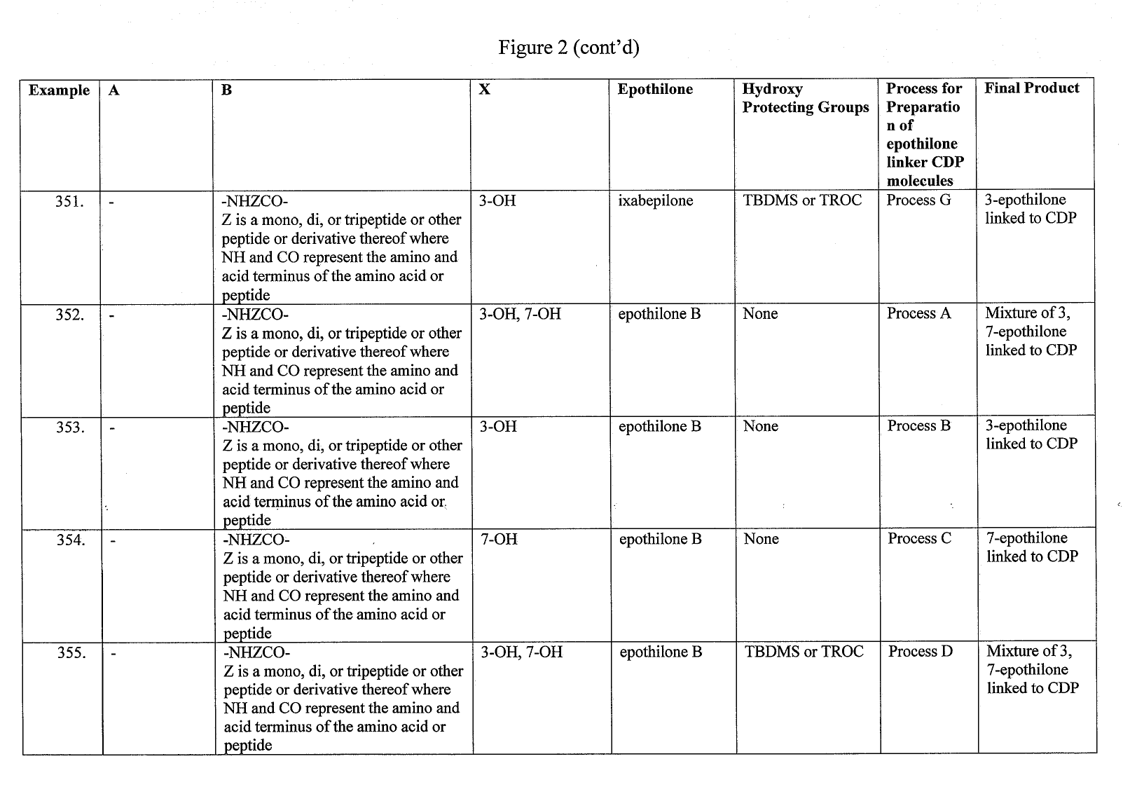

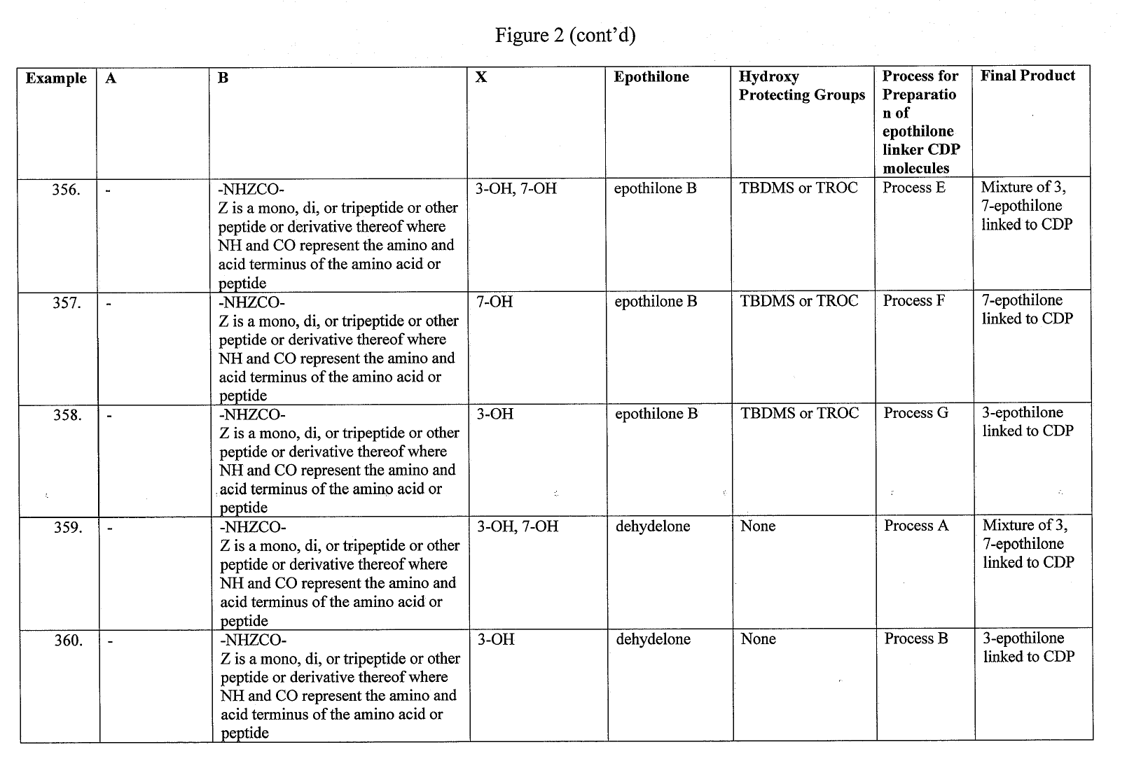

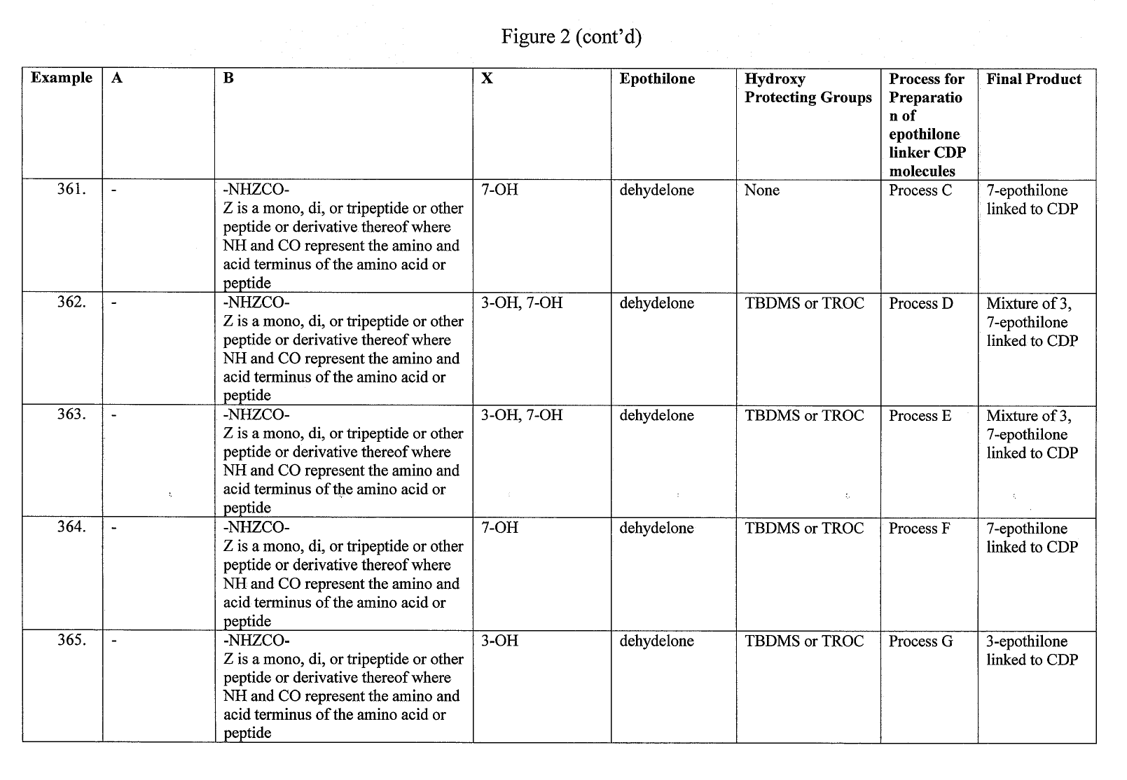

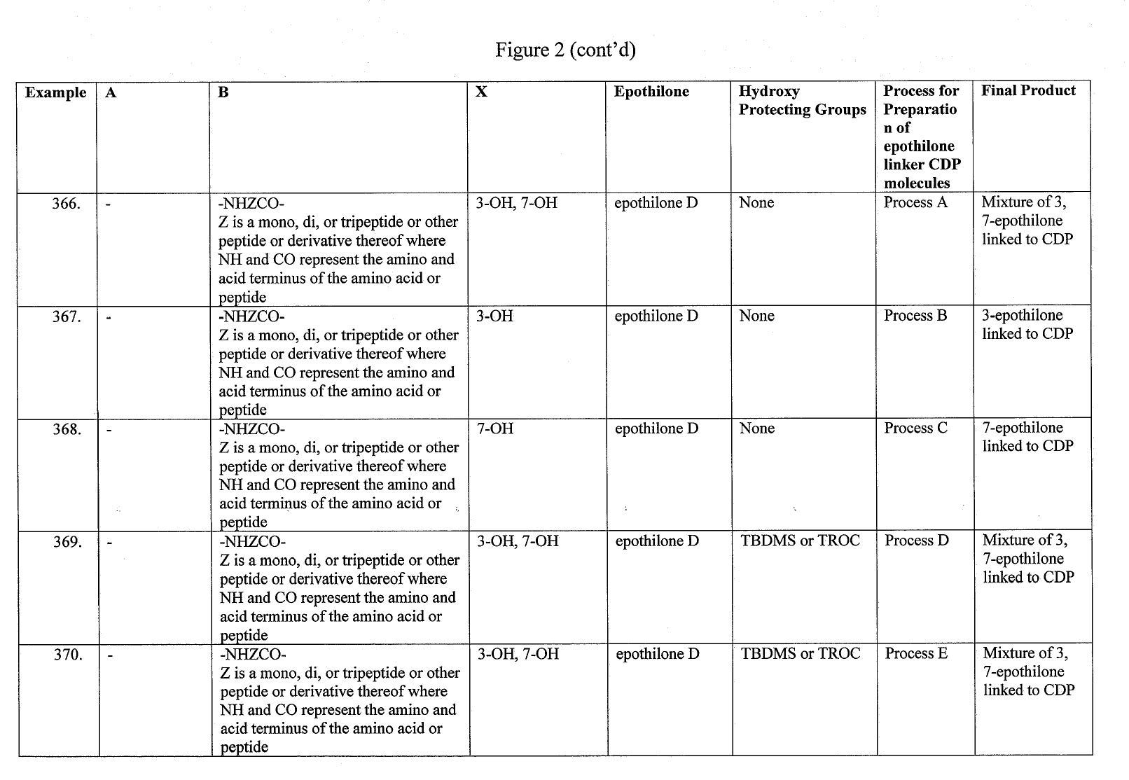

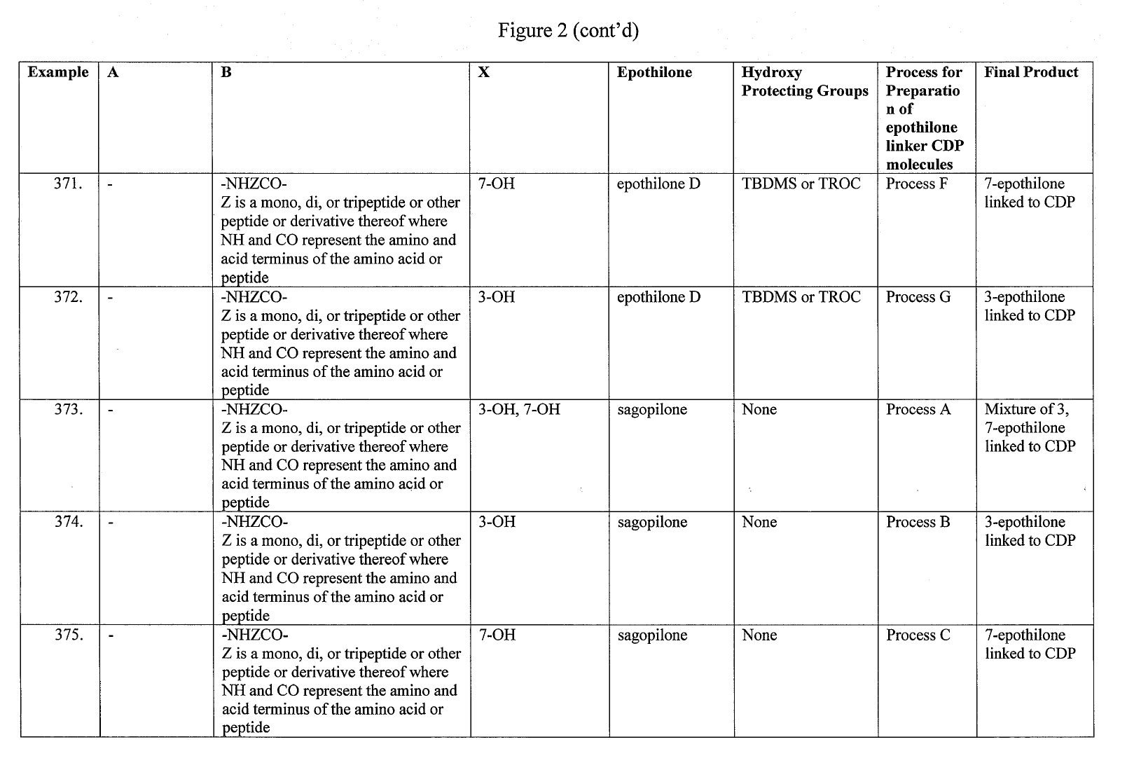

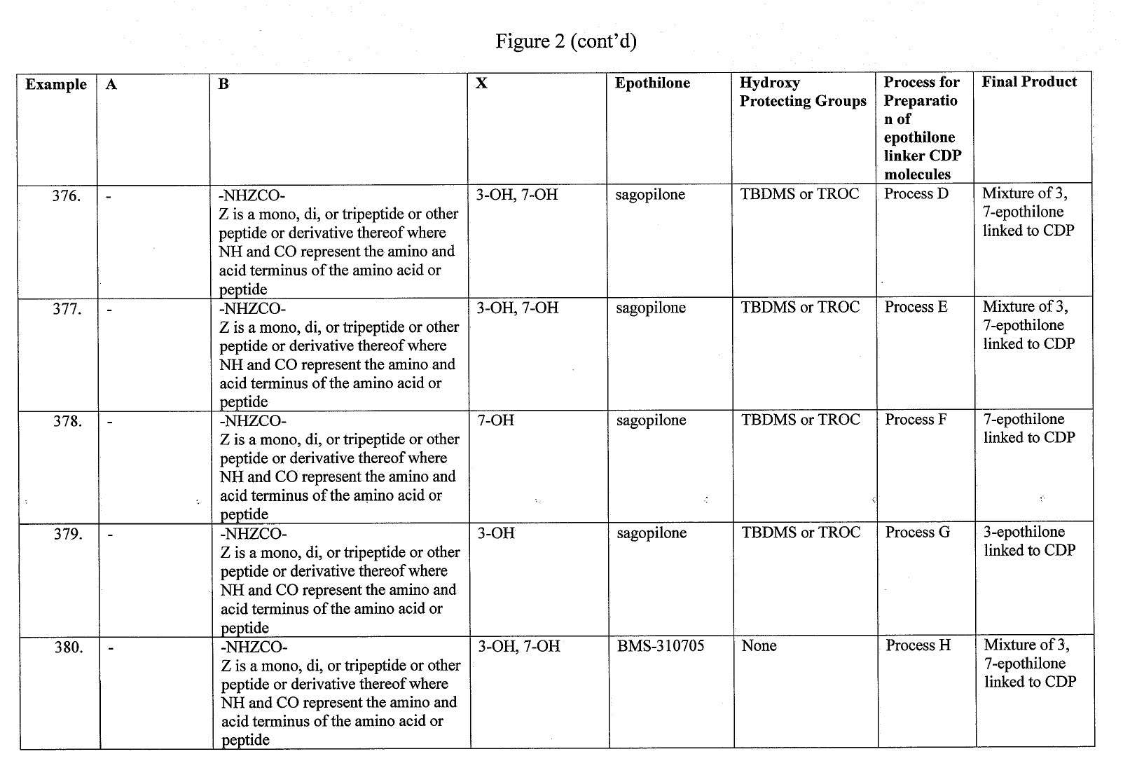

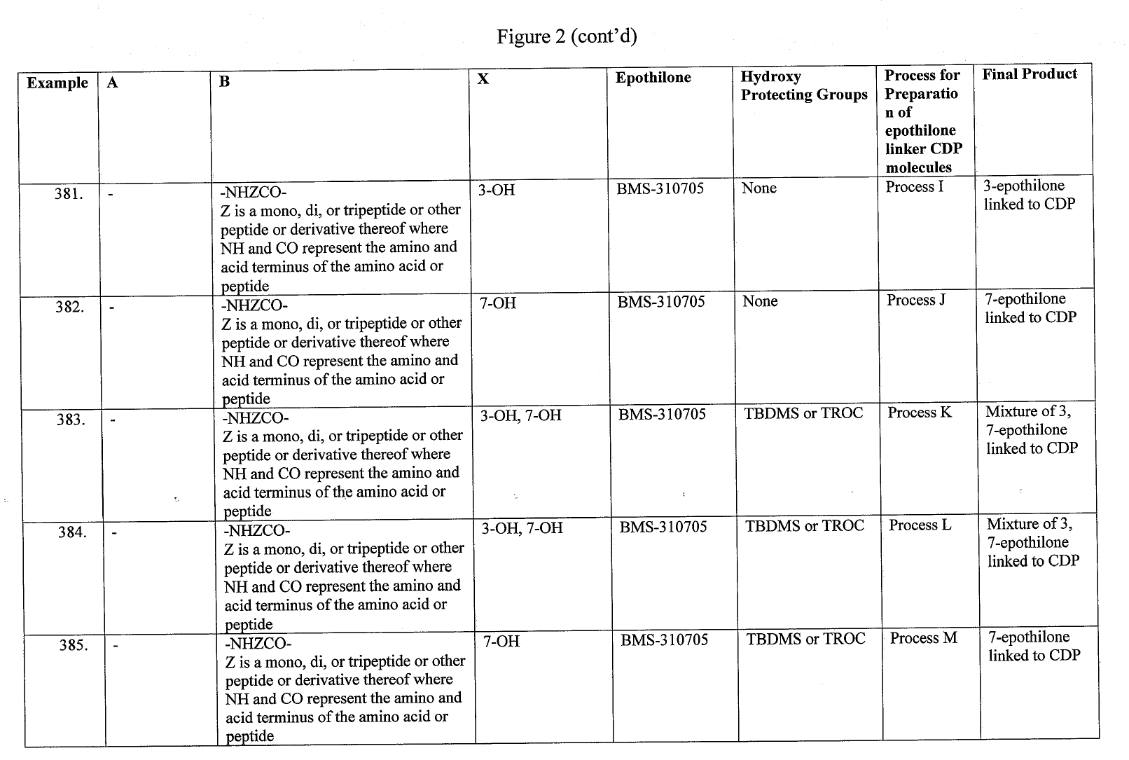

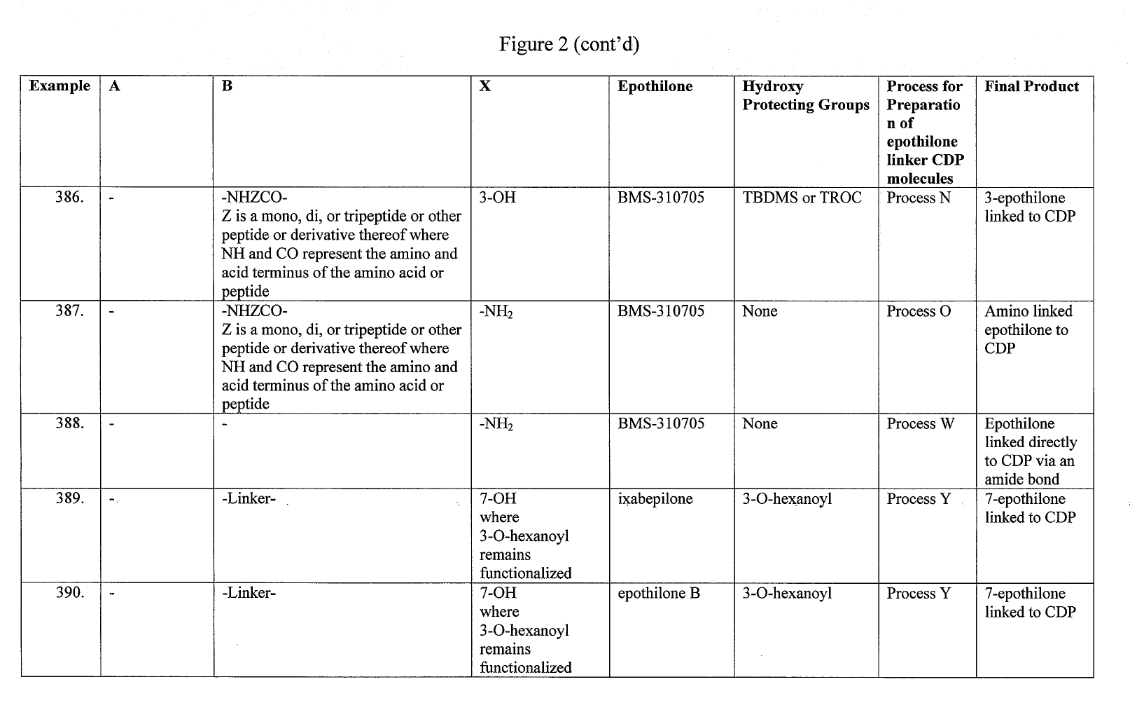

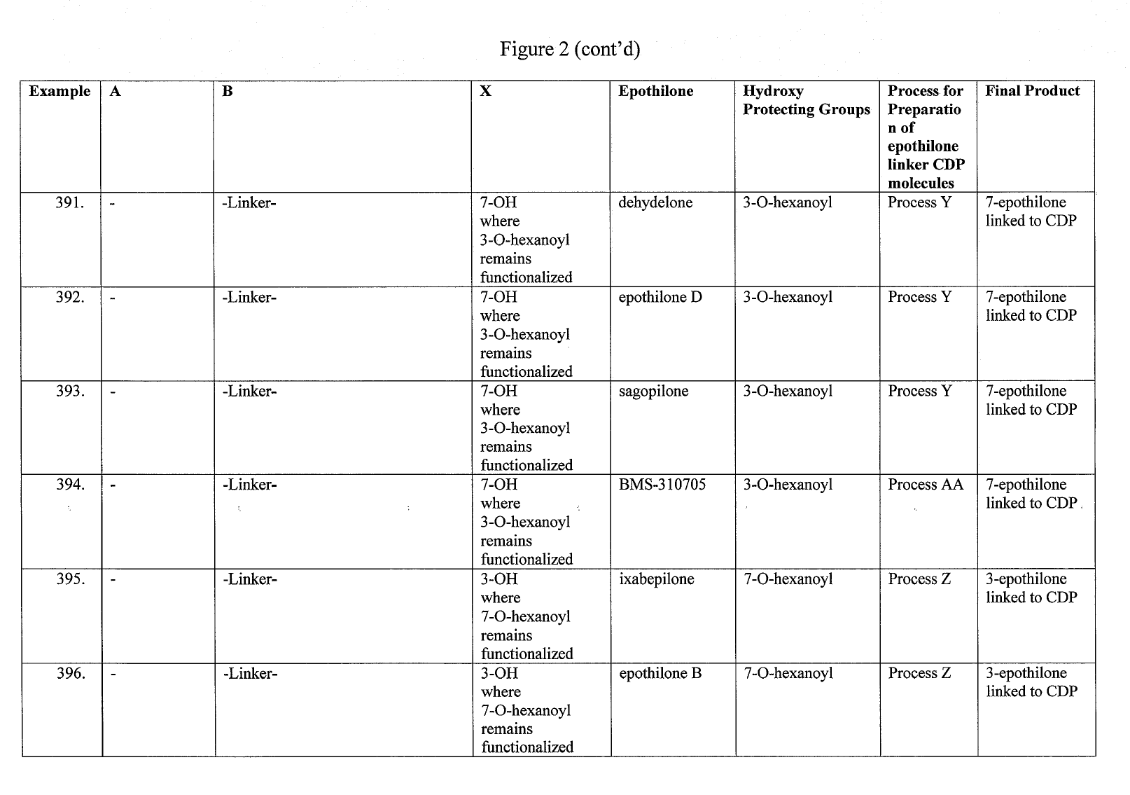

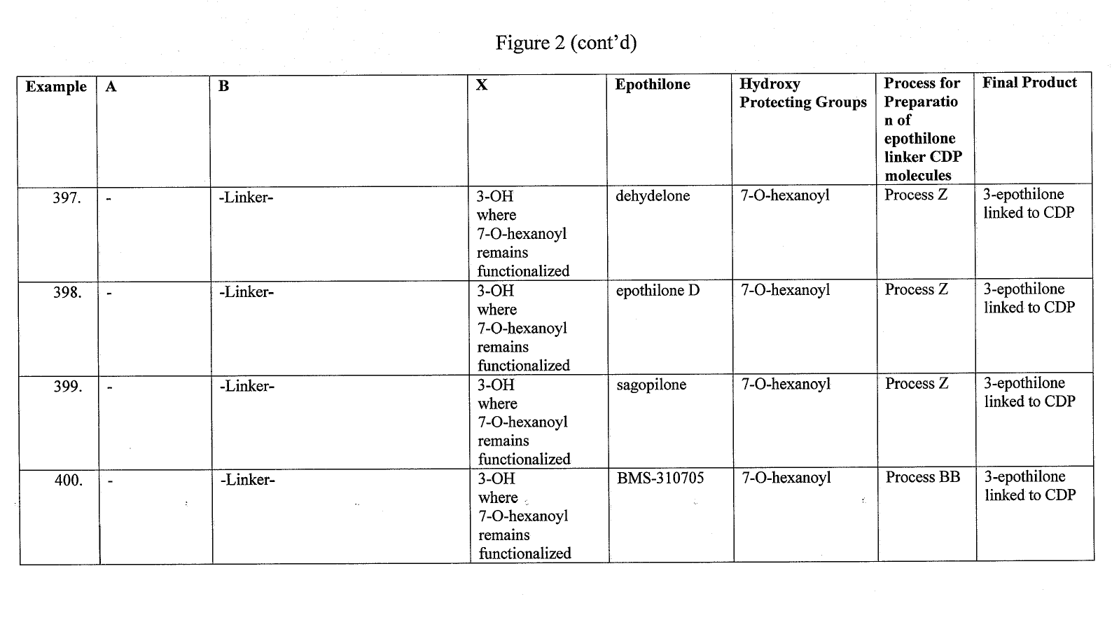

FIG. 2 is a table depicting examples of different CDP-epothilone conjugates. The CDP-epothilone conjugates in FIG. 2 are represented by the following formula:

CDP-COABX-Epothilone

In this formula,

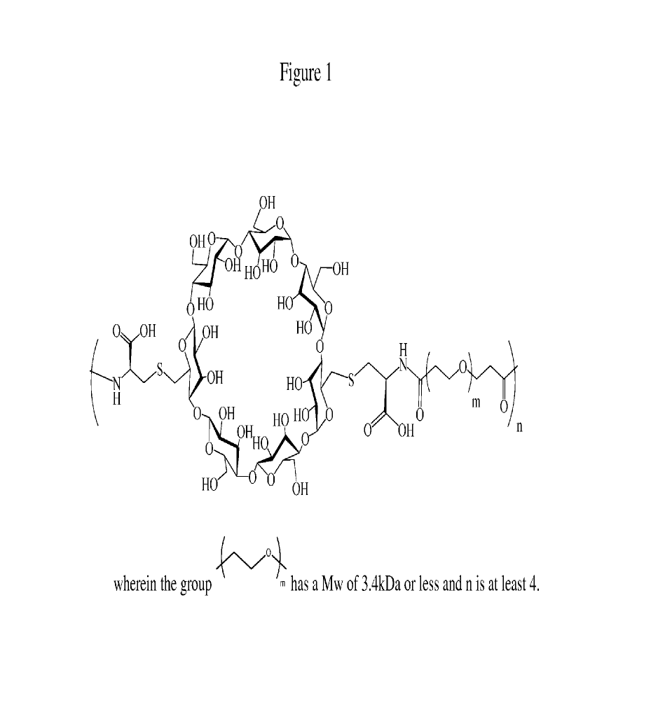

CDP is the cyclodextrin-containing polymer shown below (as well as in FIG. 1):

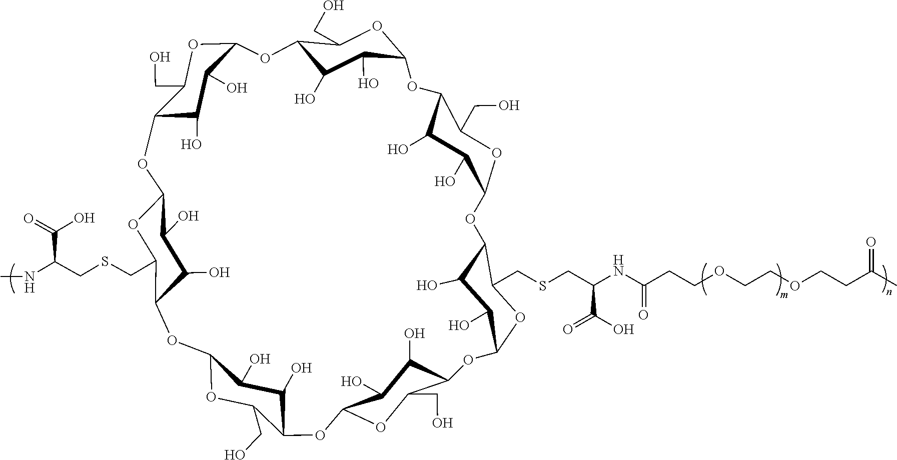

wherein the group

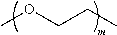

has a Mw of 3.4 kDa or less and n is at least 4, 5, 6, 7, 8, 9, 10, 11, 12, 13, 14, 15, 16, 17, 18, 19 or 20. Note that the epothilone is conjugated to the CDP through the carboxylic acid moieties of the polymer as provided above. Full loading of the epothilone onto the CDP is not required. In some embodiments, at least one, e.g., at least 2, 3, 4, 5, 6 or 7, of the carboxylic acid moieties remains unreacted with the epothilone after conjugation (e.g., a plurality of the carboxylic acid moieties remain unreacted).

CO represents the carbonyl group of the cysteine residue of the CDP;

A and B represent the link between the CDP and the epothilone. Position A is either a bond between linker B and the cysteine acid carbonyl of CDP (represented as a “—” in FIG. 2), a bond between the epothilone and the cysteine acid carbonyl of CDP (represented as a “—” in FIG. 2) or depicts a portion of the linker that is attached via a bond to the cysteine acid carbonyl of the CDP. Position B is either not occupied (represented by “—” in FIG. 2) or represents the linker or the portion of the linker that is attached via a bond to the epothilone; and

X represents the heteroatom to which the linker is coupled on the epothilone.

As provided in FIG. 2, the column with the heading “Epothilone” indicates which epothilone is included in the CDP-epothilone conjugate.

The three columns on the right of the table in FIG. 2 indicate respectively, what, if any, protecting groups are used to protect the X on the epothilone, the process for producing the CDP-epothilone conjugate, and the final product of the process for producing the CDP-epothilone conjugate.

The processes referred to in FIG. 2 are given a letter representation, e.g., Process A, Process B, Process C, etc. as seen in the second column from the right. The steps for each these processes respectively are provided below.

Process A: Couple the protected linker of position B to the epothilone, deprotect the linker and couple to CDP via the carboxylic acid group of the CDP to afford a mixture of 3- and 7-linked epothilone to CDP.

Process B: Couple the protected linker of position B to the epothilone, isolate 3-linked epothilone, and deprotect the linker and couple to CDP via the carboxylic acid group of the CDP to afford a 3-linked epothilone to CDP.

Process C: Couple the protected linker of position B to the epothilone, isolate 7-linked epothilone, deprotect the linker and couple to CDP via the carboxylic acid group of the CDP to afford a 7-linked epothilone to CDP.

Process D: Protect the epothilone, couple the protected linker of position B to an unprotected hydroxyl group of the epothilone, deprotect the linker and the epothilone hydroxyl protecting group, and couple to CDP via the carboxylic acid group of the CDP to afford a mixture of 3- and 7-linked epothilone to CDP.

Process E: Protect the epothilone, couple the protected linker of position B to an unprotected hydroxyl group of the epothilone, deprotect the linker protecting group, couple the linker to CDP via the carboxylic acid group of the CDP, and deprotect the hydroxylprotecting group to afford a mixture of 3- and 7-linked epothilone to CDP.

Process F: Protect the epothilone, isolate the 3-protected epothilone, couple the 3-protected epothilone to the protected linker of position B, deprotect linker and hydroxyl protecting group of the epothilone, and couple to CDP via the carboxylic acid group of the CDP to afford a 7-linked epothilone to CDP.

Process G: Protect the epothilone, isolate the 7-protected epothilone, couple to the protected linker of position B, deprotect linker and hydroxylprotecting group of the epothilone, and couple to CDP via the carboxylic acid group of the CDP to afford 3-linked epothilone to CDP.

Process H: Protect an amino group of the epothilone, couple the protected linker of position B to the epothilone, deprotect linker, couple to CDP via the carboxylic acid group of the CDP to afford a mixture of 3- and 7-linked epothilone to CDP, and deprotect the amino group of the epothilone.

Process I: Protect an amino group of the epothilone, couple the protected linker of position B to the epothilone, isolate the 3-linked epothilone, deprotect the linker, couple to CDP via the carboxylic acid group of the CDP to afford 3-linked epothilone to CDP, and deprotect the amino group of the epothilone.

Process J: Protect an amino group of the epothilone, couple the protected linker of position B to the epothilone, isolate the 7-linked epothilone, deprotect the linker, couple to CDP via the carboxylic acid group of the CDP to afford 7-linked epothilone to CDP, and deprotect the amino group of the epothilone.

Process K: Protect an amino group and a hydroxyl group of the epothilone, couple the protected linker of position B to an unprotected hydroxyl group of the epothilone, deprotect the linker and the hydroxyl group of the epothilone, couple to CDP via the carboxylic acid group of the CDP to afford a mixture of 3- and 7-linked epothilone to CDP, and deprotect the amino group of the epothilone.

Process L: Protect epothilone amino group and hydroxyl group, couple the protected linker of position B to unprotected hydroxyl group, deprotect linker protecting group, couple to CDP, deprotect hydroxylprotecting group to afford a mixture of 3- and 7-linked epothilone to CDP, and deprotect the amino group of the epothilone.

Process M: Protect an amino group and a hydroxyl group of the epothilone, isolate 3-protected epothilone, couple the epothilone to the linker of position B, deprotect the linker and the hydroxyl group of the epothilone, couple to CDP via the carboxylic acid group of the CDP to afford 7-linked epothilone to CDP, and deprotect the amino group of the epothilone.

Process N: Protect an amino group and a hydroxyl group of the epothilone, isolate 7-protected epothilone, couple the epothilone to the linker of position B, deprotect the linker and the hydroxyl group of the epothilone, couple to CDP via the carboxylic acid group of the CDP to afford 3-linked epothilone to CDP, and deprotect the amino group of the epothilone.

Process O: Couple the protected linker of position B to an amino group of epothilone, deprotect the linker, and couple to CDP via the carboxylic acid group to afford NH-linked epothilone to CDP.

Process P: Couple the activated linker of position B to the epothilone, and couple to CDP containing linker of position A via the linker of A to afford a mixture of 3- and 7-linked epothilone to CDP.

Process Q: Couple the activated linker of position B to the epothilone, isolate the 3-linked epothilone, and couple to the CDP containing linker of position A via the linker of A to afford the 3-linked epothilone to CDP.

Process R: Couple the activated linker of position B, isolate the 7-linked epothilone, and couple to the CDP containing linker of position A via the linker of A to afford 7-linked epothilone to CDP.

Process S: Protect the epothilone, couple the activated linker of position B to an unprotected hydroxyl group of the epothilone, deprotect the hydroxyl group of the epothilone, and couple to the CDP containing linker of position A via the linker of A to afford a mixture of 3- and 7-linked epothilone to CDP.

Process T: Protect the epothilone, couple the activated linker of position B to an unprotected hydroxyl group of the epothilone, couple to the CDP containing linker of position A via the linker of A, and deprotect hydroxyl group of the epothilone to afford a mixture of 3- and 7-linked epothilone to CDP.

Process U: Protect the epothilone, isolate the 3-protected epothilone, couple the epothilone to the activated linker of position B, deprotect the hydroxylprotecting group of the epothilone, and couple to the CDP containing linker of position A to afford the 7-linked epothilone to CDP.

Process V: Protect the epothilone, isolate the 7-protected epothilone, couple to the activated linker of position B, deprotect the hydroxyl group of the epothilone, and couple to CDP containing linker of position A via the linker of A to afford the 3-linked epothilone to CDP.

Process W: Couple the epothilone directly to CDP via the free amino group of the epothilone to the carboxylic acid group of the CDP to form NH-linked epothilone to CDP.

Process X: Couple the activated linker of position B to an amino group of epothilone, and couple to CDP containing linker of position A via the linker of A to form NH-linked epothilone to CDP.

Process Y: Protect the epothilone, isolate the 3-protected epothilone, couple the epothilone to the linker of position B, deprotect the linker, and couple to CDP via the carboxylic acid group of CDP to afford the 7-linked epothilone to CDP.

Process Z: Protect the epothilone, isolate the 7-protected epothilone, couple to the protected linker of position B, deprotect linker, and couple to CDP via the carboxylic acid group of CDP to afford the 3-linked epothilone to CDP.

Process AA: Protect the amino and hydroxyl groups of the epothilone, isolate 3-protected epothilone, couple to the protected linker of position B, deprotect the linker, and couple to CDP via the carboxylic acid group of CDP to afford 7-linked epothilone to CDP.

Process BB: Protect the amino and hydroxyl groups of the epothilone, isolate 7-protected epothilone, couple to the protected linker of position B, deprotect the linker, and couple to CDP via the carboxylic acid group of the CDP to afford 3-linked epothilone to CDP.

Process CC: Protect an amino group of the epothilone, couple the activated linker of position B to the epothilone, couple to CDP containing linker of position A via the linker of A to afford a mixture of 3- and 7-linked epothilone to CDP, and deprotect the amino group of the epothilone.

Process DD: Protect an amino group of the epothilone, couple the activated linker of position B to the epothilone, isolate the 3-linked epothilone, couple to the CDP containing linker of position A via the linker of A to afford the 3-linked epothilone to CDP, and deprotect the amino group of the epothilone.

Process EE: Protect an amino group of the epothilone, couple the activated linker of position B, isolate the 7-linked epothilone, couple to the CDP containing linker of position A via the linker of A to afford 7-linked epothilone to CDP, and deprotect the amino group of the epothilone.

Process FF: Protect an amino group and a hydroxyl group of the epothilone, couple the activated linker of position B to an unprotected hydroxyl group of the epothilone, deprotect the hydroxyl group of the epothilone, couple to the CDP containing linker of position A via the linker of A to afford a mixture of 3- and 7-linked epothilone to CDP, and deprotect the amino group of the epothilone.

Process GG: Protect an amino group and a hydroxyl group of the epothilone, couple the activated linker of position B to an unprotected hydroxyl group of the epothilone, couple to the CDP containing linker of position A via the linker of A, deprotect hydroxyl group of the epothilone to afford a mixture of 3- and 7-linked epothilone to CDP, and deprotect the amino group of the epothilone.

Process HH: Protect an amino group and a hydroxyl group of the epothilone, isolate the 3-protected epothilone, couple the epothilone to the activated linker of position B, deprotect the hydroxylprotecting group of the epothilone, couple to the CDP containing linker of position A to afford the 7-linked epothilone to CDP, and deprotect the amino group of the epothilone.

Process II: Protect an amino group and a hydroxyl group of the epothilone, isolate the 7-protected epothilone, couple to the activated linker of position B, deprotect the hydroxyl group of the epothilone, couple to CDP containing linker of position A via the linker of A to afford the 3-linked epothilone to CDP, and deprotect the amino group of the epothilone.

As shown specifically in FIG. 2, the CDP-epothilone conjugates can be prepared using a variety of methods known in the art, including those described herein. In some embodiments, the CDP-epothilone conjugates can be prepared using no protecting groups on the epothilone (see, e.g., examples 1, 2, and 3). For example, the CDP-epothilone conjugates can be prepared as a mixture (e.g., where there are two free hydroxyl groups on the epothilone) at the time the epothilone is coupled to the CDP or the linker The mixture can be coupled with a linker, e.g., a linker of position A, which is attached to the cysteine acid carbonyl of the CDP. The mixture may also be directly coupled with the CDP, i.e., the cysteine acid carbonyl of the CDP.

In some embodiments, the CDP-epothilone conjugates can be prepared using a protecting group on a hydroxyl group of the epothilone that is not used as a point of attachment to the CDP. When a linker is present, e.g., a linker of position B (see, e.g., examples 6 and 7), the linker can be coupled to the epothilone at an unprotected point of attachment, e.g., at an unprotected hydroxyl group of the epothilone. In one embodiment, the epothilone can be deprotected and a linker of position B can be coupled to CDP via linker of position A. When a linker of position A is present, it can be attached to cysteine acid carbonyl of the CDP. Position A may also be a bond, and therefore, the coupling of the epothilone and/or epothilone linker B may be directly with the CDP, i.e., the cysteine acid carbonyl of the CDP.

In some embodiments, the CDP-epothilone conjugates can be prepared using a prodrug protecting group on a hydroxyl group of the epothilone that is not used as a point of attachment to the CDP. When linker of position B is present, the linker can be coupled to the epothilone without deprotecting the epothilone. For example, the prodrug can be an ester group that remains on a hydroxyl group of the epothilone and a different hydroxyl group of the epothilone can be used as the point of attachment to the CDP (see, e.g., examples 289-400 of FIG. 2). In some embodiments, the protected epothilone can be coupled to the CDP via a linker of position A. When position A includes a linker, the linker at position A is attached to the cysteine acid carbonyl of the CDP. Position A may also be a bond, and therefore, the coupling may be directly with the CDP, i.e., the cysteine acid carbonyl of the CDP.

One or more protecting groups can be used in the processes described above to make the CDP-epothilone conjugates described herein. A protecting group can be used to control the point of attachment of the epothilone and/or epothilone linker to position A. In some embodiments, the protecting group is removed and, in other embodiments, the protecting group is not removed. If a protecting group is not removed, then it can be selected so that it is removed in vivo (e.g., acting as a prodrug). An example is hexanoic acid which has been shown to be removed by lipases in vivo if used to protect a hydroxyl group in doxorubicin. Protecting groups are generally selected for both the reactive groups of the epothilone and the reactive groups of the linker that are not targeted to be part of the coupling reaction. The protecting group should be removable under conditions which will not degrade the epothilone and/or linker material. Examples include t-butyldimethylsilyl (“TBDMS”) and TROC (derived from 2,2,2-trichloroethoxy chloroformate). Carboxybenzyl (“CBz”) can also be used in place of TROC if there is selectivity seen for removal over olefin reduction. This can be addressed by using a group which is more readily removed by hydrogentation such as -methoxybenzyl OCO—. Other protecting groups may also be acceptable. One of skill in the art can select suitable protecting groups for the products and methods described herein.

Although the products in FIG. 2 corresponding to processes E, L, T, and FF result in a mixture of 3- and 7-linked epothilone to CDP. These processes can be readily modified to produce a product having an epothilone linked by a single group, e.g., linked either through the 3-position only or 7-position only. For example, a 3-linked epothilone to CDP can be produced in methods E, L, T, and FF by separating and isoloating a pure isomer of the 7-protected epothilone prior to coupling of the epothilone to the CDP; and a 7-linked epothilone to CDP can be produced in methods E, L, T, and FF by separating and isoloating a pure isomer of the 3-protected epothilone prior to coupling of the epothilone to the CDP.

CDP-Epothilone Conjugate Characteristics

In some embodiments, the CDP and/or CDP-epothilone conjugates as described herein have polydispersities less than about 3, or even less than about 2.

One embodiment of the present invention provides an improved delivery of certain epothilones by covalently conjugating them to a CDP. Such conjugation improves the aqueous solubility and hence the bioavailability of the epothilone. Accordingly, in one embodiment of the invention, the epothilone is a hydrophobic compound with a log P>0.4, >0.6, >0.8, >1, >2, >3, >4, or even >5. In other embodiments, an epothilone may be attached to another compound, such as an amino acid, prior to covalently attaching the conjugate onto the CDP.

The CDP-epothilone conjugates described herein preferably have molecular weights in the range of 10,000 to 500,000; 30,000 to 200,000; or even 70,000 to 150,000 amu. In certain embodiments as disclosed herein, the compound has a number average (Mₙ) molecular weight between 1,000 to 500,000 amu, or between 5,000 to 200,000 amu, or between 10,000 to 100,000 amu. One method to determine molecular weight is by gel permeation chromatography (“GPC”), e.g., mixed bed columns, CH₂Cl₂ solvent, light scattering detector, and off-line do/dc. Other methods are known in the art.

In certain embodiments as disclosed herein, the CDP-epothilone conjugate is biodegradable or bioerodable.

In certain embodiments as disclosed herein, the epothilone or prodrug thereof makes up at least 3% (e.g., at least about 5%) by weight of the compound. In certain embodiments, the therapeutic agent or prodrug thereof makes up at least 20% by weight of the compound. In certain embodiments, the therapeutic agent or prodrug thereof makes up at least 5%, 10%, 15%, or at least 20% by weight of the compound.

In other embodiments, the CDP-epothilone conjugate may be a flexible or flowable material. When the CDP used is itself flowable, the CDP composition of the invention, even when viscous, need not include a biocompatible solvent to be flowable, although trace or residual amounts of biocompatible solvents may still be present.

When a solvent is used to facilitate mixing or to maintain the flowability of the CDP-epothilone conjugate, it should be non-toxic, otherwise biocompatible, and should be used in relatively small amounts. Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

In certain embodiments, the CDP-epothilone conjugates are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

In certain embodiments, the CDP-epothilone conjugates described herein, upon contact with body fluids, undergo gradual degradation. The life of a biodegradable polymer in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with an epothilone or other material, release of the epothilone or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the epothilone or any other material associated with the polymer.

A variety of factors may affect the desired rate of hydrolysis of CDP-epothilone conjugates, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as an epothilone or other material loaded in the CDP-epothilone conjugates of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different CDP-epothilone conjugates of the present invention may be compared by subjecting them to such a protocol. In certain instances, it may be necessary to process polymeric systems in the same fashion to allow direct and relatively accurate comparisons of different systems to be made. For example, the present invention teaches several different methods of formulating the CDP-epothilone conjugates. Such comparisons may indicate that any one CDP-epothilone conjugate releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the present invention and release rate protocols.

In certain embodiments, when formulated in a certain manner, the release rate for CDP-epothilone conjugates of the present invention may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any incorporated material may be 10,000 ng/day/mg, or even higher. In certain instances, materials incorporated and characterized by such release rate protocols may include therapeutic agents, fillers, and other substances.

In another aspect, the rate of release of any material from any CDP-epothilone conjugate of the present invention (e.g., the epothilone) may be presented as the half-life of such material in the matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

Physical Structures of the CDP-Epothilone Conjugates

The CDP-epothilone conjugates may be formed in a variety of shapes. For example, in certain embodiments, the CDP-epothilone conjugates may be formed into a particle (e.g., a nanoparticle). In one embodiment, the CDP-epothilone conjugate self assembles into a nanoparticle. In one embodiment, the CDP-epothilone conjugate self assembles into a nanoparticle in an aqueous solution, e.g., water.

In addition to intracellular delivery of an epothilone, it also possible that nanoparticles comprising a CDP-epothilone conjugate described herein may undergo endocytosis, thereby obtaining access to the cell. The frequency of such an endocytosis process will likely depend on the size of any nanoparticle.

In one embodiment, the surface charge of the particle is neutral, or slightly negative. In some embodiments, the zeta potential of the particle surface is from about −80 mV to about 50 mV.

Conjugate Number

Conjugate number, as used herein, is the number of cyclodextrin containing polymer (“CDP”) therapeutic agent conjugate molecules (e.g., a CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate), present in a particle or nanoparticle. For purposes of determining conjugate number, a particle or nanoparticle is an entity having one, or typically, more than one CDP therapeutic agent conjugate molecules, which, at the concentration suitable for administration to humans, behaves as a single unit in any of water, e.g., water at neutral pH, PBS, e.g., PBS at pH 7.4, or in a formulation in which it will be administered to patients. For purposes of calculating conjugate number, a CDP therapeutic agent conjugate molecule is a single CDP polymer with its covalently linked therapeutic agent.

Methods disclosed herein provide for evaluating a particle, e.g., a nanoparticle, or preparation of particles, e.g., nanoparticles, wherein said particles, e.g., nanoparticles, comprise a CDP therapeutic agent conjugate. Generally, the method comprises providing a sample comprising a plurality of said particles, e.g., nanoparticles, determining a value for the number of CDP therapeutic agent conjugates in a particle, e.g., nanoparticle, in the sample, to thereby evaluate a preparation of particles, e.g., nanoparticles.

Typically the value for a particle will be a function of the values obtained for a plurality of particles, e.g., the value will be the average of values determined for a plurality of particles.

In embodiments the method further comprises comparing the determined value with a reference value. The comparison can be used in a number of ways. By way of example, in response to a comparison or determination made in the method, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale. E.g., based on the result of the determination, or upon comparison to a reference standard, the batch from which the sample is taken can be processed, e.g., as just described.

In one embodiment, the CDP-epothilone conjugate forms or is provided as a particle (e.g., a nanoparticle) having a conjugate number described herein. By way of example, a CDP-epothilone conjugate forms, or is provided in, a nanoparticle having a conjugate number of: 1 or 2 to 25; 1 or 2 to 20; 1 or 2 to 15; 1 or 2 to 10; 1 to 3; 1 to 4; 1 to 5; 1 to 6; 1 to 7; 1 to 10; 2 to 3; 2 to 4; 2 to 5; 2 to 6; 2 to 7; 2 to 10; 3 to 4; 3 to 5; 3 to 6; 3 to 7; 3 to 10; 10 to 15; 15-20; or 20-25.

In an embodiment the conjugate number is 2 to 4 or 2 to 5.

In an embodiment the conjugate number is 1, 2, 3, 4, 5, 6, 7, 8, 9, or 10.

In an embodiment the nanoparticle forms, or is provided in, a preparation of nanoparticles, e.g., a pharmaceutical preparation, wherein at least 40, 50, 60, 70, 80, 90 or 95% of the particles in the preparation have a conjugate number provided herein. In an embodiment the nanoparticle forms, or is provided in, a preparation of nanoparticles, e.g., a pharmaceutical preparation, wherein at least 60% of the particles in the preparation have a conjugate number of 1-5 or 2-5.

In an embodiment the conjugate number is from 1-100; 25 to 100; 50 to 100; 75-100; 25 to 75, 25 to 50, or 50 to 75; 25 to 40; 25 to 50; 30 to 50; 30 to 40; or 30 to 75.

In an embodiment, the CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate) forms, or is provided as, a particle (e.g., a nanoparticle), wherein at least 60% of the particles in the preparation have a conjugate number of 1-100; 25 to 100; 50 to 100; 75-100; 25 to 75, 25 to 50, or 50 to 75; 25 to 40; 25 to 50; 30 to 50; 30 to 40; or 30 to 75.

In another aspect, the invention features, a method of evaluating a particle or a preparation of particles, wherein said particles, comprise one or a plurality of CDP therapeutic agent conjugate molecules, e.g., a CDP-epothilone conjugate (e.g., a CDP-ixabepilone conjugate, a CDP-epothilone B conjugate, a CDP-epothilone D conjugate, a CDP-BMS310705 conjugate, CDP-dehydelone conjugate, or a CDP-ZK-EPO conjugate). The method comprises:

providing a sample comprising one or a plurality of said particles;

determining a value for the number of CDP conjugate molecules in a particle in said sample (the conjugate number),

thereby evaluating a preparation of particles.

In an embodiment the method comprises one or both of:

a) comparing said determined value with a reference value, e.g., a range of values, or

b) responsive to said determination, classifying said particles,

In an embodiment the particle is a nanoparticle.

In an embodiment the method further comprises comparing said determined value with a reference standard. The reference value can be selected from a value, e.g., a range, provided herein, e.g., 1 or 2 to 8, 1 or 2 to 7, 1 or 2 to 6, 1 or 2 to 5, or 2-4.

The reference value can be selected from a value, e.g., a range, provided herein, e.g., 1-100; 25 to 100; 50 to 100; 75-100; 25 to 75, 25 to 50, or 50 to 75; 25 to 40; 25 to 50; 30 to 50; 30 to 40; or 30 to 75.

In an embodiment, responsive to said comparison, a decision or step is taken, e.g., a production parameter in a process for making a particle is altered, the sample is classified, selected, accepted or discarded, released or withheld, processed into a drug product, shipped, moved to a different location, formulated, e.g., formulated with another substance, e.g., an excipient, labeled, packaged, released into commerce, or sold or offered for sale.

In an embodiment said CDP therapeutic agent (e.g., eptothilone) conjugate is selected from those disclosed in herein.

In an embodiment said therapeutic agent is selected from those disclosed herein.

In an embodiment said particle is selected from those disclosed in herein.

In an embodiment, the determined value for conjugate number is compared with a reference, and responsive to said comparison said particle or preparation of particles is classified, e.g., as suitable for use in human subjects, not suitable for use in human subjects, suitable for sale, meeting a release specification, or not meeting a release specification.

As discussed above, conjugate number is defined as the number of CDP-therapeutic agent (e.g., epothilone) conjugate molecules that self-assemble into a particle or nanoparticle, thus

C_{J}=[CDP-therapeutic agent (e.g., epothilone) conjugatel/P (or NP)

where Cj is conjugate number, [CDP-therapeutic agent (e.g., epothilone) conjugate]/is the number of CDP-therapeutic agent (e.g., epothilone) conjugate molecules, and P (or NP) is a single particle (or nanoparticle).

In order to arrive and conjugate number one determines the size of a particle, e.g., by dynamic light scattering. The size should be viscosity-adjusted size. The hydrodynamic volume of a CDP-therapeutic agent (e.g., epothilone) conjugate, or a molecule of similar molecular weight, is determined, to provide an expected hydrodynamic volume. Comparison of the expected hydrodynamic volume for the CDP-therapeutic agent (e.g., epothilone) conjugate with the volume for a particle of determined size provides conjugate number.

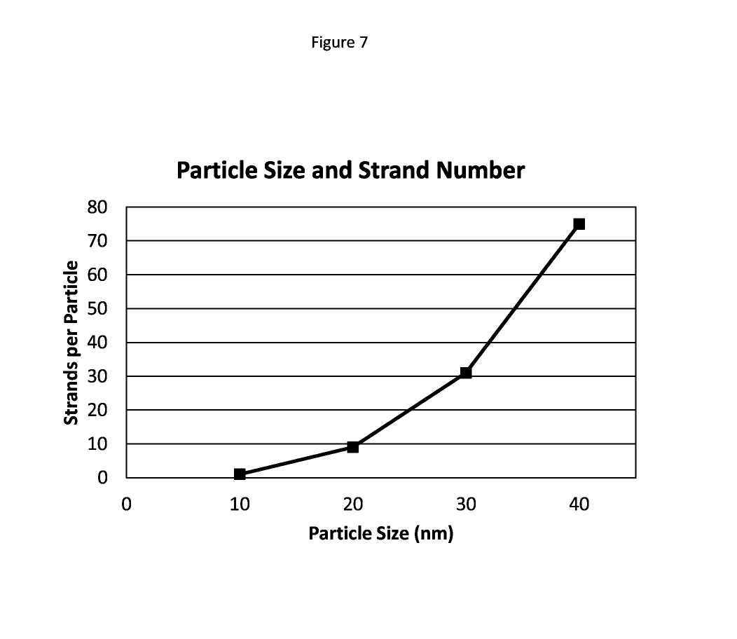

The determination of conjugate number is demonstrated with CRLX101, in which camptothecin is coupled to the CDP backbone. In the case of CRLX101, a number of fundamental assumptions are made in postulating nanoparticle characteristics. First, macromolecular volume estimates are based on work done with bovine serum albumin (BSA), a biological macromolecule of similar size to CRLX101 (BSA MS=67 kDa, 101 MW=66.5 kDa). It has been demonstrated that a single strand of BSA has a hydrodynamic diameter of 9.5 nm Simple volume calculations yield a volume of 3589 nm³. Extending this to CRLX 101 with an average 30 nm particle, gives a volume of 33,485 nm³. With a particle size of 5-40 nm the conjugate number is 1-30. FIG. 7 shows a calculated strand dependence on particle size.

Polymer Polydispersity. CRLX101 molecules fall within a range of molecular weights, with molecules of varying weight providing varying contributions to the particle diameter and conjugate number. Particles could form which are made up of strands which are larger and smaller than the average. Strands may also associate to a maximum size which could be shear-limited.

Particle Shape. Particle shape is assumed to be roughly spherical, and driven by either (or both) the hydrophobic region created by the CDP-therapeutic agent conjugate, or by guest-host complexation with pendant therapeutic agent molecules making inclusion complexes with CDs from adjacent strands. One critical point of note is that as a drug product, the NPs are in a somewhat controlled environment as they are characterized. Upon administration, myriad possibilities exist for interaction with endogenous substances: inclusion complexes of circulating small molecules, metal ion complexation with the PEG subunits, etc. Any one of these are all of them in concert could dramatically alter the NP structure and function.

### CDPs, Methods of Making Same, and Methods of Conjugating CDPs to Epothilones

Generally, the CDP-epothilone conjugates described herein can be prepared in one of two ways: monomers bearing epothilones, targeting ligands, and/or cyclodextrin moieties can be polymerized, or polymer backbones can be derivatized with epothilones, targeting ligands, and/or cyclodextrin moieties.

Thus, in one embodiment, the synthesis of the CDP-epothilone conjugates can be accomplished by reacting monomers M-L-CD and M-L-D (and, optionally, M-L-T), wherein

CD represents a cyclic moiety, such as a cyclodextrin molecule, or derivative thereof;

L, independently for each occurrence, may be absent or represents a linker group;

D, independently for each occurrence, represents the same or different epothilone or prodrug thereof;

T, independently for each occurrence, represents the same or different targeting ligand or precursor thereof; and

M represents a monomer subunit bearing one or more reactive moieties capable of undergoing a polymerization reaction with one or more other M in the monomers in the reaction mixture, under conditions that cause polymerization of the monomers to take place.

In some embodiments, one or more of the epothilone moieties in the CDP-epothilone conjugate can be replaced with another therapeutic agent, e.g., another anticancer agent or anti-inflammatory agent.

In certain embodiments, the reaction mixture may further comprise monomers that do not bear CD, T, or D moieties, e.g., to space the derivatized monomer units throughout the polymer.

In an alternative embodiment, the invention contemplates synthesizing a CDP-epothilone conjugate by reacting a polymer P (the polymer bearing a plurality of reactive groups, such as carboxylic acids, alcohols, thiols, amines, epoxides, etc.) with grafting agents X-L-CD and Y-L-D (and, optionally, Z-L-T), wherein

CD represents a cyclic moiety, such as a cyclodextrin molecule, or derivative thereof;

L, independently for each occurrence, may be absent or represents a linker group;

D, independently for each occurrence, represents the same or different epothilone or prodrug thereof;

T, independently for each occurrence, represents the same or different targeting ligand or precursor thereof;

X, independently for each occurrence, represents a reactive group, such as carboxylic acids, alcohols, thiols, amines, epoxides, etc., capable of forming a covalent bond with a reactive group of the polymer; and

Y and Z, independently for each occurrence, represent inclusion hosts or reactive groups, such as carboxylic acids, alcohols, thiols, amines, epoxides, etc., capable of forming a covalent bond with a reactive group of the polymer or inclusion complexes with CD moieties grafted to the polymer, under conditions that cause the grafting agents to form covalent bonds and/or inclusion complexes, as appropriate, with the polymer or moieties grafted to the polymer.

In some embodiments, one or more of the epothilone moieties in the CDP-epothilone conjugate can be replaced with another therapeutic agent, e.g., another anticancer agent or anti-inflammatory agent.

For example, if the CDP includes alcohols, thiols, or amines as reactive groups, the grafting agents may include reactive groups that react with them, such as isocyanates, isothiocyanates, acid chlorides, acid anhydrides, epoxides, ketenes, sulfonyl chlorides, activated carboxylic acids (e.g., carboxylic acids treated with an activating agent such as PyBrOP, carbonyldiimidazole, or another reagent that reacts with a carboxylic acid to form a moiety susceptible to nucleophilic attack), or other electrophilic moieties known to those of skill in the art. In certain embodiments, a catalyst may be needed to cause the reaction to take place (e.g., a Lewis acid, a transition metal catalyst, an amine base, etc.) as will be understood by those of skill in the art.

In certain embodiments, the different grafting agents are reacted with the polymer simultaneously or substantially simultaneously (e.g., in a one-pot reaction), or are reacted sequentially with the polymer (optionally with a purification and/or wash step between reactions).

Another aspect of the present invention is a method for manufacturing the linear or branched CDPs and CDP-epothilone conjugates as described herein. While the discussion below focuses on the preparation of linear cyclodextrin molecules, one skilled in the art would readily recognize that the methods described can be adapted for producing branched polymers by choosing an appropriate comonomer precursor.

Accordingly, one embodiment of the invention is a method of preparing a linear CDP. According to the invention, a linear CDP may be prepared by copolymerizing a cyclodextrin monomer precursor disubstituted with an appropriate leaving group with a comonomer precursor capable of displacing the leaving groups. The leaving group, which may be the same or different, may be any leaving group known in the art which may be displaced upon copolymerization with a comonomer precursor. In a preferred embodiment, a linear CDP may be prepared by iodinating a cyclodextrin monomer precursor to form a diiodinated cyclodextrin monomer precursor and copolymerizing the diiodinated cyclodextrin monomer precursor with a comonomer precursor to form a linear CDP having a repeating unit of formula I or II, provided in the section entitles “CDP-Epothilone conjugates” or a combination thereof, each as described above. In some embodiments, the cyclodextrin moiety precursors are in a composition, the composition being substantially free of cyclodextrin moieties having other than two positions modified to bear a reactive site (e.g., 1, 3, 4, 5, 6, or 7). While examples presented below discuss iodinated cyclodextrin moieties, one skilled in the art would readily recognize that the present invention contemplates and encompasses cyclodextrin moieties wherein other leaving groups such as alkyl and aryl sulfonate may be present instead of iodo groups. In a preferred embodiment, a method of preparing a linear cyclodextrin copolymer of the invention by iodinating a cyclodextrin monomer precursor as described above to form a diiodinated cyclodextrin monomer precursor of formula IVa, IVb, IVc or a mixture thereof:

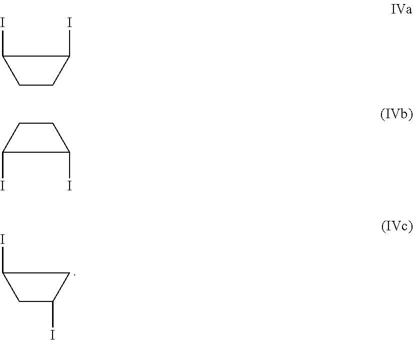

In some embodiments, the iodine moieties as shown on the cyclodextrin moieties are positioned such that the derivatization on the cyclodextrin is on the A and D glucopyranose moieties. In some embodiments, the iodine moieties as shown on the cyclodextrin moieties are positioned in such that the derivatization on the cyclodextrin is on the A and C glucopyranose moieties. In some embodiments, the iodine moieties as shown on the cyclodextrin moieties are positioned in such that the derivatization on the cyclodextrin is on the A and F glucopyranose moieties. In some embodiments, the iodine moieties as shown on the cyclodextrin moieties are positioned in such that the derivatization on the cyclodextrin is on the A and E glucopyranose moieties.

The diiodinated cyclodextrin may be prepared by any means known in the art. (Tabushi et al. J. Am. Chem. 106, 5267-5270 (1984); Tabushi et al. J. Am. Chem. 106, 4580-4584 (1984)). For example, β-cyclodextrin may be reacted with biphenyl-4,4′-disulfonyl chloride in the presence of anhydrous pyridine to form a biphenyl-4,4′-disulfonyl chloride capped β-cyclodextrin which may then be reacted with potassium iodide to produce diiodo-β-cyclodextrin. The cyclodextrin monomer precursor is iodinated at only two positions. By copolymerizing the diiodinated cyclodextrin monomer precursor with a comonomer precursor, as described above, a linear cyclodextrin polymer having a repeating unit of Formula Ia, Ib, or a combination thereof, also as described above, may be prepared. If appropriate, the iodine or iodo groups may be replaced with other known leaving groups.

Also according to the invention, the iodo groups or other appropriate leaving group may be displaced with a group that permits reaction with a comonomer precursor, as described above. For example, a diiodinated cyclodextrin monomer precursor of formula IVa, IVb, IVc or a mixture thereof may be aminated to form a diaminated cyclodextrin monomer precursor of formula Va, Vb, Vc or a mixture thereof:

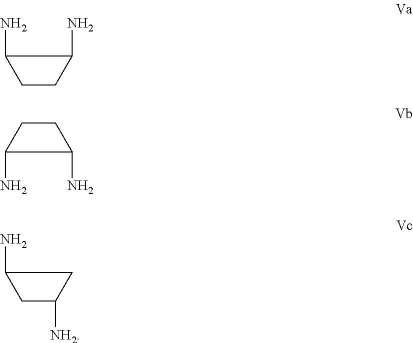

In some embodiments, the amino moieties as shown on the cyclodextrin moieties are positioned such that the derivatization on the cyclodextrin is on the A and D glucopyranose moieties. In some embodiments, the amino moieties as shown on the cyclodextrin moieties are positioned in such that the derivatization on the cyclodextrin is on the A and C glucopyranose moieties. In some embodiments, the amino moieties as shown on the cyclodextrin moieties are positioned in such that the derivatization on the cyclodextrin is on the A and F glucopyranose moieties. In some embodiments, the amino moieties as shown on the cyclodextrin moieties are positioned in such that the derivatization on the cyclodextrin is on the A and E glucopyranose moieties.

The diaminated cyclodextrin monomer precursor may be prepared by any means known in the art. (Tabushi et al. Tetrahedron Lett. 18:11527-1530 (1977); Mungall et al., J. Org. Chem. 16591662 (1975)). For example, a diiodo-β-cyclodextrin may be reacted with sodium azide and then reduced to form a diamino-β-cyclodextrin). The cyclodextrin monomer precursor is aminated at only two positions. The diaminated cyclodextrin monomer precursor may then be copolymerized with a comonomer precursor, as described above, to produce a linear cyclodextrin copolymer having a repeating unit of formula I-II as provided in the section entitles “CDP-Epothilone conjugates” or a combination thereof, also as described above. However, the amino functionality of a diaminated cyclodextrin monomer precursor need not be directly attached to the cyclodextrin moiety. Alternatively, the amino functionality or another nucleophilic functionality may be introduced by displacement of the iodo or other appropriate leaving groups of a cyclodextrin monomer precursor with amino group containing moieties such as, for example, HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine to form a diaminated cyclodextrin monomer precursor of formula Vd, Ve, Vf or a mixture thereof:

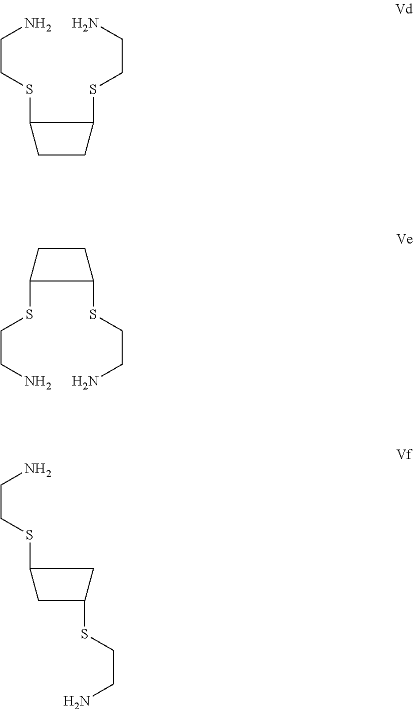 [SMILES: NCCSC1CCC1SCCN ; NCCSC1CCC1SCCN ; NCCSC1CC(SCCN)C1]

In some embodiments, the —SCH₂CH₂NH₂ moieties as shown on the cyclodextrin moieties are positioned such that the derivatization on the cyclodextrin is on the A and D glucopyranose moieties. In some embodiments, the —SCH₂CH₂NH₂ moieties as shown on the cyclodextrin moieties are positioned in such that the derivatization on the cyclodextrin is on the A and C glucopyranose moieties. In some embodiments, the —SCH₂CH₂NH₂ moieties as shown on the cyclodextrin moieties are positioned in such that the derivatization on the cyclodextrin is on the A and F glucopyranose moieties. In some embodiments, the —SCH₂CH₂NH₂ moieties as shown on the cyclodextrin moieties are positioned in such that the derivatization on the cyclodextrin is on the A and E glucopyranose moieties.

A linear oxidized CDP may also be prepared by oxidizing a reduced linear cyclodextrin-containing copolymer as described below. This method may be performed as long as the comonomer does not contain an oxidation sensitive moiety or group such as, for example, a thiol.

A linear CDP of the invention may be oxidized so as to introduce at least one oxidized cyclodextrin monomer into the copolymer such that the oxidized cyclodextrin monomer is an integral part of the polymer backbone. A linear CDP which contains at least one oxidized cyclodextrin monomer is defined as a linear oxidized cyclodextrin copolymer or a linear oxidized cyclodextrin-containing polymer. The cyclodextrin monomer may be oxidized on either the secondary or primary hydroxyl side of the cyclodextrin moiety. If more than one oxidized cyclodextrin monomer is present in a linear oxidized cyclodextrin copolymer of the invention, the same or different cyclodextrin monomers oxidized on either the primary hydroxyl side, the secondary hydroxyl side, or both may be present. For illustration purposes, a linear oxidized cyclodextrin copolymer with oxidized secondary hydroxyl groups has, for example, at least one unit of formula VIa or VIb:

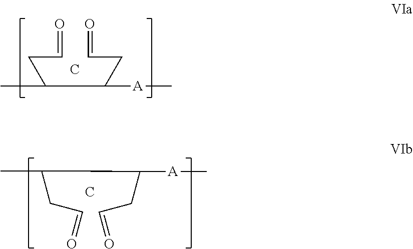

In formulae VIa and VIb, C is a substituted or unsubstituted oxidized cyclodextrin monomer and the comonomer (i.e., shown herein as A) is a comonomer bound, i.e., covalently bound, to the oxidized cyclodextrin C. Also in formulae VIa and VIb, oxidation of the secondary hydroxyl groups leads to ring opening of the cyclodextrin moiety and the formation of aldehyde groups.

A linear oxidized CDP copolymer may be prepared by oxidation of a linear cyclodextrin copolymer as discussed above. Oxidation of a linear cyclodextrin copolymer of the invention may be accomplished by oxidation techniques known in the art. (Hisamatsu et al., Starch 44:188-191 (1992)). Preferably, an oxidant such as, for example, sodium periodate is used. It would be understood by one of ordinary skill in the art that under standard oxidation conditions that the degree of oxidation may vary or be varied per copolymer. Thus in one embodiment of the invention, a CDP may contain one oxidized cyclodextrin monomer. In another embodiment, substantially all cyclodextrin monomers of the copolymer would be oxidized.

Another method of preparing a linear oxidized CDP involves the oxidation of a diiodinated or diaminated cyclodextrin monomer precursor, as described above, to form an oxidized diiodinated or diaminated cyclodextrin monomer precursor and copolymerization of the oxidized diiodinated or diaminated cyclodextrin monomer precursor with a comonomer precursor. In a preferred embodiment, an oxidized diiodinated cyclodextrin monomer precursor of formula VIIa, VIIb, VIIc, or a mixture thereof:

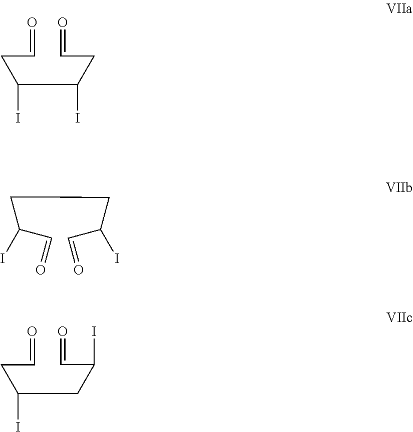 [SMILES: O=CCC(I)C(I)CC=O ; O=CC(I)CCC(I)C=O ; O=CCC(I)CC(I)C=O]

may be prepared by oxidation of a diiodinated cyclodextrin monomer precursor of formulae IVa, IVb, IVc, or a mixture thereof, as described above. In another preferred embodiment, an oxidized diaminated cyclodextrin monomer precursor of formula VIIIa, VIIIb, VIIIc or a mixture thereof:

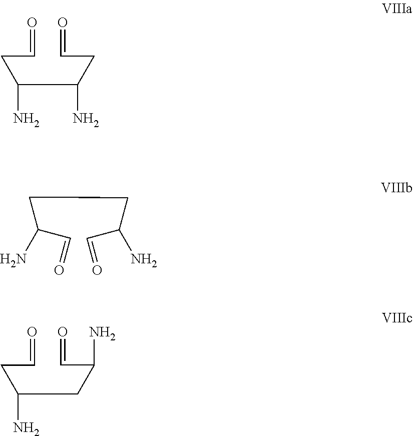 [SMILES: NC(CC=O)C(N)CC=O ; NC(C=O)CCC(N)C=O ; NC(C=O)CC(N)CC=O]

may be prepared by amination of an oxidized diiodinated cyclodextrin monomer precursor of formulae VIIa, VIIb, VIIc, or a mixture thereof, as described above. In still another preferred embodiment, an oxidized diaminated cyclodextrin monomer precursor of formula IXa, IXb, IXc or a mixture thereof:

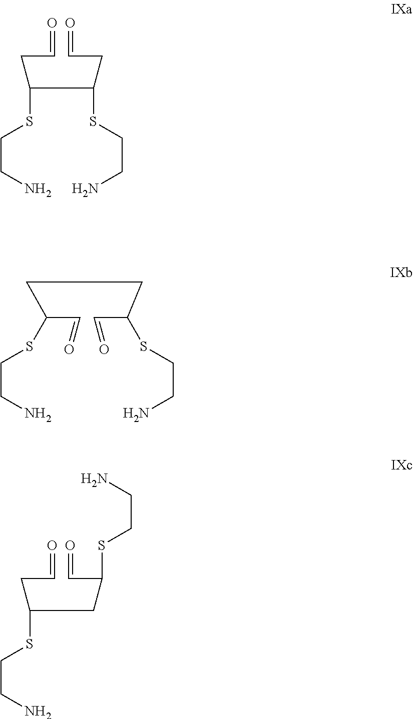 [SMILES: NCCSC(CC=O)C(CC=O)SCCN ; NCCSC(C=O)CCC(C=O)SCCN ; NCCSC(C=O)CC(CC=O)SCCN]

may be prepared by displacement of the iodo or other appropriate leaving groups of an oxidized cyclodextrin monomer precursor disubstituted with an iodo or other appropriate leaving group with the amino or other nucleophilic group containing moiety such as, e.g. HSCH₂CH₂NH₂ (or a di-nucleophilic molecule more generally represented by HW—(CR₁R₂)ₙ—WH wherein W, independently for each occurrence, represents O, S, or NR₁; R₁ and R₂, independently for each occurrence, represent H, (un)substituted alkyl, (un)substituted aryl, (un)substituted heteroalkyl, (un)substituted heteroaryl) with an appropriate base such as a metal hydride, alkali or alkaline carbonate, or tertiary amine

Alternatively, an oxidized diiodinated or diaminated cyclodextrin monomer precursor, as described above, may be prepared by oxidizing a cyclodextrin monomer precursor to form an oxidized cyclodextrin monomer precursor and then diiodinating and/or diaminating the oxidized cyclodextrin monomer, as described above. As discussed above, the cyclodextrin moiety may be modified with other leaving groups other than iodo groups and other amino group containing functionalities. The oxidized diiodinated or diaminated cyclodextrin monomer precursor may then be copolymerized with a comonomer precursor, as described above, to form a linear oxidized cyclodextrin copolymer of the invention.

A linear oxidized CDP may also be further modified by attachment of at least one ligand to the copolymer. The ligand is as described above.

In some embodiments, a CDP comprises: cyclodextrin moieties, and comonomers which do not contain cyclodextrin moieties (comonomers), and wherein the CDP comprises at least four, five six, seven, eight, nine, ten, eleven, twelve, thirteen, fourteen, fifteen, sixteen, seventeen, eighteen, nineteen or twenty cyclodextrin moieties and at least four, five six, seven, eight, nine, ten, eleven, twelve, thirteen, fourteen, fifteen, sixteen, seventeen, eighteen, nineteen or twenty comonomers.

In some embodiments, the at least four, five six, seven, eight, etc., cyclodextrin moieties and at least four, five six, seven, eight, nine, ten, eleven, twelve, thirteen, fourteen, fifteen, sixteen, seventeen, eighteen, nineteen or twenty comonomers alternate in the water soluble linear polymer.

In some embodiments, the cyclodextrin moieties comprise linkers to which therapeutic agents may be further linked.

In some embodiments, the CDP has no epothilones attached. In some embodiments, the CDP has a plurality (i.e., more than one) of epothilones attached (e.g., through a linker). In some embodiments, the epothilones are attached via a second linker

In some embodiments, the comonomer is a compound containing residues of least two functional groups through which reaction and thus linkage of the cyclodextrin monomers is achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the residues of the two functional groups are the same and are located at termini of the comonomer. In some embodiments, a comonomer contains one or more pendant groups with at least one functional group through which reaction and thus linkage of an epothilone can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient epothilone such that up to at least 5%, 10%, 15%, 20%, 25%, 30%, or even 35% by weight of the CDP (e.g., the water soluble linear polymer), when conjugated, is epothilone.

In some embodiments, the molecular weight of the CDP is 10,000-500,000 Da, e.g., about 30,000 to about 100,000 Da.

In some embodiments, the cyclodextrin moieties make up at least about 2%, 5%, 10%, 11%, 12%, 13%, 14%, 15%, 16%, 17%, 18%, 19%, 20%, 30%, 50% or 80% of the polymer by weight.

In some embodiments, the CDP is made by a method comprising providing cyclodextrin moiety precursors modified to bear one reactive site at each of exactly two positions, and reacting the cyclodextrin moiety with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and comonomer is produced.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain.

In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR₁, O or S), —OC(O)—, —C(═O)O—, —NR₁—, —NR₁CO—, —C(O)NR₁—, —S(O)ₙ— (wherein n is 0, 1, or 2), —OC(O)—NR₁—, —NR₁—C(O)—NR₁—, —NR₁—C(NR₁)—NR₁—, and —B(OR₁)—; and R₁, independently for each occurrence, represents H or a lower alkyl.

In some embodiments, the CDP is a polymer of the following formula:

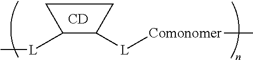

wherein each L is independently a linker, each comonomer is independently a comonomer described herein, and n is at least 4, 5, 6, 7, 8, 9, 10, 11, 12, 13, 14, 15, 16, 17, 18, 19 or 20. In some embodiments, the molecular weight of the comonomer is from about 2000 to about 5000 Da (e.g., from about 3000 to about 4000 Da (e.g., about 3400 Da).

In some embodiments, the CDP is a polymer of the following formula:

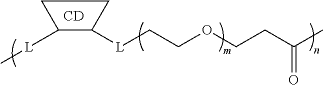

wherein each L is independently a linker,

wherein the group

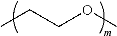

has a Mw of 3.4 kDa or less and n is at least 4, 5, 6, 7, 8, 9, 10, 11, 12, 13, 14, 15, 16, 17, 18, 19 or 20.

In some embodiments,

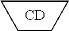

is alpha, beta or gamma cyclodextrin, e.g., beta cyclodextrin.

In some embodiments, each L independently comprises an amino acid or a derivative thereof. In some embodiments, at least one L comprises cysteine or a derivative thereof. In some embodiments, each L comprises cysteine. In some embodiments, each L is cysteine and the cysteine is connected to the CD by way of a thiol linkage.

In some embodiments, the CDP is a polymer of the following formula:

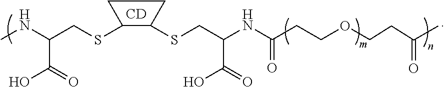

wherein the group

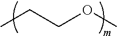

has a Mw of 3.4 kDa or less and n is at least 4, 5, 6, 7, 8, 9, 10, 11, 12, 13, 14, 15, 16, 17, 18, 19 or 20.

In some embodiments,

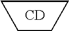

is alpha, beta or gamma cyclodextrin, e.g., beta cyclodextrin.

In some embodiments, the CDP is a polymer of the following formula:

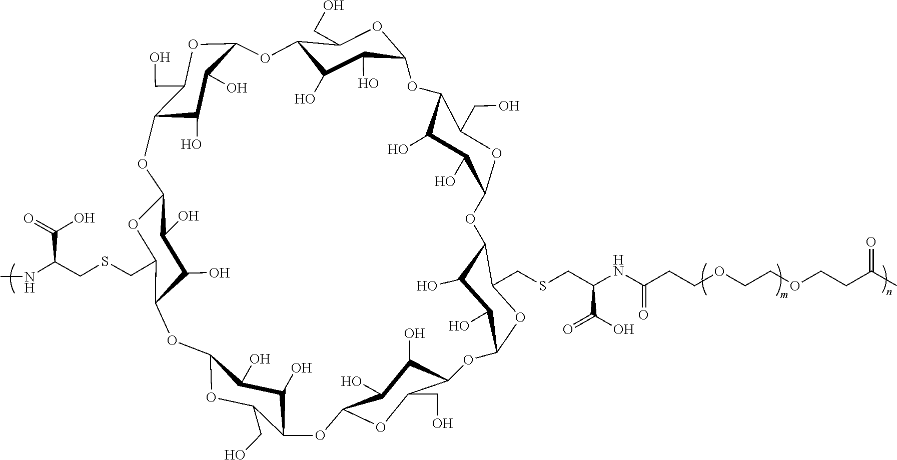

wherein the group

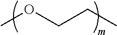

has a Mw of 3.4 kDa or less and n is at least 4, 5, 6, 7, 8, 9, 10, 11, 12, 13, 14, 15, 16, 17, 18, 19 or 20.

In some embodiments, the group

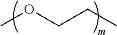

has a Mw of 3.4 kDa and the Mw of the compound as a whole is from 27 kDa to 99.6 kDa.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four, five six, seven, eight, etc., cyclodextrin moieties and at least four, five six, seven, eight, etc., comonomers.

In some embodiments, the at least four, five, six, seven or eight cyclodextrin moieties and at least four, five, six, seven or eight comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin comonomers comprise linkers to which epothilones may be further linked. In some embodiments, the epothilones are linked via second linkers.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient epothilone such that up to at least 3%, 5%, 10%, 15%, 20%, 25%, 30%, or even 35% by weight of the CDP, when conjugated, is epothilone.

In some embodiments, the CDP has a molecular weight of 10,000-500,000. In some embodiments, the cyclodextrin moieties make up at least about 2%, 5%, 10%, 20%, 30%, 50% or 80% of the CDP by weight.

In some embodiments, the CDP comprises a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, the CDP comprises a comonomer selected from the group consisting of: polyglycolic acid and polylactic acid chain. the CDP comprises a comonomer selected from the group consisting of a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁—, —NR₁—C(O)—NR₁—, —NR₁—C(NR₁)—NR₁—, and —B(OR₁)—; and R₁, independently for each occurrence, represents H or a lower alkyl.

In some embodiments, a CDP of the following formula can be made by the scheme below:

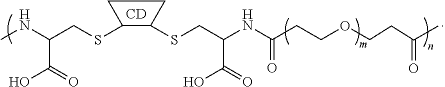

providing a polymer of formula A and formula B:

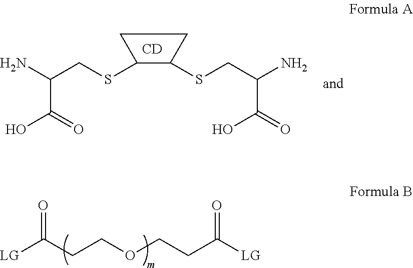

wherein LG is a leaving group;
and contacting the polymers under conditions that allow for the formation of a covalent bond between the polymers of formula A and B, to form a polymer of the following formula:

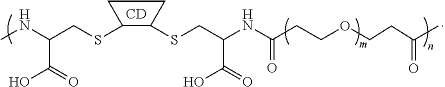

wherein the group

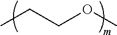

has a Mw of 3.4 kDa or less and n is at least four.

In some embodiments, Formula B is

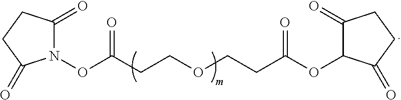

In some embodiments, the group

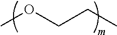

has a Mw of 3.4 kDa and the Mw of the compound is from 27 kDa to 99.6 kDa.

In some embodiments, the polymers of formula A and formula B are contacted in the presence of a base. In some embodiments, the base is an amine containing base. In some embodiments, the base is DEA.

In some embodiments, a CDP of the following formula can be made by the scheme below:

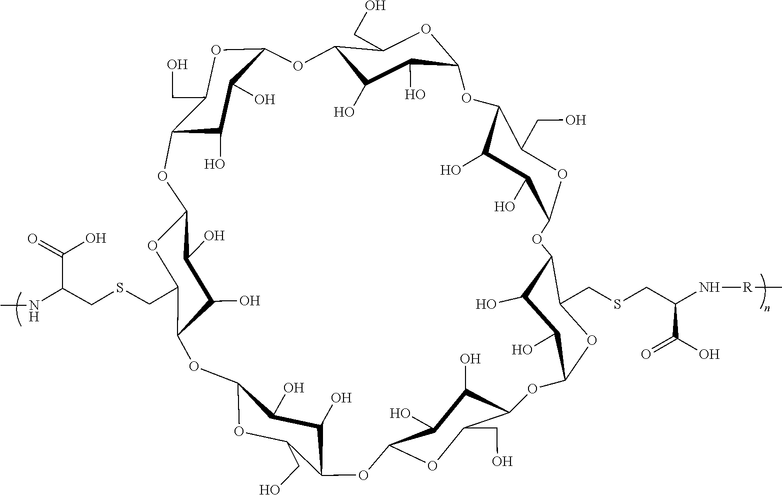

wherein R is of the form:

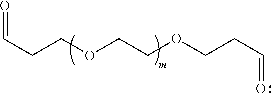

comprising the steps of:

reacting a compound of the formula below:

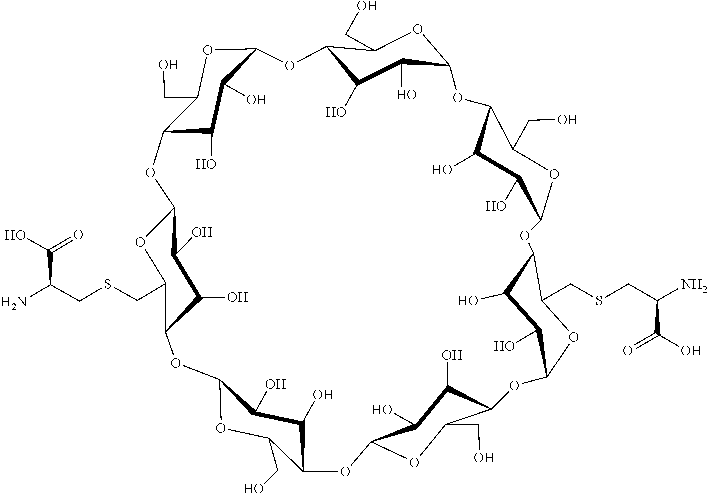 [SMILES: N[C@H](CSCC1O[C@H]2O[C@@H]3C(CO)O[C@H](O[C@@H]4C(CO)O[C@H](O[C@@H]5C(CO)O[C@H](O[C@@H]6C(CSC[C@@H](N)C(=O)O)O[C@H](O[C@@H]7C(CO)O[C@@H](O[C@@H]8C(CO)O[C@@H](O[C@H]1C(O)[C@@H]2O)C(O)[C@H]8O)[C@@H](O)C7O)C(O)[C@H]6O)C(O)[C@H]5O)[C@@H](O)C4O)[C@@H](O)C3O)C(=O)O]

with a compound of the formula below:

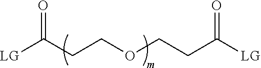

wherein the group

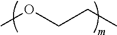

has a Mw of 3.4 kDa or less and n is at least four,

in the presence of a non-nucleophilic organic base in a solvent.

In some embodiments,

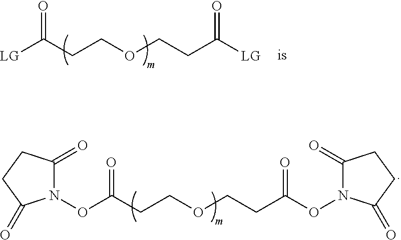

In some embodiments, the solvent is a polar aprotic solvent. In some embodiments, the solvent is DMSO.

In some embodiments, the method also includes the steps of dialysis; and lyophylization.

In some embodiments, a CDP provided below can be made by the following scheme:

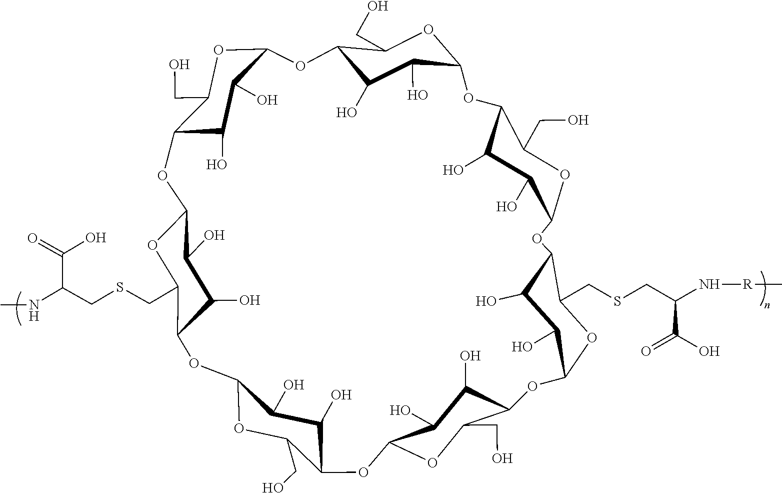

wherein R is of the form:

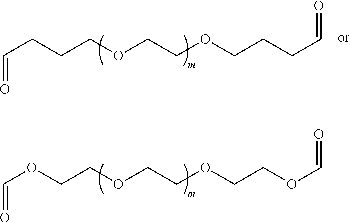

comprising the steps of:
reacting a compound of the formula below:

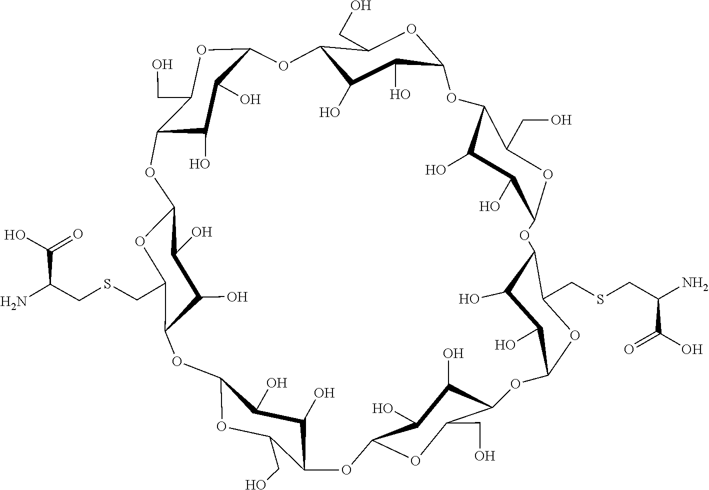 [SMILES: N[C@H](CSCC1O[C@H]2O[C@@H]3C(CO)O[C@H](O[C@@H]4C(CO)O[C@H](O[C@@H]5C(CO)O[C@H](O[C@@H]6C(CSC[C@@H](N)C(=O)O)O[C@H](O[C@@H]7C(CO)O[C@@H](O[C@@H]8C(CO)O[C@@H](O[C@H]1C(O)[C@@H]2O)C(O)[C@H]8O)[C@@H](O)C7O)C(O)[C@H]6O)C(O)[C@H]5O)[C@@H](O)C4O)[C@@H](O)C3O)C(=O)O]

with a compound of the formula below:

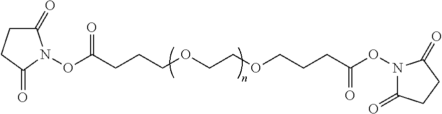

wherein the group

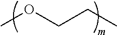

has a Mw of 3.4 kDa or less and n is at least four,

or with a compound provided below:

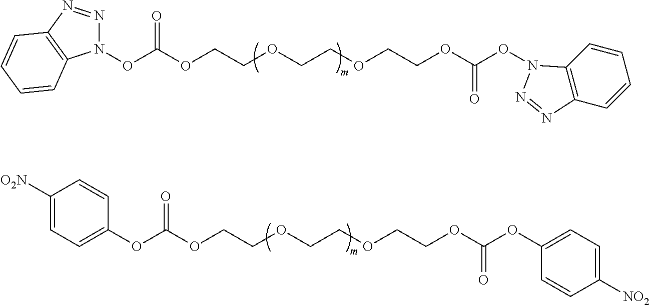

wherein the group

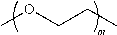

has a Mw of 3.4 kDa;

in the presence of a non-nucleophilic organic base in DMSO;

and dialyzing and lyophilizing the following polymer

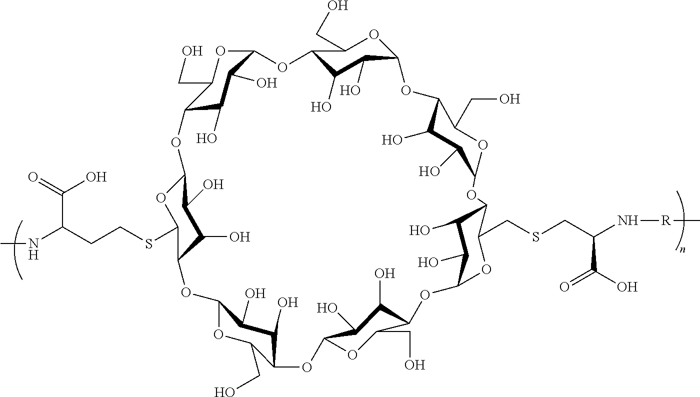

A CDP described herein may be attached to or grafted onto a substrate. The substrate may be any substrate as recognized by those of ordinary skill in the art. In another preferred embodiment of the invention, a CDP may be crosslinked to a polymer to form, respectively, a crosslinked cyclodextrin copolymer or a crosslinked oxidized cyclodextrin copolymer. The polymer may be any polymer capable of crosslinking with a CDP (e.g., polyethylene glycol (PEG) polymer, polyethylene polymer). The polymer may also be the same or different CDP. Thus; for example, a linear CDP may be crosslinked to any polymer including, but not limited to, itself, another linear CDP, and a linear oxidized CDP. A crosslinked linear CDP may be prepared by reacting a linear CDP with a polymer in the presence of a crosslinking agent. A crosslinked linear oxidized CDP may be prepared by reacting a linear oxidized CDP with a polymer in the presence of an appropriate crosslinking agent. The crosslinking agent may be any crosslinking agent known in the art. Examples of crosslinking agents include dihydrazides and disulfides. In a preferred embodiment, the crosslinking agent is a labile group such that a crosslinked copolymer may be uncrosslinked if desired.

A linear CDP and a linear oxidized CDP may be characterized by any means known in the art. Such characterization methods or techniques include, but are not limited to, gel permeation chromatography (GPC), matrix assisted laser desorption ionization-time of flight mass spectrometry (MALDI-TOF Mass spec), ¹H and ¹³C NMR, light scattering and titration.

The invention also provides a cyclodextrin composition containing at least one linear CDP and at least one linear oxidized CDP as described above. Accordingly, either or both of the linear CDP and linear oxidized CDP may be crosslinked to another polymer and/or bound to a ligand as described above. Therapeutic compositions according to the invention contain an epothilone and a linear CDP or a linear oxidized CDP, including crosslinked copolymers. A linear CDP, a linear oxidized CDP and their crosslinked derivatives are as described above. The epothilone may be any synthetic, semi-synthetic or naturally occurring biologically active epothilone, including those known in the art.

One aspect of the present invention contemplates attaching an epothilone to a CDP for delivery of an epothilone. The present invention discloses various types of linear, branched, or grafted CDPs wherein an epothilone is covalently bound to the polymer. In certain embodiments, the epothilone is covalently linked via a biohydrolyzable bond, for example, an ester, amide, carbamates, or carbonate.

An exemplary synthetic scheme for covalently bonding a derivatized CD to an epothilone is shown in Scheme I.

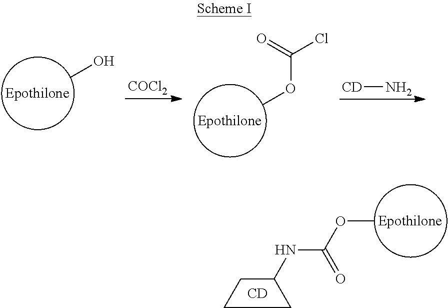

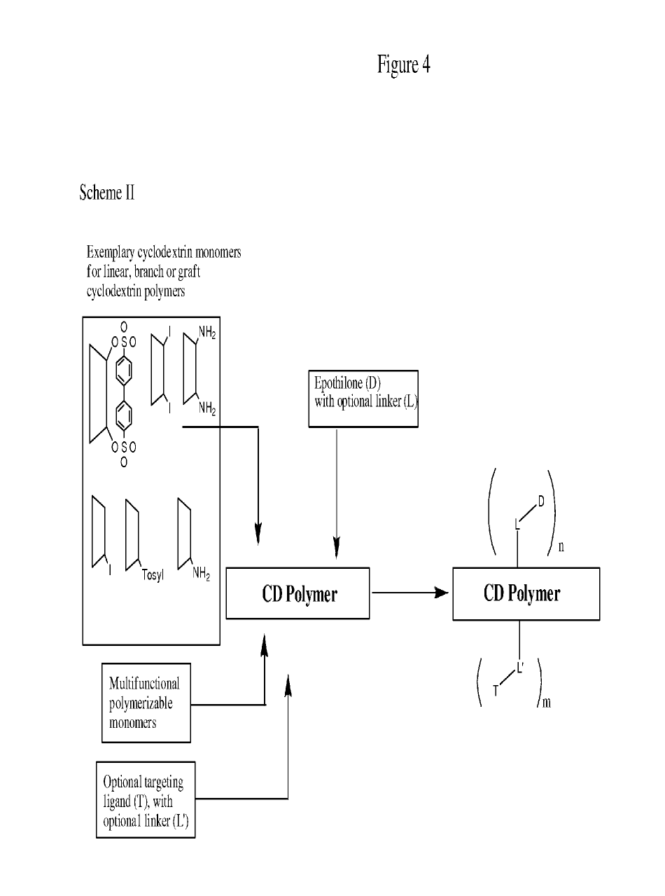

A general strategy for synthesizing linear, branched or grafted cyclodextrin-containing polymers (CDPs) for loading an epothilone, and an optional targeting ligand is shown in FIG. 4.

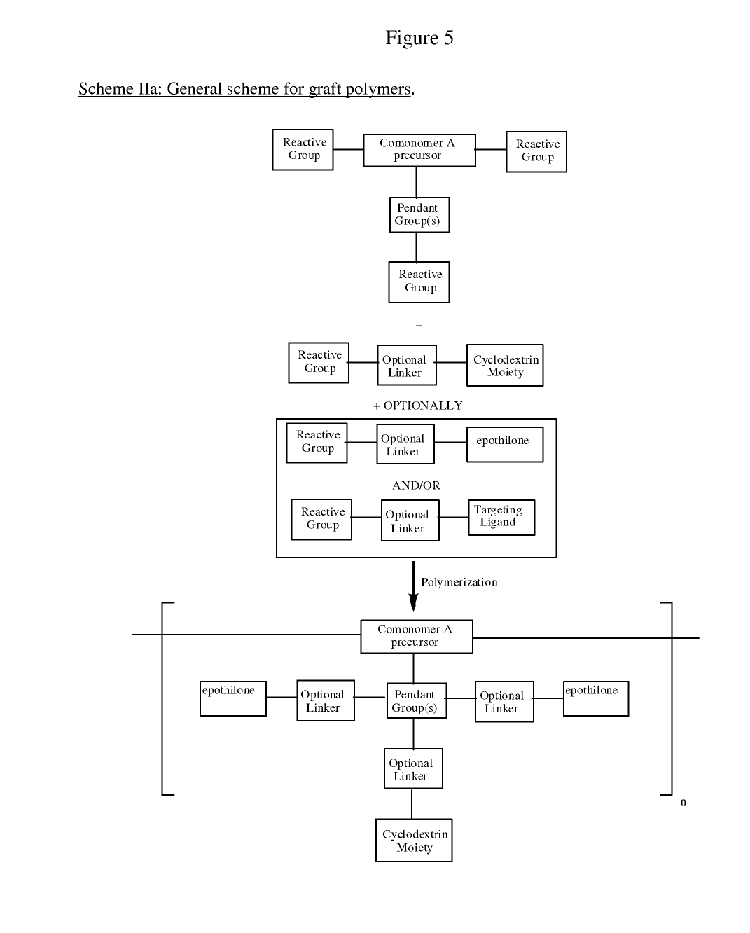

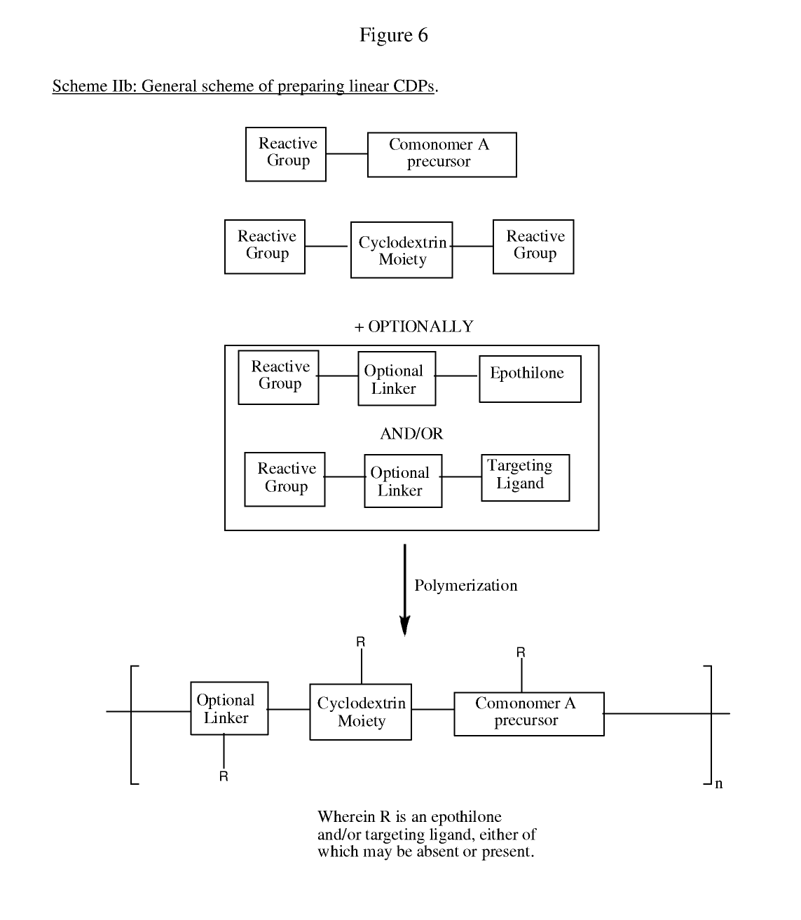

To illustrate further, comonomer precursors (shown in the scheme of FIG. 5 as A), cyclodextrin moieties, epothilones, and/or targeting ligands may be assembled as shown in Schemes IIa-IIb in FIGS. 5 and 6. Note that in schemes IIa-IIb, in any given reaction there may be more than one comonomer precursor, cyclodextrin moiety, therapeutic agent (i.e., epothilone) or targeting ligand that is of the same type or different. Furthermore, prior to polymerization, one or more comonomer precursor, cyclodextrin moiety, therapeutic agent or targeting ligand may be covalently linked with each other in one or more separate step. The scheme as provided above includes embodiments, where not all available positions for attachment of the epothilone are occupied on the CDP. For example, in some embodiments, less than all of the available points of attachments are reacted, leaving less than 100% yield of the epothilone onto the polymer. Accordingly, the loading of the epothilone onto the polymer can vary. This is also the case regarding a targeting agent when a targeting agent is included.

Scheme IIa: General Scheme for Graft Polymers.

The comonomer A precursor, cyclodextrin moiety, epothilone and optional targeting ligand are as defined above. Furthermore, one skilled in the art may choose from a variety of reactive groups, e.g., hydroxyls, carboxyls, halides, amines, and activated ethenes, ethynes, or aromatic groups in order achieve polymerization. For further examples of reactive groups are disclosed in Advanced Organic Chemistry: Reactions, Mechanisms, and Structure, 5th Edition, 2000.

In some embodiments, one or more of the epothilone moieties in the CDP-epothilone conjugate can be replaced with another therapeutic agent, e.g., another anticancer agent or anti-inflammatory agent.

Scheme IIb: General Scheme of Preparing Linear CDPs.

One skilled in the art would recognize that by choosing a comonomer A precursor that has multiple reactive groups polymer branching can be achieved.

In some embodiments, one or more of the epothilone moieties in the CDP-epothilone conjugate can be replaced with another therapeutic agent, e.g., another anticancer agent or anti-inflammatory agent.

Examples of different ways of synthesizing CDP-epothilone conjugates are shown in Schemes III-VIII below. In each of Schemes III-VIII, one or more of the epothilone moieties in the CDP-epothilone conjugate can be replaced with another therapeutic agent, e.g., another anticancer agent or anti-inflammatory agent.

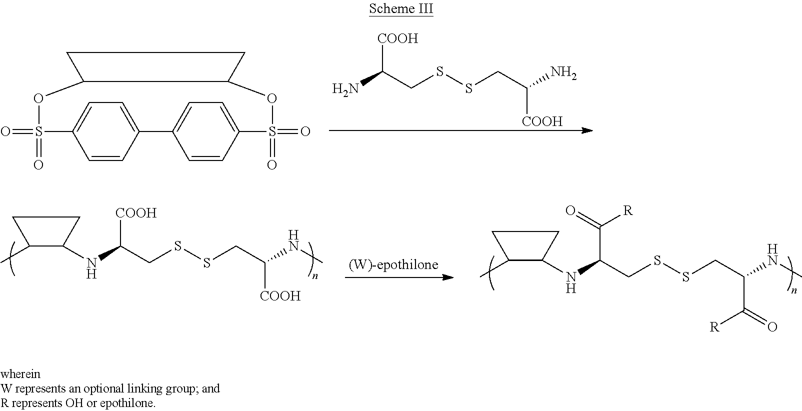

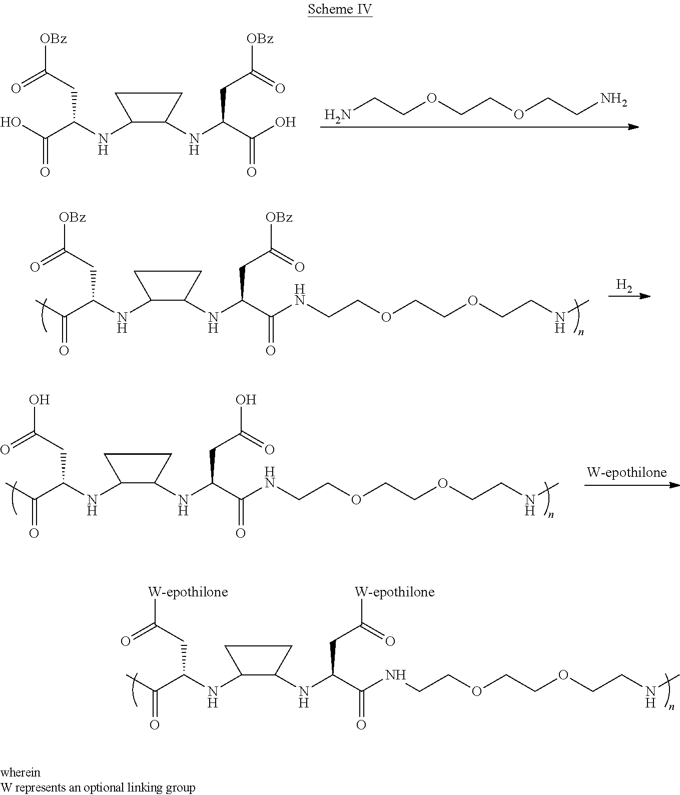

Scheme IV, as provided above, includes embodiments where W-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

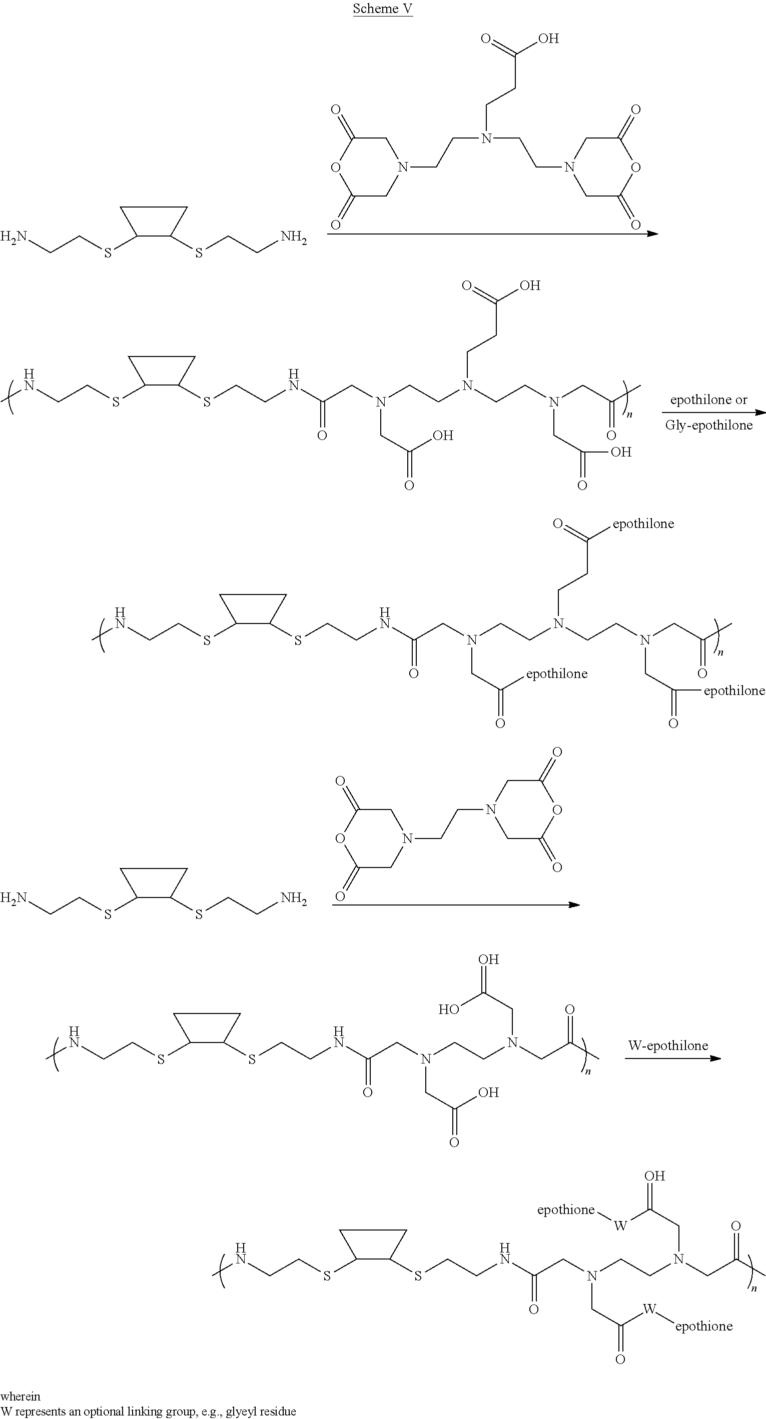

Scheme V, as provided above, includes embodiments where W-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

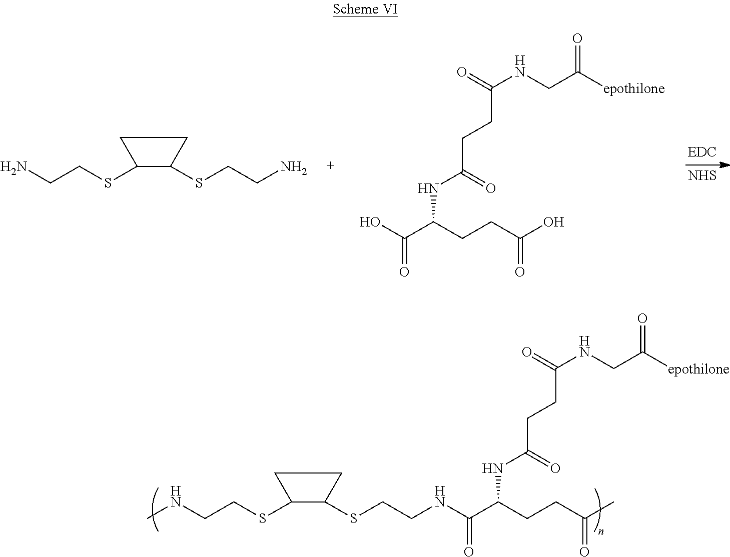

Scheme VI, as provided above, includes embodiments where epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

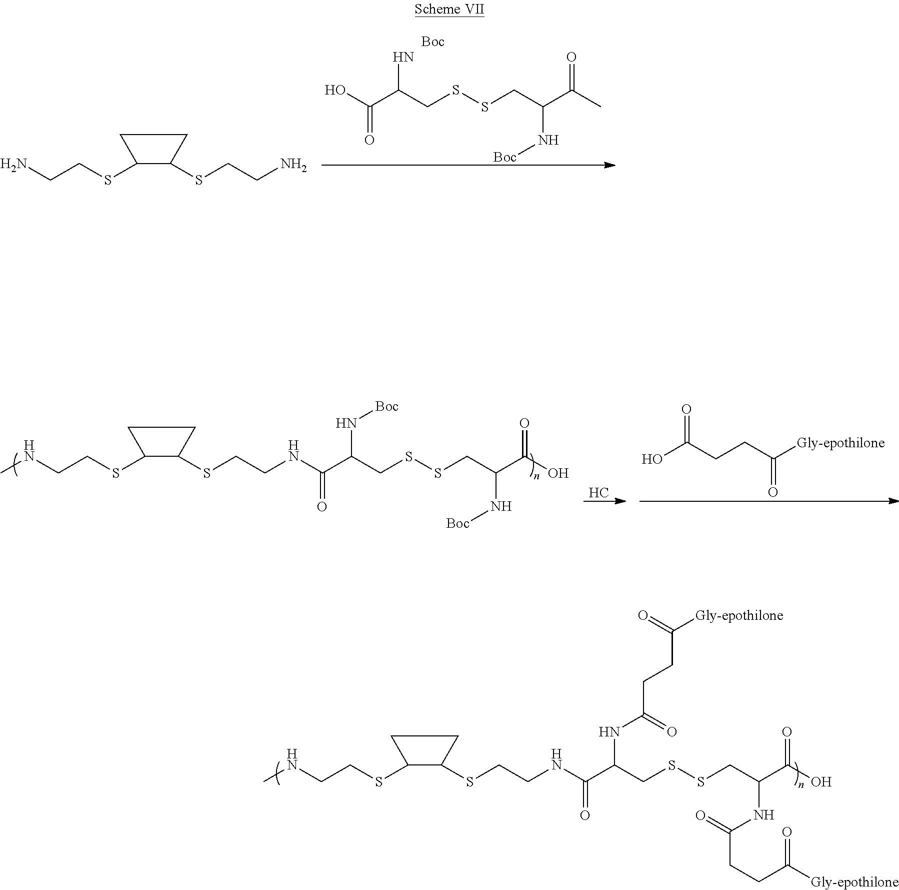

Scheme VII, as provided above, includes embodiments where gly-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

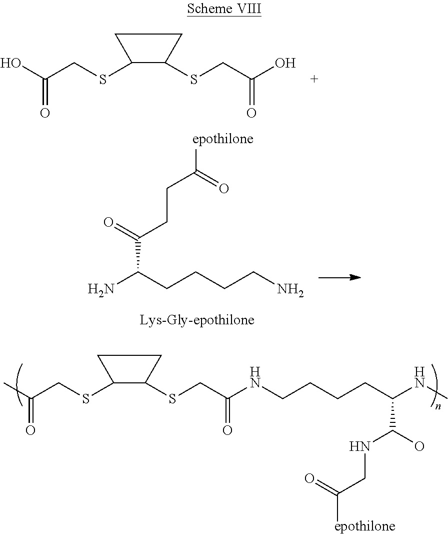

Scheme VIII, as provided above, includes embodiments where epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

Additional examples of methods of synthesizing CDP-epothilone conjugates are shown in Schemes IX-XIV below. In each of Schemes IX-XIV, one or more of the epothilone moieties in the CDP-epothilone conjugate can be replaced with another therapeutic agent, e.g., another anticancer agent or anti-inflammatory agent.

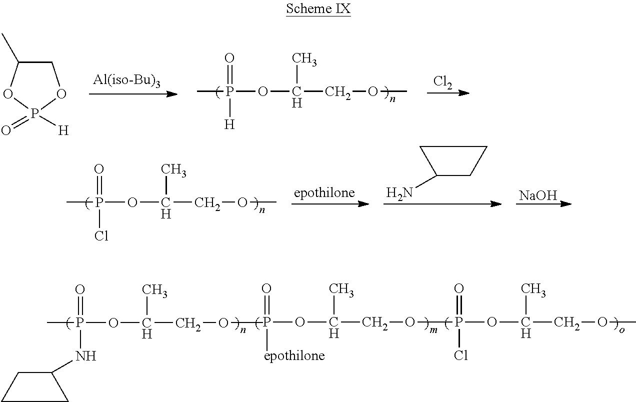

Scheme IX, as provided above, includes embodiments where epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

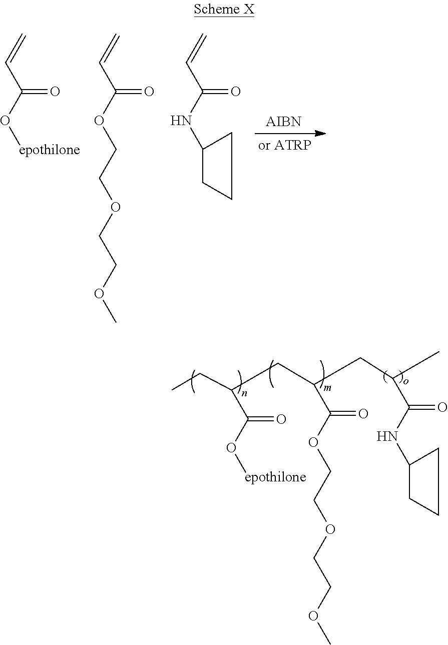

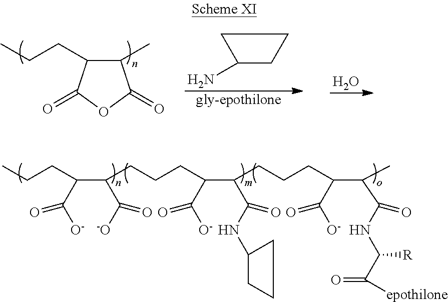

Scheme XI, as provided above, includes embodiments where gly-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

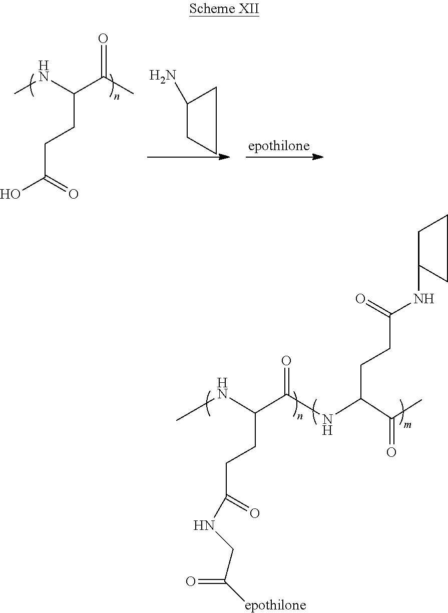

Scheme XII, as provided above, includes embodiments where epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

The present invention further contemplates CDPs and CDP-conjugates synthesized using CD-biscysteine monomer and a di-NHS ester such as PEG-DiSPA or PEG-BTC as shown in Schemes XIII-XIV below.

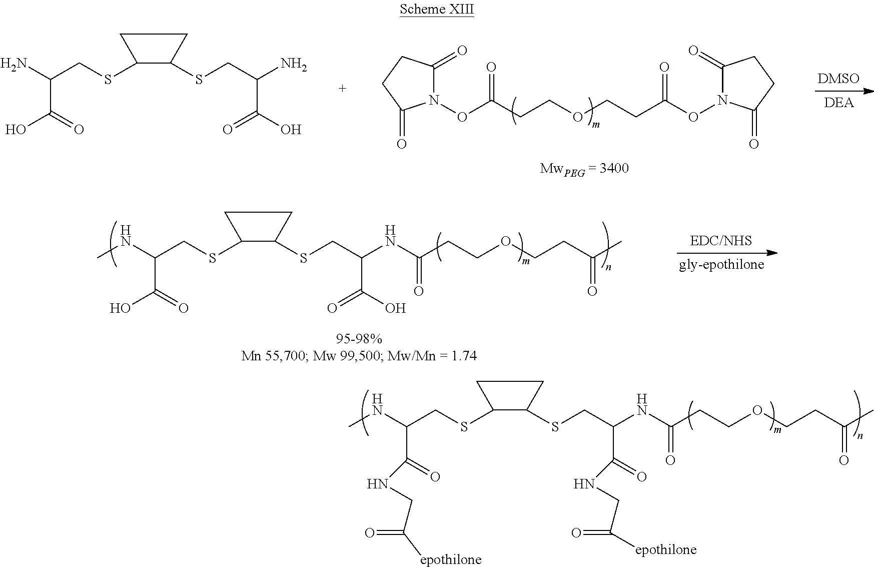

Scheme XIII, as provided above, includes embodiments where gly-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

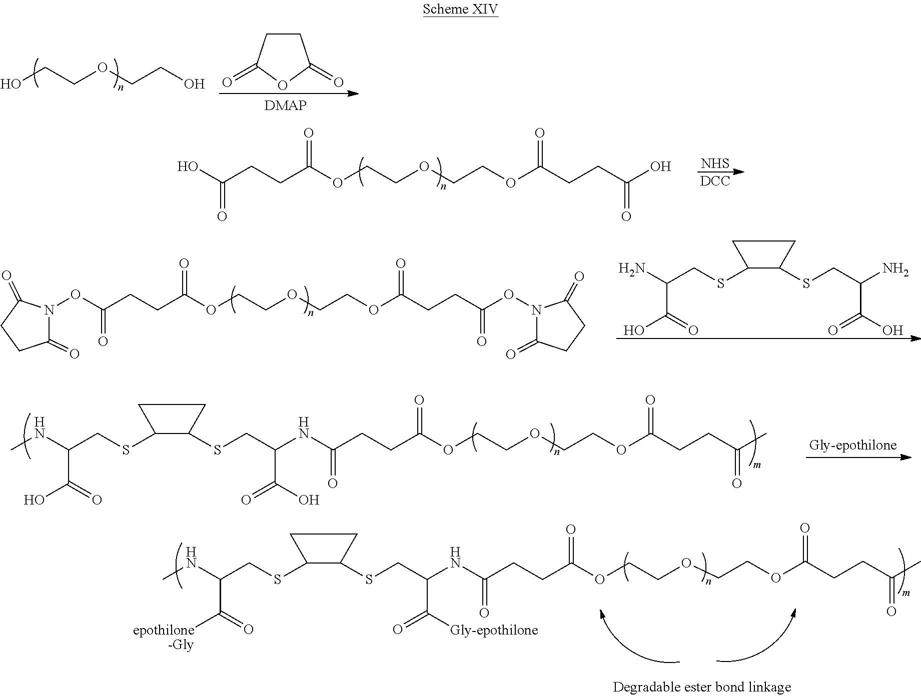

Scheme XIV, as provided above, includes embodiments where gly-epothilone is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary.

In some embodiments, a CDP-epothilone conjugate can be made by providing a CDP comprising cyclodextrin moieties and comonomers which do not contain cyclodextrin moieties (comonomers), wherein the cyclodextrin moieties and comonomers alternate in the CDP and wherein the CDP comprises at least four, five, six, seven, eight, etc. cyclodextrin moieties and at least four, five, six, seven, eight, etc. comonomers; and attaching an epothilone to the CDP.

In some embodiments, one or more of the epothilone moieties in the CDP-epothilone conjugate can be replaced with another therapeutic agent, e.g., another anticancer agent or anti-inflammatory agent.

In some embodiments, the epothilone is attached via a linker In some embodiments, the epothilone is attached to the water soluble linear polymer through an attachment that is cleaved under biological conditions to release the epothilone. In some embodiments, the epothilone is attached to the water soluble linear polymer at a cyclodextrin moiety or a comonomer. In some embodiments, the epothilone is attached to the water soluble linear polymer via an optional linker to a cyclodextrin moiety or a comonomer.

In some embodiments, the cyclodextrin moieties comprise linkers to which therapeutic agents are linked. In some embodiments, the cyclodextrin moieties comprise linkers to which therapeutic agents are linked via a second linker

In some embodiments, the CDP is made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. In some embodiments, the CDP is conjugated with an epothilone to provide a CDP-epothilone conjugate.

In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the epothilone is attached to the CDP via a linker In some embodiments, the linker is cleaved under biological conditions.

In some embodiments, the epothilone makes up at least 5%, 10%, 15%, 20%, 25%, 30%, or even 35% by weight of the CDP-epothilone conjugate.

In some embodiments, the comonomer comprises polyethylene glycol of molecular weight 3,400 Da, the cyclodextrin moiety comprises beta-cyclodextrin, the theoretical maximum loading of epothilone on the CDP-epothilone conjugate is 13%, and epothilone is 6-10% by weight of the CDP-epothilone conjugate.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent is achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C₁-C₁₀ alkyl, or arylalkyl optionally containing one or more heteroatoms within the chain or ring.

In some embodiments, the cyclodextrin moiety comprises an alpha, beta, or gamma cyclodextrin moiety.

In some embodiments, the epothilone is poorly soluble in water.

In some embodiments, the solubility of the epothilone is <5 mg/ml at physiological pH.

In some embodiments, the epothilone is a hydrophobic compound with a log P>0.4, >0.6, >0.8, >1, >2, >3, >4, or >5. In some embodiments, the epothilone is hydrophobic and is attached via a second compound.

In some embodiments, administration of the CDP-epothilone conjugate to a subject results in release of the epothilone over a period of at least 6 hours. In some embodiments, administration of the CDP-epothilone conjugate to a subject results in release of the epothilone over a period of 6 hours to a month. In some embodiments, upon administration of the CDP-epothilone conjugate to a subject the rate of epothilone release is dependent primarily upon the rate of hydrolysis as opposed to enzymatic cleavage.

In some embodiments, the CDP-epothilone conjugate has a molecular weight of 10,000-500,000.

In some embodiments, the cyclodextrin moieties make up at least about 2%, 5%, 10%, 20%, 30%, 50% or 80% of the polymer (i.e., the CDP-epothilone conjugate polymer) by weight.

In some embodiments, a the CDP includes a comonomer selected from the group consisting of: an alkylene chain, polysuccinic anhydride, poly-L-glutamic acid, poly(ethyleneimine), an oligosaccharide, and an amino acid chain. In some embodiments, a comonomer comprises a polyethylene glycol chain. In some embodiments, a comonomer comprises a polyglycolic acid or polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X)— (wherein X is NR₁, O or S), —OC(O)—, —C(═O)O—, —NR₁—, —NR₁CO—, —C(O)NR₁—, —S(O), (wherein n is 0, 1, or 2), —OC(O)—NR₁—, —NR₁—C(O)—NR₁—, —NR₁—C(NR₁)—NR₁—, and —B(OR₁)—; and R₁, independently for each occurrence, represents H or a lower alkyl.

In some embodiments, a CDP-polymer conjugate of the following formula can be made as follows:

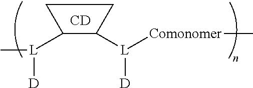

providing a polymer of the formula below:

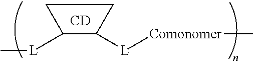

and coupling the polymer with a plurality of D moieties, wherein each D is independently absent or an epothilone, to provide:

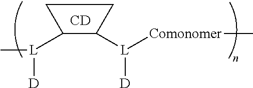

wherein the comonomer has a Mw of 2000 to 5000 Da (e.g., 3000 to 4000 Da, e.g., about 3.4 kDa) and n is at least 4, 5, 6, 7, 8, 9, 10, 11, 12, 13, 14, 15, 16, 17, 18, 19 or 20.

In some embodiments, one or more of the epothilone moieties in the CDP-epothilone conjugate can be replaced with another therapeutic agent, e.g., another anticancer agent or anti-inflammatory agent.

In some embodiments, a CDP-polymer conjugate of the following formula can be made as follows:

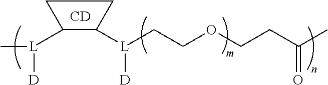

providing a polymer of the formula below:

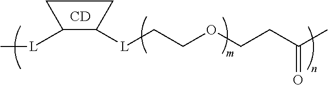

and coupling the polymer with a plurality of D moieties, wherein each D is independently absent or an epothilone, to provide:

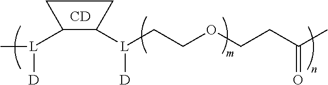

wherein the group

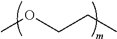

has a Mw of 3.4 kDa or less and n is at least 4, 5, 6, 7, 8, 9, 10, 11, 12, 13, 14, 15, 16, 17, 18, 19 or 20.

In some embodiments, one or more of the epothilone moieties in the CDP-epothilone conjugate can be replaced with another therapeutic agent, e.g., another anticancer agent or anti-inflammatory agent.

The reaction scheme as provided above includes embodiments where D is absent in one or more positions as provided above. This can be achieved, for example, when less than 100% yield is achieved when coupling the epothilone to the polymer and/or when less than an equivalent amount of epothilone is used in the reaction. Accordingly, the loading of the epothilone, by weight of the polymer, can vary, for example, the loading of the epothilone can be at least about 3% by weight, e.g., at least about 55, at least about 8%, at least about 10%, at least about 13%, at least about 15%, or at least about 20%.

In some embodiments, a CDP-polymer conjugate of the following formula can be made as follows:

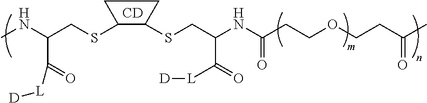

providing a polymer below:

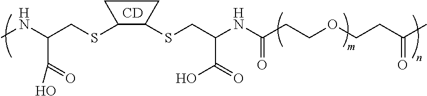

and coupling the polymer with a plurality of L-D moieties, wherein L is a linker or absent and D is an epothilone, to provide:

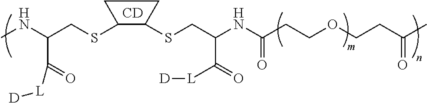

wherein the group

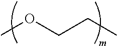

has a Mw of 3.4 kDa or less and n is at least 4, 5, 6, 7, 8, 9, 10, 11, 12, 13, 14, 15, 16, 17, 18, 19 or 20.

In some embodiments, one or more of the epothilone moieties in the CDP-epothilone conjugate can be replaced with another therapeutic agent, e.g., another anticancer agent or anti-inflammatory agent.

The reaction scheme as provided above includes embodiments where L-D is absent in one or more positions as provided above. This can be achieved, for example, when less than 100% yield is achieved when coupling the epothilone-linker to the polymer and/or when less than an equivalent amount of epothilone-linker is used in the reaction. Accordingly, the loading of the epothilone, by weight of the polymer, can vary, for example, the loading of the epothilone can be at least about 3% by weight, e.g., at least about 5%, at least about 8%, at least about 10%, at least about 13%, at least about 15%, or at least about 20%.

In some embodiments, at least a portion of the L moieties of L-D is absent. In some embodiments, each L is independently an amino acid or derivative thereof (e.g., glycine).

In some embodiments, the coupling of the polymer with the plurality of L-D moieties results in the formation of a plurality of amide bonds.

In certain instances, the CDPs are random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

Formulations

In another aspect, the present invention provides pharmaceutically acceptable compositions, which include a CDP-epothilone conjugate described herein and a pharmaceutically acceptable carrier.

The pharmaceutical compositions of this invention may be in a variety of forms. These include, for example, liquid, semi-solid and solid dosage forms, such as liquid solutions (e.g., injectable and infusible solutions), dispersions or suspensions. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the CDP-epothilone conjugate is administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, the pharmaceutical composition can include plasticizers and stabilizing agents. In certain embodiments, additives such as plasticizers and stabilizing agents are selected for their biocompatibility. In certain embodiments, the additives are lung surfactants, such as 1,2-dipalmitoylphosphatidycholine (DPPC) and L-.alpha.-phosphatidylcholine (PC).

A pharmaceutical composition containing a CDP-epothilone conjugate featured herein may further contain one or more adjuvant substances, such as fillers, thickening agents or the like. In other embodiments, materials that serve as adjuvants may be associated with the CDP matrix. Such additional materials may affect the characteristics of the CDP matrix that results.

For example, fillers, such as bovine serum albumin (BSA) or mouse serum albumin (MSA), may be associated with the CDP matrix. In certain embodiments, the amount of filler may range from about 0.1 to about 50% or more by weight of the CDP matrix, or about 2.5, 5, 10, 25, or 40 percent. Incorporation of such fillers may affect the biodegradation of the CDP material and/or the sustained release rate of any encapsulated substance. Other fillers known to those of skill in the art, such as carbohydrates, sugars, starches, saccharides, celluloses and polysaccharides, including mannitose and sucrose, may be used in certain embodiments of the present invention.

In certain embodiments, the composition can include an excipient. A particular excipient may be selected based on its melting point, solubility in a selected solvent (e.g., a solvent that dissolves the polymer and/or the therapeutic agent), and the resulting characteristics of the nanoparticles.

Excipients may comprise a few percent, about 5%, 10%, 15%, 20%, 25%, 30%, 40%, 50%, or higher percentage of the subject compositions.

Buffers, acids and bases may be incorporated in the subject compositions to adjust their pH. Agents to increase the diffusion distance of agents released from the CDP matrix may also be included.

Other materials may be used to advantage or to control the desired release rate of an epothilone for a particular treatment protocol. For example, if the sustained release is too slow for a particular application, a pore-forming agent may be added to generate additional pores in the matrix. Any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the CDP system.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances.

Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and PVP. The size and extent of the pores may be varied over a wide range by changing the molecular weight and percentage of pore-forming agent incorporated into the CDP system.

The charge, lipophilicity or hydrophilicity of any subject CDP matrix may be modified by attaching in some fashion an appropriate compound to the surface of the matrix. For example, surfactants may be used to enhance wettability of poorly soluble or hydrophobic compositions. Examples of suitable surfactants include dextran, polysorbates and sodium lauryl sulfate. In general, surfactants are used in low concentrations, generally less than about 5%.

Binders are adhesive materials that may be incorporated in polymeric formulations to bind and maintain matrix integrity. Binders may be added as dry powder or as solution. Sugars and natural and synthetic polymers may act as binders.

Materials added specifically as binders are generally included in the range of about 0.5%-15% w/w of the matrix formulation. Certain materials, such as microcrystalline cellulose, also used as a spheronization enhancer, also have additional binding properties.

Various coatings may be applied to modify the properties of the matrices.

Three exemplary types of coatings are seal, gloss and enteric coatings. Other types of coatings having various dissolution or erosion properties may be used to further modify subject matrices behavior, and such coatings are readily known to one of ordinary skill in the art.

The seal coat may prevent excess moisture uptake by the matrices during the application of aqueous based enteric coatings. The gloss coat generally improves the handling of the finished matrices. Water-soluble materials such as hydroxypropylcellulose may be used to seal coat and gloss coat implants. The seal coat and gloss coat are generally sprayed onto the matrices until an increase in weight between about 0.5% and about 5%, often about 1% for a seal coat and about 3% for a gloss coat, has been obtained.

Enteric coatings consist of polymers which are insoluble in the low pH (less than 3.0) of the stomach, but are soluble in the elevated pH (greater than 4.0) of the small intestine. Polymers such as EUDRAGIT™, RohmTech, Inc., Malden, Mass., and AQUATERIC™, FMC Corp., Philadelphia, Pa. may be used and are layered as thin membranes onto the implants from aqueous solution or suspension or by a spray drying method. The enteric coat is generally sprayed to a weight increase of about 1% to about 30%, preferably about 10 to about 15% and may contain coating adjuvants such as plasticizers, surfactants, separating agents that reduce the tackiness of the implants during coating, and coating permeability adjusters.

The present compositions may additionally contain one or more optional additives such as fibrous reinforcement, colorants, perfumes, rubber modifiers, modifying agents, etc. In practice, each of these optional additives should be compatible with the CDP-epothilone conjugate and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

The CDP-epothilone conjugates described herein can be administered in various pharmaceutical compositions, depending on the disorder to be treated and the age, condition and body weight of the subject, as is well known in the art. For example, where the compounds are to be administered orally, they may be formulated as tablets, capsules, granules, powders or syrups; or for parenteral administration, they may be formulated as injections (intravenous, intramuscular or subcutaneous) or drop infusion preparations. These compositions can be prepared by conventional means, and, if desired, the active ingredient may be mixed with any conventional additive, such as an excipient, a binder, a disintegrating agent, a lubricant, a corrigent, a solubilizing agent, a suspension aid, an emulsifying agent or a coating agent. The dosage will vary depending on the symptoms, age and body weight of the subject, the nature and severity of the disorder to be treated or prevented, the route of administration and the form of the drug. Preferably, the CDP-epothilone is administered such that the desired dose of the epothilone is provided to the subject, e.g., a dose described herein.

In one embodiment, the CDP-epothilone conjugate is provided in lyophilized form and is reconstituted prior to administration to a subject. The lyophilized CDP-epothilone conjugate can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant to maintain physical and chemical stability by protecting the particle and active from damage from crystal formation and the fusion process during freeze-drying. The lyoprotectant can be one or more of sucrose, lactose, trehalose, mannitol, glucose, dextran and sorbitol.

In some embodiments, the lyophilized CDP-epothilone conjugate is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution having a concentration of about 2 mg/mL to about 4 mg/mL of the CDP-epothilone conjugate. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject epothilone analogs with a third vial containing sufficient parenteral diluent to prepare the final concentration for administration. A typical diluent is Lactated Ringer's Injection. The final concentration for administration would be for example, contain from about 0.1 mg/mL to about 0.9 mg/mL, e.g., 0.2 mg/mL to about 0.6 mg/mL, of the CDP-epothilone conjugate.

The final dilution of the reconstituted CDP-epothilone conjugate may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The precise time of administration and/or amount of the CDP-epothilone conjugate that will yield the most effective results in terms of efficacy of treatment in a given subject will depend upon the activity, pharmacokinetics, and bioavailability of a particular compound, physiological condition of the subject (including age, sex, disease type and stage, general physical condition, responsiveness to a given dosage and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

As used herein, “pharmaceutically acceptable carrier” includes any and all solvents, dispersion media, isotonic and absorption delaying agents, and the like that are physiologically compatible. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

Compositions useful in the methods featured in the present disclosure include those suitable for oral or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a CDP-epothilone conjugate(s) with the carrier and, optionally, one or more accessory ingredients. In general, the formulations are prepared by uniformly and intimately bringing into association a CDP-epothilone conjugate with liquid carriers, or finely divided solid carriers, or both, and then, if necessary, shaping the product.

Compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an epothilone as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-epothilone conjugates may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration comprise one or more CDP-epothilone conjugate(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the epothilone from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the CDP-epothilone conjugate(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the therapeutic polymer conjugate(s) of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

In one aspect, the invention features a kit for providing a CDP-epothilone conjugate. The kit includes a CDP-epothilone conjugated for use in treatment a subject, such as, a human; and instructions for using the conjugate. In one embodiment, the CDP-epothilone conjugate is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, the kit provides the CDP-epothilone conjugate in solution form.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-epothilone conjugate and the second therapeutic agent are in separate containers, and in another embodiment, the CDP-epothilone conjugate and the second therapeutic agent are packaged in the same container.

In one embodiment, the kit includes instructions to use the CDP-epothilone conjugate, such as for treatment of a subject. The instructions can include methods for reconstituting or diluting the CDP-epothilone conjugate composition for use with a particular subject or in combination with a particular chemotherapeutic agent. The instructions can also include methods for reconstituting or diluting the polymer conjugate composition for use with a particular means of administration, such as by i.v. infusion.

In another embodiment, the kit includes instructions for treating a subject with a particular indication, such as a particular cancer, or a cancer at a particular stage. For example, the instructions can be for a cancer or cancer stage described herein. The instructions may also address treatment of a subject who has been non-responsive to a first line therapy, such as taxane, epothilone or capecitabine, or who is already receiving a first line therapy, e.g., capecitabine. In another embodiment, the instructions will describe treatment of selected subjects with the CDP-epothilone conjugate. For example, the instructions can describe treatment of one or more of: a subject with hepatic impairment, such as having decreased transaminase (AST and/or ALT) levels; a subject having diabetes; a subject who has experienced neuropathy from treatment with a chemotherapeutic agent such as a taxane, a platinum-based agent, a vinca alkaloid or an epothilone; a subject having moderate to severe neuropathy; a subject who has experienced an infusion site reaction to treatment with an epothilone; a subject who is currently being treated or who will be treated with a CYP3A4 inhibitor; a subject who is currently being administered or will be administered an anti-depressant, e.g., St. John's wort; a subject who is 65 or older; a subject who has or is at risk for having an adverse cardiac reaction; a subject who has received an epothilone treatment and has a platelet count less than a standard; a subject who has received an epothilone and has a neutrophil count less than a standard; and a subject having one or more symptom of febrile neutropenia.

Each component of the kit can be enclosed within an individual container and all of the various containers can be within a single package.

Dosages and Dosage Regimens.

The CDP-epothilone conjugate(s) described herein can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the epothilone-containing polymer is administered to a subject at a dosage of, e.g., about 0.1 to 100 mg/m², about 5 to 90 mg/m², about 10 to 70 mg/m², e.g., about 15, 20, 25, 30, 35, 40, 45, 50, 55, 60, or 65 mg/m². Administration can be at regular intervals, such as every 1, 2, 3, 4, or 5 days, or weekly, or every 2, 3, 4, 5, 6, or 7 weeks. The administration can be over a period of from about 10 minutes to about 3 hours, e.g., from about 30 minutes to about 2 hours, from about 45 minutes to 90 minutes 30 minutes, 1 hour, 2 hours, 3 hours, 4 hours, 5 hours or more. In one embodiment, the epothilone containing polymer is administered as a bolus infusion, e.g., over a period of 15 minutes, 10 minutes, 5 minutes or less. In one embodiment, the CDP-epothilone is administered in an amount such the desired dose of the epothilone is administered. Preferably the dose of the epothilone is a dose described herein.

In one embodiment, the subject receives 1, 2, 3, up to 10 treatments, or more, or until the disorder or a symptom of the disorder is cured, healed, alleviated, relieved, altered, remedied, ameliorated, palliated, improved or affected. For example, the subject receive an infusion every 1, 2, 3 or 4 weeks until the disorder or a symptom of the disorder are cured, healed, alleviated, relieved, altered, remedied, ameliorated, palliated, improved or affected.

In one embodiment, a subject is administered a CDP-epothilone conjugate in combination with a second agent, after the subject develops resistance to, or fails to respond to a first line therapy.

In another embodiment, the subject is administered a CDP-epothilone conjugate in the absence of a second agent, after the subject develops resistance to, or fails to respond to the first line therapy.

Combination Therapies.

The CDP-epothilone conjugates may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-epothilone conjugate and the at least one additional therapeutic agent can be administered simultaneously, in the same or in separate compositions, or sequentially. For sequential administration, the CDP-epothilone conjugate can be administered first, and the additional agent can be administered second, or the order of administration can be reversed.

In some embodiments, the CDP-epothilone conjugate is administered in combination with other therapeutic treatment modalities, including surgery, radiation, cryosurgery, and/or thermotherapy. Such combination therapies may advantageously utilize lower dosages of the administered epothilone and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the CDP-epothilone conjugate is administered with at least one additional therapeutic agent, such as a chemotherapeutic agent. In certain embodiments, the CDP-epothilone conjugate is administered in combination with one or more additional chemotherapeutic agent, e.g., with one or more chemotherapeutic agents described herein.

In some embodiments, the CDP-epothilone conjugate is administered in combination with an antiangiogenic and/or antivascular agent such as matrix metalloproteinase pathway inhibitors, and VEGF pathway inhibitors, such as anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin). Anti-Her-2 antibodies (e.g., trastuzumab (Herceptin®) and other antibodies from Genentech), EGFR pathway inhibitors, such as EKB-569, erlotinib (Tarceva®) and lapatinib (Tykerb®), and anti-EGFR antibodies (e.g., cetuximab (Erbitux®), panitumumab (Vectibix®), and gefitinib (Iressa®)), PDGF pathway inhibitors, MEK-1 kinase inhibitors, MAPK kinase inhibitors, Met kinase inhibitors, PI3 inhibitors, and src inhibitors are also suitable for use in combination therapies featured in the invention. mTor inhibitors such as rapamycin (Rapamune®), and analogs and derivatives thereof are also suitable chemotherapy agents. Other suitable mTOR inhibitors include AP23573 (also known as ridaforolimus, deforolimus, or MK-8669), CCI-779 (also known as temsirolimus) (Torisel®) and SDZ-RAD.

Other classes of chemotherapeutic agents that can be administered in combination with the CDP-epothilone include, e.g., the following:

Alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

Antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gencitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gencitabine (Gemzar®).

Vinca alkaloids: vinblastine (Velban®, Velsar®), vincristine (Vincasar®, Oncovin®), vindesine (Eldisine®), vinorelbine (Navelbine®).

Platinum-based agents: carboplatin (Paraplat®, Paraplatin®), cisplatin (Platinol®), oxaliplatin (Eloxatin®).

Anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

Topoisomerase inhibitors: topotecan (Hycamtin®), irinotecan (Camptosar®), etoposide (Toposar®, VePesid®), teniposide (Vumon®), lamellarin D, camptothecin (e.g., IT-101)

A taxane: paclitaxel (Taxol®), docetaxel (Taxotere®).

Antibiotics: actinomycin (Cosmegen®), bleomycin (Blenoxane®), hydroxyurea (Droxia®, Hydrea®), mitomycin (Mitozytrex®, Mutamycin®).

Immunomodulators: lenalidomide (Revlimid®), thalidomide (Thalomid®).

Immune cell antibodies: alemtuzamab (Campath®), gemtuzumab (Myelotarg®), rituximab (Rituxan®), tositumomab (Bexxar®).

Proteosome inhibitor: bortezomib (Velcade®).

Interferons (e.g., IFN-alpha (Alferon®, Roferon-A®, Intron®-A) or IFN-gamma (Actimmune®))

Interleukins. IL-1, IL-2 (Proleukin®), IL-24, IL-6 (Sigosix®), IL-12.

An HSP90 inhibitor (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

Anti-androgens which include, without limitation nilutamide (Nilandron®) and bicalutamide (Caxodex®).

Antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

Anti-hypercalcaemia agents which include without limitation gallium (III) nitrate hydrate (Ganite®) and pamidronate disodium (Aredia®).

Apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

Calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

Cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

Cyclin dependent kinase inhibitors which include without limitation olomoucine and its derivatives, purvalanol B, roascovitine (Seliciclib®), indirubin, kenpaullone, purvalanol A and indirubin-3′-monooxime

Cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, erlotinib (Tarceva®), gefitinib (Iressa®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

Farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

Glycogen synthase kinase-3 (GSK3) inhibitors which include without limitation indirubin-3′-monooxime

Histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

Imidazotetrazinones which include without limitation temozolomide (Methazolastone®, Temodar® and its derivatives (e.g., as disclosed generically and specifically in U.S. Pat. No. 5,260,291) and Mitozolomide.

Insulin tyrosine kinase inhibitors which include without limitation hydroxyl-2-naphthalenylmethylphosphonic acid.

c-Jun-N-terminal kinase (INK) inhibitors which include without limitation pyrazoleanthrone and epigallocatechin gallate.

Mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9,1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

Phosphatidylinositol 3-kinase inhibitors which include without limitation wortmannin, and quercetin dihydrate.

Phosphatase inhibitors which include without limitation cantharidic acid, cantharidin, and L-leucinamide.

Protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

Polyamine Synthesis Inhibitors which include without limitation DMFO.

Proteasome Inhibitors which include, without limitation aclacinomycin A, gliotoxin, PS-341, MLN 341, bortezomib and bortezomib (Velcade®).

PTP1B inhibitors which include without limitation L-leucinamide.

Protein Tyrosine Kinase Inhibitors which include, without limitation tyrphostin Ag 216, tyrphostin Ag 1288, tyrphostin Ag 1295, geldanamycin, genistein and 7H-pyrollo[2,3-d]pyrimidine derivatives of formula I as generically and specifically described in PCT Publication No.: WO 03/013541 and U.S. Publication No.: 2008/0139587:

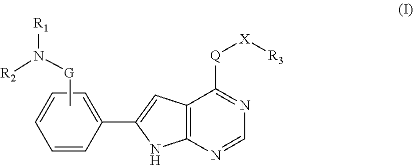

Publication No.: 2008/0139587 discloses the various substituents, e.g., R₁, R₂, etc.
SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

Retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

Serine/Threonine kinase inhibitors which include without limitation 2-aminopurine.

Sterol biosynthesis inhibitors which include without limitation squalene epoxidase and CYP2D6.

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiment, the CDP-epothilone conjugate is administered instead of another microtubule affecting agent, e.g., instead of a microtubule affecting agent as a first line therapy or a second line therapy. For example, the CDP-epothilone conjugate can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steriod can be administered in combination with a CDP-epothilone conjugate. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-epothilone conjugate is administered in combination with an anti-microbial (e.g., leptomycin B).

In another embodiment, the CDP-epothilone is administered in combination with an agent or procedure to mitigate potential side effects from the epothilone compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-epothilone is administered in combination with an immunosuppressive agent Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a CDP-epothilone is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary chemotherapeutic agents that may be administered in combination with a CDP-epothilone conjugate include, bevacizumab (Avastin®), cisplatin (Platinol®), carboplatin (Paraplat®, Paraplatin®), irinotecan (Camptosar®), floxuridine (FUDF®), 5-fluorouracil (5FU) (Adrucil®, Efudex®, Fluoroplex®), leucovorin (Wellcovorin®), capecitabine (Xeloda®), gemcitabine (Gemzar®), oxaliplatin (Eloxatin®), mitoxantrone (Novantrone®), prednisone (Delta-Dome®, Deltasone®, Liquid Pred®, Lisacort®, Meticorten®, Orasone®, Prednicen-M®, Sk-Prednisone®, Sterapred®), estramustine (Emcyt®), sunitinib (Sutent®), temsirolimus (Torisel®), sorafenib (Nexavar®), everolimus (Afinitor®), cetuximab (Erbitux®), pemetrexed (ALIMTA®), erlotinib (Tarceva®), daunorubicin (Cerubidine®, Rubidomycin®), doxorubicin (Adriamycin®), trastuzumab (Herceptin®), or tamoxifen (Nolvadex®). Exemplary combinations of agents that can be administered with a CDP-epothilone conjugate include, e.g., bevacizumab (Avastin®) and interferon; 5FU (Adrucil®, Efudex®, Fluoroplex®) and leucovorin (Wellcovorin®); UFT (Uftoral®) and Leucovorin (Wellcovorin®); cisplatin (Platinol®) and pemetrexed (ALIMTA®); cisplastin (Platinol®) and vinorelbine (Navelbine®); cisplastin (Platinol®) and gemcitabine (Gemzar®); cisplastin (Platinol®) and vinblastine (Velban®, Velsar®); cisplastin (Platinol®), dacarbazine (DTIC-Dome®) and vinblastine (Velban®, Velsar®); cisplastin (Platinol®), temozolomide (Methazolastone®, Temodar®) and vinblastine (Velban®, Velsar®); cisplatin (Platinol®) and 5FU (Adrucil®, Efudex®, Fluoroplex®); oxaliplatin (Eloxatin®) and irinotecan (Camptosar®); 5FU (Adrucil®, Efudex®, Fluoroplex®), irinotecan (Camptosar®), and leucovorin (Wellcovorin®); 5FU (Adrucil®, Efudex®, Fluoroplex®), irinotecan (Camptosar®), oxaliplatin (Eloxatin®), and leucovorin (Wellcovorin®); 5FU (Adrucil®, Efudex®, Fluoroplex®) and radiation; 5FU (Adrucil®, Efudex®, Fluoroplex®), radiation and cisplatin (Platinol®); oxaliplatin (Eloxatin®), 5FU (Adrucil®, Efudex®, Fluoroplex®), and leucovorin (Wellcovorin®); capecitabine (Xeloda®), oxaliplatin (Eloxatin®), and bevacizumab (Avastin®); capecitabine (Xeloda®), irinotecan (Camptosar®), and bevacizumab (Avastin®); capecitabine (Xeloda®) and bevacizumab (Avastin®); irinotecan (Camptosar®) and bevacizumab (Avastin®); cetuximab (Erbutux®) and bevacizumab (Avastin®); cetuximab (Erbutux®), irinotecan (Camptosar®) and bevacizumab (Avastin®); panitumumab (Vectibix®) and bevacizumab (Avastin®); 5FU (Adrucil®, Efudex®, Fluoroplex®), leucovorin (Wellcovorin®) and bevacizumab (Avastin®); 5FU (Adrucil®, Efudex®, Fluoroplex®), leucovorin (Wellcovorin®), oxaliplatin (Eloxatin®) and bevacizumab (Avastin®); 5FU (Adrucil®, Efudex®, Fluoroplex®), leucovorin (Wellcovorin®), irinotecan (Camptosar®) and bevacizumab (Avastin®); 5FU (Adrucil®, Efudex®, Fluoroplex®), oxaliplatin (Eloxatin®), irinotecan (Camptosar®), leucovorin (Wellcovorin®) and bevacizumab (Avastin®); and UFT (Uftoral®), irinotecan (Camptosar®) and leucovorin (Wellcovorin®).

When formulating the pharmaceutical compositions featured in the invention the clinician may utilize preferred dosages as warranted by the condition of the subject being treated. For example, in one embodiment, a CDP-epothilone conjugate may be administered at a dosing schedule described herein, e.g., once every one, two three four, five, or six weeks.

Also, in general, a CDP-epothilone conjugate, and an additional chemotherapeutic agent(s) do not have to be administered in the same pharmaceutical composition, and may, because of different physical and chemical characteristics, have to be administered by different routes. For example, the CDP-epothilone conjugate may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a CDP-epothilone conjugate is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, camptothecin (e.g., IT-101)); and a platinum-based agent (e.g., cisplatin (Platinol®), carboplatin (Paraplat®, Paraplatin®), oxaliplatin (Eloxatin®)).

In another embodiment, the CDP-epothilone conjugate is administered once every two weeks in combination with one or more additional chemotherapeutic agent that is administered orally. For example, the CDP-epothilone conjugate can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The actual dosage of the CDP-epothilone conjugate and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In one embodiment, the CDP-epothilone can be administered at a dose that includes 0.5 to 300 mg/m² of an epothilone, e.g., 2.5 mg/m² to 30 mg/m², 9 to 280 mg/m², 0.5 to 100 mg/m², 0.5 to 35 mg/m², 25 to 90 mg/m². Preferably, the CDP-epothilone conjugate is administered at a dosage described herein.

In some embodiments, when a CDP-epothilone conjugate is administered in combination with one or more additional chemotherapeutic agent, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml×min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

The disclosure also encompasses a method for the synergistic treatment of cancer wherein a CDP-epothilone conjugate is administered in combination with an additional chemotherapeutic agent or agents.

The particular choice of polymer conjugate and anti-proliferative cytotoxic agent(s) or radiation will depend upon the diagnosis of the attending physicians and their judgment of the condition of the subject and the appropriate treatment protocol.

If the CDP-epothilone conjugate and the chemotherapeutic agent(s) and/or radiation are not administered simultaneously or essentially simultaneously, then the initial order of administration of the CDP-epothilone conjugate, and the chemotherapeutic agent(s) and/or radiation, may be varied. Thus, for example, the CDP-epothilone conjugate may be administered first followed by the administration of the chemotherapeutic agent(s) and/or radiation; or the chemotherapeutic agent(s) and/or radiation may be administered first followed by the administration of the CDP-epothilone conjugate. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-epothilone conjugate, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Indications

The disclosed CDP-epothilone conjugates are useful in evaluating or treating proliferative disorders, e.g., treating a tumor and metastases thereof wherein the tumor or metastases thereof is a cancer described herein. The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer, childhood; esophageal cancer; esophageal cancer, childhood; gallbladder cancer; gastric (stomach) cancer; gastric (stomach) cancer, childhood; hepatocellular (liver) cancer, adult (primary); hepatocellular (liver) cancer, childhood (primary); extrahepatic; pancreatic cancer; pancreatic cancer, childhood; sarcoma, rhabdomyosarcoma; pancreatic cancer, islet cell; rectal cancer; and small intestine cancer;

Endocrine cancers such as islet cell carcinoma (endocrine pancreas); adrenocortical carcinoma; adrenocortical carcinoma, childhood; gastrointestinal carcinoid tumor; parathyroid cancer; pheochromocytoma; pituitary tumor; thyroid cancer; thyroid cancer, childhood; multiple endocrine neoplasia syndrome, childhood; and carcinoid tumor, childhood;

Eye cancers such as intraocular melanoma; and retinoblastoma;

Musculoskeletal cancers such as Ewing's family of tumors; osteosarcoma/malignant fibrous histiocytoma of the bone; rhabdomyosarcoma, childhood; soft tissue sarcoma, adult; soft tissue sarcoma, childhood; clear cell sarcoma of tendon sheaths; and uterine sarcoma;

Breast cancer such as breast cancer and pregnancy; breast cancer, childhood; and breast cancer, male;

Neurologic cancers such as brain stem glioma, childhood; brain tumor, adult; brain stem glioma, childhood; cerebellar astrocytoma, childhood; cerebral astrocytoma/malignant glioma, childhood; ependymoma, childhood; medulloblastoma, childhood; pineal and supratentorial primitive neuroectodermal tumors, childhood; visual pathway and hypothalamic glioma, childhood; other childhood brain cancers; adrenocortical carcinoma; central nervous system lymphoma, primary; cerebellar astrocytoma, childhood; neuroblastoma; craniopharyngioma; spinal cord tumors; central nervous system atypical teratoid/rhabdoid tumor; central nervous system embryonal tumors; andsupratentorial primitive neuroectodermal tumors, childhood and pituitary tumor;

Genitourinary cancers such as bladder cancer; bladder cancer, childhood; kidney cancer; ovarian cancer, childhood; ovarian epithelial cancer; ovarian low malignant potential tumor; penile cancer; prostate cancer; renal cell cancer, childhood; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as extracranial germ cell tumor, childhood; extragonadal germ cell tumor; ovarian germ cell tumor; and testicular cancer;

Head and neck cancers such as lip and oral cavity cancer; oral cancer, childhood; hypopharyngeal cancer; laryngeal cancer; laryngeal cancer, childhood; metastatic squamous neck cancer with occult primary; mouth cancer; nasal cavity and paranasal sinus cancer; nasopharyngeal cancer; nasopharyngeal cancer, childhood; oropharyngeal cancer; parathyroid cancer; pharyngeal cancer; salivary gland cancer; salivary gland cancer, childhood; throat cancer; and thyroid cancer;

Hematologic/blood cell cancers such as a leukemia (e.g., acute lymphoblastic leukemia, adult; acute lymphoblastic leukemia, childhood; acute myeloid leukemia, adult; acute myeloid leukemia, childhood; chronic lymphocytic leukemia; chronic myelogenous leukemia; and hairy cell leukemia); a lymphoma (e.g., AIDS-related lymphoma; cutaneous T-cell lymphoma; Hodgkin's lymphoma, adult; Hodgkin's lymphoma, childhood; Hodgkin's lymphoma during pregnancy; non-Hodgkin's lymphoma, adult; non-Hodgkin's lymphoma, childhood; non-Hodgkin's lymphoma during pregnancy; mycosis fungoides; sezary syndrome; T-cell lymphoma, cutaneous; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; thymoma, childhood; thymic carcinoma; bronchial adenomas/carcinoids; pleuropulmonary blastoma; non-small cell lung cancer; and small cell lung cancer;

Skin cancers such as Kaposi's sarcoma; Merkel cell carcinoma; melanoma; and skin cancer, childhood;

Other childhood cancers and cancers of unknown primary site;

and metastases of the aforementioned cancers can also be treated or prevented in accordance with the methods described herein.

The CDP-epothilone conjugates described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a CDP-epothilone conjugate and a second therapeutic agent. Various combinations are described herein. The combination can reduce the development of tumors, reduces tumor burden, or produce tumor regression in a mammalian host.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### General Experimental Procedures

All of the anhydrous solvents, HPLC grade solvents and other common organic solvents will be purchased from commercial suppliers (e.g., Sigma-Aldrich) and used without further purification. Parent polymer, Poly-CD-PEG, will be synthesized as previously described (Cheng, Khin et al. (2003) Bioconjug. Chem. 14(5):1007-17). Ixabepilone, KOS-1584, sagopilone and BMS310705 will be purchased from a commercial supplier: Hangzhou onicon corporation, China; ACC corporation, CA, USA; Tocric Biosciences, MO, USA; or Molocon Corporation, ON, Canada. De-ionized water (18-MΩ-cm) will be obtained by passing in-house de-ionized water through a Milli-Q Bioicel Water System (Millipore) or a Barnstead E-pure purification system (Thermo Fisher Scientific, Waltham, Mass.). NMR spectra will be recorded on a Varian Inova 400 MHz spectrometer (Palo Alto, Calif.). Mass spectral (MS) analysis will be performed on Bruker FT-MS 4.7 T electrospray mass spectrometer. MWs of the polymer samples will be analyzed on a Agilent 1200 RI coupled with Viscotek 270 LALS-RALS system. Ixabepilone, ixabepilone derivatives, polymer-ixabepilone conjugates, KOS-1584, KOS-1584 derivatives, polymer-KOS-1584 conjugates, sagopilone, sagopilone derivatives, polymer-sagopilone conjugates, epothilone, epothilone derivatives and polymer-epothilone conjugates will be analyzed with a C-18 reverse phase column (Zorbax eclipse) on a Agilent 1100 HPLC system using ammonium bicarbonate buffer (pH 8) and acetonitrile. Particle size measurement will be carried out on a Zetasizer nano-zs (Serial # mal1017190 Malvern Instruments, Worcestershire, UK).

### Example 1

### Synthesis of a C-3 Derivative of CDP-C(O)—O-Ixabepilone

Method A: Directly Attach Linker to Epothilone, Separate Mixture, Deprotect and then Couple to CDP

### Step 1: Synthesis of Ixabepilone-ε-TROC-aminohexanoate (Scheme 1)

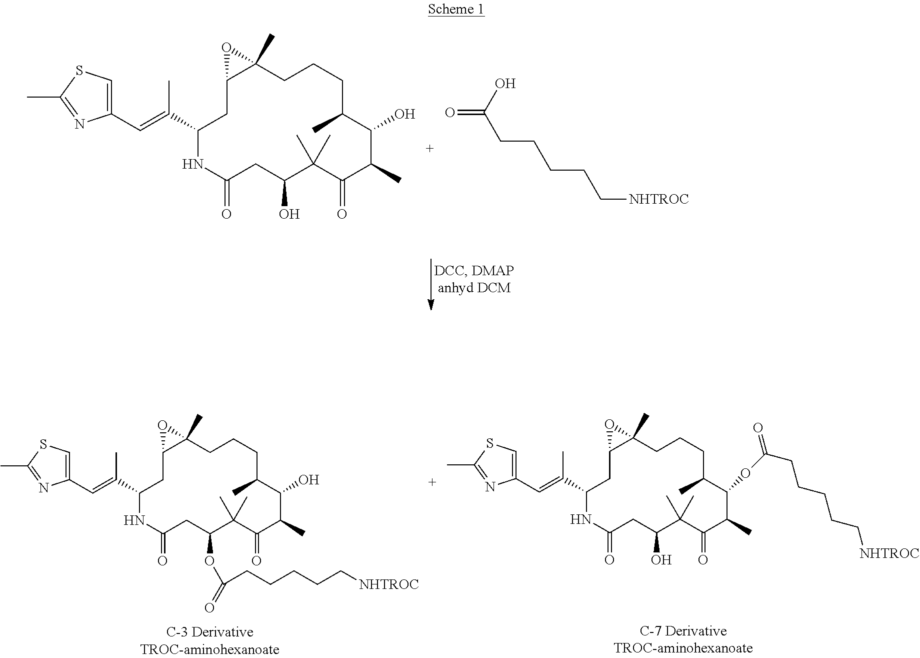

Ixabepilone (20 mg, 0.039 mmol) and ε-TROC-aminohexanoic acid (16.3 mg, 0.0585 mmol) will be dissolved in anhydrous DCM (10 mL) under N₂. To the resulting clear solution, DCC (13.4 mg, 0.065 mmol) and DMAP (7.9 mg, 0.065 mmol) will be added (Scheme 1). The reaction mixture will then be stirred for 12 h at room temperature. The solvent will subsequently be evaporated and the resulting residue dissolved in a minimum amount of chloroform. The desired C-3 and C-7 derivatives can be isolated via purification using flash column chromatography with chloroform/methanol as the mobile phase. The derivatives are to be analyzed by electron spray mass spectroscopy (m/z), HPLC and ¹H-NMR. The C-3 derivative of Ixabepilone-ε-TROC-aminohexanoate is used as an example in the following synthetic steps.

### Step 2: Synthesis of Ixabepilone-ε-aminohexanoate (Scheme 2)

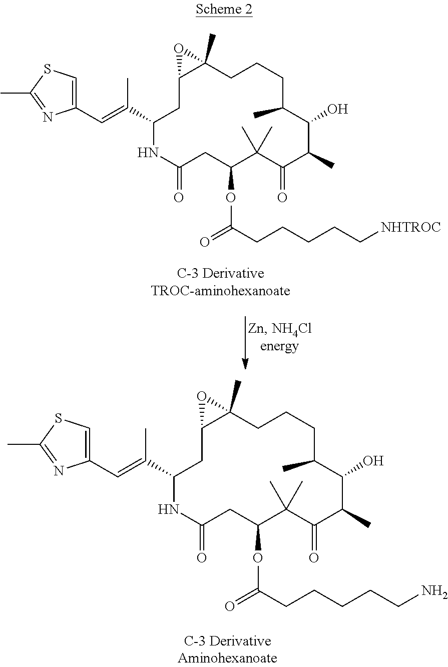

The C-3 derivative of Ixabepilone-ε-TROC-aminohexanoate (15 mg, 0.019 mmol) and ammonium chloride (100 mg, 1.88 mmol) will be combined and mixed in 3 ml of water. While stirring vigorously, Zn powder (98 mg, 1.51 mmol) will be added with the input of energy (e.g., heat, sonication, microwave or ultraviolet irradiation) (Martin et al. (2000) Angewandte Chemie International Edition 39 (3), 581-583) and stirred for an additional 20 min. The resulting solution will be filtered to remove zinc oxide and then washed with hot water. The product will be extracted in dichloromethane and dried over MgSO₄. Evaporation of the organic solvent will be followed by purification of the crude product via flash chromatography. The purified product will then be analyzed by electron spray mass spectroscopy (m/z), HPLC and ¹H-NMR.

### Step 3: Synthesis of CDP-C(O)—O-Ixabepilone (Scheme 3)

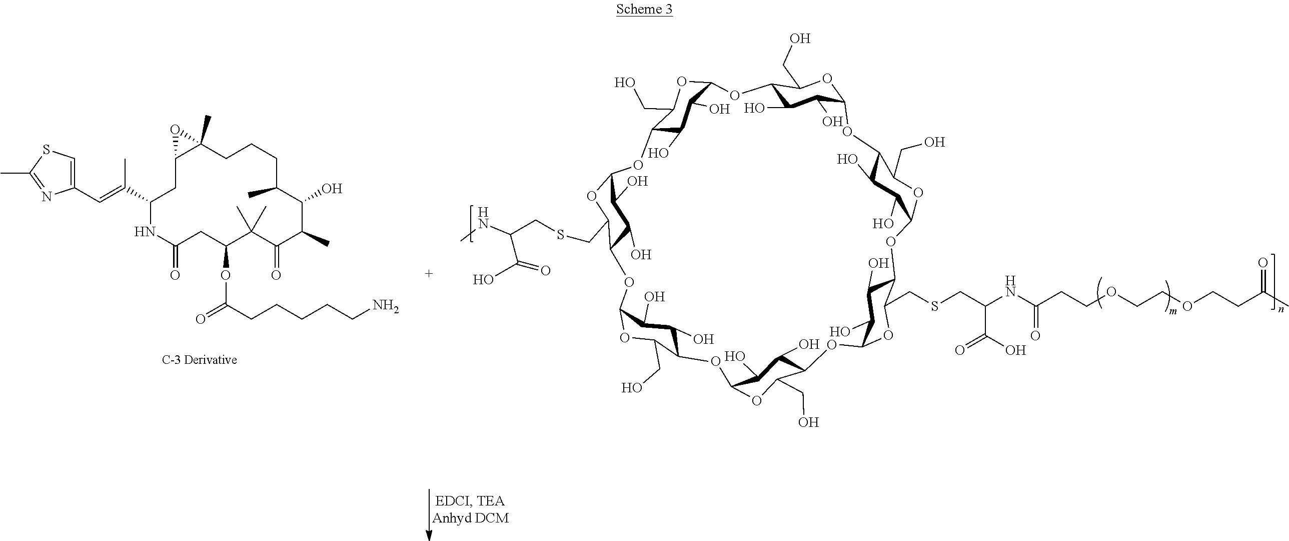

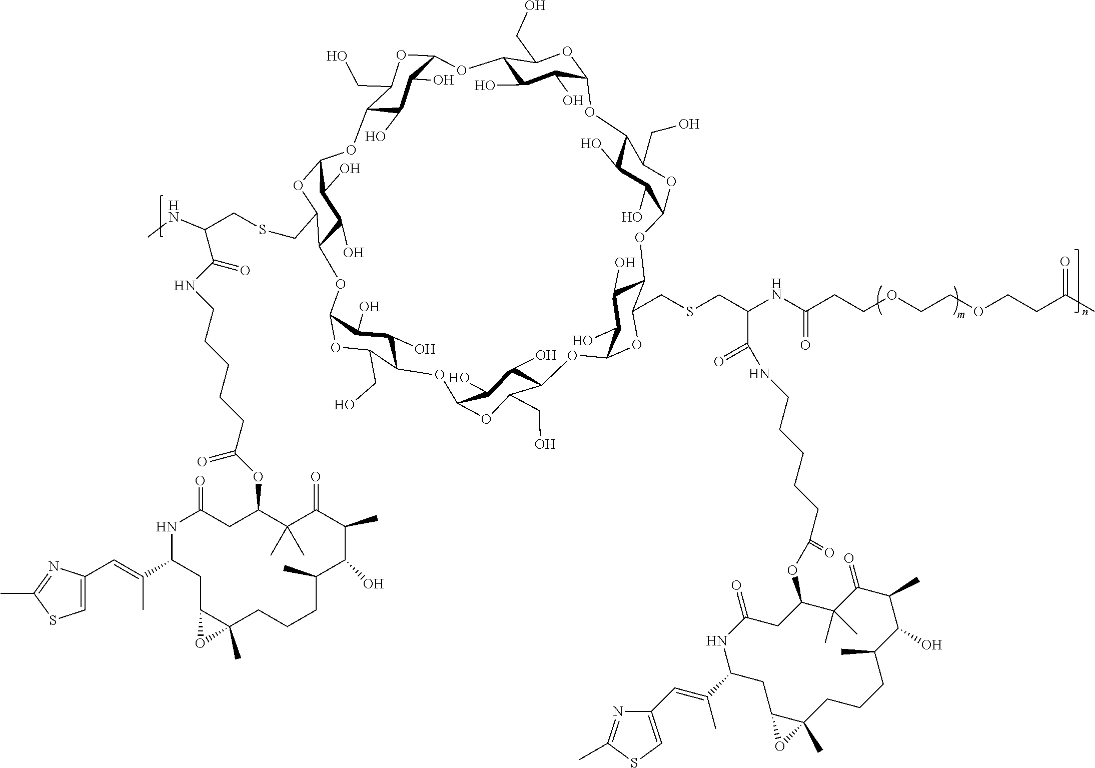

CDP-COOH (50 mg, 0.011 mmol) will be dissolved in MeOH (2.0 mL). The C-3 derivative of Ixabepilone-ε-aminohexanoate (14.7 mg, 0.024 mmol) will subsequently be added to the mixture and stirred for a few minutes to obtain a clear solution. EDCI (6.1 mg, 0.032 mmol) and TEA (3.8 mg, 0.038 mmol) will be added and the reaction stirred at ambient temperature for 3 h (Scheme 3). The resulting reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will then be washed with acetone (1 mL) twice, dissolved in nanopure water (3 mL) and then filtered through a 0.2 μm filter membrane and lyophilized to afford CDP-C(O)-β-Ixabepilone. Loading will be determined by UV/Vis spectrometry with a standard curve. The particle size will be determined by Zetasizer.

Method B: Selectively Protect with Silyl Protecting Group, Addition of Linker, Followed by Deprotection and then Conjugation with CDP

### Step 1: Synthesis of 3-tert-butyldimethylsilyl Ixabepilone or 7-tert-butyldimethylsilyl Ixabepilone (Scheme 4)

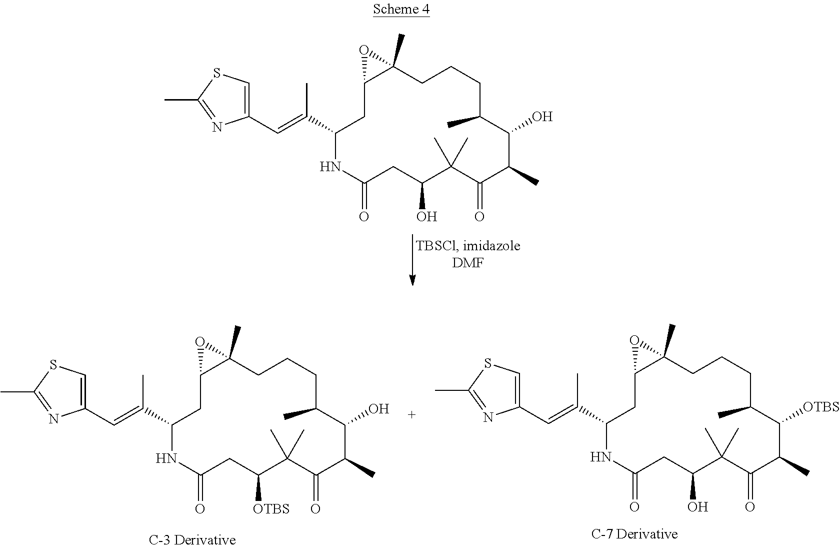

Ixabepilone (20 mg, 0.039 mmol) and tert-butyldimethylsilyl chloride (8.3 mg, 0.055 mmol) will be mixed in anhydrous DMF (5 mL) under N₂ atm. To the resulting clear solution, imidazole (10.7 mg, 0.158 mmol) will be added (Scheme 4) and the reaction will be allowed to stir at ambient temperature for 24 h. The solvent will be evaporated and the residue dissolved in a minimum amount of chloroform. The desired C-3 and C-7 derivatives will be isolated following purification of the crude product via flash column chromatography with chloroform/methanol as the mobile phase. The derivatives will be analyzed by electron spray mass spectroscopy (m/z), HPLC and ¹H-NMR. The C-3 derivative of TBS-Ixabepilone is used as an example in the following synthetic steps.

### Step 2: Synthesis of 3-(tert-butyldimethylsilyl)-7-(TROC-aminohexan)-Ixabepilone-oate (Scheme 5)

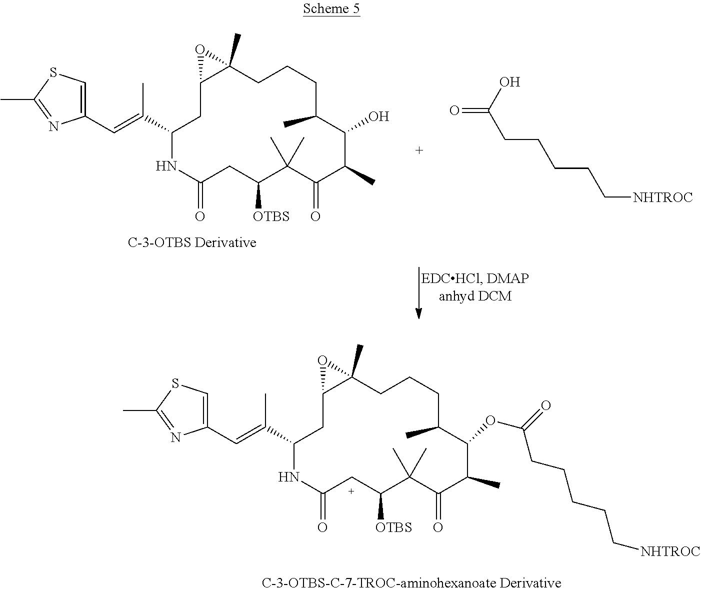

7-tert-butyldimethylsilyl Ixabepilone (20 mg, 0.032 mmol) and ε-TROC-aminohexanoic acid (12.0 mg, 0.039 mmol) will be stirred together in anhydrous DCM (2 mL) under N₂. To the resulting clear solution, EDC.HCl (11.1 mg, 0.058 mmol) and DMAP (7.08 mg, 0.058 mmol) will be added (Scheme 5). The reaction mixture is then stirred for 12 h at 22° C. The solvent is subsequently evaporated and the resulting residue dissolved in a minimum amount of chloroform. The crude product will be purified via flash column chromatography with chloroform/methanol as the mobile phase. The product will be analyzed by electron spray mass spectroscopy (m/z), HPLC and ¹H-NMR.

### Step 3: Synthesis of 7-(aminohexan)-Ixabepilone-oate (Scheme 6)

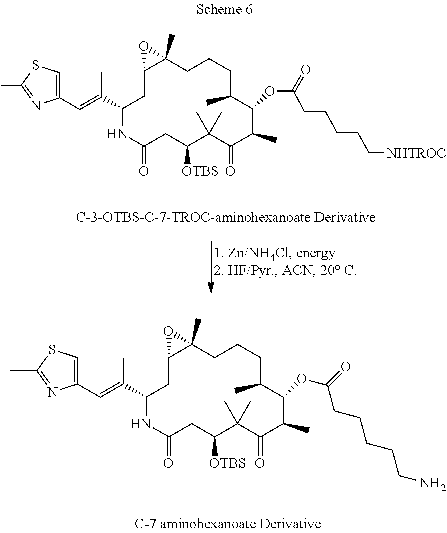

3-(tert-butyldimethylsilyl)-7-(TROC-aminohexan)-Ixabepilone-oate will be deprotected using Zn/NH₄Cl with the input of energy (e.g., heat, sonication, microwave or ultraviolet irradiation), followed by a solution of acetonitrile and HF/pyridine. The final product will be purified via flash column chromatography with chloroform/methanol as the mobile phase. 3-(aminohexan)-Ixabepilone-oate will be analyzed by electron spray mass spectroscopy (m/z), HPLC and ¹H-NMR.

### Step 4: Synthesis of CDP-C(O)—O-Ixabepilone (Scheme 7)

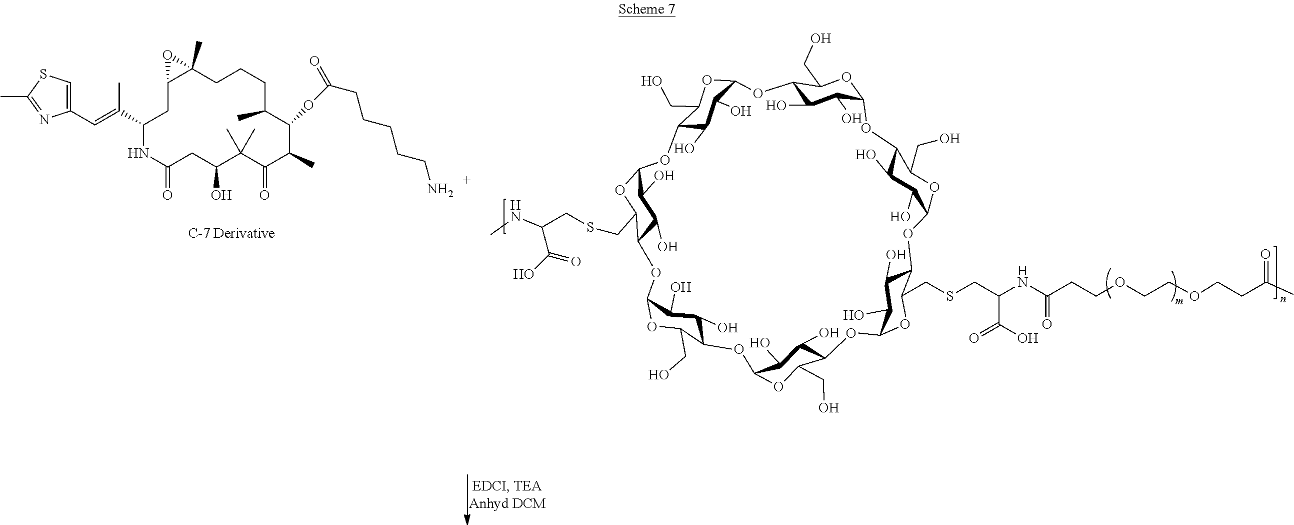

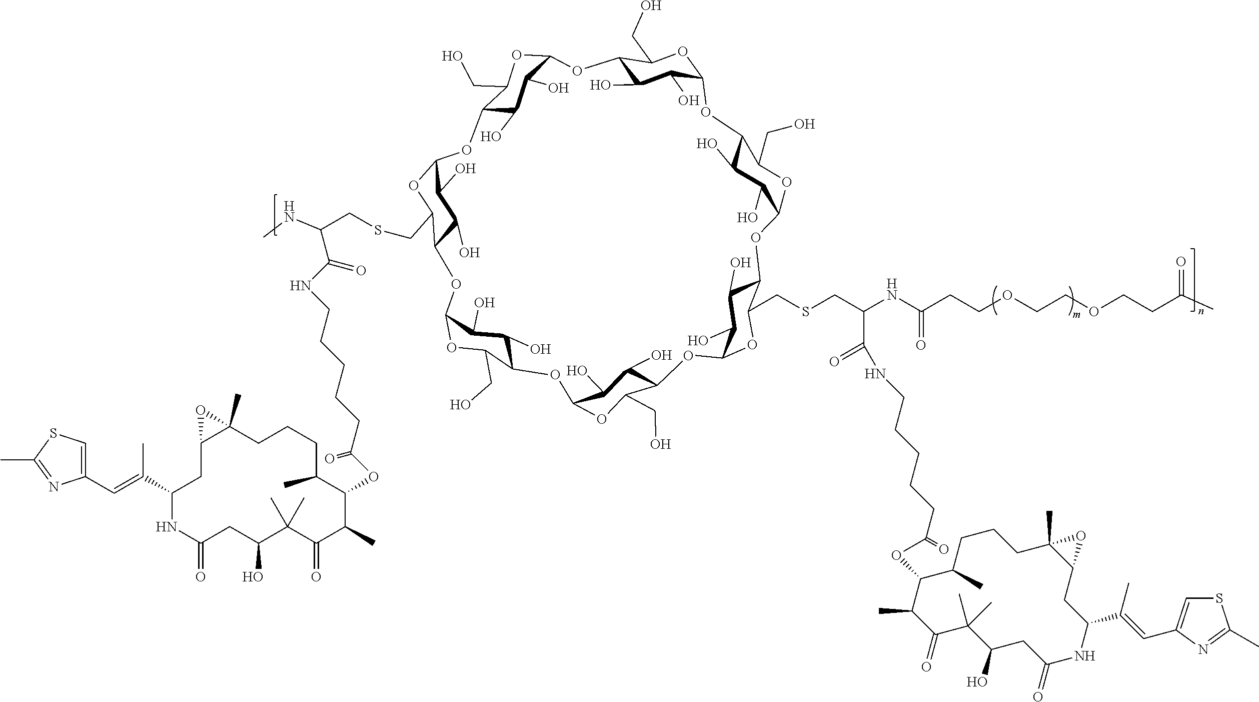

CDP-COOH (50 mg, 0.011 mmol) will be dissolved in MeOH (2.0 mL). C-7 derivative of Ixabepilone-ε-aminohexanoate (14.7 mg, 0.024 mmol) will subsequently be added to the mixture and stirred for a few minutes to obtain a clear solution. EDCI (6.1 mg, 0.032 mmol) and TEA (3.8 mg, 0.038 mmol) will then be added and the reaction stirred at ambient temperature for 3 h (Scheme 7). The reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will be washed with acetone (1 mL) twice, dissolved in nanopure water (3 mL) and then filtered through a 0.2 μm filter membrane and lyophilized to afford CDP-C(O)—O-Ixabepilone.

### Example 2

### Synthesis of a C-7 Derivative of CDP-C(O)—O-Ixabepilone

Method A: Directly Attach Linker to Epothilone, Separate Mixture, Deprotect and then Couple to CDP

### Step 1: Synthesis of Ixabepilone-ε-TROC-aminohexanoate (Scheme 8)

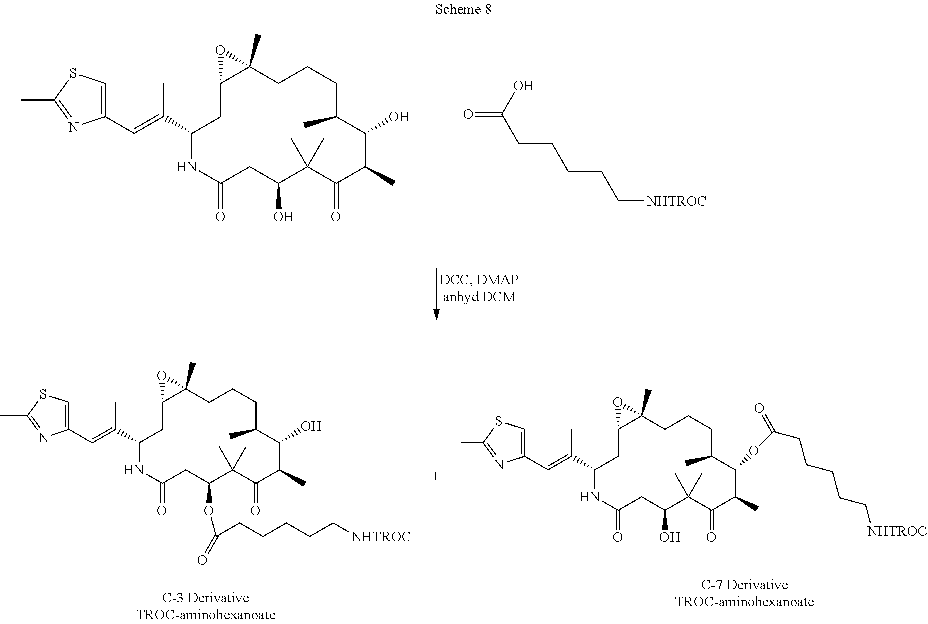

Ixabepilone (20 mg, 0.039 mmol) and ε-TROC-aminohexanoic acid (16.3 mg, 0.0585 mmol) will be dissolved in anhydrous DCM (10 mL) under N₂. To the resulting clear solution, DCC (13.4 mg, 0.065 mmol) and DMAP (7.9 mg, 0.065 mmol) will be added (Scheme 8). The reaction mixture will then be stirred for 12 h at room temperature. The solvent will subsequently be evaporated and the resulting residue dissolved in a minimum amount of chloroform. The desired C-3 and C-7 derivatives can be isolated via purification using flash column chromatography with chloroform/methanol as the mobile phase. The derivatives are to be analyzed by electron spray mass spectroscopy (m/z), HPLC and ¹H-NMR. The C-7 derivative of Ixabepilone-ε-TROC-aminohexanoate is used as an example in the following synthetic steps.

### Step 2: Synthesis of habepilone-ε-aminohexanoate (Scheme 9)

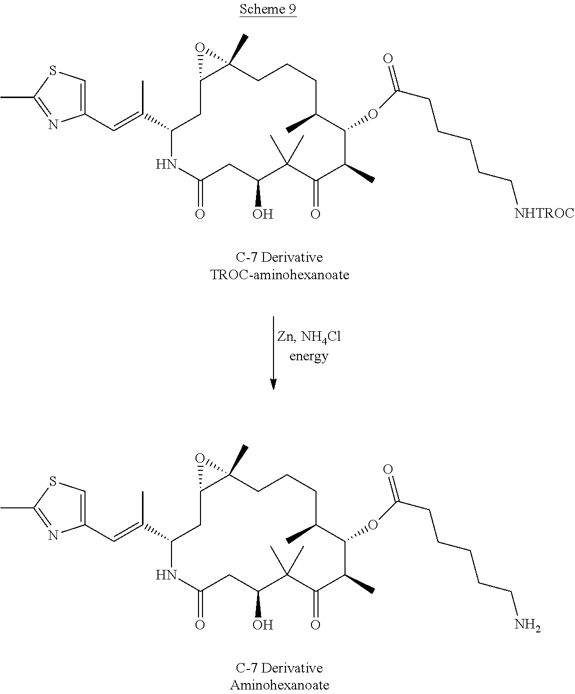

The C-7 derivative of Ixabepilone-ε-TROC-aminohexanoate (15 mg, 0.019 mmol) and ammonium chloride (100 mg, 1.88 mmol) will be combined and mixed in 3 ml of water. While stirring vigorously, Zn powder (98 mg, 1.51 mmol) will be added with the input of energy (e.g., heat, sonication, microwave or ultraviolet irradiation (Martin et al. (2000) Angewandte Chemie International Edition, 39 (3):581-583) and stirred for an additional 20 min. The resulting solution will be filtered to remove zinc oxide and then washed with hot water. The product will be extracted in dichloromethane and dried over MgSO₄. Evaporation of the organic solvent will be followed by purification of the crude product via flash chromatography. The purified product will then be analyzed by electron spray mass spectroscopy (m/z), HPLC and ¹H-NMR.

### Step 3: Synthesis of CDP-C(O)—O-Ixabepilone (Scheme 10)

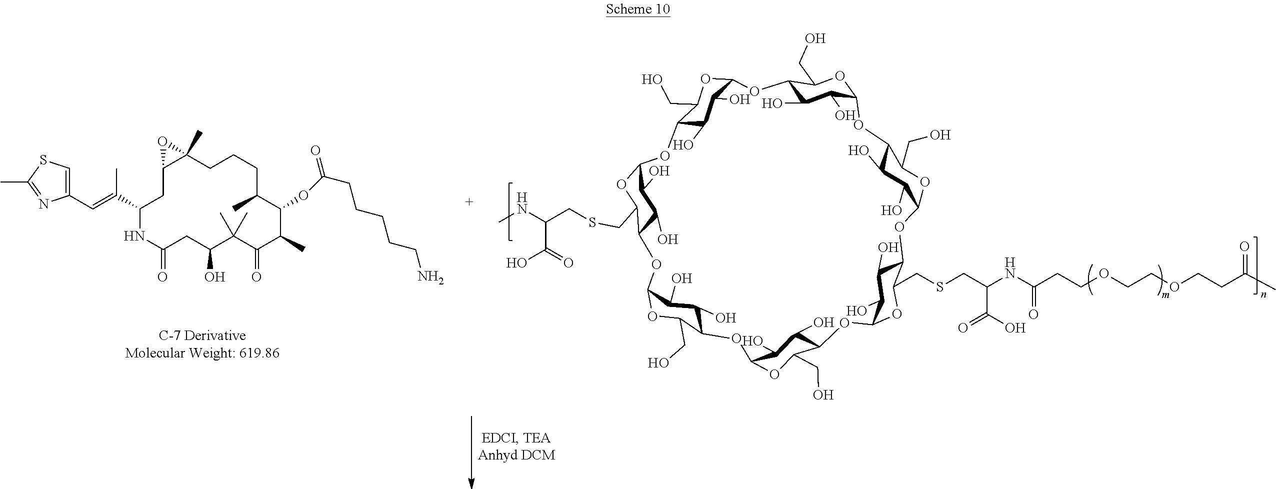

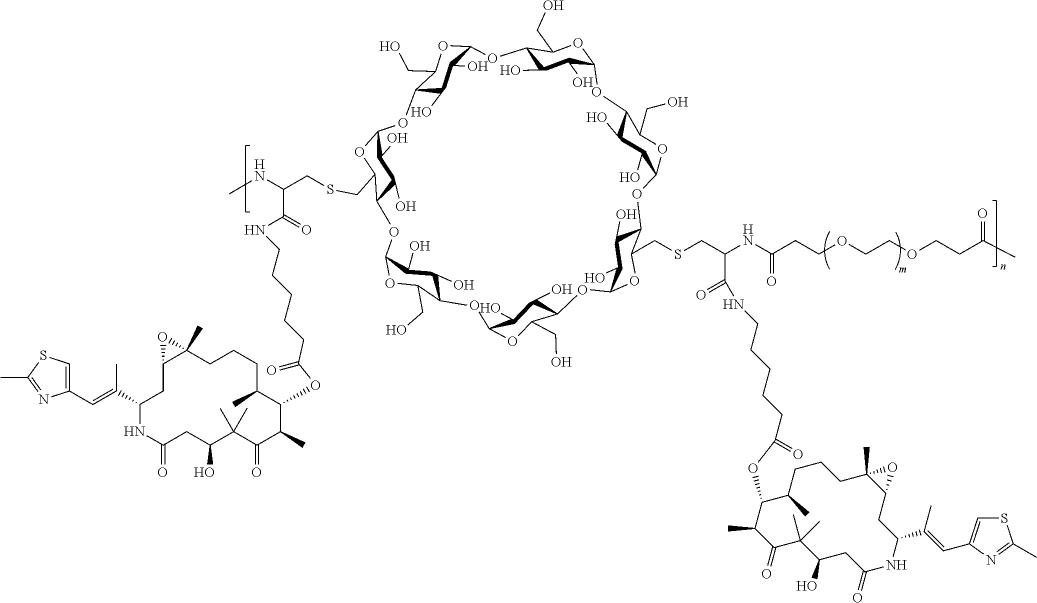

CDP-COOH (50 mg, 0.011 mmol) will be dissolved in MeOH (2.0 mL). The C-7 derivative of Ixabepilone-ε-aminohexanoate (14.7 mg, 0.024 mmol) will subsequently be added to the mixture and stirred for a few minutes to obtain a clear solution. EDCI (6.1 mg, 0.032 mmol) and TEA (3.8 mg, 0.038 mmol) will be added and the reaction stirred at ambient temperature for 3 h (Scheme 10). The resulting reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will then be washed with acetone (1 mL) twice, dissolved in nanopure water (3 mL) and then filtered through a 0.2 μm filter membrane and lyophilized to afford CDP-C(O)—O-Ixabepilone. Loading will be determined by UV/Vis spectrometry with a standard curve. The particle size will be determined by Zetasizer.

Method B: Selectively Protect with Silyl Protecting Group, Addition of Linker, Followed by Deprotection and then Conjugation with CDP

### Step 1: Synthesis of 3-tert-butyldimethylsilyl Ixabepilone or 7-tert-butyldimethylsilyl Ixabepilone (Scheme 11)

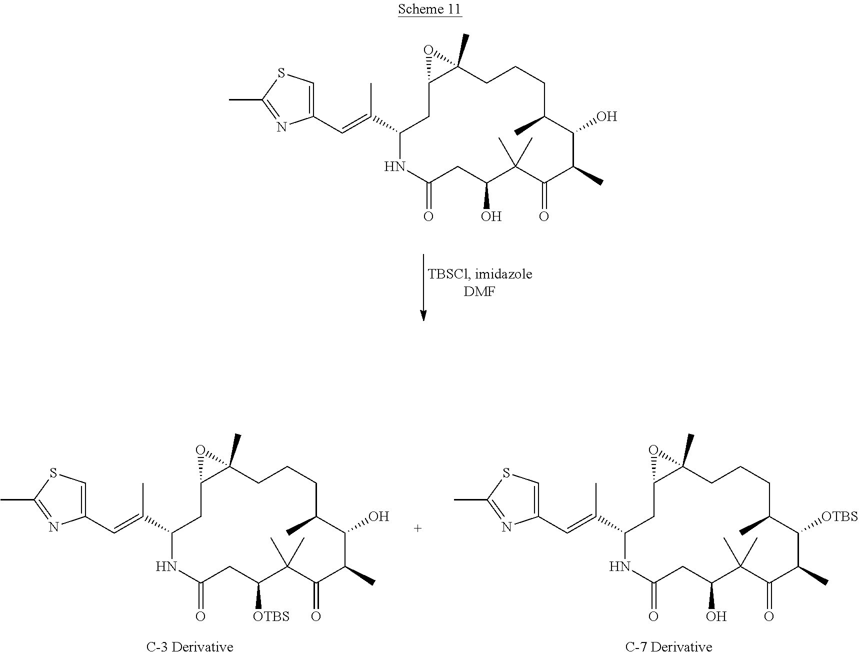 [SMILES: C/C(=C\c1csc(C)n1)[C@@H]1C[C@@H]2O[C@]2(C)CCC[C@H](C)[C@H](O)[C@@H](C)C(=O)C(C)(C)[C@@H](O)CC(=O)N1 ; C/C(=C\c1csc(C)n1)[C@@H]1C[C@@H]2O[C@]2(C)CCC[C@H](C)[C@H](O)[C@@H](C)C(=O)C(C)(C)[C@@H](O[Si](C)(C)C(C)(C)C)CC(=O)N1 ; C/C(=C\c1csc(C)n1)[C@@H]1C[C@@H]2O[C@]2(C)CCC[C@H](C)[C@H](O[Si](C)(C)C(C)(C)C)[C@@H](C)C(=O)C(C)(C)[C@@H](O)CC(=O)N1]

Ixabepilone (20 mg, 0.039 mmol) and tert-butyldimethylsilyl chloride (8.3 mg, 0.055 mmol) will be mixed in anhydrous DMF (5 mL) under N₂ atm. To the resulting clear solution, imidazole (10.7 mg, 0.158 mmol) will be added (Scheme 11) and the reaction will be allowed to stir at ambient temperature for 24 h. The solvent will be evaporated and the residue dissolved in a minimum amount of chloroform. The desired C-3 and C-7 derivatives will be isolated following purification of the crude product via flash column chromatography with chloroform/methanol as the mobile phase. The derivatives will be analyzed by electron spray mass spectroscopy (m/z), HPLC and ¹H-NMR. The C-7 derivative of TBS-Ixabepilone is used as an example in the following synthetic steps.

### Step 2: Synthesis of 7-(tert-butyldimethylsilyl)-3-(TROC-aminohexan)-Ixabepilone-oate (Scheme 12)

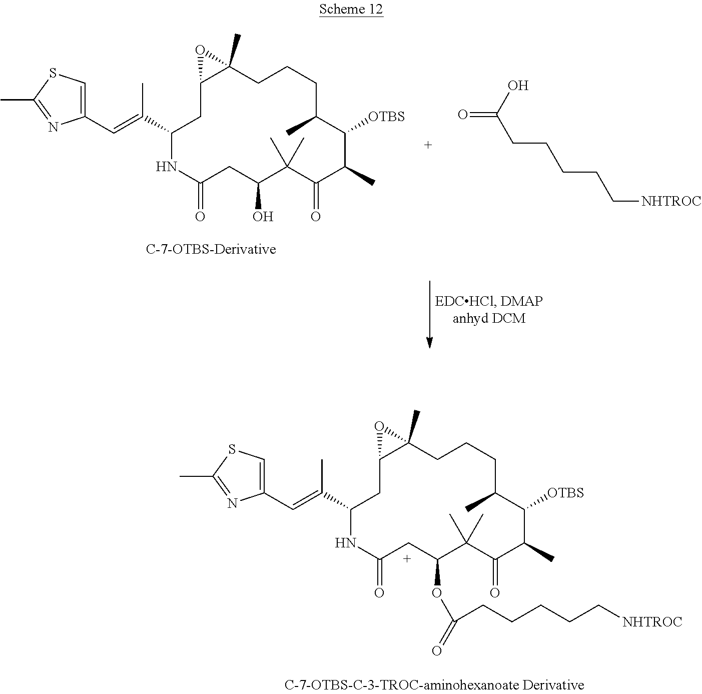

7-tert-butyldimethylsilyl Ixabepilone (20 mg, 0.032 mmol) and ε-TROC-aminohexanoic acid (12.0 mg, 0.039 mmol) will be stirred together in anhydrous DCM (2 mL) under N₂. To the resulting clear solution, EDC.HCl (11.1 mg, 0.058 mmol) and DMAP (7.08 mg, 0.058 mmol) will be added (Scheme 12). The reaction mixture will then be stirred for 12 h at 22° C. The solvent is subsequently evaporated and the resulting residue dissolved in a minimum amount of chloroform. The crude product will be purified via flash column chromatography with chloroform/methanol as the mobile phase. The product will be analyzed by electron spray mass spectroscopy (m/z), HPLC and ¹H-NMR.

### Step 3: Synthesis of 3-(aminohexan)-Ixabepilone-oate (Scheme 13)

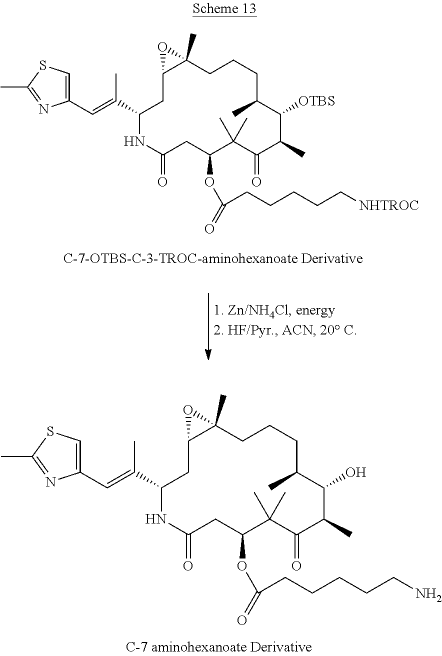

7-(tert-butyldimethylsilyl)-3-(TROC-aminohexan)-Ixabepilone-oate will be deprotected using Zn/NH₄Cl with the input of energy (e.g., heat, sonication, microwave or ultraviolet irradiation), followed by a solution of acetonitrile and HF/Pyridine. The final product will be purified via flash column chromatography with chloroform/methanol as the mobile phase. 3-(aminohexan)-Ixabepilone-oate will be analyzed by electron spray mass spectroscopy (m/z), HPLC and ¹H-NMR.

### Step 4: Synthesis of CDP-C(O)—O-Ixabepilone (Scheme 14)

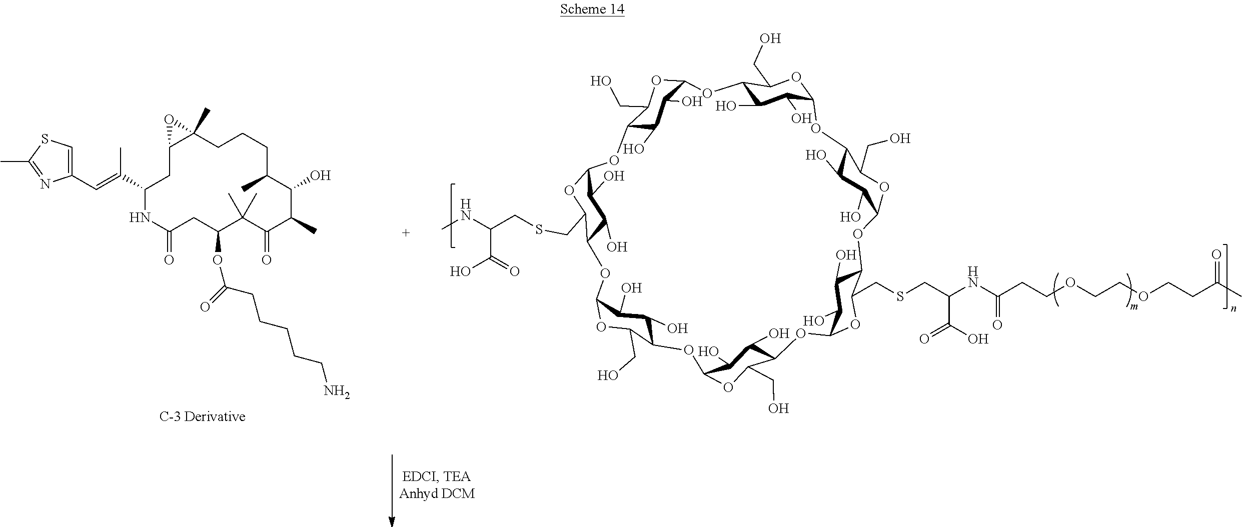

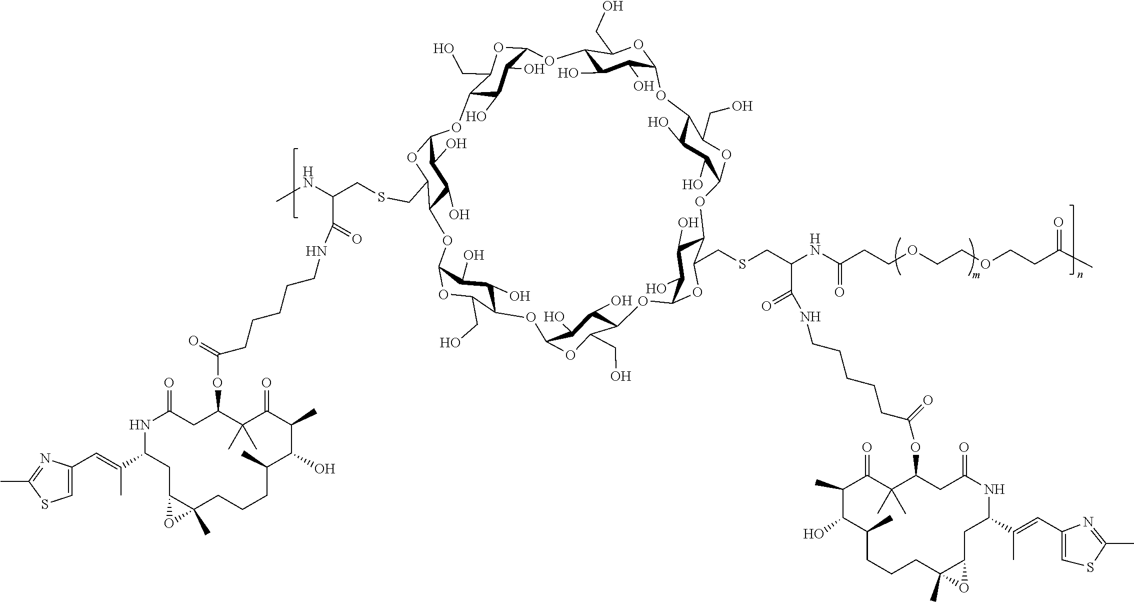

CDP-COOH (50 mg, 0.011 mmol) will be dissolved in MeOH (2.0 mL). C-3 derivative of Ixabepilone-ε-aminohexanoate (14.7 mg, 0.024 mmol) will subsequently be added to the mixture and stirred for a few minutes to obtain a clear solution. EDCI (6.1 mg, 0.032 mmol) and TEA (3.8 mg, 0.038 mmol) will then be added and the reaction stirred at ambient temperature for 3 h (Scheme 14). The reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will be washed with acetone (1 mL) twice, dissolved in nanopure water (3 mL) and then filtered through a 0.2 μm filter membrane and lyophilized to afford CDP-C(O)—O-Ixabepilone.

### Example 3

### Synthesis of CDP-Phosphonamide-Epothilone B

### Synthesis of Fmoc-NH—(CH₂)₂—PO(OH)₂

2-Aminoethylphosphonic acid (5.0 g, 0.040 mol) will be dissolved in a tetrahydrofuran/water mixture (1:1) (40 mL). To the mixture, Fmoc N-hydroxysuccinimide ester (16 g, 0.048 mmol) in THF (10 mL) will be added slowly in an ice bath and stirred for ½ h. It will be stirred at ambient temperature for an additional 2 h. The solvent will be removed under vacuum (Scheme 15).

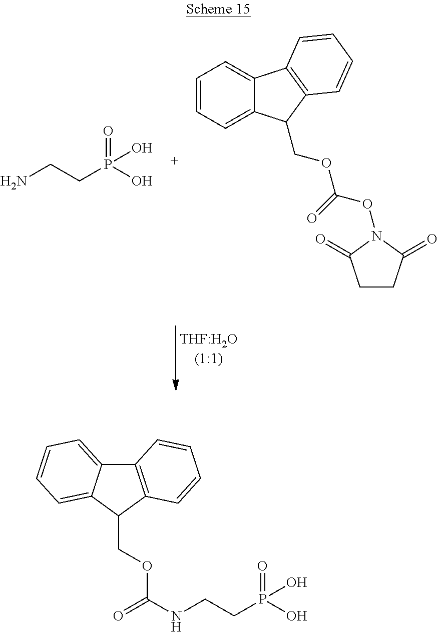 [SMILES: NCCP(=O)(O)O ; O=C(OCC1c2ccccc2-c2ccccc21)ON1C(=O)CCC1=O ; O=C(NCCP(=O)(O)O)OCC1c2ccccc2-c2ccccc21]

### Synthesis of NH₂—(CH₂)₂—PO(OH)—NH-Epothilone

Fmoc-NH—(CH₂)₂—PO(OH)₂ (3.0 g, 8.6 mmol) will be dissolved in methylene chloride (100 mL). N,N′-Dicyclohexylcarbodiimide (2.1 g, 10 mmol) and N-hydroxysuccinimide (1.2 g, 10 mmol) will be added to the solution in an ice bath. The mixture will be stirred for ½ h in an ice bath and it will be stirred at ambient temperature for additional 1 h. Epothilone B analog (5.4 g, 10 mmol) will be added to the mixture and stirred for an additional 3 h. White precipitate will be filtered off. The organic layer will be washed with brine and dried over MgSO₄. The organic layer will be removed under vacuum to yield solid product. The solid will be purified by flash column chromatography. The product will be deprotected using a piperidine in methanol mixture. The organic layer will be pumped down and used without further purification. (Scheme 16).

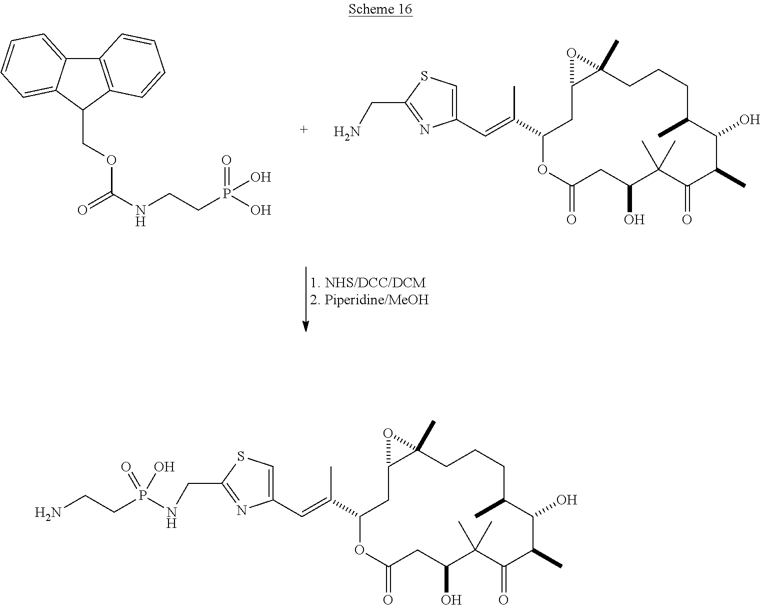

### Synthesis of CDP-NH₂—(CH₂)₂—PO(OH)—NH-Epothilone B

CDP (1.0 g, 0.21 mmol) will be dissolved in dry N,N-dimethylformamide (DMF, 20 mL). NH₂—(CH₂)₂—PO(OH)—NH-Epothilone (300 mg, 0.46 mmol), N,N-diisopropylethylamine (0.080 mL, 0.46 mmol), N-(3-Dimethylaminopropyl)-N′-ethylcarbodiimide hydrochloride (120 mg, 0.62 mmol), and N-Hydroxysuccinimide (52 mg, 0.46 mmol) will be added to the polymer solution and stirred for 4 h. The polymer will be precipitated with ethylacetate (100 mL) and rinsed with acetone (50 mL). The precipitate will be dissolved in water at pH 8 (100 mL). The solution will be dialyzed using 25,000 MWCO membrane (Spectra/Por 7) for 24 h in water. It will be filtered through 0.2 μm filters (Nalgene) and lyophilized to yield a white solid (Scheme 17).

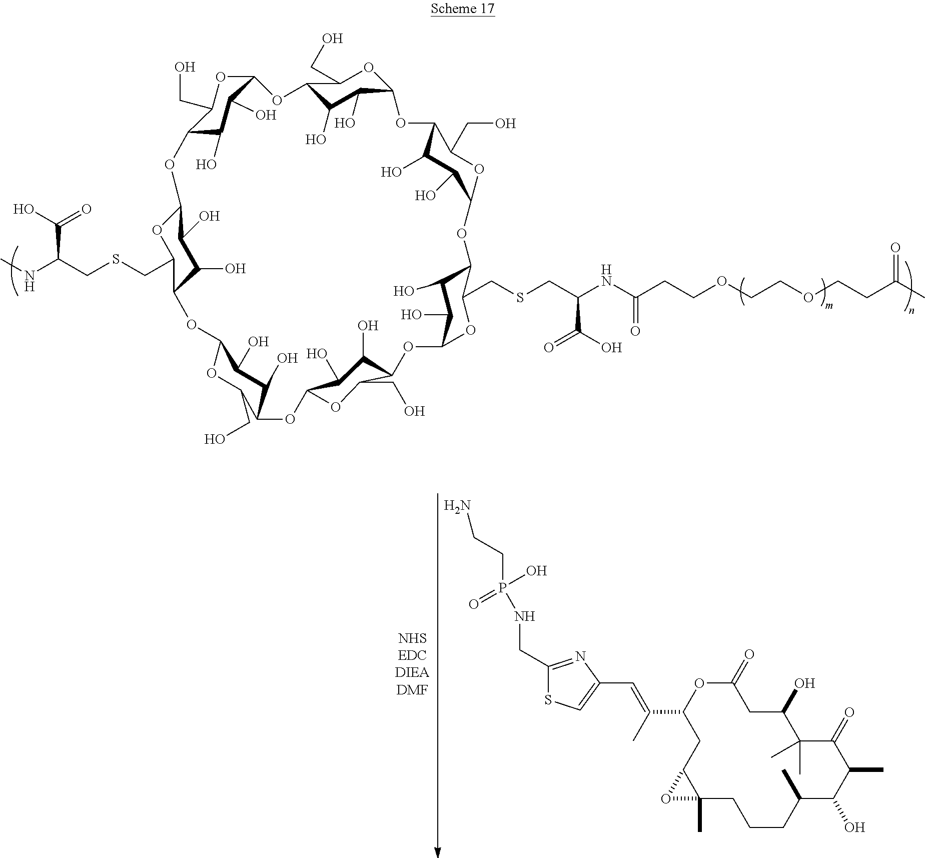

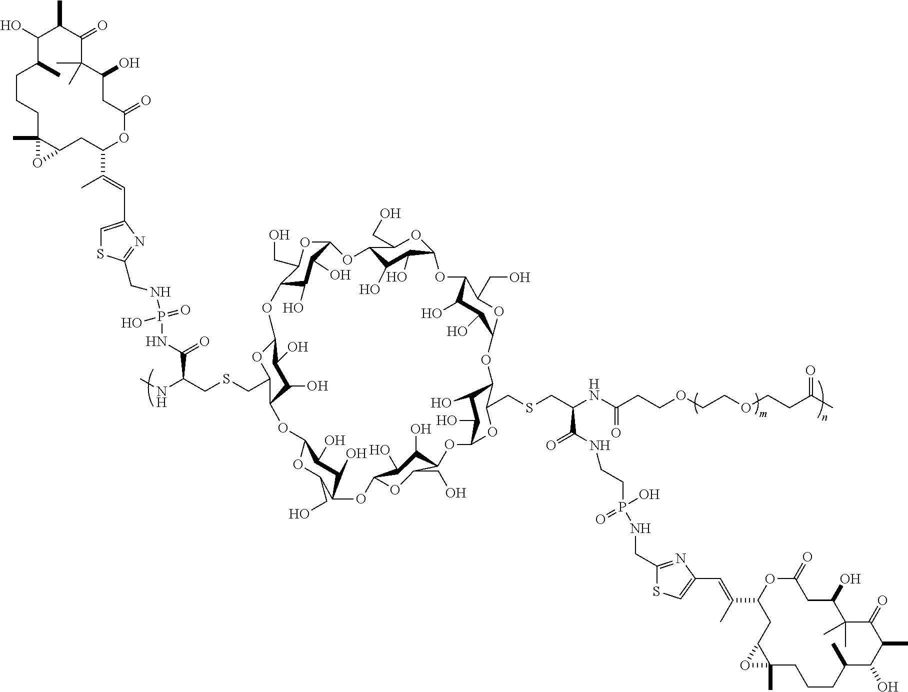

### Example 4

### Synthesis of CDP-C(O)—O-Kos-1584

Method A: Directly Attach Linker to KOS-1584, Separate Mixture, Deprotect and then Couple to CDP

### Step 1: Synthesis of KOS-1584-ε-TROC-aminohexanoate (Scheme 18)

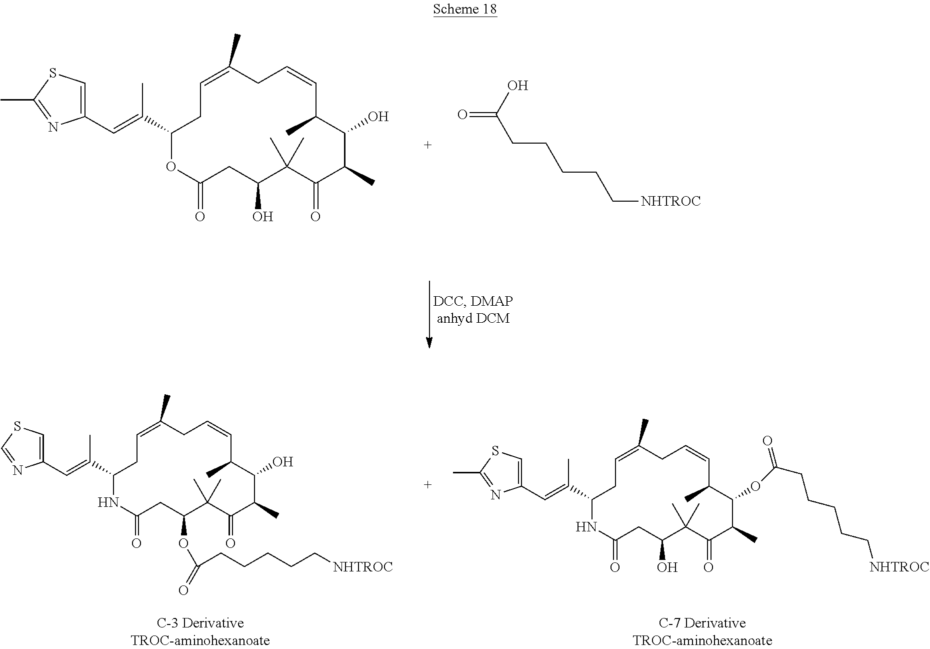

KOS-1584 (20 mg, 0.041 mmol) and ε-TROC-aminohexanoic acid (16.3 mg, 0.0585 mmol) will be dissolved in anhydrous DCM (10 mL) under N₂. To the resulting clear solution, DCC (13.4 mg, 0.065 mmol) and DMAP (7.9 mg, 0.065 mmol) will be added (Scheme 18). The reaction mixture will then be stirred for 12 h at room temperature. The solvent will subsequently be evaporated and the resulting residue dissolved in a minimum amount of chloroform. The desired C-3 and C-7 derivatives can be isolated via purification using flash column chromatography with chloroform/methanol as the mobile phase. The derivatives are to be analyzed by electron spray mass spectroscopy (m/z), HPLC and ¹H-NMR. The C-7 derivative of KOS-1584-ε-TROC-aminohexanoate is used as an example in the following synthetic steps.

### Step 2: Synthesis of KOS-1584-t-aminohexanoate (Scheme 19)

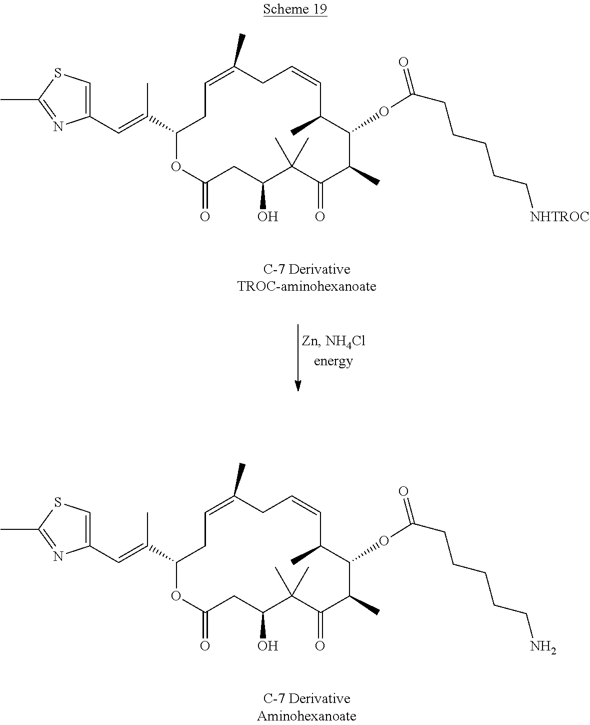

The C-7 derivative of KOS-1584-ε-TROC-aminohexanoate (15 mg, 0.019 mmol) and ammonium chloride (103 mg, 1.93 mmol) will be combined and mixed in 3 ml of water. While stirring vigorously, Zn powder (101 mg, 1.54 mmol) will be added with the input of energy (e.g., heat, sonication, microwave or ultraviolet irradiation) (Martin et al. (2000) Angewandte Chemie International Edition, 39 (3), 581-583) and stirred for an additional 20 min. The resulting solution will be filtered to remove zinc oxide and washed with hot water. The product will be extracted in dichloromethane and dried over MgSO₄. Evaporation of the organic solvent will be followed by purification of the resulting product via flash chromatography. The purified product will then be analyzed by electron spray mass spectroscopy (m/z), HPLC and ¹H-NMR.

### Step 3: Synthesis of CDP-C(O)—O-KOS-1584 (Scheme 20)

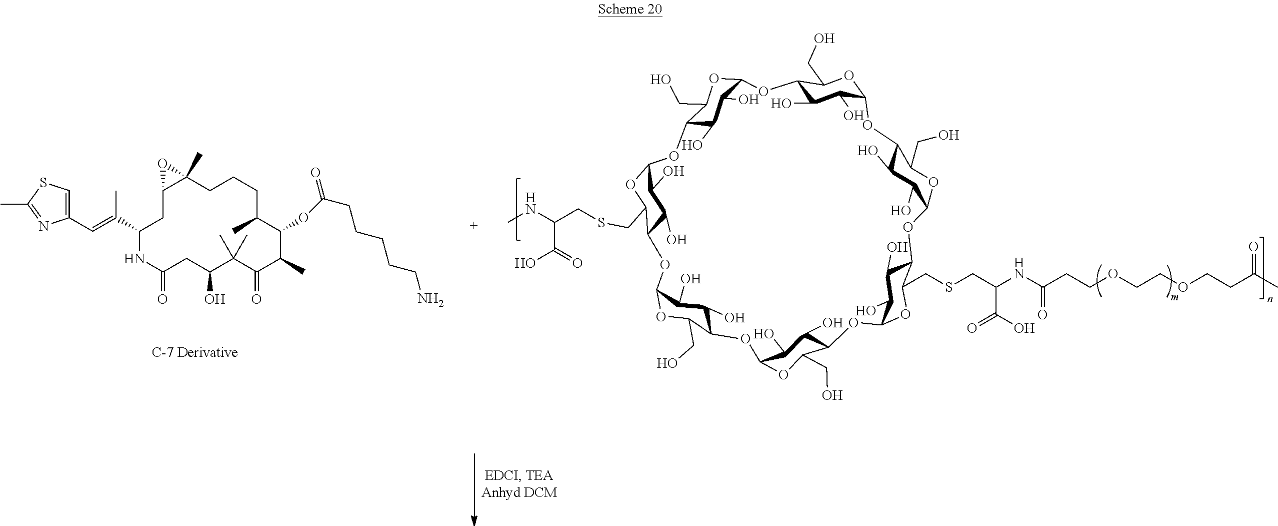

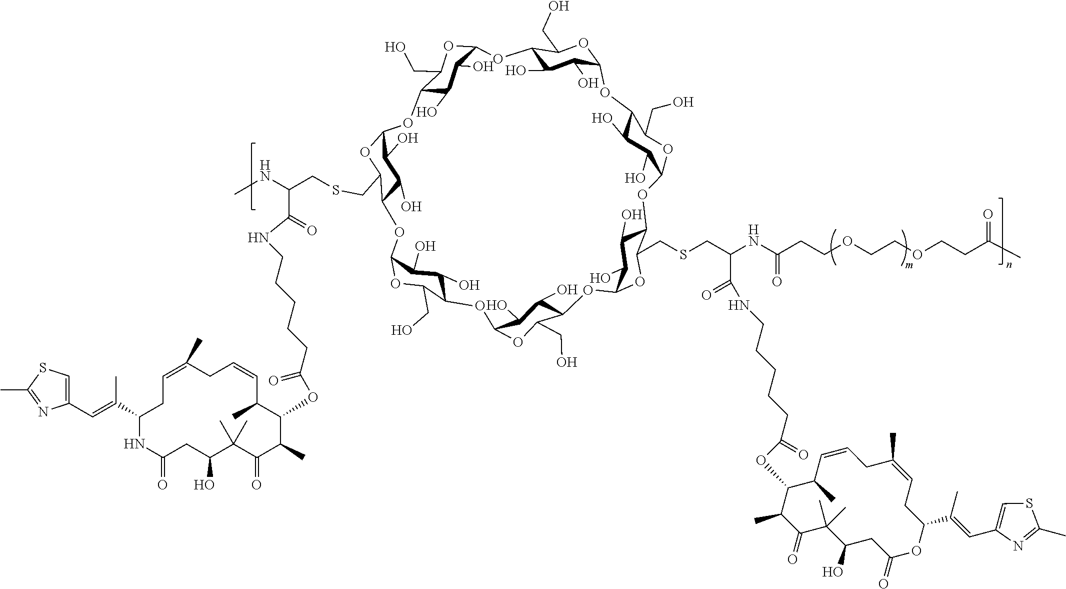

CDP-COOH (50 mg, 0.011 mmol) will be dissolved in MeOH (2.0 mL). The C-7 derivative of KOS-1584-8-aminohexanoate (14.3 mg, 0.024 mmol) will subsequently be added to the mixture and stirred for a few minutes to obtain a clear solution. EDCI (6.1 mg, 0.032 mmol) and TEA (3.8 mg, 0.038 mmol) will be added and the reaction stirred at ambient temperature for 3 h (Scheme 20). The resulting reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will then be washed with acetone (1 mL) twice, dissolved in nanopure water (3 mL) and then filtered through a 0.2 μm filter membrane and lyophilized to afford CDP-C(O)—O-KOS-1584. Loading will be determined by UV/Vis spectrometry with a standard curve and the particle size will be determined by zetasizer.

Method B: Selectively Protect with Silyl Protecting Group, Addition of Linker, Followed by Deprotection and then Conjugation with CDP

### Step 1: Synthesis of 3-tert-butyldimethylsilyl KOS-1584 or 7-tert-butyldimethylsilyl KOS-1584 (Scheme 21)

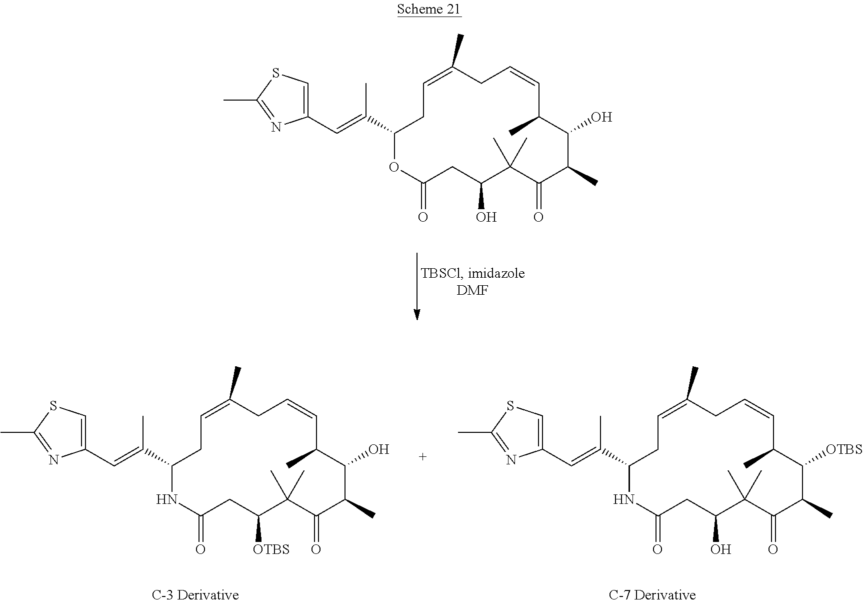

KOS-1584 (20 mg, 0.041 mmol) and tert-butyldimethylsilyl chloride (8.3 mg, 0.055 mmol) will be mixed in anhydrous DMF (5 mL) under N₂ atm (Trichloroethoxy chloroformate, TROC or any other bulky protecting group can be used instead to provide selective protection of OH group). To the resulting clear solution, imidazole (10.7 mg, 0.158 mmol) will be added (Scheme 21) and the reaction will be allowed to stir at ambient temperature for 24 h. The solvent will be evaporated and the residue dissolved in a minimum amount of chloroform. The desired C-3 and C-7 derivatives will be isolated following purification of the crude product via flash column chromatography with chloroform/methanol as the mobile phase. The derivatives will be analyzed by electron spray mass spectroscopy (m/z), HPLC and ¹H-NMR. C-7 derivative of TBS-KOS-1584 is used as an example in the following synthetic steps.

### Step 2: Synthesis of 7-(tert-butyldimethylsilyl)-3-(TROC-aminohexanoate)-KOS-1584 (Scheme 22)

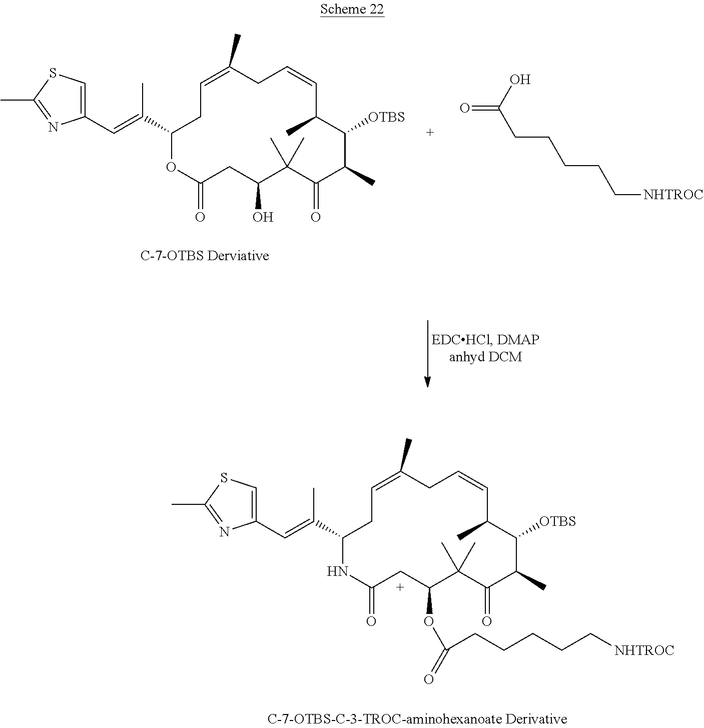

7-tert-butyldimethylsilyl KOS-1584 (20 mg, 0.032 mmol) and 8-TROC-aminohexanoic acid (12.0 mg, 0.039 mmol) will be stirred together in anhydrous DCM (2 mL) under N₂. To the resulting clear solution, EDC.HCl (11.1 mg, 0.058 mmol) and DMAP (7.08 mg, 0.058 mmol) will be added (Scheme 22). The reaction mixture will then be stirred for 12 h at 22° C. The solvent is subsequently evaporated and the resulting residue dissolved in a minimum amount of chloroform. The crude product will be purified via flash column chromatography with chloroform/methanol as the mobile phase. The product will be analyzed by electron spray mass spectroscopy (m/z), HPLC and ¹H-NMR.

### Step 3: Synthesis of 3-(aminohexanoate)-KOS-1584 (Scheme 23)

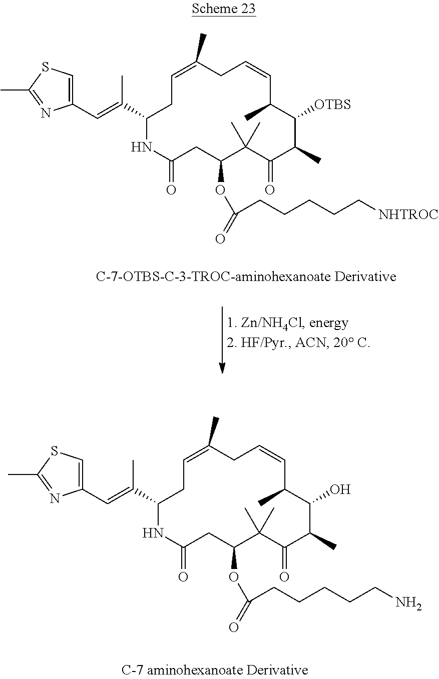

7-(tert-butyldimethylsilyl)-3-(TROC-aminohexanoate)-KOS-1584 will be deprotected using Zn/NH₄Cl with the input of energy (e.g., heat, sonication, microwave or ultraviolet irradiation), followed by a solution of acetonitrile and HF/Pyridine. The final product will be purified via flash column chromatography with chloroform/methanol as the mobile phase. 3-(aminohexanoate)-KOS-1584 will be analyzed by electron spray mass spectroscopy (m/z), HPLC and ¹H-NMR.

### Step 4: Synthesis of CDP-C(O)—O-KOS-1584 (Scheme 24)

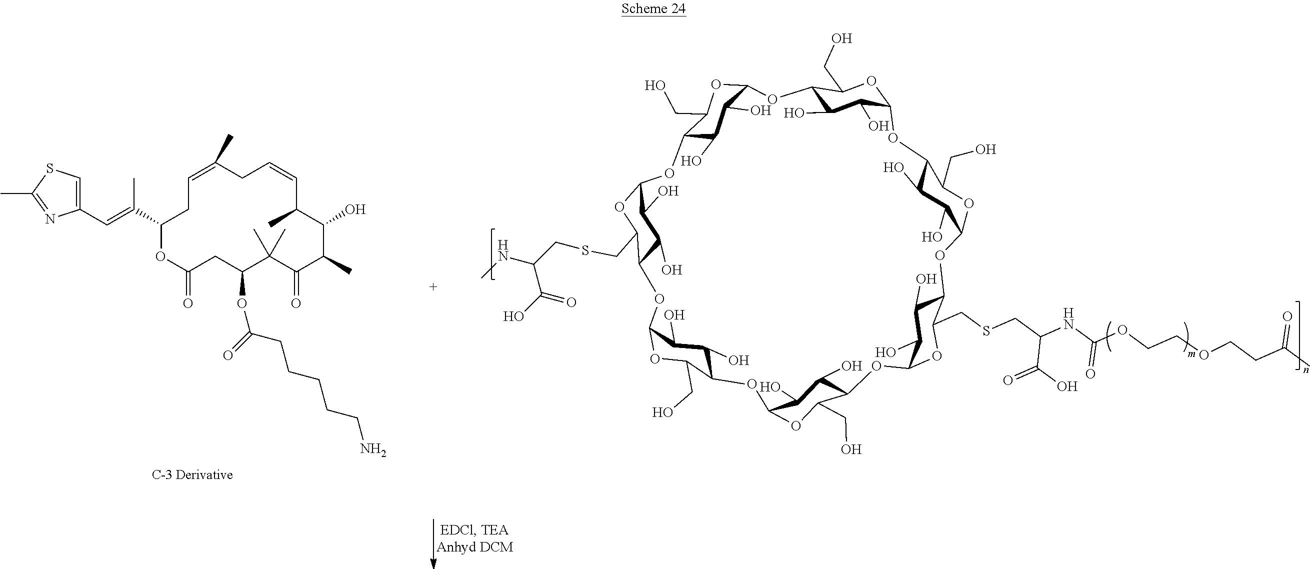

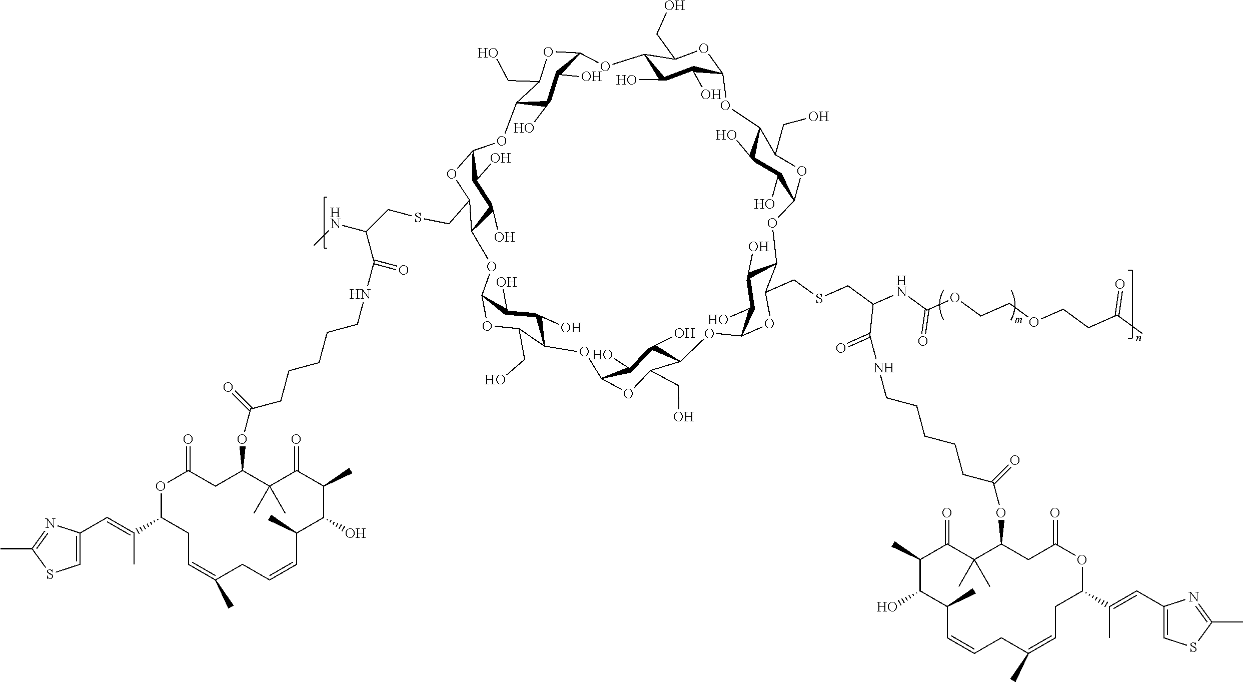

CDP-COOH (50 mg, 0.011 mmol) will be dissolved in MeOH (2.0 mL). A C-3 derivative of KOS-1584-ε-aminohexanoate (14.3 mg, 0.024 mmol) will subsequently be added to the mixture and stirred for a few minutes to obtain a clear solution. EDCI (6.1 mg, 0.032 mmol) and TEA (3.8 mg, 0.038 mmol) will then be added and the reaction stirred at ambient temperature for 3 h (Scheme 24). The reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will be washed with acetone (1 mL) twice, dissolved in nanopure water (3 mL) and then filtered through a 0.2 μm filter membrane and lyophilized to afford CDP-C(O)—O-KOS-1584. Loading will be determined by UV/Vis spectrometry with a standard curve and the particle size will be determined by zetasizer.

### Example 5

### Synthesis of CDP-Amide-Epothilone B

Method of Synthesizing CDP-Amide-Epothilone B

CDP (1.0 g, 0.21 mmol) will be dissolved in dry N,N-dimethylformamide (DMF, 20 mL). Epothilone B analog (250 mg, 0.46 mmol), N,N-Diisopropylethylamine (0.080 mL, 0.46 mmol), N-(3-Dimethylaminopropyl)-N′-ethylcarbodiimide hydrochloride (120 mg, 0.62 mmol), and N-Hydroxysuccinimide (52 mg, 0.46 mmol) will then be added to the polymer solution and stirred for 4 h. The polymer will be precipitated with ethylacetate (100 mL) and then rinsed with acetone (50 mL). The precipitate will be dissolved in pH3 water (100 mL) which is prepared by acidification with HCl. The solution will be dialyzed using 25,000 MWCO membrane (Spectra/Por 7) for 24 h at pH3 water and filtered through 0.2 μm filters (Nalgene) and lyophilized to yield a white solid (Scheme 25).

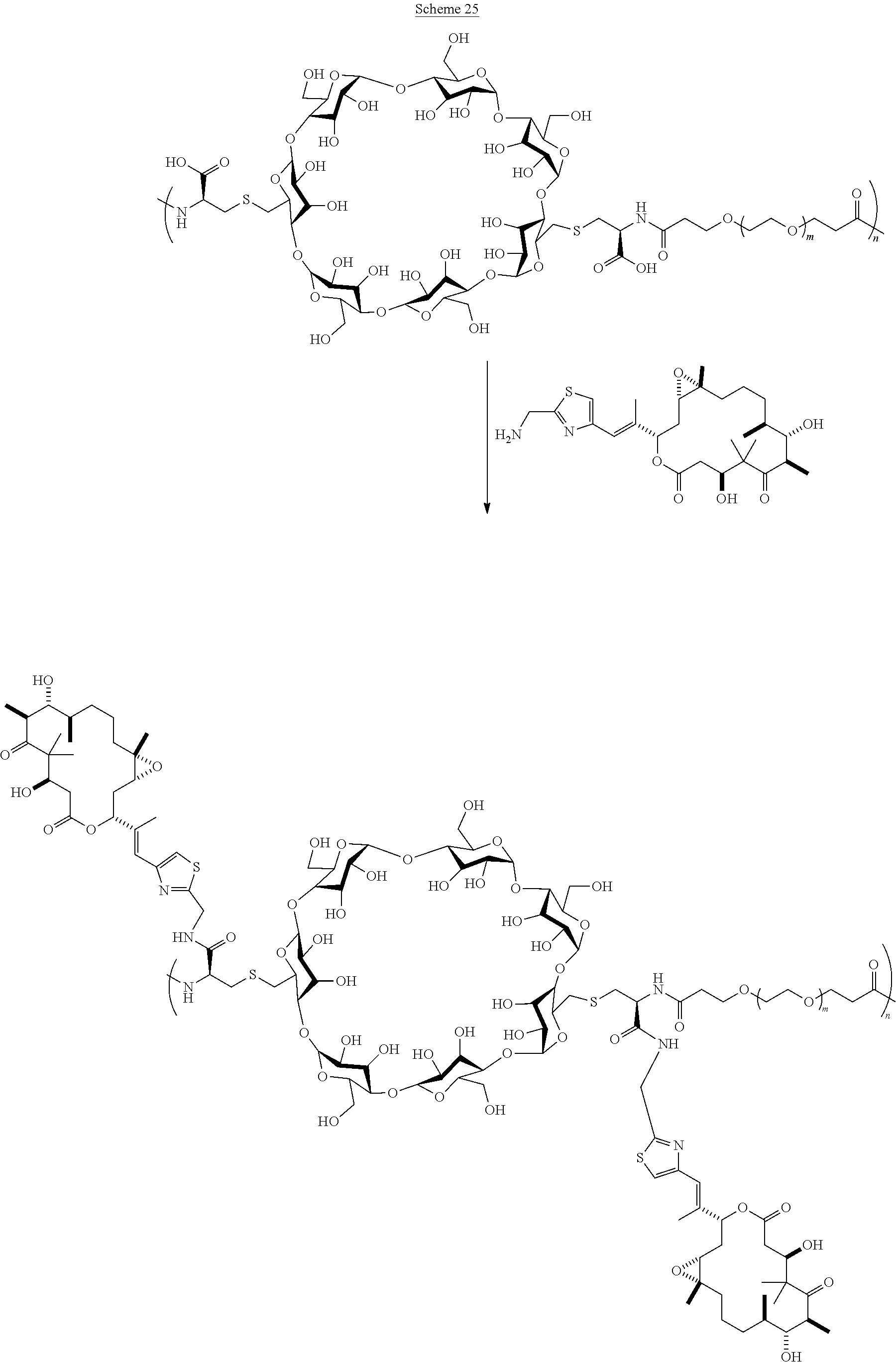

### Example 6

### Synthesis of CDP-C(O)—O-Sagopilone

Method A: Directly Attach Linker to Sagopilone, Separate Mixture, Deprotect and then Couple to CDP

### Step 1: Synthesis of Sagopilone-ε-TROC-aminohexanoate (Scheme 26)

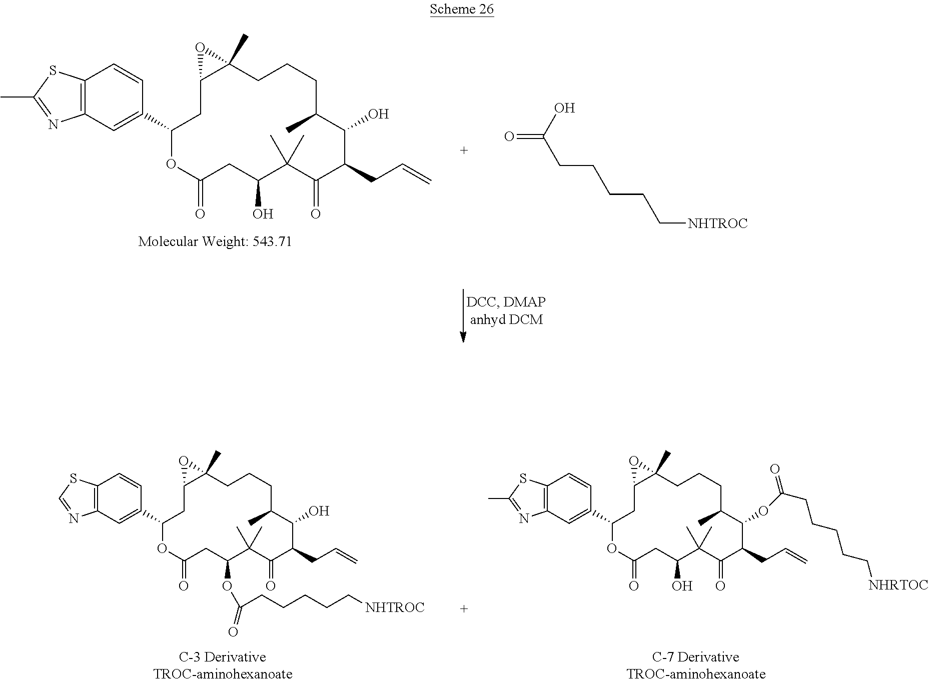

Sagopilone (20 mg, 0.037 mmol) and ε-TROC-aminohexanoic acid (16.3 mg, 0.0585 mmol) will be dissolved in anhydrous DCM (10 mL) under N₂. To the resulting clear solution, DCC (13.4 mg, 0.065 mmol) and DMAP (7.9 mg, 0.065 mmol) will be added (Scheme 26). The reaction mixture will then be stirred for 12 h at room temperature. The solvent will subsequently be evaporated and the resulting residue dissolved in a minimum amount of chloroform. The desired C-3 and C-7 derivatives can be isolated via purification using flash column chromatography with chloroform/methanol as the mobile phase. The derivatives are to be analyzed by electron spray mass spectroscopy (m/z), HPLC and ¹H-NMR. The C-7 derivative of Sagopilone-ε-TROC-aminohexanoate is used as an example in the following synthetic steps.

### Step 2: Synthesis of Sagopilone-ε-aminohexanoate (Scheme 27)

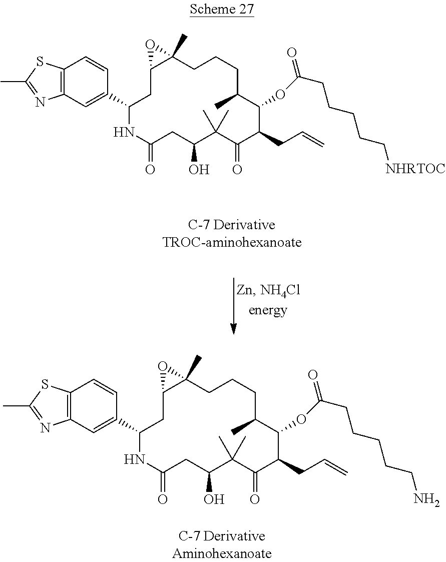

The C-7 derivative of Sagopilone-ε-TROC-aminohexanoate (15 mg, 0.018 mmol) and ammonium chloride (100 mg, 1.88 mmol) will be combined and mixed in 3 ml of water. While stirring vigorously, Zn powder (98 mg, 1.51 mmol) will be added with the input of energy (e.g., heat, sonication, microwave or ultraviolet irradiation (Martin et al. (2000) Angewandte Chemie International Edition, 39 (3), 581-583) and stirred for an additional 20 min. The resulting solution will be filtered to remove zinc oxide and washed with hot water. The product will be extracted in dichloromethane and dried over MgSO₄. Evaporation of the organic solvent will be followed by purification of the resulting product via flash chromatography. The purified product will then be analyzed by electron spray mass spectroscopy (m/z), HPLC and ¹H-NMR.

### Step 3: Synthesis of CDP-C(O)—O-Sagopilone (Scheme 28)

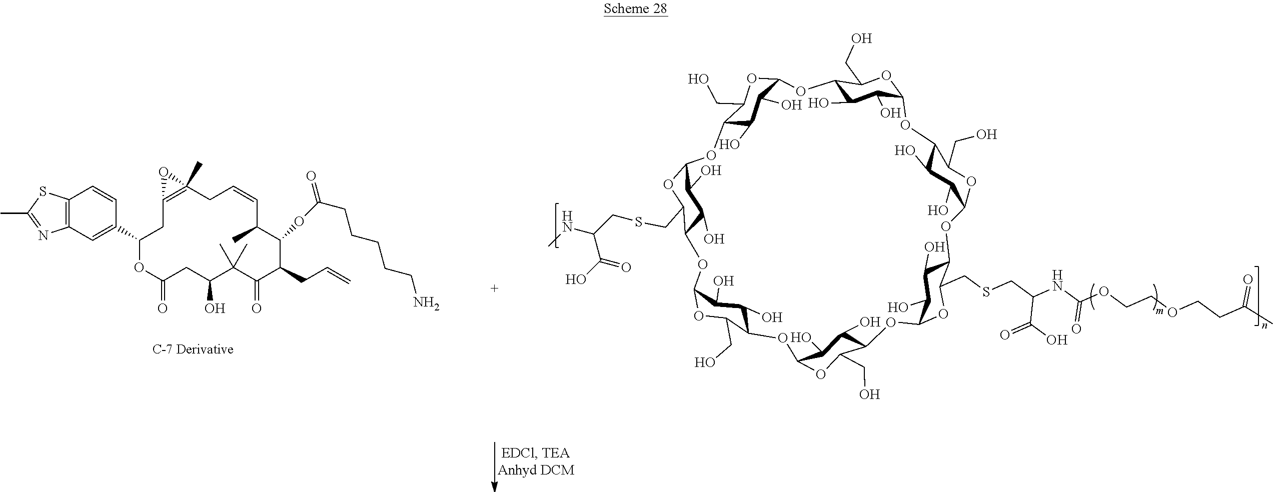

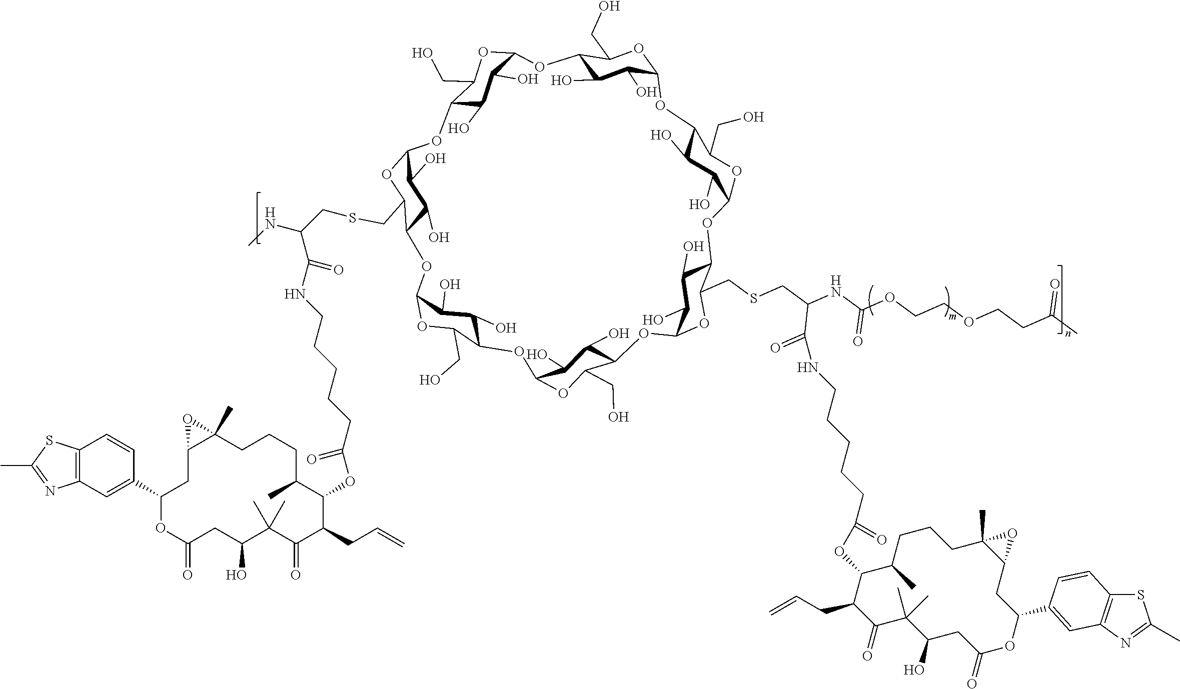

CDP-COOH (50 mg, 0.011 mmol) will be dissolved in MeOH (2.0 mL). The C-7 derivative of Sagopilone-ε-aminohexanoate (15.6 mg, 0.024 mmol) will subsequently be added to the mixture and stirred for a few minutes to obtain a clear solution. EDCI (6.1 mg, 0.032 mmol) and TEA (3.8 mg, 0.038 mmol) will be added and the reaction stirred at ambient temperature for 3 h (Scheme 28). The resulting reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will then be washed with acetone (1 mL) twice, dissolved in nanopure water (3 mL) and then filtered through a 0.2 μm filter membrane and lyophilized to afford CDP-C(O)—O-Sagopilone. Loading will be determined by UV/Vis spectrometry with a standard curve. The particle size is determined by zetasizer.

Method B: Selectively Protect with Silyl Protecting Group, Addition of Linker, Followed by Deprotection and then Conjugation with CDP

### Step 1: Synthesis of 3-tert-butyldimethylsilyl Sagopilone or 7-tert-butyldimethylsilyl Sagopilone (Scheme 29)

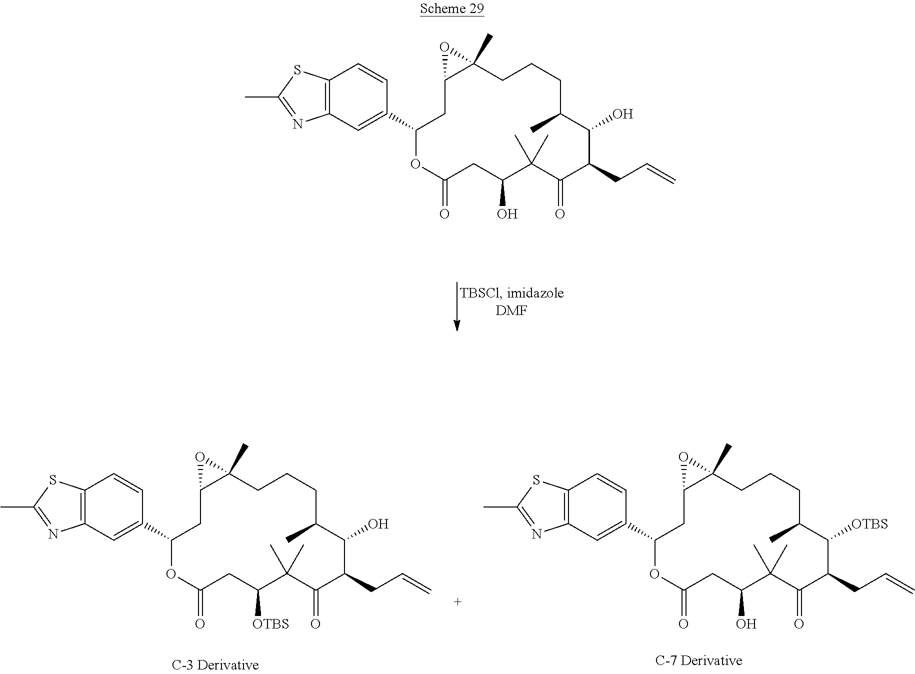 [SMILES: C=CC[C@H]1C(=O)C(C)(C)[C@@H](O)CC(=O)O[C@H](c2ccc3sc(C)nc3c2)C[C@@H]2O[C@]2(C)CCC[C@H](C)[C@@H]1O ; C=CC[C@H]1C(=O)C(C)(C)[C@@H](O[Si](C)(C)C(C)(C)C)CC(=O)O[C@H](c2ccc3sc(C)nc3c2)C[C@@H]2O[C@]2(C)CCC[C@H](C)[C@@H]1O ; C=CC[C@H]1C(=O)C(C)(C)[C@@H](O)CC(=O)O[C@H](c2ccc3sc(C)nc3c2)C[C@@H]2O[C@]2(C)CCC[C@H](C)[C@@H]1O[Si](C)(C)C(C)(C)C]

Sagopilone (20 mg, 0.037 mmol) and tert-butyldimethylsilyl chloride (8.3 mg, 0.055 mmol) will be mixed in anhydrous DMF (5 mL) under N₂ atm (Trichloroethoxy chloroformate, TROC, or any other bulky protecting group can be used instead to provide selective protection of OH group). To the resulting clear solution, imidazole (10.7 mg, 0.158 mmol) will be added (Scheme 4) and the reaction will be allowed to stir at ambient temperature for 24 h. The solvent will be evaporated and the residue dissolved in a minimum amount of chloroform. The desired C-3 and C-7 derivatives will be isolated following purification of the crude product via flash column chromatography with chloroform/methanol as the mobile phase. The derivatives will be analyzed by electron spray mass spectroscopy (m/z), HPLC and ¹H-NMR. The C-7 derivative of TBS-Sagopilone is used as an example in the following synthetic steps.

### Step 2: Synthesis of 7-(tert-butyldimethylsilyl)-3-(TROC-aminohexanoante)-Sagopilone (Scheme 30)

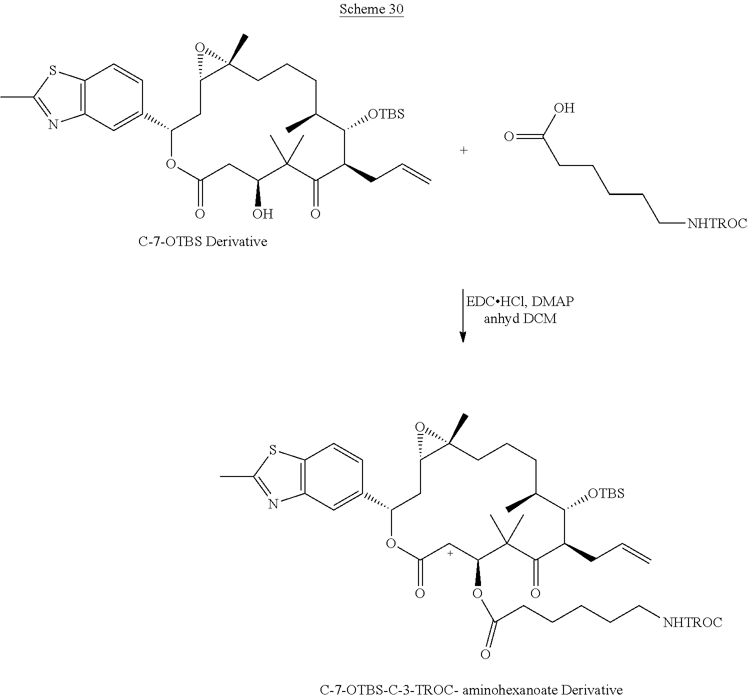

7-tert-butyldimethylsilyl Sagopilone (20 mg, 0.030 mmol) and ε-TROC-aminohexanoic acid (12.0 mg, 0.039 mmol) will be stirred together in anhydrous DCM (2 mL) under N₂. To the resulting clear solution, EDC.HCl (11.1 mg, 0.058 mmol) and DMAP (7.08 mg, 0.058 mmol) will be added (Scheme 30). The reaction mixture is then stirred for 12 h at 22° C. The solvent is subsequently evaporated and the resulting residue dissolved in a minimum amount of chloroform. The crude product will be purified via flash column chromatography with chloroform/methanol as the mobile phase. The product will be analyzed by electron spray mass spectroscopy (m/z), HPLC and ¹H-NMR.

### Step 3: Synthesis of 3-(aminohexanoate)-Sagopilone (Scheme 31)

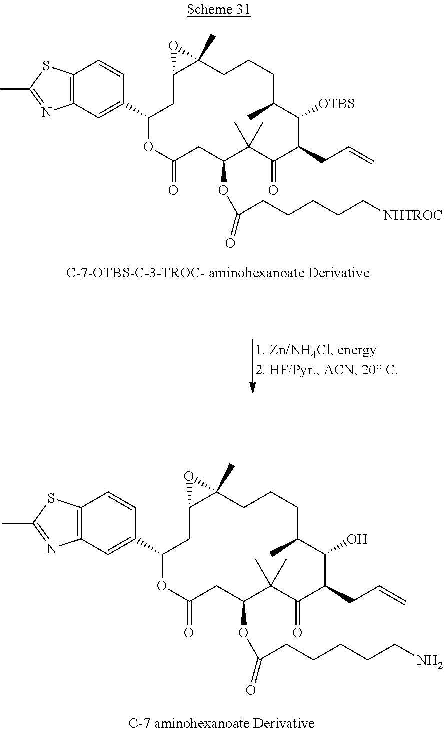

7-(tert-butyldimethylsilyl)-3-(TROC-aminohexan)-Sagopilone-oate will be deprotected using Zn/NH₄Cl with the input of energy (e.g., heat, sonication, microwave or ultraviolet irradiation), followed by a solution of acetonitrile and HF/Pyridine. The final product will be purified via flash column chromatography with chloroform/methanol as the mobile phase. 3-(aminohexan)-Sagopilone-oate will be analyzed by electron spray mass spectroscopy (m/z), HPLC and ¹H-NMR.

### Step 4: Synthesis of Poly-CD-Hex-C(O)—O-Sagopilone (CDP-C(O)—O-Sagopilone) (Scheme 32)

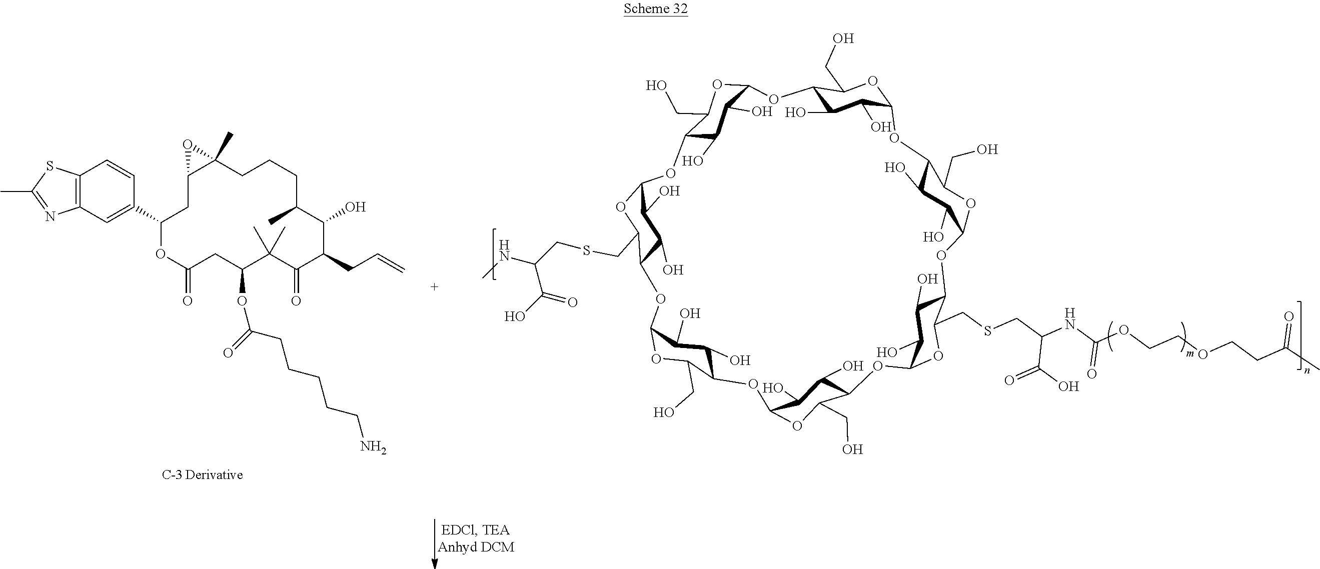

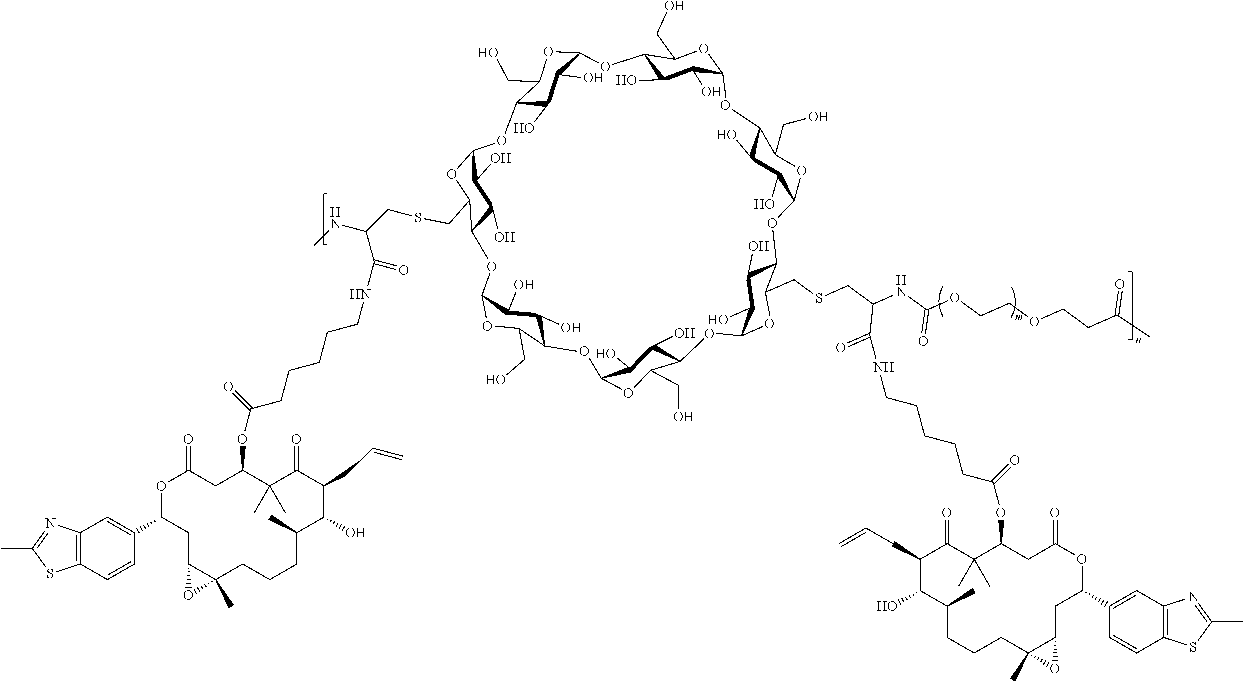

CDP-COOH (50 mg, 0.011 mmol) will be dissolved in MeOH (2.0 mL). A C-3 derivative of Sagopilone-ε-aminohexanoate (15.5 mg, 0.024 mmol) will subsequently be added to the mixture and stirred for a few minutes to obtain a clear solution. EDCI (6.1 mg, 0.032 mmol) and TEA (3.8 mg, 0.038 mmol) are then added and the reaction stirred at ambient temperature for 3 h (Scheme 32). The reaction mixture will be reduced to 0.1 mL of solution and precipitated in Et₂O (1.5 mL). The polymer conjugate will be redissolved in DMF (0.1 mL) and added to acetone (1.5 mL) to precipitate out the polymer conjugate. The polymer conjugate will be washed with acetone (1 mL) twice, dissolved in nanopure water (3 mL) and then filtered through a 0.2 μm filter membrane and lyophilized to afford CDP-C(O)—O-Sagopilone. Loading will be determined by UV/Vis spectrometry with a standard curve. The particle size will be determined by zetasizer.

### Example 7

### Synthesis of CDP-SS-Ixabepilone (Carbonate)

Synthesis of CDP-SS-Py

A mixture of CDP, (67 kD, 2.0 g, 0.41 mmole), pyridine dithioethylamine hydrochloric salt (180 mg, 0.83 mmole), N-(3-dimethylaminopropyl)-N′-ethylcarbodiimide hydrochloride (EDCI, 240 mg, 1.2 mmole), and N-hydroxysuccinimide (NHS, 95 mg, 0.83 mmole) will be dissolved in anhydrous N,N-dimethylformamide (DMF, 20 mL) and followed by addition of anhydrous N,N-diisopropylethylamine (DIEA, 0.14 mL, 0.83 mmole). The reaction mixture will be stirred under argon at room temperature for 4 h. The mixture will then be added to ethyl acetate (EtOAc, 100 mL) to precipitate the polymer. In order to clean up the polymer further without dialysis, multiple crashouts will be carried out to purify the polymer. The Polymer will be dissolved back in methanol (MeOH, 20 mL) and precipitated in diethyl ether (Et₂O, 100 mL). Purification by reprecipitation will be carried out twice. The polymer will then be dried under vacuum to yield a white solid (Scheme 33).

Synthesis of CDP-SH

CDP-SS-Py (200 mg, 0.042 mmol) will be redissolved in MeOH (2 mL). Dithiothreitol (DTT, 130 mg, 0.83 mmol) will be added to the reaction mixture and stirred for 1 h (Scheme 33). It will then be precipitated in Et₂O (20 mL). The polymer will be purified by multiple reprecipitation. It will be dissolved in MeOH (2 mL) and precipitated in Et₂O (20 mL). This process will be repeated twice. The polymer will be dried under vacuum to yield a white solid.

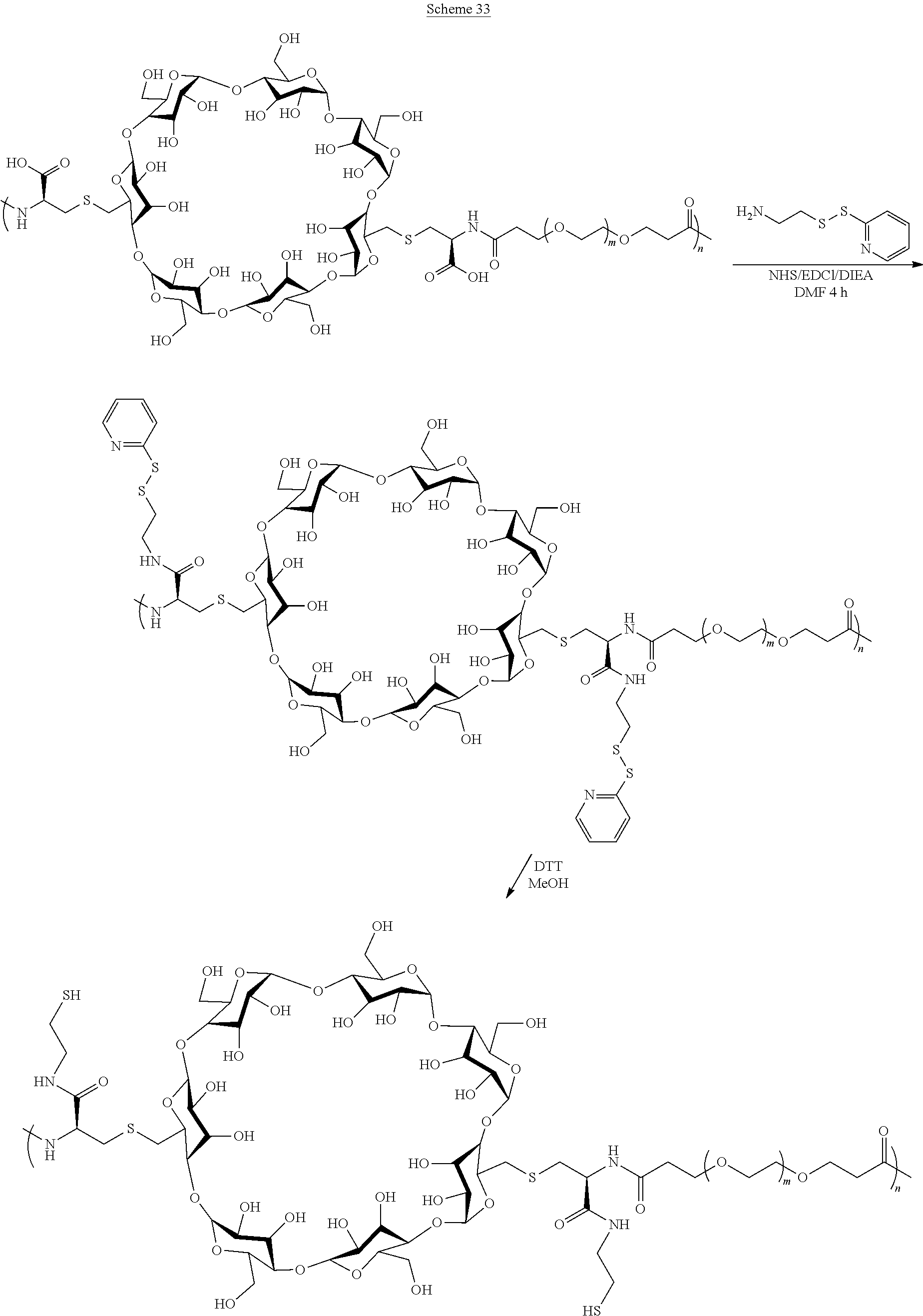

### Synthesis of pyridin-2-yldisulfanyl ethyl ester derivative of Ixabepilone

Ixabepilone (5 mg, 0.0099 mmol) will be in dichloromethane (CH₂Cl₂, 1.5 mL). Triethylamine (TEA, 5.6 μL, 0.040 mmol) and 20% phosgene in toluene (9.8 μL, 0.020 mmol) will be added to the mixture and stirred for ½ h. The mixture will be purged with Ar to remove any excess phosgene. Pyridine dithioethanol (3.7 mg, 0.020 mmole), 4-dimethylaminopyridine

(DMAP, 1.2 mg, 0.0099 mmol) and TEA (2.8 μL, 0.020 mmol) will be added and stirred for an additional one hour (Scheme 34). It will then be pumped down to dryness and purified by flash column chromatography with dichloromethane and methanol (9:1) ratio to yield a white solid.

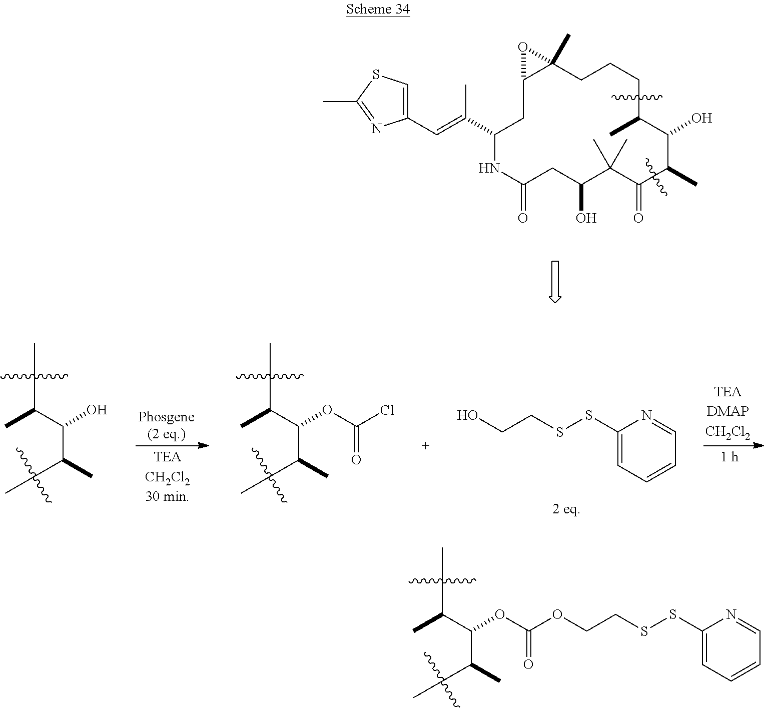

Synthesis of CDP-SS-Ixabepilone.

CDP-SH (32 mg, 0.0070 mmole) will be dissolved in MeOH (1.0 mL). Pyridin-2-yldisulfanyl ethyl ester derivative of Ixabepilone (5 mg, 0.070 mmol) will be added to the mixture and stirred for 1 h. N-ethyl maleimide (NEM, 8.7 mg, 0.070 mmole) will then be added to quench the reaction and stirred for an additional hour (Scheme 35). The reaction mixture will be reduced to 0.1 mL of solution and subsequently precipitated in Et₂O (1 mL). The polymer conjugate will be redissolved in DMF (0.1 mL) and added to acetone (1 mL) to precipitate out the polymer conjugate. The polymer conjugate will be washed with acetone (1 mL) twice. It will be dissolved in nanopure water (3 mL) and then filtered through 0.2 μm filter membrane and lyophilized to afford CDP-Ixabepilone. In instances where a mixture of isomers is formed (e.g., acylation at the 3- and/or 7-position), the isomeric products can be separated (e.g., using flash chromatography).

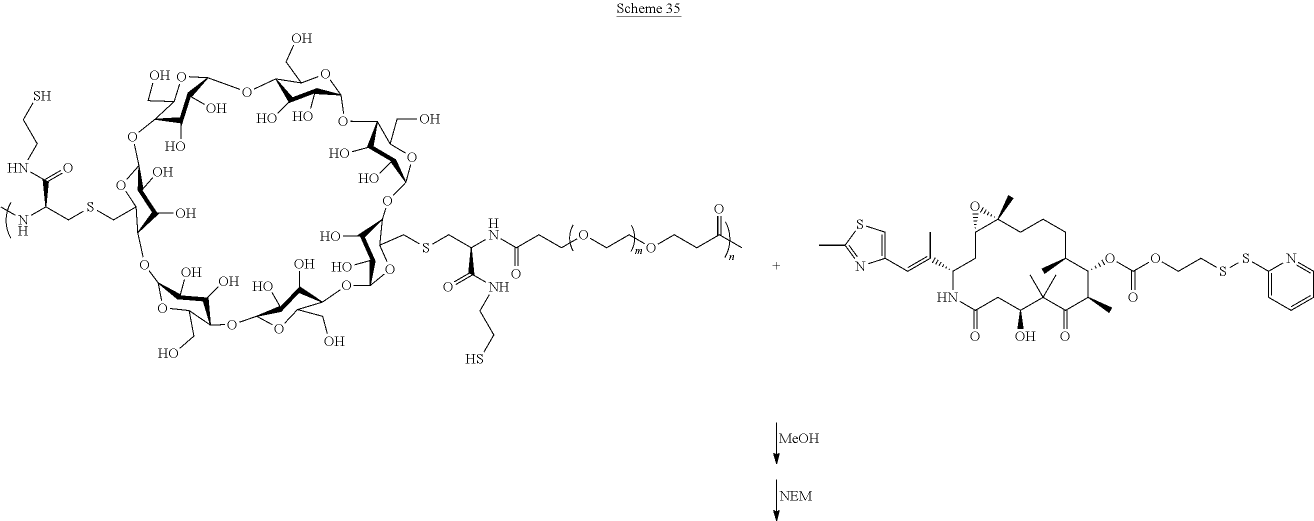

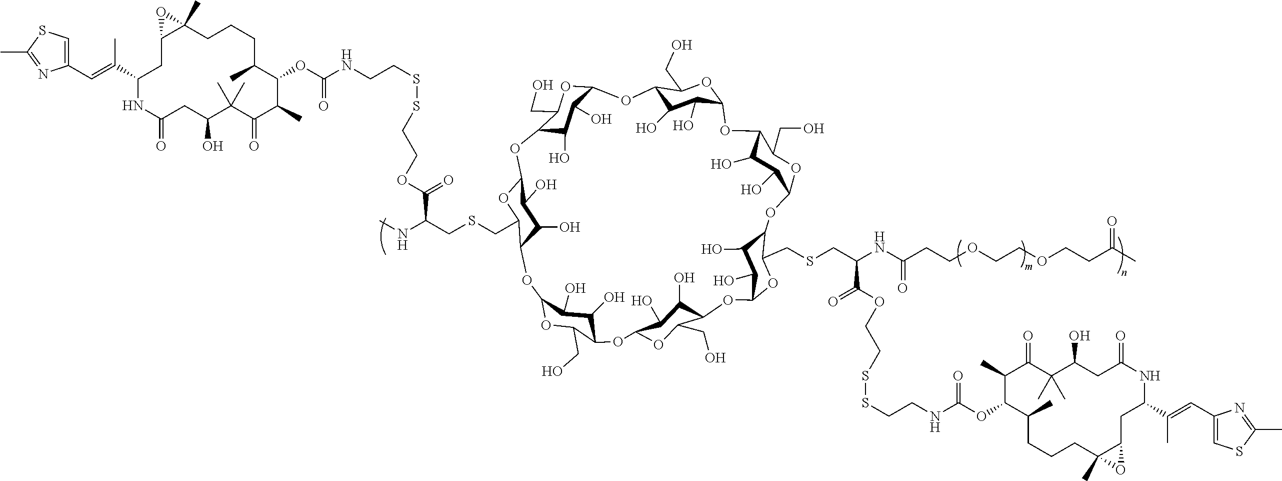

### Example 8

### Synthesis of CDP-SS-Ixabepilone (Carbamate)

Synthesis of CDP-SS-Py

A mixture of CDP, (67 kD, 2.0 g, 0.41 mmole), pyridine dithioethylamine hydrochloric salt (180 mg, 0.83 mmole), N-(3-dimethylaminopropyl)-N′-ethylcarbodiimide hydrochloride (EDCI, 240 mg, 1.2 mmole), and N-hydroxysuccinimide (NHS, 95 mg, 0.83 mmole) were dissolved in anhydrous N,N-dimethylformamide (DMF, 20 mL) and followed by addition of anhydrous N,N-diisopropylethylamine (DIEA, 0.14 mL, 0.83 mmole). The reaction mixture was stirred under argon at room temperature for 4 h. The mixture was then added to ethyl acetate (EtOAc, 100 mL) to precipitate the polymer. In order to clean up the polymer further without dialysis, multiple crashouts were carried out. The polymer was dissolved back in methanol (MeOH, 20 mL) and precipitated in diethyl ether (Et₂O, 100 mL). Purification by reprecipitation was carried out twice. The polymer was then dried under vacuum to yield a white solid (Scheme 36).

Synthesis of CDP-SH

CDP-SS-Py (200 mg, 0.042 mmol) was redissolved in MeOH (2 mL). Dithiothreitol (DTT, 130 mg, 0.83 mmol) was added to the reaction mixture and stirred for 1 h (Scheme 36). It was then precipitated in Et₂O (20 mL). The polymer was purified by multiple reprecipitation. It was dissolved in MeOH (2 mL) and precipitated in Et₂O (20 mL) twice. The polymer was dried under vacuum to yield a white solid.

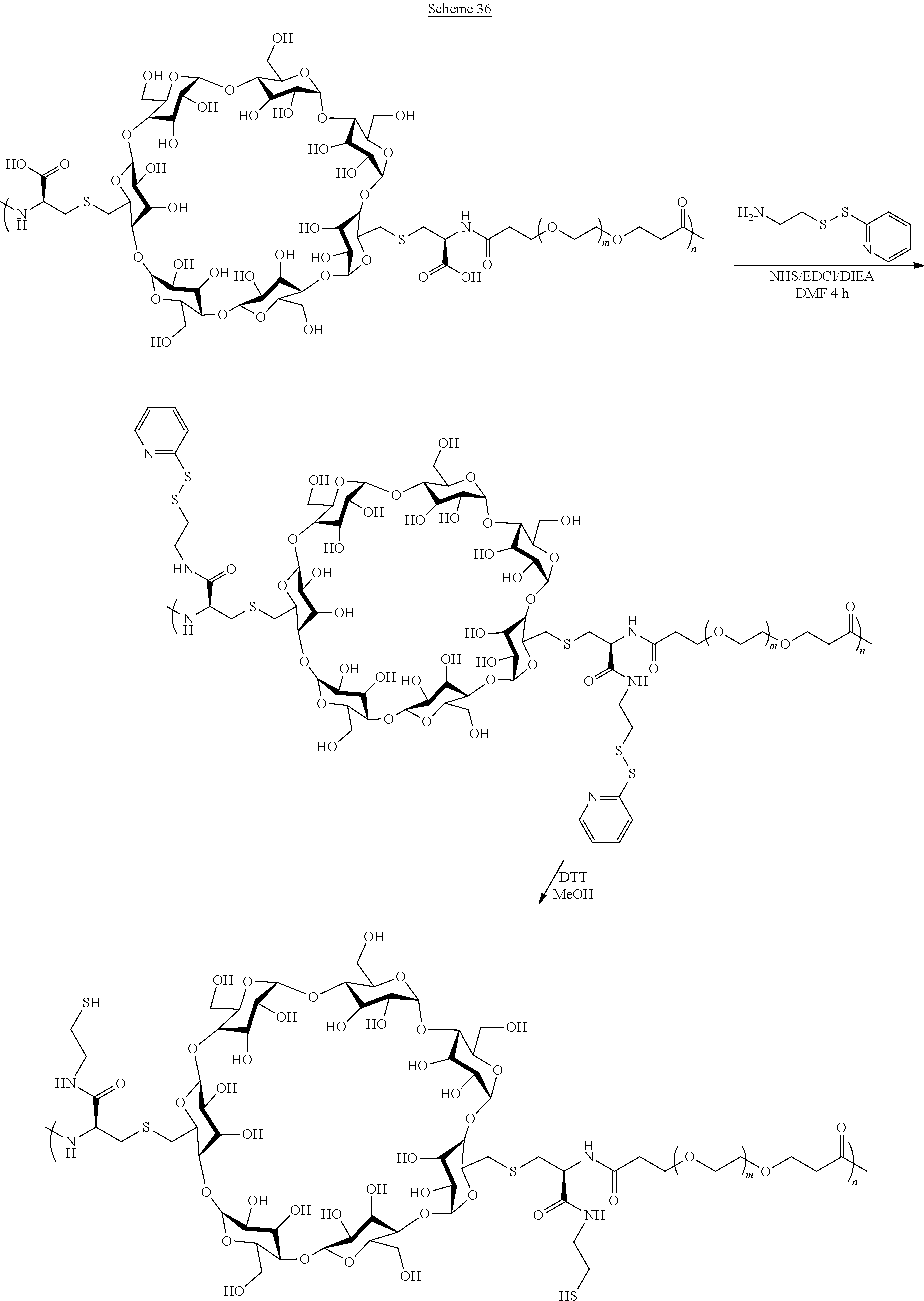

### Synthesis of pyridin-2-yldisulfanyl ethyl amide derivative of Ixabepilone

Ixabepilone (5 mg, 0.0099 mmol) was dissolved in dichloromethane (CH₂Cl₂, 1.5 mL). Triethylamine (TEA, 5.6 μL, 0.040 mmol) and 20% phosgene in toluene (9.8 μL, 0.020 mmol) were added to the mixture and stirred for ½ h. The mixture was purged with Ar to remove any excess phosgene. Pyridine dithioethylamine hydrochloric salt (3.7 mg, 0.020 mmole) and DIEA (2.8μ, 0.020 mmole) were added and stirred for an additional hour (Scheme 37). It was then pumped down to dryness and purified by flash column chromatography with dichloromethane and methanol (9:1) to yield a white solid (5.2 mg, 49% Yield). It was confirmed by electron spray mass spectrometry (m/z expected 718.99; Found 741.48 M+Na).

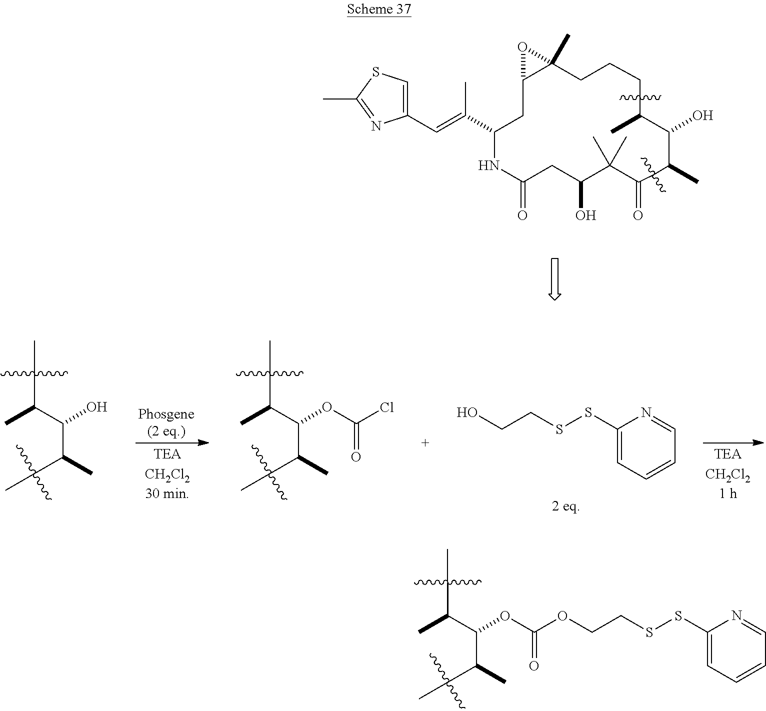

Synthesis of CDP-SS-Ixabepilone.

CDP-SH (32 mg, 0.0070 mmole) was dissolved in MeOH (1.0 mL). Pyridin-2-yldisulfanyl ethyl amide derivative of Ixabepilone (5 mg, 0.070 mmol) was added to the mixture and stirred for 1 h. N-ethyl maleimide (NEM, 8.7 mg, 0.070 mmole) was then added to quench the reaction and stirred for an additional hour (Scheme 38). The reaction mixture was reduced to 0.1 mL of solution and precipitated in Et₂O (1 mL). The polymer conjugate was redissolved in DMF (0.1 mL) and added to acetone (1 mL) to precipitate out the polymer conjugate. The polymer conjugate was washed with acetone (1 mL) twice. It was dissolved in nanopure water (3 mL) and then filtered through a 0.2 μm filter membrane and lyophilized to afford CDP-Ixabepilone (19 mg, 58% Yield). Loading was determined to be 11.2% w/w by UV/Vis spectrometry with standard curve. The particle size is determined to be 49.0 nm. In instances where a mixture of isomers is formed (e.g., acylation at the 3- and/or 7-position), the isomeric products are separated (e.g., using flash chromatography).

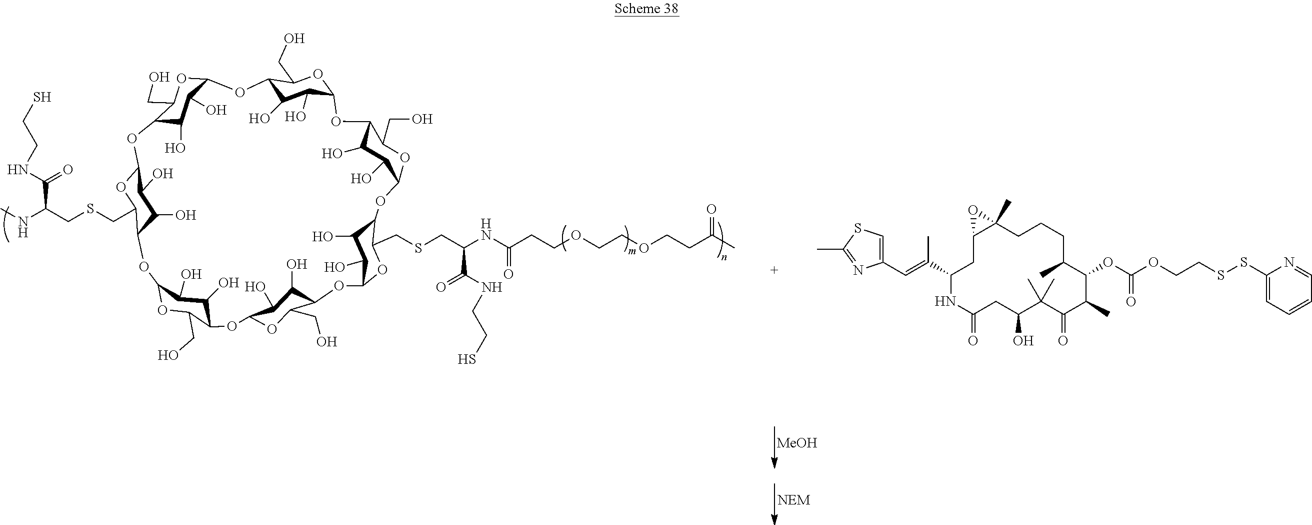

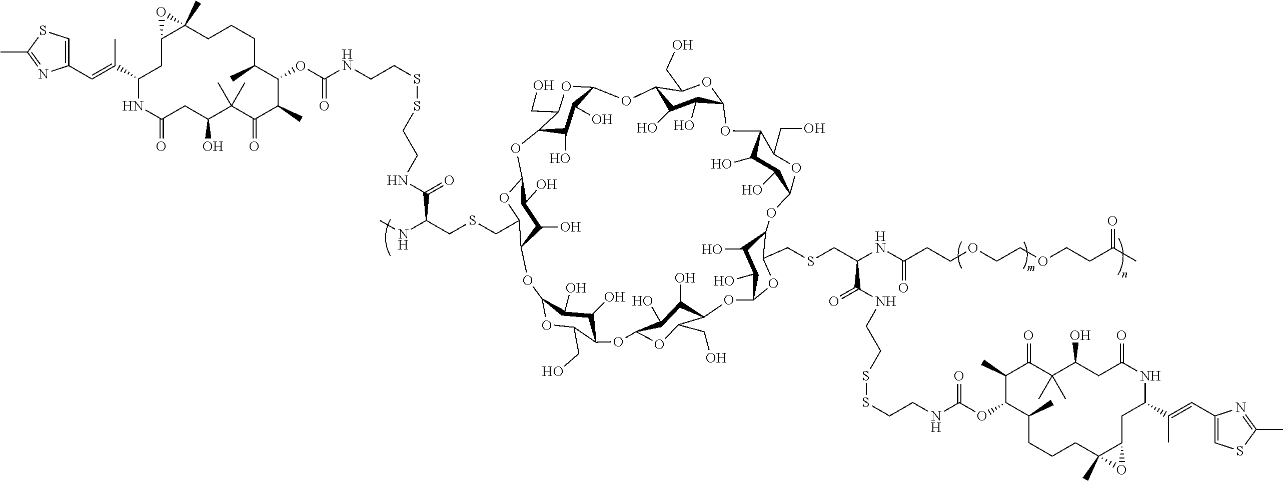

Other embodiments are in the claims.

1. A method of treating a cancer in a subject, the method comprising: administering a composition that comprises a CDP-epothilone conjugate to a subject in an amount effective to treat the disorder, wherein the CDP-epothilone conjugate has the structure 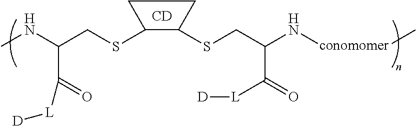
wherein each L is independently a linker or absent, each D is independently an epothilone or —OH, each comonomer comprises polyethylene glycol (PEG), and n is at least 4, provided that the CDP-epothilone conjugate comprises at least one epothilone selected from the group consisting of epothilone B, ixabepilone, BMS310705, epothilone D, dehydelone, and sagopilone, to thereby treat the cancer.

2.-7. (canceled)

8. The method of claim 1, wherein the composition is administered in combination with one or more additional chemotherapeutic agents.

9.-36. (canceled)

37. The method of claim 1, wherein the subject is administered at least one additional dose of the composition.

38. The method of claim 1, wherein the composition is administered once every one, two, three, four, five or six weeks.

39. (canceled)

40. The method of claim 8, wherein the additional chemotherapeutic agent is selected from the following: an antimetabolite; an anthracycline; a vinca alkaloid; a topoisomerase inhibitor; and a platinum-based agent.

41.-42. (canceled)

43. The method of claim 1, wherein the cancer is a chemotherapeutic sensitive, a chemotherapeutic refractory, a chemotherapeutic resistant, or a relapsed cancer.

44. The method of claim 1, wherein the cancer is refractory to, resistant to or relapsed during or after, treatment with, one or more of: a taxane, an anthracycline, an antimetabolite, a vinca alkaloid, a topoisomerase inhibitor and a platinum-based agent.

45.-48. (canceled)

49. The method of claim 1, wherein the cancer is selected from the group consisting of breast cancer, prostate cancer, lung cancer, ovarian cancer, melanoma, and colorectal cancer.

50. A method of treating cancer, the method comprising providing a subject that has cancer and has been treated with a chemotherapeutic agent which did not effectively treat the cancer or which had an unacceptable side effect, and
administering a composition comprising a CDP-epothilone conjugate to the subject in an amount effective to treat the cancer, to thereby treat the cancer.

51.-53. (canceled)

54. The method of claim 50, wherein the cancer is selected from the group consisting of breast cancer, prostate cancer, renal cell carcinoma, lung cancer, ovarian cancer, melanoma, and colorectal cancer.

55.-59. (canceled)

60. The method of claim 1, wherein the cancer is renal cell carcinoma.

61.-68. (canceled)

69. The method of claim 54, wherein the lung cancer is non small cell lung cancer or small cell lung cancer.

70.-76. (canceled)

77. The method of claim 1, wherein the cancer is advanced or metastatic melanoma.

78. (canceled)

79. The method of claim 54, wherein the melanoma is advanced or metastatic melanoma.

80. The method of claim 43, wherein the cancer is advanced or metastatic colorectal cancer.

81. (canceled)

82. The method of claim 54, wherein the colorectal cancer is advanced or metastatic colorectal cancer.

83.-92. (canceled)

93. The method of claim 1, wherein the additional chemotherapeutic agent is an H1 antagonist or an H2 antagonist.

94.-124. (canceled)

125. A compound of the following formula: 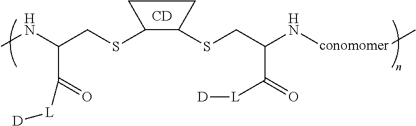
wherein each L is independently a linker or absent, each D is independently an epothilone or —OH, each comonomer comprises polyethylene glycol (PEG), and n is at least 4, provided that the compound comprises at least one epothilone selected from the group consisting of epothilone B, ixabepilone, BMS310705, epothilone D, dehydelone, and sagopilone.
